(19) United States
(12) Patent Application Publication
(10) Pub. No.: US 2026/0224566 A1
(43) Pub. Date: Aug. 6, 2026

(54) Obicetrapib and Ezetimibe Combination Treatment and Fixed Dose Pharmaceutical Compositions

(71) Applicant: NewAmsterdam Pharma B.V., Naarden (NL)

(72) Inventors: Sheng Cui, Lexington, MA (US); Joanne Lesley Craig, Southampton (GB); Johannes Jacob Pieter Kastelein, Blaricum (NL); Marc Ditmarsch, Den Haag (NL); Michael Harvey Davidson, Sunny Isles, FL (US); Andreas René Rötheli, Wellesley, MA (US); Christopher J. Borths, Sherborn, MA (US); Muneki Kishida, Osaka (JP); Valeriya Nikolayevna Smolenskaya, West Lafayette, IN (US); Douglas Fitzgerald Kling, Lebanon, NJ (US)

(21) Appl. No.: 19/154,743

(22) PCT Filed: Jan. 31, 2024
(86) PCT No.: PCT/EP2024/052337

§ 371 Date: Aug. 7, 2025

Related U.S. Application Data
(60) Provisional application No. 63/483,574, filed on Feb. 7, 2023.
(60) Provisional application No. 63/501,313, filed on May 10, 2023.
(60) Provisional application No. 63/617,356, filed on Jan. 3, 2024.

(30) Foreign Application Priority Data
Aug. 22, 2023 (WO) PCT/EP2023/073000

(51) Int. Cl. A61K 31/506; A61K 31/397; A61P 3/06

(52) U.S. Cl. CPC A61K 31/506; A61K 31/397; A61P 3/06

(57) ABSTRACT

The present disclosure relates to combinations of obicetrapib and ezetimibe, or their salts, solvates or derivatives thereof. The disclosure further describes the use of ezetimibe and obicetrapib, e.g. in the form of a fixed dose combination product, for preparation of medicaments and method of treatment of subjects requiring reduction in LDL-cholesterol or those suffering from hyperlipidemia or mixed dyslipidemia.

### TECHNICAL FIELD

The present disclosure relates to a fixed dose pharmaceutical composition comprising obicetrapib and ezetimibe, and its use for preparation of medicaments and treatment of subjects requiring reduction of LDL cholesterol or in patients with heterozygous familial hypercholesterolemia (HeFH) and/or with established atherosclerotic cardiovascular disease (ASCVD).

### BACKGROUND

Despite advances in treatment, cardiovascular disease (CVD) is still a leading cause of death globally, with over 17 million deaths annually. For many years it has been known that abnormal cholesterol levels have been associated with increased risk of cardiovascular disease (CVD), such as cardiomyopathy, atherosclerosis and myocardial infarction. In particular, individuals presenting with high levels of low-density lipoprotein (LDL) cholesterol and very-low-density lipoprotein (VLDL) cholesterol combined with low levels of high-density lipoprotein (HDL) cholesterol were observed to be at the highest risk of developing a cardiovascular disease.

The lowering of low-density lipoprotein cholesterol (LDL-C) is the primary target of therapy in the primary and secondary prevention of cardiovascular events. Although statin therapy is the mainstay for LDL-C lowering, a significant percentage of patients prescribed these agents either do not achieve target blood lipid levels with statin therapy or have partial or complete intolerance to them. To reduce the risk of a recurrent non-fatal or fatal cardiovascular disease, such patients are advised to take combinations of alternative lipid lowering agents.

Updates to risk based LDL-C goals inevitably mean that greater use of combination therapies in addition to (high intensity) statins will be needed. Currently available oral add on lipid lowering therapies reduce LDL-C individually by up to (about) 25%. The costs and inconvenience of more potent injectable therapies have limited their uptake. As a result, there remains a high-unmet need for new effective, safe oral therapies as an adjunct to statin therapy.

### SUMMARY

As will be explained in much more detail herein below, the present inventors have found that remarkable improvements in blood lipid profiles are attained with obicetrapib and ezetimibe combination treatment.

More in particular, the present inventors have found that remarkable improvements in blood lipid profiles are attained with obicetrapib and ezetimibe combination treatment, even in subjects that do not adequately respond to (high intensity) statin treatment. More in particular, as described in the experimental part of this document, it has now been shown, in a phase 2b clinical trial (‘ROSE2’; NCT05266586), that an obicetrapib (10 mg) and ezetimibe (10 mg) combination was well tolerated and achieved a median reduction in LDL-C of 59%, which is clearly indicative of a supra additive (i.e. synergistic) effect. In particular, patients treated with Obicetrapib achieved a median reduction of LDL-C of 39%, meaning that ezetimibe, added on top of obicetrapib, resulted in an additional/incremental (median) reduction of LDL-C of about 32%. This (greatly) exceeds LDL-C reductions normally attained with ezetimibe: with ezetimibe mono-therapy LDL-C levels are typically reduced by 15-22% (in hyperlipidemic patients), while in combination with statins, ezetimibe typically provides an incremental reduction in LDL-C levels of 15-20% (see, for instance, Catapano et al. European Heart Journal (2016) 37, 2999-3058). Significant improvements in ApoB, and Lp(a) levels were also demonstrated in the trial.

To further elucidate the effects of the combination treatment, further studies were carried out using a standard human CETP transgenic knock-in mouse. As can be inferred from the experimental results reported herein, Obicetrapib alone and the combination with ezetimibe reduce non-HDL-C levels by increased VLDL lipolysis, increased VLDL clearance and elevated LDL receptor levels accompanied by an enhanced fecal bile acid and neutral sterol excretion.

Using the same human CETP transgenic knock-in mouse, it has further been demonstrated that obicetrapib alone, and in combination with ezetimibe, reduces atherosclerotic lesion prevalence, size, and severity.

The data mouse model data further support the supra additivity of Obicetrapib and ezetimibe in improving blood lipid profiles and reducing ASCVD related risks in patients in need thereof. Without being bound to any particular theory, the data now generated implicates TICE in the mechanism of action of obicetrapib. More particularly, it is believed that obicetrapib, apart from inhibiting CETP mediated (net) mass Transfer of cholesterol esters from HDL to Particles in VLDL/LDL, also shunts cholesterol to the intestine via Transintestinal cholesterol Excretion (TICE), thereby increasing clearance and excretion. Addition of Ezetimibe Blocks Reabsorption of Cholesterol in the Intestine, working synergistically to enhance fecal sterol excretion.

One aspect of the invention thus relates to a fixed dose pharmaceutical composition comprising obicetrapib or a pharmaceutically acceptable salt, solvate or co-crystal thereof; ezetimibe or a pharmaceutically acceptable salt, solvate or co-crystal thereof, and, pharmaceutically acceptable excipients.

A second aspect relates to a fixed dose pharmaceutical composition comprising obicetrapib or a pharmaceutically acceptable salt, solvate or co-crystal thereof; ezetimibe or a pharmaceutically acceptable salt, solvate or co-crystal thereof, and, pharmaceutically acceptable excipients, for use in reducing LDL cholesterol in patients requiring a reduction in LDL cholesterol and/or increase in HDL cholesterol, patients with heterozygous familial hypercholesterolemia (HeFH) and/or patients with established atherosclerotic cardiovascular disease (ASCVD).

The present invention also provides methods of treating a subject in need thereof, said method comprising the concomitant treatment of the subject with obicetrapib or a pharmaceutically acceptable salt, solvate or co-crystal thereof, and ezetimibe, or a pharmaceutically acceptable salt, solvate or co-crystal thereof, preferably in the form of the fixed dose pharmaceutical composition as defined herein.

One particular aspect of the invention concerns a method for the prophylactic and/or therapeutic treatment of a subject suffering from or at risk of suffering from CVD, in particular ASCVD, said method comprising the concomitant treatment of the subject with obicetrapib, or a pharmaceutically acceptable salt, solvate or co-crystal thereof, and ezetimibe, or a pharmaceutically acceptable salt, solvate or co-crystal thereof. In a preferred embodiment of the invention, said method comprises the administration of the fixed dose pharmaceutical composition as defined herein.

A further aspect of the invention concerns a pharmaceutical composition comprising ezetimibe or a pharmaceutically acceptable salt, solvate or co-crystal thereof, and/or obicetrapib or a pharmaceutically acceptable salt, solvate or co-crystal thereof, for use in a method for the prophylactic and/or therapeutic treatment of a subject suffering from or at risk of suffering from CVD, in particular ASCVD, wherein the method comprises the concomitant treatment of the subject with ezetimibe, or a pharmaceutically acceptable salt, solvate or co-crystal thereof, and obicetrapib, or a pharmaceutically acceptable salt, solvate or co-crystal thereof. In a preferred embodiment of the invention, said pharmaceutical composition is the fixed dose pharmaceutical composition as defined herein.

A further aspect of the invention concerns a method of synergistically lowering LDL-C plasma levels in a subject in need thereof, said method comprising the concomitant treatment of said subject with ezetimibe, or a pharmaceutically acceptable salt, solvate or co-crystal thereof, and obicetrapib, or a pharmaceutically acceptable salt, solvate or co-crystal thereof. In a preferred embodiment of the invention, said method comprises the administration of the fixed dose pharmaceutical composition as defined herein.

A further aspect of the invention concerns a pharmaceutical composition comprising ezetimibe, or a pharmaceutically acceptable salt, solvate or co-crystal thereof, and/or obicetrapib or a pharmaceutically acceptable salt, solvate thereof or co-crystal thereof, for use in a method of synergistically lowering LDL-C plasma levels in a subject in need thereof, said method comprising the concomitant administration of ezetimibe, or a pharmaceutically acceptable salt, solvate or co-crystal thereof, and/or obicetrapib, or a pharmaceutically acceptable salt, solvate or co-crystal thereof. In a preferred embodiment of the invention, said method comprises the administration of the fixed dose pharmaceutical composition as defined herein.

A further aspect of the invention concerns a method of synergistically slowing the development and/or progression of CVD, more in particular ASCVD, and/or synergistically reducing the risk and/or occurrence of CVD related events, in particular ASCVD related events, in a subject in need thereof, said method comprising the concomitant administration of ezetimibe, or a pharmaceutically acceptable salt, solvate or co-crystal thereof, and obicetrapib, or a pharmaceutically acceptable salt, solvate or co-crystal thereof. In a preferred embodiment of the invention, said method comprises the administration of the fixed dose pharmaceutical composition as defined herein.

A further aspect of the invention concerns a pharmaceutical composition comprising ezetimibe, or a pharmaceutically acceptable salt, solvate or co-crystal thereof, and/or obicetrapib or a pharmaceutically acceptable salt, solvate or co-crystal thereof, for use in a method of synergistically slowing the development and/or progression of CVD, more in particular ASCVD, and/or synergistically reducing the risk and/or occurrence of CVD related events, in particular ASCVD related events, in a subject in need thereof, said method comprising the concomitant treatment of the subject with ezetimibe, or a pharmaceutically acceptable salt, solvate or co-crystal thereof, and obicetrapib, or a pharmaceutically acceptable salt, solvate or co-crystal thereof. In a preferred embodiment, said method comprises the administration of the fixed dose pharmaceutical composition as defined herein.

A further aspect of the invention concerns a method of enhancement, preferably the synergistic enhancement, of the LDL-C lowering effect of obicetrapib, or a pharmaceutically acceptable salt, solvate or co-crystal thereof, in a subject in need thereof, said method comprising the concomitant treatment of the subject with ezetimibe or a pharmaceutically acceptable salt, solvate or co-crystal thereof. In a preferred embodiment of the invention, said method comprises the administration of the fixed dose pharmaceutical composition as defined herein.

A further aspect of the invention concerns a pharmaceutical composition comprising ezetimibe or a pharmaceutically acceptable salt, solvate or co-crystal thereof, for use in a method of enhancement, preferably the synergistic enhancement, of the LDL-C lowering effect of obicetrapib, or a pharmaceutically acceptable salt, solvate or co-crystal thereof, in a subject in need thereof, said method comprising the concomitant administration of ezetimibe or a pharmaceutically acceptable salt, solvate or co-crystal thereof. In a preferred embodiment of the invention, said method comprises the administration of the fixed dose pharmaceutical composition as defined herein.

A further aspect of the invention concerns a method of enhancement, preferably the synergistic enhancement, of the therapeutic efficacy of obicetrapib, or a pharmaceutically acceptable salt, solvate or co-crystal thereof, in particular of the therapeutic efficacy in the treatment and/or prevention of CVD, more in particular ASCVD, in a subject in need thereof, said method comprising the concomitant administration of ezetimibe, or a pharmaceutically acceptable salt, solvate or co-crystal thereof. In a preferred embodiment of the invention, said method comprises the administration of the fixed dose pharmaceutical composition as defined herein.

A further aspect of the invention concerns a pharmaceutical composition comprising ezetimibe, or a pharmaceutically acceptable salt, solvate or co-crystal thereof, for use in a method of enhancement, preferably the synergistic enhancement of the therapeutic efficacy of obicetrapib or a pharmaceutically acceptable salt, solvate or co-crystal thereof, in particular of the therapeutic efficacy in the treatment and/or prevention of CVD, more in particular ASCVD, in a subject in need thereof, said method comprising the concomitant administration of ezetimibe, or a pharmaceutically acceptable salt, solvate or co-crystal thereof. In a preferred embodiment of the invention, said method comprises the administration of the fixed dose pharmaceutical composition as defined herein.

Yet, a further aspect of the invention concerns the use of obicetrapib, or a pharmaceutically acceptable salt, solvate or co-crystal thereof, and/or ezetimibe, or a pharmaceutically acceptable salt, solvate or co-crystal thereof, in the manufacture of a medicament for use in any one of the afore defined methods. In a preferred embodiment of the invention, said medicament is the fixed dose pharmaceutical composition as defined herein.

Other aspects of the invention concern a kit comprising a package containing a plurality of pharmaceutical unit dosage forms comprising or a pharmaceutically acceptable salt, hydrate or solvate thereof, such as the fixed dose pharmaceutical compositions as defined herein, as well as a leaflet containing printed instructions to repeatedly self-administer said unit dosage forms in order to treat and/or prevent CVD, in particular ASCVD, by combined obicetrapib treatment and ezetimibe therapy.

It will be understood that these aspects of the invention all involve the same compositions, the same methods of treatment, the same subjects, etc. unless specifically stated otherwise.

In certain preferred embodiments of the invention, the salt of obicetrapib, contained in the present pharmaceutical compositions, used in the present methods, contained in the unit dosage forms (comprised in the pharmaceutical kit), etc., is an amorphous calcium salt of obicetrapib.

Specific details and preferred embodiments of the afore-mentioned methods as well as of the compositions and pharmaceutical kits used therein will become evident to those skilled in the art on the basis of the following detailed description and the appended experimental part.

### Definitions

Obicetrapib, also referred to as “TA-8995”, has the following chemical name and chemical structure:

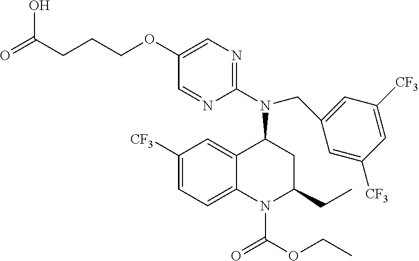 [SMILES: CCOC(=O)N1c2ccc(C(F)(F)F)cc2[C@@H](N(Cc2cc(C(F)(F)F)cc(C(F)(F)F)c2)c2ncc(OCCCC(=O)O)cn2)C[C@H]1CC]

{4-[(2-{[3,5-bis(trifluoromethyl)benzyl] [(2R,4S)-1-(ethoxycarbonyl)-2-ethyl-6-(trifluoromethyl)-1,2,3,4-tetrahydroquinolin-4-yl]amino}pyrimidin-5-yl)oxy]butanoic acid}

Ezetimibe, also referred to as “Sch-58235”, has the following chemical name and chemical structure:

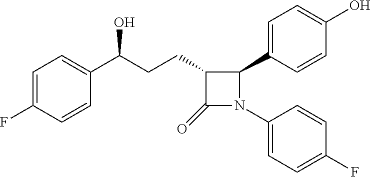 [SMILES: O=C1[C@H](CC[C@H](O)c2ccc(F)cc2)[C@@H](c2ccc(O)cc2)N1c1ccc(F)cc1]

(3R,4S)-1-(4-fluorophenyl)-3-[(3S)-3-(4-fluorophenyl)-3-hydroxypropyl]-4-(4-hydroxyphenyl)azetidin-2-one.

Both obicetrapib and ezetimibe may also be used as different salt forms, solvates or co-crystals. They may also be formulated as pro-drugs.

The term “apolipoprotein” as used herein has its conventional meaning and refers to proteins that bind lipids to form lipoproteins.

The term “apolipoprotein B” (ApoB) as used herein has its conventional meaning and refers to the protein encoded by the ApoB gene.

The term ‘pharmaceutical composition’ as used herein has its conventional meaning and refers to a composition which is pharmaceutically acceptable.

The term ‘pharmaceutically acceptable’ as used herein has its conventional meaning and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio.

The term “carrier” as used herein has its conventional meaning and refers to a pharmaceutically acceptable diluent, adjuvant, excipient or vehicle with which a pharmaceutically active ingredient is administered.

The term ‘excipient’ as used herein has its conventional meaning and refers to a pharmaceutically acceptable ingredient, which is commonly used in the pharmaceutical technology for preparing a granulate, solid or liquid oral dosage formulation.

The term ‘salt’ as used herein has its conventional meaning and includes the acid addition and base salts of a pharmaceutically active compound.

The term “solvate” as used herein has its conventional meaning and refers to a compound formed by solvation, for example as a combination of solvent molecules with molecules or ions of a solute. Well known solvent molecules include water, alcohols, nitriles and polar organic solvents.

The term “subject” as used herein refers to humans suffering from or at risk for a certain disease or disorder. The term “subject” and “patient” herein are used interchangeably.

The term ‘increased risk’ has its conventional meaning and refers to a situation where a subject, preferably a human subject, either male or female, based on his or her risk profile (including an LDL-cholesterol level above 70 mg/dL, such as above 2.6 mmol/l [100.54 mg/dL]), such that the subject is at an increased risk of suffering a cardiovascular event, compared to those with lower levels.

The term ‘treatment’ as used herein has its conventional meaning and refers to curative, palliative and prophylactic treatment.

The term ‘cardiovascular disease’ as used herein has its conventional meaning and includes clinical manifestations of arteriosclerosis, peripheral vascular disease angina, ischemia, cardiac ischemia, stroke, myocardial infarction, reperfusion injury, restenosis after angioplasty, hypertension, cerebral infarction and cerebral stroke.

The term “cardiovascular event” as used herein has its conventional meaning and refers to occurrence of myocardial infarction, stroke, coronary death or the necessity to undergo a coronary revascularization (Ference, 2017).

The term “hypercholesterolemia” as used herein has its conventional meaning and refers to the condition in which high levels of cholesterol are present in the blood.

The term “hyperlipidaemia” as used herein has its conventional meaning and refers to the condition in which there are high amounts of lipids found in the blood.

The term “mixed dyslipidemia” as used herein has its conventional meaning and refers to the condition in which there are elevations of LDL cholesterol and triglyceride levels that are accompanied by low levels of HDL cholesterol in the blood.

The term “statin intolerant” as used herein has its conventional meaning and refers to subjects inability to tolerate two or more statins, one at a low dose, due to an adverse safety effect that started or increased during statin therapy and resolved or improved when statin was discontinued, reference is in this regard also made to the similar definition approved by the FDA in the bempedoic acid (Esperion) phase III trial.

The term ‘cholesterol absorption inhibitor’ (CAI) as used herein has its conventional meaning and refers to compounds which are used to lower LDL-C by blocking enteric and biliary absorption of cholesterol. A known cholesterol absorption inhibitor is ezetimibe.

The term “cholesteryl ester transfer protein inhibitor” (CETP inhibitor) as used herein has its conventional meaning and refers to a class of compounds that inhibits the CETP receptor in mammals. A known CETP inhibitor is obicetrapib.

The term ‘unit dosage form’ has its conventional meaning and refers to a dosage form which has the capacity of being administered to a subject, preferably a human, to be effective, and which can be readily handled and packaged, remaining as a physically and chemically stable unit dose comprising the therapeutic agent, i.e. obicetrapib or combination of therapeutic agents, such as obicetrapib and ezetimibe.

The term ‘fixed dose combination’ as used herein has its conventional meaning and refers to a combination of defined doses of two or more drugs or active ingredients presented in a single dosage unit (e.g. a tablet or a capsule) and administered as such.

The term ‘free dose combination’ as used herein has its conventional meaning and refers to a combination of two drugs or active ingredients administered simultaneously but as two distinct dosage units.

The term “effective amount” or “therapeutically effective amount” refers to an amount that is sufficient to effect treatment, as defined herein, when administered to a mammal in need of such treatment. The therapeutically effective amount will vary depending upon the patient being treated, the weight and age of the patient, the severity of the disease condition, the manner of administration and the like, which can readily be determined by one of ordinary skill in the art.

Unless specifically stated otherwise, where a compound may assume alternative tautomeric, regioisomeric and/or stereoisomeric forms, all alternative isomers, are intended to be encompassed within the scope of the claimed subject matter. For example, when a compound is described as a particular optical isomer D- or L-, it is intended that both optical isomers be encompassed herein. For example, where a compound is described as having one of two tautomeric forms, it is intended that both tautomers be encompassed herein. Thus, the compounds provided herein may be enantiomerically pure, or be stereoisomeric or diastereomeric mixtures. The compounds provided herein may contain chiral centers. Such chiral centers may be of either the (R) or(S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its(S) form.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O, ¹⁸F, ³⁶Cl, ⁸²Br, ¹²³I, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with ³H, ¹⁴C and/or ¹⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

Thus, any of the embodiments described herein are meant to include, a single stereoisomer, a mixture of stereoisomers and/or an isotopic form of the compounds.

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. Unless otherwise specified, the term “about” means within plus or minus 10% of a the explicitly recited value, rounded either up or down to the nearest integer.

### BRIEF DESCRIPTION OF THE DRAWINGS

FIG. 1 Cumulative undersize curve of small-scale batch of a fixed dose combination composition of 10 mg ezetimibe and 5 mg obicetrapib

FIG. 2 Retain curve for small-scale batch of a fixed dose combination composition of 10 mg ezetimibe and 5 mg obicetrapib

FIG. 3 Comparison of dissolution profile by discriminatory dissolution method-ezetimibe at pH 6.8 for small-scale batch of a fixed dose combination composition of 10 mg ezetimibe and 5 mg obicetrapib

FIG. 4 Comparison of dissolution profile of obicetrapib by discriminatory dissolution method pH 6.8 for small-scale batch of a fixed dose combination composition of 10 mg ezetimibe and 5 mg obicetrapib

FIG. 5 Comparison of dissolution profile of ezetimibe by discriminatory dissolution-pH 4.5 for small-scale batch of a fixed dose combination composition of 10 mg ezetimibe and 5 mg obicetrapib

FIG. 6 Comparison of obicetrapib dissolution profile of the stress stability study for batch a4459/05/05-pH 6.8 for small-scale batch of a fixed dose combination composition of 10 mg ezetimibe and 5 mg obicetrapib

FIG. 7 Comparison of obicetrapib dissolution profile of the stress stability study for batch a4459/05/06-pH 6.8 for small-scale batch of a fixed dose combination composition of 10 mg ezetimibe and 5 mg obicetrapib

FIG. 8 Comparison of obicetrapib dissolution profile of the stress stability study for batch a4459/05/07-pH 6.8 for small-scale batch of a fixed dose combination composition of 10 mg ezetimibe and 5 mg obicetrapib

FIG. 9 Comparison of obicetrapib dissolution profile of the stress stability study for batch a4459/05/08-pH 6.8 for small-scale batch of a fixed dose combination composition of 10 mg ezetimibe and 5 mg obicetrapib

FIG. 10 Comparison of ezetimibe dissolution profile of the stress stability study for batch a4459/05/05-ph6.8 for small-scale batch of a fixed dose combination composition of 10 mg ezetimibe and 5 mg obicetrapib

FIG. 11 Comparison of ezetimibe dissolution profile of the stress stability study for batch a4459/05/06-pH 6.8 for small-scale batch of a fixed dose combination composition of 10 mg ezetimibe and 5 mg obicetrapib

FIG. 12 Comparison of ezetimibe dissolution profile of the stress stability study for batch a4459/05/07-pH 6.8 for small-scale batch of a fixed dose combination composition of 10 mg ezetimibe and 5 mg obicetrapib

FIG. 13 Comparison of ezetimibe dissolution profile of the stress stability study for batch a4459/05/08-pH 6.8 for small-scale batch of a fixed dose combination composition of 10 mg ezetimibe and 5 mg obicetrapib

FIG. 14 Comparison of ezetimibe dissolution profile of the stress stability study for batch a4459/05/05-pH 4.5 for small-scale batch of a fixed dose combination composition of 10 mg ezetimibe and 5 mg obicetrapib

FIG. 15 Comparison of ezetimibe dissolution profile of the stress stability study for batch a4459/05/06-pH 4.5 for small-scale batch of a fixed dose combination composition of 10 mg ezetimibe and 5 mg obicetrapib

FIG. 16 Comparison of ezetimibe dissolution profile of the stress stability study for batch a4459/05/07 pH 4.5 for small-scale batch of a fixed dose combination composition of 10 mg ezetimibe and 5 mg obicetrapib

FIG. 17 Comparison of ezetimibe dissolution profile of the stress stability study for batch a4459/05/08-pH 4.5 for small-scale batch of a fixed dose combination composition of 10 mg ezetimibe and 5 mg obicetrapib

FIG. 18 % cumulative undersize curve of small-scale batch of 10 mg 12zetimibe and 10 mg obicetrapib fixed composition

FIG. 19 Obicetrapib dissolution profiles for small scale 10 mg ezetimibe and 10 mg obicetrapib (free acid) fixed dose composition

FIG. 20 Ezetimibe dissolution profiles (50 rpm) for small scale 10 mg ezetimibe and 10 mg obicetrapib (free acid) fixed dose composition

FIG. 21 Ezetimibe dissolution profiles (75 rpm) for small scale 10 mg ezetimibe and 10 mg obicetrapib (free acid) fixed dose composition

FIG. 22 Obicetrapib prototype C 200 BN A4459/19/03 stress stability dissolution results for small scale 10 mg ezetimibe and 10 mg obicetrapib (free acid) fixed dose composition

FIG. 23 Obicetrapib prototype C scale up BN A4459/19/02 stress stability dissolution results for small scale 10 mg ezetimibe and 10 mg obicetrapib (free acid) fixed dose composition

FIG. 24 Ezetimibe prototype C scale up BN A4459/19/02 stress stability dissolution results for small scale 10 mg ezetimibe and 10 mg obicetrapib (free acid) fixed dose composition

FIG. 25 Ezetimibe prototype C scale up BN A4459/19/02 stress stability dissolution results for small scale 10 mg ezetimibe and 10 mg obicetrapib (free acid) fixed dose composition

FIG. 26 Cumulative undersize for small scale FDC1 compositions

FIG. 27 Obicetrapib dissolution profiles for FDC1 prototypes

FIG. 28 Ezetimibe dissolution profiles for FDC1 prototypes

FIG. 29 Cumulative undersize for small scale FDC2 compositions

FIG. 30 Obicetrapib dissolution profiles for small scale FDC2 compositions

FIG. 31 Ezetimibe dissolution profiles for small scale FDC2 compositions

FIG. 32 Obicetrapib dissolution profiles for small scale FDC2 coated tablets by discriminating method

FIG. 33 Obicetrapib dissolution profiles for small scale FDC2 coated tablets by QC method

FIG. 34 Ezetimibe dissolution profiles for small scale FDC2 coated tablets by QC method

FIG. 35 Obicetrapib dissolution profiles for small scale prototype 2 of FDC2 coated tablets from stress stability

FIG. 36 Ezetimibe dissolution profile for prototype 2 of FDC2 coated tablets from stress stability

FIG. 37 Cumulative undersize curve for scale-up batches

FIG. 38 Obicetrapib dissolution profile for FDC1 granule from scale up batch

FIG. 39 Ezetimibe dissolution profile for FDC1 granule from scale up batch

FIG. 40 Ezetimibe dissolution profile for FDC2 final blend from scale up batch

FIG. 41 Obicetrapib dissolution profile for uncoated tablets of FDC1 scale up batch at different compression forces

FIG. 42 Ezetimibe dissolution profiles for uncoated tablets of FDC1 scale up batch at different compression forces

FIG. 43 Obicetrapib dissolution profile for uncoated tablets of FDC2 scale up batch at different compression forces

FIG. 44 Ezetimibe dissolution profiles for uncoated tablets of FDC2 scale up batch at different compression forces

FIG. 45 Cumulative undersize curve for technical batches

FIG. 46 Obicetrapib dissolution profiles for FDC1 and FDC 2 technical batches

FIG. 47 Obicetrapib dissolution profiles for FDC1 and FDC 2 technical batches

FIG. 48 Particle size distribution (PSD) data of granules from technical batches

FIG. 49 is an x-ray powder diffraction pattern of amorphous obicetrapib hemicalcium.

FIG. 50 is an x-ray powder diffraction pattern of amorphous obicetrapib hemicalcium.

FIG. 51 is an x-ray powder diffraction pattern of amorphous obicetrapib hemicalcium.

FIG. 52 is an infrared spectrum of amorphous obicetrapib hemicalcium.

FIG. 53 is a ¹H-NMR spectrum of amorphous obicetrapib hemicalcium.

FIG. 54 is an x-ray powder diffraction pattern of crystalline obicetrapib hemicalcium.

FIG. 55 is an x-ray powder diffraction pattern stackplot from a stability study of crystalline obicetrapib hemicalcium.

FIG. 56 is an x-ray powder diffraction pattern stackplot from a stability study of amorphous obicetrapib hemicalcium.

FIG. 57 is a polarized light micrograph of amorphous obicetrapib hemicalcium.

FIG. 58 is a polarized light micrograph of crystalline obicetrapib hemicalcium.

FIG. 59 is a thermogravimetric analysis plot of amorphous obicetrapib hemicalcium.

FIG. 60 is a modulated differential scanning calorimetry thermogram (with pinhole) of amorphous obicetrapib hemicalcium.

FIG. 61 a modulated differential scanning calorimetry thermogram (with pinhole) of amorphous obicetrapib hemicalcium.

FIG. 62 is a modulated differential scanning calorimetry thermogram (with pinhole) of crystalline obicetrapib hemicalcium.

FIG. 63 is a solid-state ¹³C-NMR spectrum of amorphous and crystalline obicetrapib hemicalcium.

FIG. 64 is a solid-state ¹³C-NMR spectrum of crystalline obicetrapib hemicalcium.

FIG. 65 is a solid-state ¹³C-NMR spectrum of amorphous obicetrapib hemicalcium.

FIG. 66 is an x-ray powder diffraction pattern of crystalline obicetrapib HCl and at least partially desolvated crystalline obicetrapib HCl.

FIG. 67 is an x-ray powder diffraction pattern of crystalline obicetrapib HCl.

FIG. 68 is an x-ray powder diffraction pattern of crystalline Compound 1D.

FIG. 69 is ¹H-NMR spectrum of Compound 1D.

FIG. 70 schematically depicts the mechanism(s) of action that are presumed to be responsible for the therapeutic effects of obicetrapib, ezetimibe and the combination thereof.

FIG. 71 summarizes the design of the study into the effects of obicetrapib/ezetimibe combination treatment in a translational mouse model for hyperlipidemia and atherosclerosis (ApoE*3-Leiden.CETP transgenic mice)

FIG. 72 shows the effect of study treatments on total cholesterol and non HDL-cholesterol levels in ApoE*3-Leiden.CETP transgenic mice.

FIG. 73 shows the effect of study treatments on HDL-cholesterol and ApoA1 levels in ApoE*3-Leiden.CETP transgenic mice.

FIG. 74 shows the effect of study treatments on triglycerides levels in ApoE*3-Leiden.CETP transgenic mice.

FIG. 75 shows the effect of study treatments on lipoprotein profiles in ApoE*3-Leiden.CETP transgenic mice.

FIG. 76 shows the effect of study treatments on half-life of VLDL like particles in ApoE*3-Leiden.CETP transgenic mice.

FIG. 77 shows the effect of study treatments on liver uptake of VLDL like particles in ApoE*3-Leiden.CETP transgenic mice.

FIG. 78 shows the effect of study treatments on plasma pcsk-9 levels and hepatic LDLr levels in ApoE*3-Leiden.CETP transgenic mice.

FIG. 79 shows the effect of study treatments on liver weight and liver lipids in ApoE*3-Leiden.CETP transgenic mice.

FIG. 80 shows the effect of study treatments on fecal bile acids and fecal neutral sterols in ApoE*3-Leiden.CETP transgenic mice.

FIG. 81 shows the effect of study treatments on cholesterol balance in ApoE*3-Leiden.CETP transgenic mice.

FIG. 82 summarizes the experimental protocol for assessing the effect of co-administration of obicetrapib and ezetimibe on atherosclerotic lesion prevalence, size, and severity in ApoE*3-Leiden.CETP mice, as further described in Example 13.

FIG. 83 charts body weight (FIG. 83A) and food intake (FIG. 83B) during the course of the experiment further described in Example 13.

FIG. 84 charts plasma total cholesterol (FIG. 84A), HDL-cholesterol (FIG. 84B), and non-HDL cholesterol (FIG. 84C) during the course of the experiment described in Example 13. Measures of statistical significance are shown.

FIG. 85 is a histogram calculating the total non-HDL exposure across the entire duration of the experiment described in Example 13. Measures of statistical significance are shown.

FIG. 86 shows lipoprotein profiles of the mice in the experiment described in Example 13, with FIG. 86A showing baseline (t=0 weeks) levels of cholesterol fractions and FIG. 86B showing profiles at 28 weeks (t=28 weeks).

FIG. 87 shows measures of CETP activity (FIG. 87A) and CETP mass (FIG. 87B) in the experiment described in Example 13.

FIG. 88 illustrates the aortic root anatomic location used for assessment of atherosclerosis lesions in the mice in the experiment described in Example 13.

FIG. 89 charts total lesion area (FIG. 89A) and lesion area in the aortic root by AHA classification (FIG. 89B) in all 4 groups of mice in the experiment described in Example 13.

FIG. 90 shows lesion severity (% of segments) (FIG. 90A) and lesion number (# per cross-section) in all 4 groups of mice in the experiment described in Example 13, with measures of statistical significance.

### DETAILED DESCRIPTION

### The Fixed Dose Pharmaceutical Composition of the Invention

A first aspect relates to a fixed dose pharmaceutical composition comprising obicetrapib or a pharmaceutically acceptable salt, solvate or co-crystal thereof, ezetimibe or a pharmaceutically acceptable salt, solvate or co-crystal thereof, and one or more pharmaceutically acceptable excipients.

In one particularly preferred embodiment of the invention, the fixed dose pharmaceutical composition comprises:
(a) an LDL-C lowering amount of obicetrapib or a pharmaceutically acceptable salt, solvate or co-crystal thereof; and
(b) an LDL-C lowering amount of ezetimibe or a pharmaceutically acceptable salt, solvate or co-crystal thereof; and
(c) pharmaceutically acceptable excipients.

In one of the embodiments, upon oral administration of the said fixed dose pharmaceutical composition to a subject, 90% confidence interval for the geometric mean of the area under the curve (AUC_{0-∞} and/or AUC₀₋ₜ) and/or Cmax for obicetrapib is within a range of about 75%-125%, preferably about 80%-125%, and more preferably about 90%-110% of the area under the curve (AUC_{0-∞} and/or AUC₀₋ₜ) and/or Cmax, respectively, of obicetrapib as obtained upon oral administration of a reference pharmaceutical composition to a similar subject, wherein said reference composition comprises an equivalent dose of obicetrapib or its pharmaceutically acceptable salt, solvate or co-crystal thereof, and wherein the reference composition is administered alone, or as a simultaneous or sequential co-administration with another pharmaceutical composition comprising ezetimibe or a pharmaceutically acceptable salt, solvate or co-crystal thereof, or as a fixed-dose combination with ezetimibe or a pharmaceutically acceptable salt, solvate or co-crystal thereof.

In another embodiment, upon oral administration of the said fixed pharmaceutical composition to a subject, 90% confidence interval for the geometric mean of area under the curve (AUC_{0-∞} and/or AUC₀₋ₜ) and/or Cmax for ezetimibe and/or ezetimibe glucoronide is within a range of about 75%-125%, preferably about 80%-125%, and more preferably about 90%-110% of the area under the curve (AUC_{0-∞} and/or AUC₀₋ₜ) and/or Cmax, respectively, of ezetimibe and/or ezetimibe glucoronide, respectively, as obtained upon oral administration of a reference pharmaceutical composition to a similar subject, wherein the reference comprises an equivalent dose of ezetimibe or its pharmaceutically acceptable salt, solvate or co-crystal thereof, and wherein the reference composition is administered alone, or as a simultaneous or sequential co-administration with another pharmaceutical composition comprising obicetrapib or a pharmaceutically acceptable salt, solvate or co-crystal thereof, or as a fixed-dose combination with ezetimibe or a pharmaceutically acceptable salt, solvate or co-crystal thereof.

Ezetimibe is practically insoluble in water and with poor solubility across the physiological pH range. Achieving desired dissolution and thereby bioavailability in in vivo conditions is quite challenging for ezetimibe. This problem is further enhanced as obicetrapib slows down the rate of dissolution and the total amount of ezetimibe that can be dissolved (unpublished data). It has surprisingly been found that from the fixed dose pharmaceutical composition at least about 60%, preferably at least about 70% and more preferably at least about 80% of ezetimibe is dissolved within about 30 minutes when the said pharmaceutical composition is dissolved in a USP type II apparatus in a 500 ml solution comprising 0.45% SLS in 0.05 M sodium acetate buffer of pH 4.5 at a rotation speed of about 75 rpm at 37±0.5° C. In a preferred embodiment, it is surprisingly found that from the fixed dose pharmaceutical composition at least about 60%, preferably at least about 70% and more preferably at least about 80% of ezetimibe is dissolved within about 20 minutes when the said pharmaceutical composition is dissolved in a USP type II apparatus in a 500 ml solution comprising 0.45% SLS in 0.05 M sodium acetate buffer of pH 4.5 at a rotation speed of about 75 rpm at 37±0.5° C.

It was further surprisingly found that from the fixed dose pharmaceutical composition at least about 70%, preferably at least about 80%, more preferably at least about 85%, and even more preferably at least about 90% of obicetrapib is dissolved within about 30 minutes when the said pharmaceutical composition is dissolved in a USP type II apparatus in a 1000 ml solution comprising phosphate buffer solution of pH 6.8+0.2% w/v Polysorbate 80 at a rotation speed of about 75 rpm at 37±0.5° C. In a preferred embodiment, it is surprisingly found that from the fixed dose pharmaceutical composition at least about 70%, preferably at least about 80%, and more preferably at least about 85% of obicetrapib is dissolved within about 15 minutes when the said pharmaceutical composition is dissolved in a USP type II apparatus in a 1000 ml solution comprising phosphate buffer solution of pH 6.8+0.2% w/v Polysorbate 80 at a rotation speed of about 75 rpm at 37±0.5° C.

Ezetimibe is inherently a poorly/non-compressible API (see for example EP 2168573 A1) along with poor flowability. It is therefore very challenging for the formulation scientists to prepare a tablet formulation that not only satisfies the requirements with respect to hardness, disintegration time, friability, shape and size, but also provides the desired stability and dissolution, of ezetimibe. It has been surprisingly found that the composition not only meets the required specifications for dissolution and stability for being suitable to the claimed uses, but also qualifies the criteria's of the processability parameters, namely flowability, compressibility, disintegration time, friability, hardness, shape and size.

The fixed dose pharmaceutical composition may comprise a combination of 1 to 10 mg obicetrapib and 5 to 20 mg ezetimibe. In a preferred embodiment, the composition comprises 5 mg obicetrapib and 10 mg ezetimibe. In a more preferred embodiment the composition comprises 10 mg obicetrapib and 10 mg ezetimibe.

In a preferred embodiment, the pharmaceutical composition is provided as a unit dosage form comprising 5 mg obicetrapib and 10 mg ezetimibe. In a more preferred embodiment the composition is provided as a unit dosage form comprising 10 mg obicetrapib and 10 mg ezetimibe.

Wherever the dose of either obicetrapib or ezetimibe is mentioned in this disclosure as mg and/or in relative amounts (by weight), it means obicetrapib or ezetimib in its free form. Whenever a salt, solvate or co-crystal of ezetimibe or obicetrapib is used, for the purpose the said dose shall mean a dose equivalent to the weight of ezetimibe or obicetrapib in its free form, respectively.

In certain embodiments, the pharmaceutical composition is provided in the form of a solid oral dosage selected form caplets, minitablets, lozenges, granules, beads, pellets, tablets, capsules, pill, and the like, or liquid oral dosage forms which may be used for the pharmaceutical preparation include, but are not limited to drinks, solutions, suspensions, syrups, beverages and emulsions.

In one embodiment, the solid oral dosage form is provided as a dual component pharmaceutical composition. In a preferred embodiment, one of the components of the dual component pharmaceutical composition comprises ezetimibe and another component comprises obicetrapib. In another preferred embodiment, only one of the components of the dual component pharmaceutical composition comprises both ezetimibe and obicetrapib.

In certain embodiments, the two component composition is a bilayer tablet formulation. In a preferred embodiment, ezetimibe is present in one of the two layers and obicetrapib in the other layer of bilayer tablet.

In another embodiment, the two component system is capsule formulation. In a preferred embodiment, the capsule may have two types of granules wherein one granule type comprises ezetimibe and another granule type comprises obicetrapib. In yet another preferred embodiment, the capsule may comprise two different type of blends or minitablets each comprising ezetimibe or obicetrapib, and optionally, such blends or minitablets may be filled in two components of a capsule which are segregated from each other. In a certain embodiments, each blend or minitablet is filled in a smaller capsule or such blend is compressed into a tablet/caplet/minitablet and then the tablets/caplets/minitablets are filled in a capsule formulation.

In another embodiment, the fixed dose pharmaceutical composition is a compressed tablet formulation comprising an extragranular component and an intragranular component. In a preferred embodiment, the intragranular component comprises ezetimibe and extragranular component comprises obicetrapib. In a more preferred embodiment, the intragranular component comprises both ezetimibe and obicetrapib. In another embodiment, the intragranular component comprises obicetrapib and the extragranular component comprises ezetimibe. In yet another embodiment, the extragranular component comprises both ezetimibe and obicetrapib.

The intragranular components and extragranular components are present in a ratio from about 1:99 to about 99:1, preferably about 3:97 to about 97:3, and more preferably from about 5:95 to about 95:5. In another embodiment, intragranular components and extragranular components are present in a ratio from about 10:90 to about 90:10, preferably about 20:80 to about 80:20 or about 30:70 to about 70:30, and even more preferably about 40:60 to about 60:40 or about 50:50.

The term “Intragranular” refers to being or occurring within granules of the composition i.e. granules comprising a first set of pharmaceutically acceptable excipients including but not limited to a binder, a disintegrant, a diluent, a glidant and a solvent, and optionally one or more pharmaceutically acceptable active ingredients, in this case ezetimibe and/or obicetrapib.

The term “Extra granular” refers to addition of pharmaceutically acceptable component to a material following granulation i.e. an extra-granular fraction comprising a second set of pharmaceutically acceptable excipients including but not limited to a disintegrant, a diluent, a lubricant, a glidant or the like. Optionally, the extra-granular component may comprise one or more pharmaceutically acceptable active ingredients, in this case ezetimibe and/or obicetrapib.

The pharmaceutical composition can be obtained by a known conventional method like dry granulation, wet granulation, direct compression, roller compaction, fluidized bed granulation, rapid mixture granulation, solvent evaporation, hot-melt extrusion or the like. In a preferred embodiment, the composition is obtained by wet granulation followed by compression of the granules in a tablet formulation or filling such granules in a capsule. In one embodiment, the pharmaceutical composition comprises ezetimibe as anhydrous ezetimibe. In another embodiment, the pharmaceutical composition comprises ezetimibe as ezetimibe hydrate, preferably ezetimibe monohydrate. In yet another embodiment, the pharmaceutical composition comprises a mixture of ezetimibe anhydrous and ezetimibe hydrate, preferably ezetimibe monohydrate. The molar ratio of anhydrous ezetimibe:ezetimibe hydrate, preferably ezetimibe monohydrate, in the pharmaceutical composition could be in the range of 100:0 to 0:100, 99.09:0.01 to 0.01:99.09, 99.08:0.02 to 0.02:99.08, 99.07:0.03 to 0.03:99.07, 99.06:0.04 to 0.04:99.06, 99.05:0.05 to 0.05:99.05, 99.04:0.06 to 0.06:99.04, 99.03:0.07 to 0.07:99.03, 99.02:0.08 to 0.02:99.02, 99.01:0.09 to 0.09:99.01, 99:1 to 1:99, 98:2 to 2:98, 90:10 to 10:90, 70:30 to 30:70 or 50:50. In a preferred embodiment, the composition is substantially free of the ezetimibe hydrate and about 100% of ezetimibe is in the form of ezetimibe anhydrous. In another preferred embodiment, about 99.5% ezetimibe is present in the form of ezetimibe anhydrous and about 0.5% of ezetimibe is present in the form of ezetimibe hydrate, preferably ezetimibe monohydrate. In a more preferred embodiment, the composition is substantially free of the ezetimibe anhydrous and about 100% of ezetimibe is in the form of ezetimibe hydrate, preferably ezetimibe monohydrate.

Ezetimibe or obicetrapib or both could be present in the form of a pharmaceutically acceptable salt, solvate or a co-crystal thereof. Solvates include but are not limited to hydrates. Further, “salt” refers to a compound prepared by the reaction of an organic acid or base drug with a pharmaceutically acceptable mineral or organic acid or base; as used herein, “salt” includes hydrates and solvates of the salts. Exemplary pharmaceutically acceptable mineral or organic acids or bases are as listed in Tables 1-8 in Handbook of Pharmaceutical Salts, P. H. Stahl and C. G. Wermuth (eds.), VHCA, Zurich 2002, pp. 334-345. A pharmaceutically acceptable salt of obicetrapib or ezetimibe may be readily prepared by mixing together solutions of such compounds and the desired acid or base, as appropriate. The salt may precipitate from solution and be collected by filtration or may be recovered by evaporation of the solvent. In one embodiment, salts include, but are not limited to, hydrochloride, phosphate, sulfate, mesylate, esylate and besylate salt forms. In one of the preferred embodiments, the composition comprises obicetrapib as an alkali metal or alkali earth metal salt of obicetrapib, preferably obicetrapib sodium, obicetrapib potassium or obicetrapib calcium, and more preferably obicetrapib calcium salt. The term “co-crystal” as used herein means a crystalline material comprised of two or more unique solids at room temperature, each containing distinctive physical characteristics, such as structure, melting point and heats of fusion, with the exception that, if specifically stated, the active pharmaceutical ingredient may be a liquid at room temperature. The co-crystals may comprise a co-crystal former H-bonded to obicetrapib and/or ezetimibe. The co-crystal former may be H-bonded directly to the active pharmaceutical ingredient or may be H-bonded to an additional molecule which is bound to obicetrapib and/or ezetimibe. In one of the embodiments, a co-crystal could be made between obicetrapib and ezetimibe or their salts or solvates. Solvates of active compounds that do not further comprise a co-crystal former are not co-crystals. The co-crystals may also be a co-crystal between a co-crystal former and a salt of ezetimibe or obicetrapib or both. Other modes of molecular recognition may also be present including, pi-stacking, guest-host complexation and van der Waals interactions. Of the interactions listed above, hydrogen-bonding is the dominant interaction in the formation of the co-crystal, whereby a non-covalent bond is formed between a hydrogen bond donor of one of the moieties and a hydrogen bond acceptor of the other. In another embodiment the co-crystal comprises two co-crystal formers. Co-crystal formers include, but are not limited to a free acid, free base, or zwitter ion; a salt, an inorganic base addition salt such as sodium, potassium, lithium, calcium, magnesium, ammonium, aluminum salts or organic base addition salts, or an inorganic acid addition salts such as HBr, HCl, sulfuric, nitric, or phosphoric acid addition salts or an organic acid addition salt such as acetic, proprionic, pyruvic, malanic, succinic, malic, maleic, fumaric, tartaric, citric, benzoic, methanesulfonic, ethanesulforic, stearic or lactic acid addition salt; an anhydrate or hydrate of a free form or salt, or more specifically, for example, a hemihydrate, monohydrate, dihydrate, trihydrate, quadrahydrate, pentahydrate; or a solvate of a free form or salt. The ratio of active ingredient to co-crystal former may be stoichiometric or non-stoichiometric for the purposes. For example, 1:1, 1:1.5, 1:2 and 2:1 ratios of active ingredient (obicetrapib or ezetimibe or both, including theirs salts or solvates): co-crystal former are acceptable.

In one of the embodiments, the said fixed dose pharmaceutical composition comprises either ezetimibe or obicetrapib, or both as a micronized API. Particle size distribution for such micronized API can be determined by a skilled person using the methods commonly known in the art. These methods include but are not limited to laser diffraction (LD), dynamic light scattering (DLS), dynamic image analysis (DIA) or sieve analysis. Preferably, the method employed is laser diffraction dry powder dispersion which provides the particle size distributions by measuring the angular variation in intensity of light scattered as a laser beam passes through a dispersed particulate sample. Large particles scatter light at small angles relative to the laser beam and small particles scatter light at large angles. The angular scattering intensity data is then analyzed to calculate the size of the particles responsible for creating a cumulative undersize discrete distribution curve that gives particle size distribution by volume. The particle size from this method is usually reported as a volume equivalent sphere diameter (Dv). The most common percentiles reported are the Dv10, Dv50 and Dv90 (also referred as X₁₀, X₅₀ and X₉₀). Dv90 means 90% of the particles by volume are below a particular size & 10% above, Dv50 means 50% of the particles by volume are below a particular size & 50% above, and Dv10 means 10% of the particles by volume are below this size & 90% above.

In one of the preferred embodiments, the composition comprises micronized ezetimibe having a Dv90 not more than 10 μm, preferably in the range of 4-10 μm, more preferably not more than 8.5 μm; Dv50 not more than 4 μm, preferably in the range of about 1-4 μm more, more preferably not more than 3.8 μm, and Dv10 not more than 1 μm.

In another preferred embodiment, the composition comprises micronized obicetrapib having a Dv90 not more than 14 μm, preferably in the range of about 5-14 μm; Dv50 not more than 5 μm, preferably in the range of about 3-5 μm; and Dv10 not more than 3 μm.

The pharmaceutical composition further comprises one or more pharmaceutically acceptable excipients. Excipients include but are not limited to one or more binders, surfactants, disintegrants, glidant, lubricant, diluent, chelating agents, desiccants or absorbents. The following references which are all hereby incorporated by reference disclose techniques and excipients used to formulate oral dosage forms. See “The Handbook of Pharmaceutical Excipients”, 9th edition, Rowe et al., Eds., American Pharmaceuticals Association (2020); and “Remington: The Science and Practice of Pharmacy”, 22^{nd} edition, Gennaro, Ed., Lippincott Williams & Wilkins (2013).

The one or more binders used in the pharmaceutical composition are preferably selected from cellulose derivatives such as methylcellulose and carboxymethylcellulose, hydroxypropylmethylcellulose, hydroxypropylcellulose, hydroxyethylcellulose, gelatin, glucose, sucrose, lactose dextrose, xylitol, sorbitol, maltitol, polymethacrylates, polyvinylpyrrolidone and its copolymers, starch paste, pregelatinized starch, gum tragacanth, alginic acids and salts thereof such as sodium alginate, magnesium aluminum silicate, polyethylene glycol, guar gum, bentonites. In a preferred embodiment, the binder is polyvinylpyrrolidone or copolymers of polyvinylpyrrolidone. In a more preferred embodiment, the binder is copovidone. In an even more preferred embodiment, the binder is Kollidon 30.

The binders may typically be present in an amount from about 0.2% to about 10%, from about 0.5% to about 5%, from about 0.5% to about 2% or from about 0.5% to about 1%, preferably about 1.0±0.5% by weight of the granule composition in one embodiment and by weight of the total tablet in another embodiment.

The one or more surfactants used in the composition preferably are the surfactants having an HLB value selected from at least about 15, at least about 20, at least about 30 or at least about 40. One or more such surfactants are selected from lauric, palmitic, stearic and oleic acid or salts thereof, polyethylene glycol glycerides, polyoxyethylene monoesters, polyoxyethylethylene monostearate, polyoxyethylene monolaurate, polyoxyethylene sorbitan monooleate, polyethoxylated castor oils, polyethylene glycol having molecular weight in the range of about 2000 to 10000, propylene glycol caprylates, glycerol oleates and caprylates, esters of glycerol and fatty acids. In a preferred embodiment, one or more surfactants are selected from dioctyl sodium sulfosuccinate, Capmul PG-8, Capryol 90, Capmul MCM, polysorbate 20, Polysorbate 40 or polysorbate 80 or sodium lauryl sulphate. In a more preferred embodiment the surfactant is sodium lauryl sulphate such as Kolliphor SLS.

The surfactants typically may be present in an amount from about 0.2% to 10%, from about 0.5% to about 5%, from about 0.5% to about 2% or from about 0.5% to about 1%, preferably about 1.0±0.5% by weight of the granule composition in one embodiment and by weight of the total tablet in another embodiment.

In one of the embodiments, the composition comprises a binder: surfactant ratio in the range of about 0.05:5.0 to about 5.0:0.05, preferably from about 0.5:4.5 to about 4.5:0.5, more preferably from about 1:4 to about 4:1, even more preferably from about 1:2 to about 2:1 and most preferably about 1:1. Such ratios of binder: surfactants may be for the granule composition such as intragranular composition or the extragranular composition or for the total composition of the tablet.

The pharmaceutical composition typically further comprises one or more disintegrants selected from cross-linked polyvinylpyrrolidone, croscarmellose sodium, calcium carboxyl methylcellulose, low substituted hydroxypropyl cellulose, alginic acid, sodium alginate, microcrystalline cellulose, sodium starch glycolate or pregelatinized starch. In a preferred embodiment, the disintegrant is croscarmellose sodium or sodium starch glycolate. In a more preferred embodiment the disintegrant is sodium starch glycolate.

The disintegrants may be present in an amount from about 0.5% to about 10%, from about 1% to about 8%, from about 2% to about 5%, preferably 2% to about 3%, from about 4% to about 5%, or from about 7% to about 8% by weight of the granule composition in one embodiment and by weight of the total tablet in another embodiment.

The one or more diluents used in the pharmaceutical composition preferably are selected from the group consisting of: an inorganic phosphates like dibasic calcium phosphate, or sugars or sugar analogues and derivatives thereof in particular lactose, such as lactose monohydrate or water-free lactose, dextrose, sorbitol, mannitol, saccharose, maltodextrin, isomaltose, or celluloses like microcrystalline cellulose or powdered celluloses or the like. In a preferred embodiment, the diluent selected from Lactose such as lactose monohydrate, microcrystalline cellulose and mannitol, or a mixture thereof. In a more preferred embodiment, intragranular component comprises microcrystalline cellulose and lactose monohydrate as diluent. In another preferred embodiment, microcrystalline cellulose and mannitol are present as diluent in the extragranular component. The diluents may present in an amount from about 10% to about 95%, preferably from about 40% to about 90%, more preferably from about 60% to about 85%, even more preferably from about 70% to about 85% by weight of the granule composition in one embodiment and by weight of the total tablet in another embodiment.

The pharmaceutical composition may optionally be film-coated using techniques well known in the art such as spray coating in a conventional coating pan or a fluidized bed processor or dip coating. Alternatively, coating may also be performed using the hot melt technique. The film coat comprises film-forming polymers, one or more pharmaceutically acceptable excipients and pharmaceutically acceptable solvents. Examples of film-forming agents include, but are not limited to, cellulose derivatives such as methylcellulose, hydroxymethyl cellulose, hydroxyethyl cellulose, hydroxypropyl cellulose, hydroxymethyl ethylcellulose, hydroxypropyl methylcellulose, sodium carboxymethyl cellulose, and ethyl cellulose; polyvinyl alcohol, waxes; fat substances; or mixtures thereof. Alternatively, commercially available coating compositions comprising film forming polymers marketed under various trade names, such as Opadry®, may be used for coating.

Examples of solvents used for preparing the coating solution are selected from methyl alcohol, ethyl alcohol, isopropyl alcohol, n-butyl alcohol, acetone, acetonitrile, chloroform, methylene chloride, water, or mixtures thereof. In a preferred embodiment, the film coating is a primary alcohol free coating. Preferably, the primary alcohol free coating is a coating made using water.

Glidants present in the pharmaceutical dosage form preferably are selected from silicon dioxide, talc, magnesium stearate and the like. A preferred glidant is silicon dioxide such as Aerosil® or magnesium stearate such as Ligamed MF 2V or a mixture thereof. Glidants may typically be present in amount from about 0.1% to 10%, from about 0. % to about 5%, or from about 1% to about 2% by weight of the granule composition in one embodiment and by weight of the total tablet in another embodiment.

Lubricants present in the pharmaceutical composition are preferably selected from fatty acids or fatty acid derivatives, such as alkali and earth alkali salts of stearic, lauric and/or palmitic acid and the like. A preferred lubricant is magnesium stearate and may typically be present in amount from about 0.1% to 10%, from about 0. % to about 5% or from about 1% to about 2% by weight of the granule composition in one embodiment and by weight of the total tablet in another embodiment.

Stability is an essential quality attribute for pharmaceutical formulations that determines the shelf life of the composition during which the composition is suitable for its intended use both from an efficacy and a safety point of view. The term stability of a pharmaceutical composition of a stable pharmaceutical composition means that one or more parameters governing the physical and chemical integrity of the active pharmaceutical ingredients (APIs) remain within a pharmaceutically acceptable criteria during the shelf life of the product. Typically one or more such parameters are selected from identification of the active ingredient(s) in the composition by methods, for example, HPLC and/or UV spectroscopy; visual appearance of the composition, assay percentage of the active ingredient(s) in the composition, individual and/or total percentage of the related substances and/or impurities in the composition, content uniformity of the composition with respect to the active ingredient(s), dissolution rate, microbial limits, and the like.

Pharmaceutical compositions often lose their efficacy and/or safety over time because of the loss or degradation or conversion of the active ingredient(s) into impurities commonly known as related substance(s). A stable fixed dose pharmaceutical composition retains at least up to about 90% (w/w) of the claimed potency for ezetimibe as well as obicetrapib.

Ezetimibe is known to give rise to stability problems associated with its formulations because of interactions with excipients and/or the combination drug partner. It has been surprisingly found that the fixed dose pharmaceutical composition effectively controls the levels of individual and total related substances of ezetimibe during the preparation as well as storage of the fixed dose composition. In an embodiment, the stable fixed dose pharmaceutical composition has not more than about 5% (w/w), preferably not more than about 2% (w/w), more preferably not more than about 1% (w/w) and even more preferably not more than about 0.2% (w/w) of an individual related substance of ezetimibe; and not more than about 5% (w/w), preferably not more than about 2% (w/w), more preferably not more than about 1% (w/w), and even more preferably not more than about 0.5% (w/w) of total related substances of ezetimibe. In a preferred embodiment, the fixed dose pharmaceutical composition comprising ezetimibe and obicetrapib wherein the ezetimibe tetrahydropyran analog impurity is not more than about 2% (w/w), preferably not more than about 0.5% (w/w), more preferably not more than about 0.3% (w/w), even more preferably not more than about 0.2% (w/w).

In another embodiment, the stable fixed dose pharmaceutical composition has not more than about 5% (w/w), preferably not more than about 2% (w/w), more preferably not more than about 0.5% (w/w), even more preferably not more than about 0.3% (w/w), and most preferably not more than about 0.2% (w/w) of any unspecified individual obicetrapib related substance; and not more than about 5% (w/w), preferably not more than about 2% (w/w), more preferably not more than about 1% (w/w), and even more preferably not more than about 0.5% (w/w) of total related substances of obicetrapib.

It has surprisingly been found the pharmaceutical composition remains stable for at least up to 1 month, preferably at least up to 3 months, more preferably at least up to 6 months under stability conditions of 40° C. temperature and 75% relative humidity. In a preferred embodiment, the composition remains stable at least up to 3 months, preferably at least up to 6 months under stability conditions of 40° C. temperature and 75% relative humidity. In another preferred embodiment, the composition remains stable for at least up to 3 months, 6 months or 12 months under stability conditions of 25° C. temperature and 60% relative humidity. In yet another preferred embodiment, the composition remains stable for at least up to 6 months, 12 months, 18 months or 24 months at room temperature.

In one of the preferred embodiments, the pharmaceutical composition is a tablet formulation comprising or consisting of:
a. an intragranular component comprising:
   i. Obicetrapib calcium equivalent to 10 mg obicetrapib free acid;
   ii. Ezetimibe anhydrous or a mixture of ezetimibe anhydrous and ezetimibe monohydrate equivalent to ezetimibe 10 mg;
   iii. a binder and a surfactant in a ratio of 1:1, preferably the binder and the surfactant each are about 1±0.5% w/w of the granule of intragranular component; more preferably the binder is 1±0.5% w/w polyvidone or polyvinylpyrrolidone and the surfactant is 1±0.5% w/w sodium lauryl sulphate;
   iv. a disintegrant selected from croscarmellose sodium, pregelatinized starch or sodium starch glycolate, more preferably sodium starch glycolate; preferably the disintegrant is about 2-8% w/w the granule of intragranular component, preferable 3-6% w/w, more preferably about 4.5±0.5% w/w;
   v. One or more diluents selected from disaccharides, preferably lactose or sucrose, more preferably anhydrous lactose or lactose monohydrate, even more preferably lactose monohydrate; polysaccharides, preferably cellulose, more preferably microcrystalline cellulose; sugar alcohols, preferably sorbitol, xylitol or mannitol;
b. An extragranular component comprising:
   i. a disintegrant selected from croscarmellose sodium, pregelatinized starch or sodium starch glycolate, more preferably sodium starch glycolate, even more preferably about 4%-6% w/w sodium starch glycolate;
   ii. optionally, a lubricant, preferably magnesium stearate, more preferably about 1%-2% w/w magnesium stearate;
   iii. optionally, a glidant, preferably colloidal silicon dioxide or talk or both, more preferably about 1%-2% w/w colloidal silicon dioxide or talk or both;
   iv. optionally, one or more diluents selected from disaccharides, preferably lactose or sucrose, more preferably anhydrous lactose or lactose monohydrate, even more preferably lactose monohydrate; polysaccharides, preferably cellulose, more preferably microcrystalline cellulose; sugar alcohols, preferably sorbitol, xylitol or mannitol; more preferably mannitol or microcrystalline cellulose, even more preferably about 20% to about 50% w/w microcrystalline cellulose and about 1% to about 20% mannitol.
c. Optionally, the composition comprises a film coating, preferably the film coating is free from a primary alcohol, more preferably the film coating is free from polyethylene glycol.
   In another preferred embodiment, the pharmaceutical composition comprises tablet formulation comprising or consisting of:
a. an intragranular component comprising:
   i. Ezetimibe anhydrous or a mixture of ezetimibe anhydrous and ezetimibe hydrate equivalent to ezetimibe 10 mg;
   ii. a binder and a surfactant in a ratio of 1:1, preferably the binder and the surfactant each are about 1±0.5% w/w of the granule of intragranular component; more preferably the binder is 1±0.5% w/w polyvidone or polyvinylpyrrolidone and the surfactant is 1±0.5% w/w sodium lauryl sulphate;
   iii. a disintegrant selected from croscarmellose sodium, pregelatinized starch or sodium starch glycolate, more preferably sodium starch glycolate; preferably the disintegrant is about 2-8% w/w the granule of intragranular component, preferable 3-6% w/w, more preferably about 4.5±0.5% w/w;
   iv. One or more diluents selected from disaccharides, preferably lactose or sucrose, more preferably anhydrous lactose or lactose monohydrate, even more preferably lactose monohydrate; polysaccharides, preferably cellulose, more preferably microcrystalline cellulose; sugar alcohols, preferably sorbitol, xylitol or mannitol;
b. an extra-granular component comprising:
   i. Obicetrapib calcium equivalent to 10 mg obicetrapib free acid;
   ii. a disintegrant selected from microcrystalline cellulose, pregelatinized starch or sodium starch glycolate, more preferably sodium starch glycolate, even more preferably about 4%-6% w/w sodium starch glycollate;
   iii. optionally, a lubricant, preferably magnesium stearate, more preferably about 1% w/w magnesium stearate;
   iv. optionally, a glidant, preferably colloidal silicon dioxide or talk or both, more preferably about 1%-2% w/w colloidal silicon dioxide or talk or both;
   v. optionally, one or more diluents selected from disaccharides, preferably lactose or sucrose, more preferably anhydrous lactose or lactose monohydrate, even more preferably lactose monohydrate; polysaccharides, preferably cellulose, more preferably microcrystalline cellulose; sugar alcohols, preferably sorbitol, xylitol or mannitol; more preferably mannitol or microcrystalline cellulose, even more preferably about 20% to about 50% w/w microcrystalline cellulose and about 1% to about 20% mannitol.
c. Optionally, the composition comprises a film coating, preferably the film coating is free from a primary alcohol, more preferably the film coating is free from polyethylene glycol.

In yet another preferred embodiment, the pharmaceutical composition is a tablet formulation comprising or consisting of:
a. an intragranular component comprising:
   i. Obicetrapib calcium equivalent to 10 mg obicetrapib free acid;
   ii. a binder and a surfactant in a ratio of 1:1, preferably the binder and the surfactant each are about 1±0.5% w/w of the granule of intragranular component; more preferably the binder is 1±0.5% w/w polyvidone or polyvinylpyrrolidone and the surfactant is 1±0.5% w/w sodium lauryl sulphate;
   iii. a disintegrant selected from croscarmellose sodium, pregelatinized starch or sodium starch glycolate, more preferably sodium starch glycolate; preferably the disintegrant is about 2-8% w/w the granule of intragranular component, preferable 3-6% w/w, more preferably about 4.5±0.5% w/w;
   iv. One or more diluents selected from disaccharides, preferably lactose or sucrose, more preferably anhydrous lactose or lactose monohydrate, even more preferably lactose monohydrate; polysaccharides, preferably cellulose, more preferably microcrystalline cellulose; sugar alcohols, preferably sorbitol, xylitol or mannitol;
b. an extra-granular component comprising:
   i. Ezetimibe anhydrous or a mixture of ezetimibe anhydrous and ezetimibe hydrate equivalent to ezetimibe 10 mg
   ii. a disintegrant selected from croscarmellose sodium, pregelatinized starch or sodium starch glycolate, more preferably sodium starch glycolate; even more preferably about 4%-6% w/w sodium starch glycollate
   iii. optionally, a lubricant, preferably magnesium stearate, more preferably about 1-2% w/w magnesium stearate
   iv. optionally, a glidant, preferably colloidal silicon dioxide or talc or both, more preferably about 1-2% colloidal silicon dioxide or talc or both;
   v. optionally, one or more diluents selected from disaccharides, preferably lactose or sucrose, more preferably anhydrous lactose or lactose monohydrate, even more preferably lactose monohydrate; polysaccharides, preferably cellulose, more preferably microcrystalline cellulose; sugar alcohols, preferably sorbitol, xylitol or mannitol; more preferably mannitol or microcrystalline cellulose, even more preferably about 20% to about 50% w/w microcrystalline cellulose and about 1% to about 20% mannitol.
c. Optionally, the composition comprises a film coating, preferably the film coating is free from a primary alcohol, more preferably the film coating is free from polyethylene glycol.

Another aspect relates to a pharmaceutical composition comprising obicetrapib and ezetimibe or pharmaceutically acceptable salts, solvates or co-crystals thereof and a pharmaceutically acceptable carrier for use in the treatment of subjects requiring additional lowering of low-density lipoprotein cholesterol as an adjunct to diet and/or as maximally tolerated lipid-lowering therapy for the treatment of adults with heterozygous familial hypercholesterolemia (HeFH) or established atherosclerotic cardiovascular (CV) disease (ASCVD).

A second aspect relates to the use of a fixed dose pharmaceutical composition comprising obicetrapib or a pharmaceutically acceptable salt, solvate or co-crystal thereof, ezetimibe or a pharmaceutically acceptable salt, solvate or co-crystal thereof, and one or more pharmaceutically acceptable excipients for preparation of a medicament for treatment of subjects requiring reduction in LDL cholesterol and/or an increase in HDL cholesterol.

In one of the embodiments, the said subjects are suffering from or having hyperlipidemia or mixed dyslipidemia, heterozygous familial hypercholesterolemia (HeFH) or established atherosclerotic cardiovascular disease (ASCVD).

In one embodiment, the said subjects [are partially or completely intolerant to statins.

In one embodiment, the use of a pharmaceutical composition is for treatment of subjects requiring additional lowering of low-density lipoprotein cholesterol as an adjunct to diet and/or maximally tolerated lipid-lowering therapy for the treatment of adults with heterozygous familial hypercholesterolemia (HeFH) or established atherosclerotic cardiovascular (CV) disease (ASCVD

A third aspect relates to a method of treatment of subjects requiring reduction in LDL cholesterol and/or an increase in HDL cholesterol, wherein the method comprises administering to the said subject a therapeutically effective dose of a fixed dose pharmaceutical composition comprising obicetrapib or a pharmaceutically acceptable salt, solvate or co-crystal thereof, ezetimibe or a pharmaceutically acceptable salt, solvate or co-crystal thereof, and one or more pharmaceutically acceptable excipients.

In one of the embodiments, the said method is for the treatment of subjects suffering from or having hyperlipidemia or mixed dyslipidemia, heterozygous familial hypercholesterolemia (HeFH) or established atherosclerotic cardiovascular disease (ASCVD).

In one embodiment, the subject requires additional lowering of low-density lipoprotein cholesterol as an adjunct to diet and/or as maximally tolerated lipid-lowering therapy for the treatment of adults with heterozygous familial hypercholesterolemia (HeFH) or established atherosclerotic cardiovascular (CV) disease (ASCVD).

In one embodiment, the said are partially or completely intolerant to statins.

A fourth aspect relates to a fixed dose combination pharmaceutical composition of obicetrapib and ezetimibe, wherein the said pharmaceutical composition is considered to be suitable for the said use according to the second aspect or said method of treatment according to the third aspect, when:
a. the fixed dose pharmaceutical composition is orally administered to a subject;
b. the concentration of obicetrapib in the subject's blood is determined at one or more time points after administration to provide a set of obicetrapib concentration/time data points to provide an area-under the curve (AUC); and
c. if 90% confidence interval for the geometric mean of the area under the curve (AUC 0-∞ and/or AUC 0-t) and/or Cmax for obicetrapib is within a range of 75%-125%, preferably 80%-125%, and more preferably 90%-110% of the area under the curve (AUC0-∞ and/or AUC 0-t) and/or Cmax, respectively, of obicetrapib as obtained upon oral administration of a reference pharmaceutical composition to a similar subject, wherein said reference composition comprises an equivalent dose of obicetrapib or its pharmaceutically acceptable salt, solvate or co-crystal thereof, and wherein the reference composition is administered alone, or as a simultaneous or sequential co-administration with another pharmaceutical composition comprising ezetimibe or a pharmaceutically acceptable salt, solvate or co-crystal thereof, or as a fixed-dose combination with ezetimibe or a pharmaceutically acceptable salt, solvate or co-crystal thereof.

A fifth aspect relates to a fixed dose combination pharmaceutical composition of obicetrapib and ezetimibe, wherein the said pharmaceutical composition is considered to be suitable for the said use according to the second aspect or said method of treatment according to the third aspect, when:
a. the said fixed dose pharmaceutical composition is orally administered to a subject, ezetimibe and/or ezetimibe glucoronide in the subject's blood is determined at one or more time points after administration to provide a set of ezetimibe and/or ezetimibe glucoronide concentration/time data points, respectively, to provide an area-under the curve (AUC) for ezetimibe and/or ezetimibe glucoronide, respectively; and,
b. if 90% confidence interval for the geometric mean of the area under the curve (AUC_{0-∞} and/or AUC₀₋ₜ) and/or Cmax for ezetimibe and/or ezetimibe glucoronide is within a range of 75%-125%, preferably 80%-125%, and more preferably 90%-110% of the area under the curve (AUC_{0-∞} and/or AUC₀₋ₜ) and/or Cmax, respectively, of ezetimibe and/or ezetimibe glucoronide, respectively, as obtained upon oral administration of a reference pharmaceutical composition to a similar subject, wherein the reference composition comprises an equivalent dose of ezetimibe or its pharmaceutically acceptable salt, solvate or co-crystal thereof, and wherein the reference composition is administered alone, or as a simultaneous or sequential co-administration with another pharmaceutical composition comprising obicetrapib or a pharmaceutically acceptable salt, solvate or co-crystal thereof, or as a fixed-dose combination with ezetimibe or a pharmaceutically acceptable salt, solvate or co-crystal thereof.

In one of the embodiments for the use according to the above aspects, t for AUC 0-t is selected from 48 hours (AUC 0-48), 72 hours (AUC 0-72), 96 hours (AUC 0-96), 144 hours (AUC 0-144), 192 hours (AUC 0-192), 240 hours (AUC 0-240), 336 hours (AUC 0-336) or AUC0-∞, preferably 48 hours (AUC 0-48), and more preferably 72 hours (AUC0-72) or AUC0-∞.

In one embodiment, the subject is a healthy human subject, preferably a non-tobacco, non-nicotine using adult male or female human, more preferably of 18-65 years of age, and optionally, the said human has a body mass index of 18.5 to 29.9 Kg/m².

In another embodiment, the subject is human requiring reduction in LDL cholesterol and/or an increase in HDL cholesterol. In a preferred embodiment, the is human is suffering from or having hyperlipidemia or mixed dyslipidemia, heterozygous familial hypercholesterolemia (HeFH) or established atherosclerotic cardiovascular disease (ASCVD).

In one embodiment, the said human is partially or completely intolerant to statins.

Preferably the human subject has LDL-cholesterol levels ≥70 mg/dL, and optionally the said humans are not adequately controlled by their current lipid-modifying therapies.

For the use of a pharmaceutical composition or a method of treatment according to the other aspects, the subject in need thereof may be administered with the said composition to deliver a total daily oral dose of 5 mg obicetrapib and 10 mg ezetimibe, 10 mg obicetrapib and 10 mg ezetimibe, or 20 mg obicetrapib and 20 mg ezetimibe, preferably the subject is administered with the said composition to deliver a daily oral dose of 10 mg obicetrapib and 10 mg ezetimibe.

It has been surprisingly found that the dissolution profile of ezetimibe from the fixed dose combination was found to be non-inferior or sometime superior as compared to the commercial formulation of ezetimibe (Zetia®) which is discussed in detail in the examples section. It was also surprisingly found that the fixed dose combination composition disclosed herein is bioequivalent to a combination of monotherapy drugs co-administered to human subjects. The confidence intervals (90%) on the geometric mean ratios for AUCo-t, AUCo-∞ and Cmax for obicetrapib, ezetimibe and ezetimibe glucoronide from two of the representative compositions—FDC1 and FDC2 were found to be within a range of 75%-125%, preferably 80%-125%, and more preferably 90%-110% of AUCo-t, AUCo-∞ and Cmax of obicetrapib, ezetimibe and ezetimibe glucoronide, respectively as obtained from co-administration of single drug formulations of same dose of ezetimibe and obicetrapib, which is discussed in detail in the examples section below.

The fixed dose combination pharmaceutical composition of obicetrapib and ezetimibe will be illustrated further by means of the non-limiting examples herein below.

### The Methods of Treatment of the Invention

As explained herein before, the invention provides methods for the curative and/or prophylactic treatment of a subject in need thereof. More in particular, the invention provides methods for the treatment and/or prevention of cardiovascular disease, in particular Atherosclerotic cardiovascular disease, in such subjects, using the compositions as defined herein. The invention further provides methods for the treatment and/or prevention of one or more symptoms associated with (atherosclerotic) cardiovascular disease, in such subjects, using the compositions as defined herein. The invention further provides methods for the treatment and/or prevention of one or more pathologies associated with and/or caused by (atherosclerotic) cardiovascular disease, in such subjects, using the compositions as defined herein. The invention further provides methods for the treatment and/or prevention of one or more aetiological factors associated with (atherosclerotic) cardiovascular disease, such as elevated LDL-C levels and/or elevated ApoB levels, in such subjects, using the compositions as defined herein. The invention further provides methods for mitigating and/or ameliorating resistance or hypo-responsiveness to statin therapy, in particular high intensity statin therapy, in such subjects, using the compositions as defined herein.

The terms “treat”, “treating” or “treatment”, when used in conjunction with a specific disease or symptom (for example: “method of treating disease . . . ”) refers to curing, alleviating or abrogating said disease and/or accompanying symptoms, diminishing extent of disease, stabilizing (i.e. not worsening) the state of disease, delaying or slowing of disease progression, ameliorating the disease state, prolonging survival (as compared to expected survival without treatment), etc. The terms “prevent”, “preventing” or “prevention”, as used herein, refer to reducing the risk for a subject to acquire a disease and/or accompanying symptoms, delaying the moment a subject acquires disease, etc. The terms “treat”, “treating” or “treatment”, when used in relation to a patient or subject (for example: “method of treating a subject”), typically refers to the act of administering a therapeutic compound to said patient or subject for whatever therapeutic and/or prophylactic purpose.

The term “cardiovascular disease” as used herein has its conventional meaning as referring to a disease or condition in which the function of a subject's cardiovascular system becomes impaired. Examples of cardiovascular diseases include thromboembolic disorders (e.g., arterial cardiovascular thromboembolic disorders, venous cardiovascular thromboembolic disorders, or thromboembolic disorders in the chambers of the heart); atherosclerosis; hypertensive heart disease; coronary artery disease; carotid artery disease; stroke; peripheral artery disease involving atherosclerosis; restenosis; arteritis; myocarditis; cardiovascular inflammation; vascular inflammation; coronary heart disease (CHD); unstable angina (UA); unstable refractory angina; stable angina (SA); chronic stable angina; acute coronary syndrome (ACS); myocardial infarction (first or recurrent); acute myocardial infarction (AMI); myocardial infarction; ischemic heart disease; cardiac ischemia; ischemia; ischemic sudden death; transient ischemic attack; stroke; peripheral occlusive arterial disease; venous thrombosis; deep vein thrombosis; thrombophlebitis; arterial embolism; coronary arterial thrombosis; cerebral arterial thrombosis, cerebral embolism; kidney embolism; pulmonary embolism; etc.

As used herein, the term “atherosclerotic cardiovascular disease” refers to a specific subset of cardiovascular diseases that include atherosclerosis as a component or precursor to the particular type of cardiovascular disease. Atherosclerosis is a chronic inflammatory response that occurs in the walls of arterial blood vessels associated with retained LDL-C. It involves the formation of atheromatous plaques that can lead to narrowing (“stenosis”) of the artery, and can eventually lead to partial or complete closure of the arterial opening and/or plaque ruptures. Thus atherosclerotic diseases or disorders include the consequences of atheromatous plaque formation and rupture including, without limitation, stenosis or narrowing of arteries, heart failure, aneurysm formation including aortic aneurysm, aortic dissection, and ischemic events such as myocardial infarction and stroke.

In particularly preferred embodiments, the atherosclerotic cardiovascular disease and/or pathology associated with atherosclerotic cardiovascular disease that may advantageously be treated and/or prevented according to the present invention is selected from the group consisting of arteriosclerosis, peripheral vascular disease, hyperlipidemia, mixed dyslipidemia betalipoproteinemia, hypoalphalipoproteinemia, hypercholesteremia, hypertriglyceridemia, familial-hypercholesteremia, angina, ischemia, cardiac ischemia, stroke, myocardial infarction, reperfusion injury, restenosis after angioplasty, hypertension, cerebral infarction and cerebral stroke.

In another particularly preferred embodiments, the atherosclerotic cardiovascular disease and/or pathology associated with atherosclerotic cardiovascular disease that may advantageously be treated and/or prevented according to the present invention is heterozygous familial hypercholesterolemia (HeFH).

In another particularly preferred embodiments, the atherosclerotic cardiovascular disease and/or pathology associated with atherosclerotic cardiovascular disease that may advantageously be treated and/or prevented according to the present invention is established ASCVD.

In another particularly preferred embodiments, the atherosclerotic cardiovascular disease and/or pathology associated with atherosclerotic cardiovascular disease that may advantageously be treated and/or prevented according to the present invention is non-familial hypercholesterolemia.

In another particularly preferred embodiments, the atherosclerotic cardiovascular disease and/or pathology associated with atherosclerotic cardiovascular disease that may advantageously be treated and/or prevented according to the present invention is primary hyperlipidemia.

In another particularly preferred embodiments, the atherosclerotic cardiovascular disease and/or pathology associated with atherosclerotic cardiovascular disease that may advantageously be treated and/or prevented according to the present invention is primary hypercholesterolemia.

As will be apparent from the present teachings, the methods of the present invention are effective in and/or intended for reducing and/or normalizing LDL-C plasma levels. More in particular, the methods are effective in and/or intended for reducing LDL-C plasma levels, with at least 5%, from baseline, wherein baseline is defined as start of the treatment with obicetrapib and ezetimibe, more preferably at least 10%, at least 15%, at least 20%, at least 25%, at least 30%, at least 35%, at least 40%, at least 45% or at least 50%. In further embodiments, the methods are effective in and/or intended for reducing LDL-C plasma levels, with at least 5 mg/dL, from baseline, wherein baseline is defined as start of the treatment with obicetrapib and ezetimibe, more preferably at least 10 mg/dL, at least 15 mg/dL, at least 20 mg/dL, at least 25 mg/dL, at least 30 mg/dL, at least 35 mg/dL or at least 40 mg/dL. In further embodiments, the methods are effective in and/or intended for reducing LDL-C plasma levels, with at least 45 mg/dL or at least 50 mg/dL. In further embodiments, the methods are effective in and/or intended for reducing LDL-C plasma levels, to a level below 85 mg/dL, preferably below 80 mg/dL, below 75 mg/dL, below 70 mg/dL, below 65 mg/dL, below 60 mg/dL, below 55 mg/dL or below 50 mg/dL.

As will be apparent from the present teachings, the administration of ezetimibe (or a pharmaceutically acceptable salt, solvate or co-crystal thereof) in addition to obicetrapib (or a pharmaceutically acceptable salt, solvate or co-crystal thereof) results in remarkable enhancement, notably supra-additive or synergistic enhancement, of LDL-C reduction. More in particular, the present methods of administering ezetimibe (or a pharmaceutically acceptable salt, solvate or co-crystal thereof), in order to enhance the LDL-C lowering effect of obicetrapib as defined herein, are effective in and/or intended for further reducing LDL-C plasma levels, with at least 20%, as compared to methods based on therapy with obicetrapib, or a pharmaceutically acceptable salt, solvate or co-crystal thereof, alone (or, at least, without ezetimibe), more preferably at least 22.5%, at least 25%, at least 26%, at least 27%, at least 28%, at least 29% or at least 30%. In further embodiments, these methods are effective in and/or intended for further reducing LDL-C plasma levels, with at least 20 mg/dL as compared to methods based on therapy with obicetrapib, or a pharmaceutically acceptable salt, solvate or co-crystal thereof, alone (or, at least, without ezetimibe), more preferably at least 22.5 mg/dL, at least 25 mg/dL, at least 27.5 mg/dL, at least 30 mg/dL, at least 32.5 mg/dL or at least 35 mg/dL.

In preferred embodiments of the invention, the methods are effective in and/or intended for reducing and/or normalizing ApoB plasma levels. More in particular, the methods are effective in and/or intended for reducing ApoB plasma levels, with at least 5%, from baseline, wherein baseline is defined as start of the treatment with obicetrapib and ezetimibe, more preferably at least 10%, at least 15%, at least 20%, at least 22.5%, at least 25% or at least 27.5%. In further embodiments, the methods are effective in and/or intended for reducing ApoB plasma levels, with at least 5 mg/dL, from baseline, wherein baseline is defined as start of the treatment with obicetrapib and ezetimibe, more preferably at least 5 mg/dL, at least 10 mg/dL, at least 15 mg/dL, at least 20 mg/dL, at least 22.5 mg/dL, at least 25 mg/dL or at least 27.5 mg/dL. In further embodiments, the methods are effective in and/or intended for reducing ApoB plasma levels, to a level below 80 mg/dL, preferably below 75 mg/dL, below 70 mg/dL, below 65 mg/dL, below 60 mg/dL, below 57.5 mg/dL or below 55 mg/dL.

In preferred embodiments of the invention, the methods are effective in and/or intended for reducing and/or normalizing Lp(a) plasma levels. More in particular, the methods are effective in and/or intended for reducing Lp(a) plasma levels, with at least 5%, from baseline, wherein baseline is defined as start of the treatment with obicetrapib and ezetimibe, more preferably at least 7.5%, at least 10%, at least 12.5%, at least 15%, at least 17.5% or at least 20%. In further embodiments, the methods are effective in and/or intended for reducing Lp(a) plasma levels, with at least 5 nmol/L, from baseline, wherein baseline is defined as start of the treatment with obicetrapib and ezetimibe, more preferably at least 10 nmol/L, at least 15 nmol/L, at least 20 nmol/L, at least 25 nmol/L, at least 30 nmol/L, at least 35 nmol/L or at least 40 nmol/L. In further embodiments, the methods are effective in and/or intended for reducing Lp(a) plasma levels, to a level below 110 nmol/L, preferably below 105 nmol/L, below 100 nmol/L, below 95 nmol/L, below 90 nmol/L, below 85 nmol/L or below 80 nmol/L.

In some embodiments of the invention, the methods are effective in and/or intended for mitigating and/or ameliorating resistance or hypo-responsiveness to statin therapy, in particular high intensity statin therapy. High intensity statin therapy is a term conventionally used in the art to denote the regimens based on the highest allowed dosages of the statins having the highest efficacy in reducing LDL-C, notably regimens that typically display a LDL-C reduction ≥50% in normally responsive subjects. Of the statins currently used in clinical practice, only 20 mg (daily) or 40 mg (daily) of rosuvastatin and 40 mg (daily) or 80 mg (daily) of atorvastatin meet the criteria. In the context of the present invention hypo-responsiveness to HIS therapy means that a subject receiving HIS therapy fails to reach a 35% LDL-C reduction, preferably it means that a subject receiving HIS therapy fails to reach a 30% LDL-C reduction, a 25% LDL-C reduction, a 20% LDL-C reduction, a 15% LDL-C reduction, or a 10% LDL-C reduction. Mitigating and/or ameliorating hypo-responsiveness to HIS therapy, means that the difference between the subject's response (LDL-C reduction) and the (average) response of normo-responsive subjects is reduced. In further embodiments of the invention, the methods are effective in and/or intended for normalizing the responsiveness to statin therapy.

As explained herein before, the methods of the invention are directed at the treatment and/or prevention of a subject suffering from or at risk of suffering from CVD, in particular ASCVD.

The term “a subject” refers to a living organism, typically a mammal, in particular a human subject, suffering from or prone to a disease or condition that can be treated by using the composition provided herein.

In particularly preferred embodiments of the invention, the subject is a subject that has been diagnosed with CVD, in particular ASCVD.

In particularly preferred embodiments of the invention, the subject, is a subject preferably an adult, that has established ASCVD.

In particularly preferred embodiments of the invention, the subject, is a subject preferably an adult, that has HeFH.

In particularly preferred embodiments of the invention, the subject, is a subject preferably an adult, that has mixed dyslipidemia.

In further preferred embodiments of the invention, the subject is a subject that is considered to be at risk, typically at above-average risk, of developing CVD, in particular ASCVD, as can e.g. be judged by healthcare professionals.

In preferred embodiments of the invention, the subject is a subject suffering from one or more conditions known to bear a causal and/or epidemiological correlation with the occurrence of (AS)CVD, such as diabetes, hypertension, hypercholesterolemia, including, overweight/obesity, metabolic syndrome, etc. In further preferred embodiments of the invention, the subject is a subject that is genetically predisposed to develop (AS)CVD. In further preferred embodiments of the invention, the subject is a subject prone to develop (AS)CVD as a consequence of life-style/habitual factors, such as unhealthy diet, lack of exercise, alcohol consumption, smoking.

In accordance with a preferred embodiment of the invention, the subject to be treated has elevated plasma levels of LDL-C, typically an LDL-C plasma level of at least 70 mg/dL, more preferably at least 75 mg/dL, at least 80 mg/dL, at least 85 mg/dL, at least 90 mg/dL, at least 95 mg/dL or at least 100 mg/dL. Furthermore, in accordance with preferred embodiments of the invention, the subject has an LDL-C plasma level that is at least 125% of the average LDL-C plasma level in healthy subjects, e.g. at least 150%, at least 175%, or at least 200%. Normal LDL-C (reference) values typically depend on gender and age.

In accordance with another preferred embodiment of the invention, the subject to be treated is a subject that is unable to reach LDL-C goals with the maximum tolerated dose of a statin.

In accordance with another preferred embodiment of the invention, the subject to be treated is a subject that is intolerant to statin and is unable to reach LDL-C goals with ezetimibe alone.

In accordance with another preferred embodiment of the invention, the subject to be treated is a subject that is unable to reach LDL-C goals with the maximum tolerated dose of a statin in addition to ezetimibe.

As used herein, the term ‘LDL-C goal’ refers to the level of plasma LDL-C that is considered to significantly reduce the risk of cardiovascular events, compared to the patients baseline level, and, on that basis, is selected as the target level to be reached with lipid-lowering therapy. For instance, The European Society of Cardiology (ESC)/European Atherosclerosis Society (EAS) guidelines, recommend tailoring treatment to each patient's level of cardiovascular risk and have set an LDL-C goal of <70 mg/dL for patients considered to be at very high cardiovascular risk. Patients in this risk category have documented cardiovascular disease (e.g., prior myocardial infarction [MI]), or a 10% or greater 10-year risk of fatal cardiovascular disease, and in randomized controlled trials, the guideline-specified LDL-C goal has been shown to reduce the risk of recurrent cardiovascular events. Hence, in certain embodiments of the invention, the term ‘LDL-C goals’ refers to an LDL-C plasma level of <70 mg/dL. In preferred embodiments, the subject to be treated is a subject that is unable to reach an LDL-C plasma level of <70 mg/dL, with the maximum tolerated dose of a statin, with ezetimibe or with the maximum tolerated dose of a statin in combination with ezetimibe.

In accordance with another embodiment of the invention, the subject to be treated is a subject requiring additional lowering of LDL-C as an adjunct to diet and/or to maximally tolerated lipid-lowering therapy, wherein said lipid-lowering therapy is not obicetrapib/ezetimibe combination therapy.

In accordance with a preferred embodiment of the invention, the subject to be treated has elevated plasma levels of ApoB, typically an ApoB plasma level of at least 70 mg/dL, more preferably at least 75 mg/dL, at least 80 mg/dL, at least 85 mg/dL, at least 90 mg/dL, at least 95 mg/dL or at least 100 mg/dL. Furthermore, in accordance with preferred embodiments of the invention, the subject has an ApoB plasma level that is at least 125% of the average ApoB plasma level in healthy subjects, e.g. at least 150%, at least 175%, or at least 200%. Normal ApoB (reference) values typically depend on gender and age.

In accordance with a preferred embodiment of the invention, the subject to be treated has elevated plasma levels of non-HDL-C, typically a non-HDL-C plasma level of at least 100 mg/dL, more preferably at least 105 mg/dL, at least 110 mg/dL, at least 115 mg/dL, at least 120 mg/dL, at least 125 mg/dL or at least 130 mg/dL. Furthermore, in accordance with preferred embodiments of the invention, the subject has a non-HDL-C plasma level that is at least 125% of the average non-HDL-C plasma level in healthy subjects, e.g. at least 150%, at least 175%, or at least 200%. Normal non-HDL-C (reference) values typically depend on gender and age.

In one embodiment of the invention, the subject is human male. In another embodiment of the invention, the subject is human female.

In further preferred embodiments of the invention, the subject is at increased risk based on age, such as a subject being over 35 years of age, over 40 years of age, over 45 years of age, over 50 years of age, over 55 years of age, over 60 years of age, over 65 years of age or over 70 years of age; typically in combination with one or more other risk factors as defined herein.

In accordance with certain embodiments of the invention, the subjects to be treated display hypo-responsiveness to statin therapy, in particular HIS therapy. High intensity statin therapy is a term conventionally used in the art, to denote the regimens based on the highest allowed dosages of statins having the highest efficacy in reducing LDL-C, notably regimens that typically display a LDL-C reduction ≥50% in normally responsive subjects. In current clinical practice, only rosuvastatin 20 mg/day or 40 mg/day and atorvastatin 40 mg/day or 80 mg/day are considered HIS therapy. In preferred embodiments of the invention, the subject is a subject that is receiving HIS therapy and fails to reach a 35% LDL-C reduction, preferably a subject receiving HIS therapy that fails to reach a 30% LDL-C reduction, a 25% LDL-C reduction, a 20% LDL-C reduction, a 15% LDL-C reduction, or a 10% LDL-C reduction. In preferred embodiments of the invention, the subject's hypo-responsiveness to statin therapy, in particular HIS therapy, is established after at least 1 month of (continuous) HIS therapy, more preferably at least 2 months, at least 3 months, at least 4 months, at least 5 months or at least 6 months.

The various aspects of the present invention as defined herein, all relate to methods of treatment involving the administration, typically the repeated administration, of a composition comprising obicetrapib (or a salt or solvate or co-crystal thereof), preferably any composition as defined herein before. In particularly preferred embodiments of the invention, the methods of treatment comprise the administration, typically the repeated administration, of a composition comprising an LDL-C lowering amount of obicetrapib or a salt or solvate or co-crystal thereof.

Hence, in particularly preferred embodiments of the invention, the method comprises the administration of obicetrapib in a dose of at least 1 mg, preferably at least 2 mg, at least 3 mg, at least 4 mg, at least 5 mg, at least 6 mg, at least 7 mg, at least 8 mg, or at least 9 mg, e.g. about 10 mg; or a salt, solvate or co-crystal of obicetrapib in the equipotent dose. In accordance with the various aspects of the invention, the method comprises the administration of obicetrapib in a dose of 100 mg or less, more preferably 75 mg or less, 50 mg or less, 40 mg or less, 30 mg or less, 20 mg or less, 15 mg or less, 12.5 mg or less, 12 mg or less, or 11 mg; or a salt, solvate or co-crystal of obicetrapib in the equipotent dose. In accordance with the various aspects of the invention, the method comprises the administration of obicetrapib in a dose within the range of 1-100 mg, 2-50 mg, 3-50 mg, 4-25 mg, 4.5-15 mg or 5-10 mg; or a salt, solvate or co-crystal of obicetrapib in the equipotent dose. In certain preferred embodiments, the method comprises the administration of obicetrapib in a dose of 1, 2, 3, 4, 5, 6, 7, 8, 9, 10, 11, 12, 13, 14, 15, 16, 17, 18, 19 or 20 mg; or a salt, solvate or co-crystal of obicetrapib in the equipotent dose. In certain particularly preferred embodiments, the method comprises the administration obicetrapib in a dose of 5, 7.5, 10, 12.5 or 15 mg; or a salt, solvate or co-crystal of obicetrapib in the equipotent dose.

In particularly preferred embodiments of the invention, the treatment comprises the repeated administration of the composition containing obicetrapib or a salt, hydrate or solvate thereof, preferably in a dose within the ranges defined herein before. In particularly preferred embodiments of the invention, the treatment comprises the repeated administration of the composition, preferably in a dose within the ranges defined herein before, at a frequency of at least once every two days or at least once every day. In particularly preferred embodiments of the invention, the treatment comprises the repeated administration of the composition, preferably in the dose as defined herein before, at a frequency of once to four times every day. In particularly preferred embodiments of the invention, the method comprises the once or twice daily administration of the composition containing obicetrapib or a salt, hydrate or solvate thereof, in the dose ranges as defined here above, most preferably twice daily.

Hence, in particularly preferred embodiments of the invention, the method comprises the administration of obicetrapib at a daily dosage of at least 1 mg, preferably at least 2 mg, at least 3 mg, at least 4 mg, at least 5 mg, at least 6 mg, at least 7 mg, at least 8 mg, or at least 9 mg, e.g. about 10 mg; or a salt, solvate or co-crystal of obicetrapib in the equipotent dosage. In accordance with the various aspects of the invention, the method comprises the administration of obicetrapib at a daily dosage of 100 mg or less, more preferably 75 mg or less, 50 mg or less, 40 mg or less, 30 mg or less, 20 mg or less, 15 mg or less, 12.5 mg or less, 12 mg or less, or 11 mg; or a salt, solvate or co-crystal of obicetrapib in the equipotent dosage. In accordance with the various aspects of the invention, the method comprises the administration of obicetrapib at a daily dosage within the range of 1-100 mg, 2-50 mg, 3-50 mg, 4-25 mg, 4.5-15 mg or 5-10 mg; or a salt, solvate or co-crystal of obicetrapib in the equipotent dosage. In certain preferred embodiments, the method comprises the administration of obicetrapib at a daily dosage of 1, 2, 3, 4, 5, 6, 7, 8, 9, 10, 11, 12, 13, 14, 15, 16, 17, 18, 19 or 20 mg; or a salt, solvate or co-crystal of obicetrapib in the equipotent dosage. In certain particularly preferred embodiments, the method comprises the administration of obicetrapib at a daily dosage of 4 of 5, 7.5, 10, 12.5 or 15 mg; or a salt, solvate or co-crystal of obicetrapib in the equipotent dosage.

As will be apparent to those skilled in the art, based on the present teachings, the methods of the invention further comprise the concurrent treatment with ezetimibe (or a salt or solvate or co-crystal thereof). In particularly preferred embodiments of the invention, the methods of treatment comprise the administration, typically the repeated administration, of a composition comprising an LDL-C lowering amount of ezetimibe or a salt or solvate or co-crystal thereof.

To this end, ezetimibe and obicetrapib (or a therapeutically acceptable salt, solvate or co-crystal thereof) may be administered at or around the same time, sequentially or concurrently, or they may be administered at different time points. In preferred embodiments of the invention, the frequency and administration intervals of obicetrapib and ezetimibe are equal, more preferably each is administered once daily, still more preferably at the same time of the day, sequentially or concurrently as two separate unit dosage forms, preferably in the form of the fixed dose combination product as defined herein. In preferred embodiments, the methods of the invention comprise the administration of ezetimibe at a daily dosage of 1-30 mg, 2-25 mg, 3-20 mg, 4-17.5 mg, or 5-15 mg e.g. 1, 2, 3, 4, 5, 6, 7, 8, 9, 10, 11, 12, 13, 14, 15, 16, 17, 18, 19 or 20 mg, most preferably about 10 mg; or a salt, solvate or co-crystal of ezetimibe, in the equipotent dosage.

As will be apparent to those skilled in the art, based on the present teachings, the methods of the invention, in some embodiments, further comprises the concurrent treatment with a HMG CoA reductase inhibitor, preferably concurrent HIS therapy. To this end, the HMG CoA reductase inhibitor and obicetrapib (or a therapeutically acceptable salt, solvate or co-crystal thereof) may be administered at or around the same time, sequentially or concurrently, or they may be administered at different time points. In preferred embodiments of the invention, the frequency and administration intervals of obicetrapib and the HMG CoA reductase inhibitor are equal, more preferably each is administered once daily, still more preferably at the same time of the day, sequentially or concurrently as two separate unit dosage forms, or in the form of a fixed dose combination product. In preferred embodiments, the methods of the invention comprise the administration of rosuvastatin at a daily dosage of 10-50 mg, 15-45 mg, 17.5-42.5 mg, or 20-40 mg, e.g. 15, 16, 17, 18, 19, 20, 21, 22, 23, 24, 25, 35, 36, 37, 38, 39, 40, 41, 42, 43, 44, or 45 mg, most preferably about 20 mg or 40 mg; or a salt, solvate or co-crystal of rosuvastatin in the equipotent dosage. In preferred embodiments, the methods of the invention comprise the administration of atorvastatin at a daily dosage of 30-90 mg, 35-85 mg, 37.5-82.5 mg, or 40-80 mg, e.g. 35, 36, 37, 38, 39, 40, 41, 42, 43, 44, 45, 75, 76, 77, 78, 79, 80, 81, 82, 83, 84 or 85 mg, most preferably about 40 mg or 80 mg; or a salt, solvate or co-crystal of atorvastatin in the equipotent dosage. In certain preferred embodiments, the methods of the invention do not comprise the concomitant treatment with a HMG CoA reductase inhibitor.

As will be apparent to the skilled person, on the basis of the present teachings, the daily doses indicated herein may be contained in a single unit dosage form as well as in a plurality of unit dosage forms. In a most preferred embodiment of the invention, the method comprises the administration of obicetrapib (or a salt, hydrate or solvate thereof) in the dosages recited herein once daily. However, methods are also envisaged comprising the administration of 2 unit dose forms, each comprising approximately half of the daily dosage as indicated above, at certain pre-determined moments during the day, e.g. one in the morning, such as shortly after the subject wakes up, and one in the evening, such as around the time the subject has his evening meal or goes to sleep. Embodiments wherein unit dosage forms are used comprising higher amounts of obicetrapib and/or ezetimibe than the daily dose indicated herein are also contemplated. This may e.g. involve the use of extended release dosage forms that remain in the body and keep releasing the active ingredient for a sufficiently long time.

In embodiments, methods and/or compositions for use according to the invention are provided, wherein the methods and/or use comprise the administration, preferably the repeated administration, of obicetrapib and ezetimibe (or a salt, hydrate or solvate thereof), preferably in the form of the fixed dose pharmaceutical composition as defined herein, to the subject, at a dose and frequency effective to reduce the subject's LDL-C plasma levels, the subject's ApoB plasma levels and/or the subjects Lp(a) plasma levels, more preferably to accomplish a reduction in one or more of the subject's LDL-C plasma levels, the subject's ApoB plasma levels and/or the subjects Lp(a) plasma levels within the ranges recited herein elsewhere. In particularly preferred embodiments of the invention, these treatments comprise the repeated administration of obicetrapib and ezetimibe (or a salt, hydrate or solvate of obicetrapib and/or ezetimibe), preferably in the form of the fixed dose pharmaceutical composition as defined herein, in accordance with the above-defined regimens, during a period of at least one month, at least three months, at least four months, at least six months, at least nine months, at least one year, at least two year, at least three year, at least 5 year, at least 10 year, at least 20 year, at least 30 year. There is no particular upper limit; treatment may be continued for as long as it is deemed beneficial to the subject's overall health and well-being (as determined by appropriately qualified healthcare professional), e.g. for the rest of the subject's life.

### The Pharmaceutical Kits of the Invention

Another aspect of the invention is directed to a pharmaceutical kit comprising a package containing a plurality of unit dosage forms and a leaflet, wherein said unit dosage forms contain the pharmaceutical composition according to the invention and wherein said leaflet contains printed instructions to repeatedly self-administer said unit dosage forms in order to accomplish any of the therapeutic objectives as defined herein, such as to treat and/or prevent any cardiac disease or dysfunction as defined herein.

In accordance with embodiments of the invention, the pharmaceutical kit comprises a container, such as a cardboard box, holding one or more blister packs, said one or more blister packs containing a plurality of solid unit dosage forms as defined herein before, preferably a plurality of tablets as defined herein before. In particularly preferred embodiments of the invention, the pharmaceutical kit comprises at least 5, at least 8, at least 10, at least 12 of at least 15 of said unit dosage forms, e.g. 4, 5, 6, 7, 8, 9, 10, 11, 12, 13, 14, 15, 16, 17, 18, 19 or 20 of said unit dosage forms. In one embodiment of the invention, the pharmaceutical kit only comprises unit dosage forms as defined herein that contain obicetrapib as the sole active ingredient. In one embodiment of the invention, the pharmaceutical kit only comprises a plurality of unit dosage forms as defined herein that contain obicetrapib (or a salt, hydrate or solvate thereof) as the sole active ingredient and a plurality, preferably an equal number, of unit dosage forms that contain ezetimibe as the sole active, typically in the dose amounts recited herein elsewhere. In one embodiment of the invention, the pharmaceutical kit only comprises a plurality of unit dosage forms as defined, wherein each unit dosage form comprises obicetrapib (or a salt, hydrate or solvate thereof) and ezetimibe (or a salt, hydrate or solvate thereof), more preferably a plurality of the fixed dose pharmaceutical composition as defined herein. In some embodiments, pharmaceutical kits as defined herein may further comprise a plurality of unit dosage forms that contain a HMG CoA reductase inhibitor as the sole active, preferably atorvastatin or rosuvastatin (or a salt, hydrate or solvate thereof), typically in the dose amounts recited herein elsewhere.

In accordance with the invention, the pharmaceutical kit comprises a leaflet inserted into the container, typically a patient information leaflet containing printed information, which information may include a description of the form and composition of the unit dosage forms contained in the kit, an indication of the therapeutic indications for which the product is intended, instructions as to how the product is to be used and information and warnings concerning adverse effects and contraindications associated with the use. It will be understood by those of average skill in the art, based on the information presented herein, that the leaflet that is part of the kit according to the invention, will typically contain the information concerning the therapeutic indications, uses, treatment regimens, etc. as described here above in relation to the methods of treatment of the present invention. In particularly preferred embodiments of the invention, the leaflet contains printed instructions to repeatedly (self-) administer the unit dosage forms in order to treat and/or prevent CVD, in particular ASCVD.

The methods of treatment of the invention, based on obicetrapib and ezetimibe combination therapy will be illustrated further by means of the non-limiting examples herein below.

### The Amorphous Calcium Salt Form of Obicetrapib

In certain preferred embodiments of the invention, obicetrapib, as contained in the present pharmaceutical compositions, as used in the present methods, as contained in the unit dosage forms (comprised in the pharmaceutical kit), etc., is a salt form of obicetrapib, more particularly an amorphous obicetrapib calcium salt, in particular, amorphous obicetrapib hemicalcium.

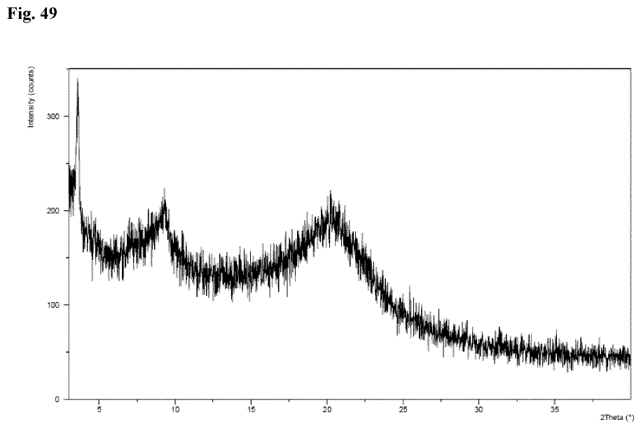

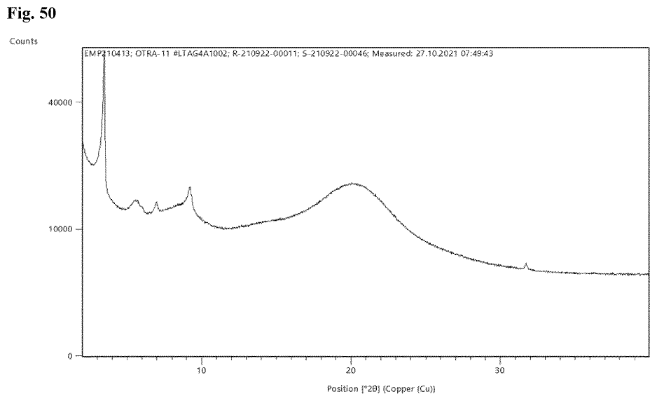

The amorphous obicetrapib hemicalcium of the disclosure is different from and can be distinguished from the crystalline obicetrapib hemicalcium disclosed in U.S. Pat. No. 7,872,126. A common technique used to distinguish crystalline from amorphous materials is x-ray powder diffraction. However, this technique has limitations, especially when the crystalline material is disordered. In the case of amorphous obicetrapib hemicalcium, x-ray powder diffraction patterns of two different lots of amorphous obicetrapib hemicalcium are provided in FIG. 49 and FIG. 50. These patterns have the familiar “halo” type features that are associated with amorphous materials. The x-ray powder diffraction pattern from FIG. 50 has peaks at about 3.4°2θ, about 7.0°2θ, and about 9.2°2θ. Similarly, another sample of FIG. 51 has x-ray powder diffraction peaks also at about 3.4°2θ, about 7.0°2θ, and about 9.2°2θ. The x-ray powder diffraction patterns of any of FIG. 49 or FIG. 50 or FIG. 51 may be used to characterize amorphous obicetrapib hemicalcium, provided, however, occasionally, a sharp higher angle peak is present, such as at about 31.7°2θ is found (such as in FIG. 50), and that peak, when present, is due to sodium chloride. In FIG. 51, in another sample of amorphous obicetrapib hemicalcium, peaks at about 3.4°2θ, about 7.0°2θ, and about 9.2°2θ were identified. The peak at about 5.6°2θ in FIG. 51 was determined to be due to Kapton foil, which was used in the measurement setup as explained in Example 11.20. The x-ray powder pattern of crystalline obicetrapib hemicalcium as prepared in Example 11.16 is shown in FIG. 54. It too exhibits halo-like behavior which, for a crystalline compound, may be indicative of disorder.

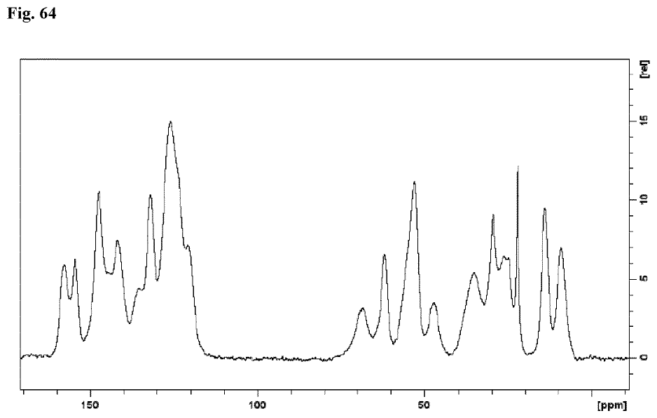

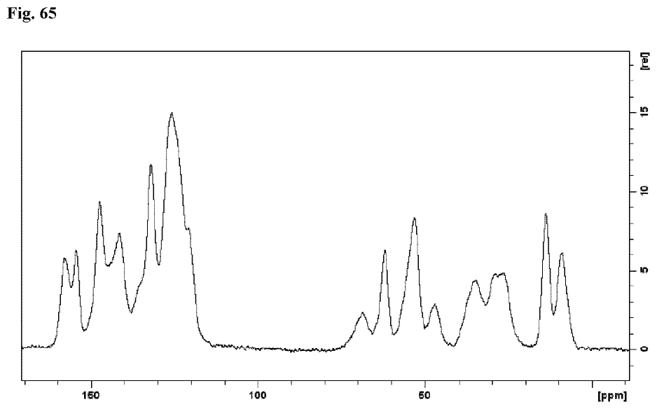

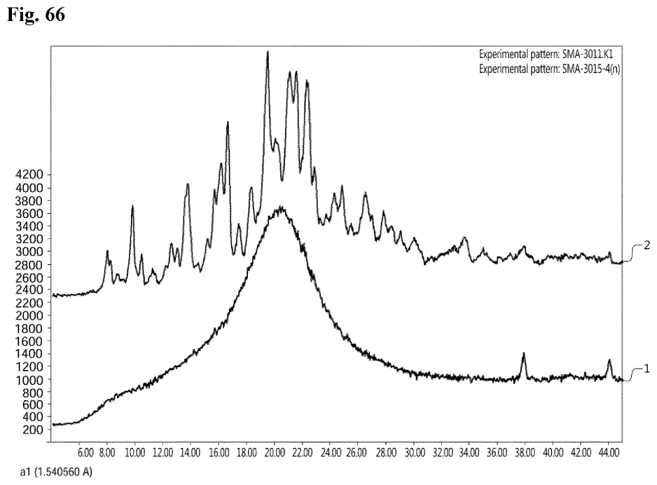

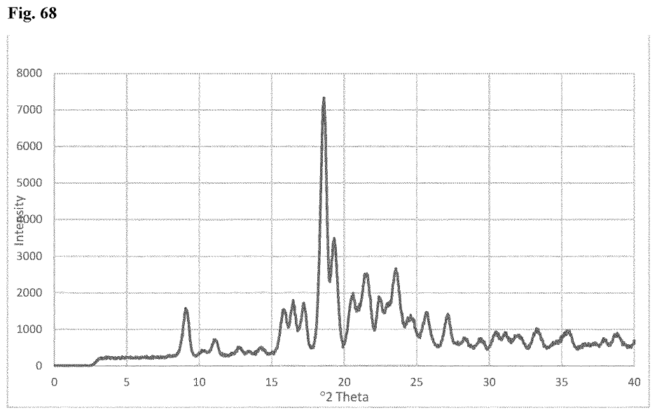

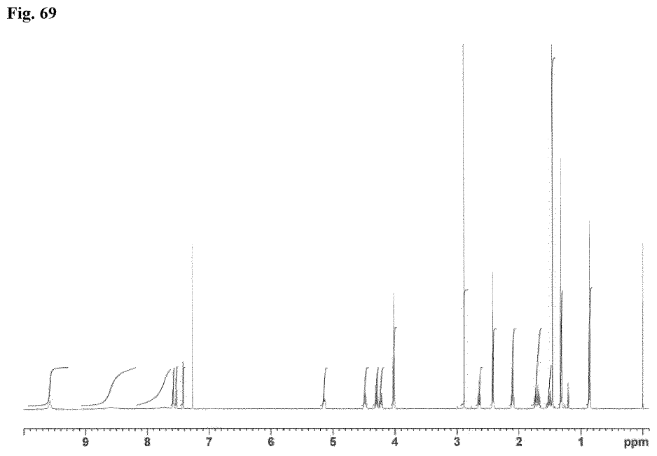

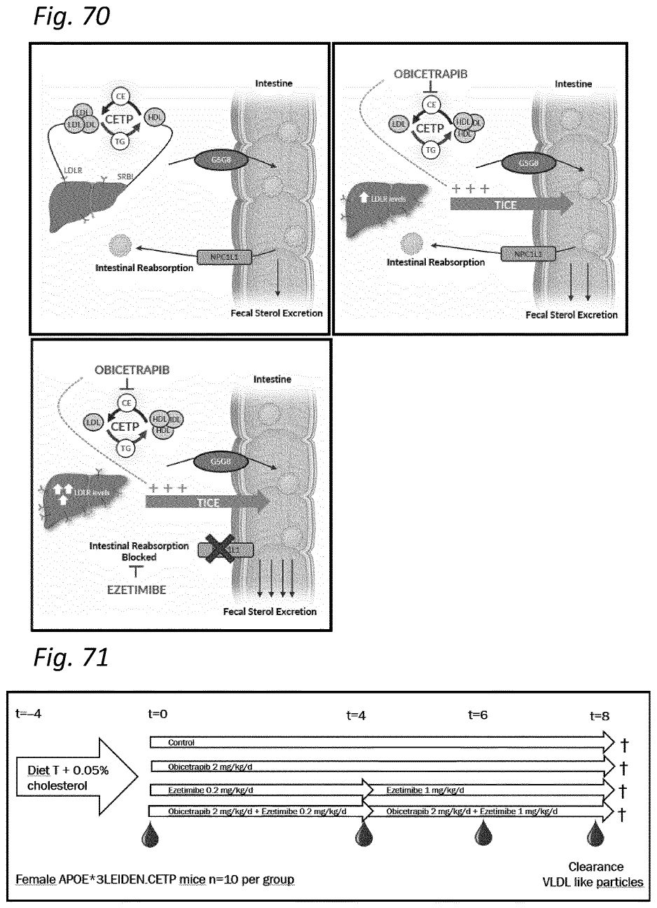

Example 11.18, Example 11.19, Example 11.20, and Example 11.21 set forth various x-ray powder diffraction procedures. The procedure of Example 11.18 was generally used to collect the data set forth in FIGS. 49, 54, 55, and 56; Example 11.19 was generally used for FIG. 50; Example 11.20 was used generally used for FIG. 51; and Example 11.21 was generally used for FIGS. 66, 67, and 68 (with FIG. 68 being for Compound 1D rather than crystalline obicetrapib HCl).

The use of the term “amorphous” in “amorphous obicetrapib hemicalcium” does not mean that the material has no order whatsoever. As shown by the presence of peaks in the x-ray powder diffraction pattern, there is still some order in the sample. Thus, as used herein, the term “amorphous” in “amorphous obicetrapib hemicalcium” does not mean that the x-ray powder diffraction pattern must contain purely an amorphous halo (but may contain halo-like features). Rather, it means that there is disorder, but the amorphous phase is distinguishable from the crystalline phase as discussed below.

Another technique which may be used to distinguish crystalline materials from amorphous materials is polarized light microscopy (“PLM”). In PLM, a material is viewed through polarized light, and by viewing the material through cross-polarizers, one can differentiate between materials that are anisotropic (e.g., crystals) or isotropic (e.g., amorphous compounds). Anisotropic materials, when exposed to polarized light through cross polarizers, exhibit birefringence which manifests itself by exhibiting color change through cross polarizers. Isotropic materials, on the other hand, do not show birefringence and exhibit no color change when exposed to polarized light.

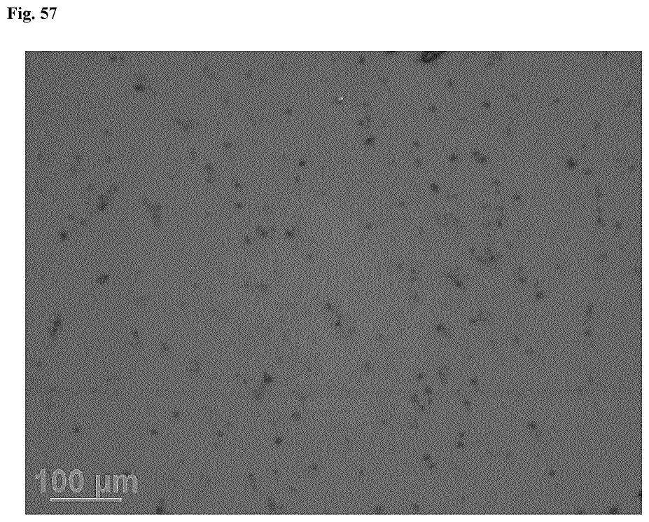

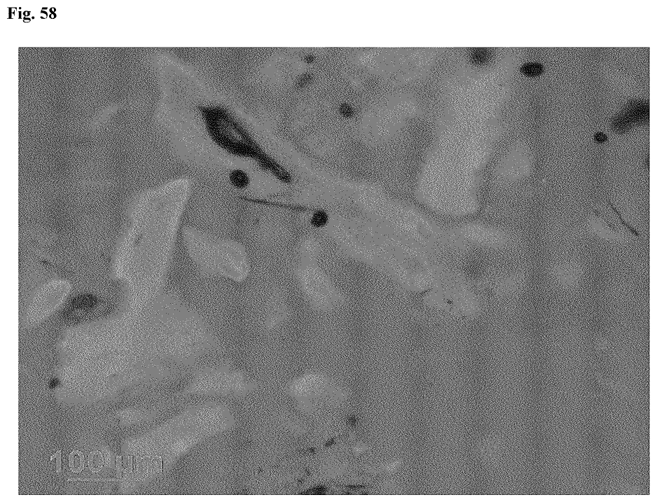

In FIG. 57, amorphous obicetrapib hemicalcium was analyzed by polarized light microscopy as set forth in Example 11.17. As FIG. 57 shows, the materials under study do not birefringe indicating that the material is amorphous. By comparison, FIG. 58 is a polarized light micrograph of crystalline obicetrapib hemicalcium made in accordance with Example 11.16. Notably, the particles shown in FIG. 58 (which is in black and white) exhibits a much brighter contrast. In the corresponding color version, that figure is multicolored. Thus, FIG. 58 indicates crystallinity. In addition, the crystals in FIG. 58 are larger than the particles provided in the amorphous obicetrapib hemicalcium polarized light micrograph of FIG. 57. Accordingly, PLM and/or the lack of birefringence can be used to characterize amorphous obicetrapib hemicalcium.

Other techniques can further be used to distinguish amorphous obicetrapib hemicalcium from crystalline obicetrapib hemicalcium, and therefore can be used to characterize amorphous obicetrapib hemicalcium. One such technique is modulated differential scanning calorimetry also referred to as “mDSC.” The difference in the amount of heat necessary to increase the temperature of a sample, as compared to a reference, is measured as a function of temperature and may be measured using modulated Differential Scanning calorimetry (mDSC). In an mDSC thermogram, one can also measure a glass transition temperature which can be used to characterize an amorphous material. In FIG. 60, for which the procedure is described in Example 11.25, the mDSC thermogram of amorphous obicetrapib hemicalcium was measured using an open sample holder allowing for volatile gases to escape during a measurement. In FIG. 60, the opening was done by piercing a lid on the pan so as to create a pinhole. A glass transition temperature of about 110° C. was recorded for this sample.

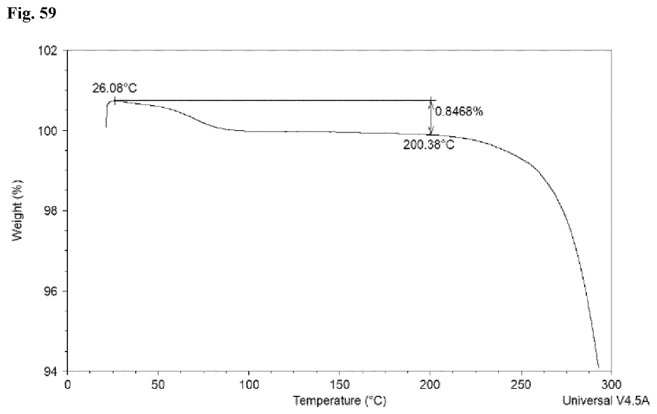

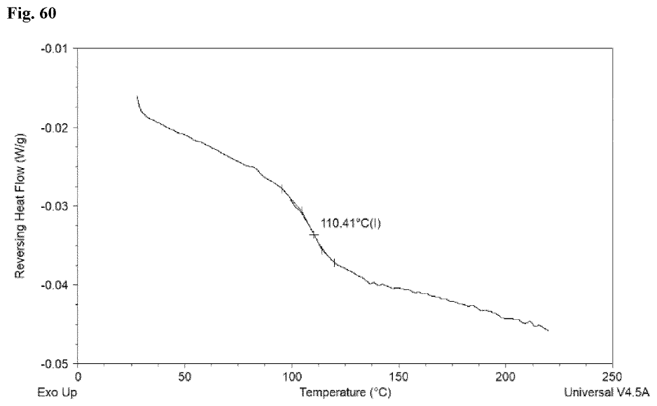

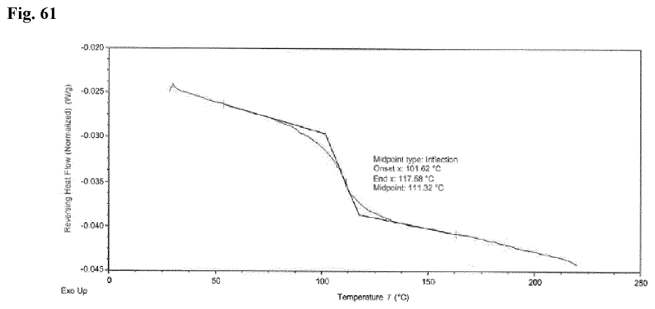

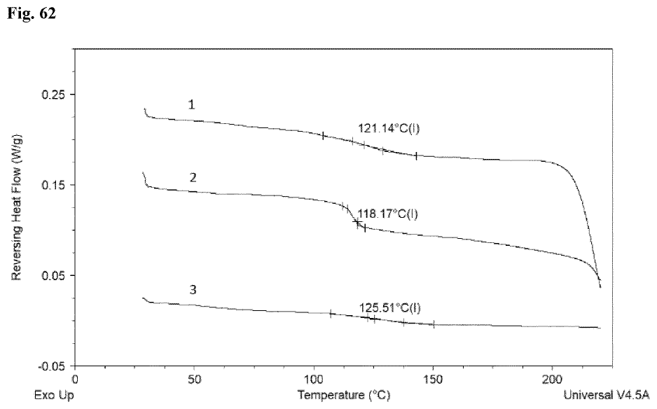

With respect to thermal measurements, the term “about” generally refers to a variability of plus or minus 1° C. By comparison, crystalline obicetrapib hemicalcium has a higher glass transition temperature under the same conditions, and three measurements in FIG. 62 indicate a range between about 118° C. and about 125.5° C. In some embodiments, the glass transition temperature of amorphous obicetrapib hemicalcium is between about 109° C. and 112° C. when measured with a pinhole. In one sample, at Example 11.26, the glass transition temperature of amorphous obicetrapib hemicalcium was found to be about 111° C. (111.32° C. at the midpoint) and is shown in FIG. 61. The onset was measured to be about 102° C. (101.62° C.) and the endpoint about 118° C. (117.58° C.)

The glass transition temperature of amorphous obicetrapib hemicalcium may also be measured using mDSC with a closed pan. The type of sample preparation may affect the measured glass transition temperature. In such cases, the glass transition temperature decreases to temperatures of less than about 100° C. and in particular between about 70° C. and about 92° C. depending on humidity.

Other thermal techniques may also be used to analyze and characterize amorphous obicetrapib hemicalcium such as thermogravimetric analysis (TGA). FIG. 59 is a thermogravimetric analysis thermogram of amorphous obicetrapib hemicalcium showing a weight loss of less than 1% when heated to about 200° C. Such weight losses may be, for example, between about 0.8% and about 0.95% including between about 0.84% and about 0.92%. In FIG. 59, the weight loss was determined to be about 0.85%. This particular material was found to have a water content of about 1.5%. In some embodiments, the water content of may be higher and include a range from about 0% to about 5% water by weight, including up to about 4% by weight, up to about 3% by weight, and between about 0.5% and 1.5% by weight.

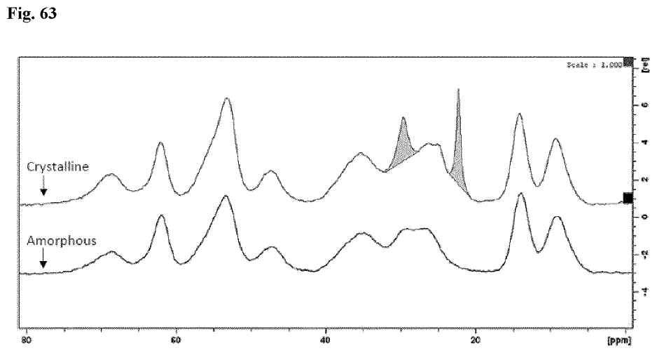

Solid-state ¹³C-NMR spectroscopy is another technique which may be used to characterize amorphous materials. FIG. 63 shows a solid-state ¹³C-NMR spectrum of both crystalline and amorphous obicetrapib hemicalcium with FIG. 64 and FIG. 65 showing the crystalline and amorphous obicetrapib hemicalcium separately. There are at least two differences in the spectra. The crystalline phase has a peak at about 22.1 ppm and which not present in the amorphous phase. In addition, a peak at about 29.5 ppm in the crystalline phase is pronounced while not nearly so in the amorphous phase. Thus, the absence of a solid-state ¹³C-NMR peak at about 22.1 ppm and/or the absence of a pronounced peak at about 29.5 ppm may be used to characterize amorphous obicetrapib hemicalcium. In addition, a solid-state ¹³C-NMR spectrum substantially the same as that of FIG. 65 may be used to characterize amorphous obicetrapib hemicalcium. The absence of a peak in this context does not mean there is necessarily no intensity of, for example, 22.1 ppm or 29.5 ppm, but rather the intensity is not pronounced as it is in the crystalline obicetrapib hemicalcium ¹³C-NMR spectrum.

The properties of crystalline materials also typically differ from those of amorphous materials. Thermodynamically, crystalline materials are more physically stable than amorphous materials. Accordingly, there is a thermodynamic driving force to convert amorphous compounds into crystalline ones. Under accelerated stress conditions, if there would be a physical conversion of solid form, one would therefore generally expect it to be from amorphous to crystalline. However, with obicetrapib hemicalcium, the reverse is the case.

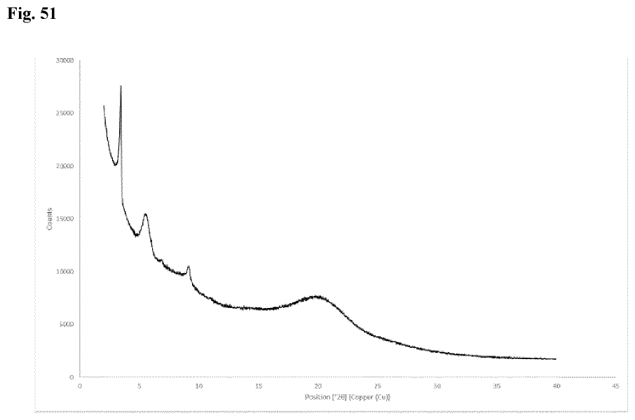

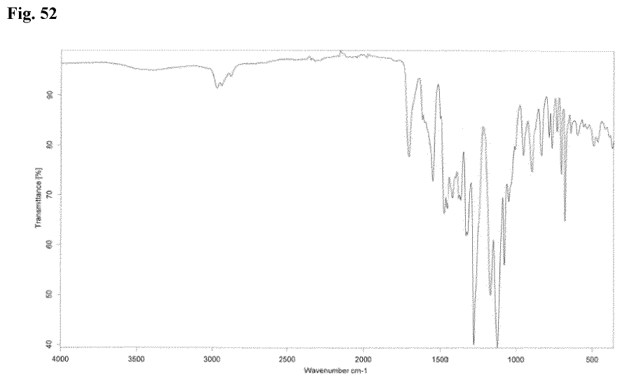

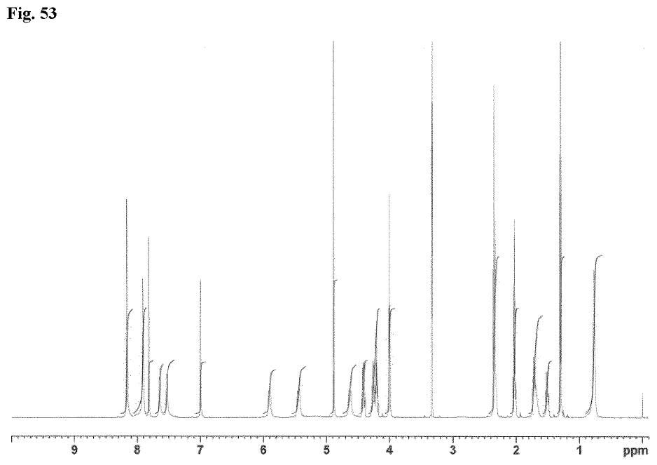

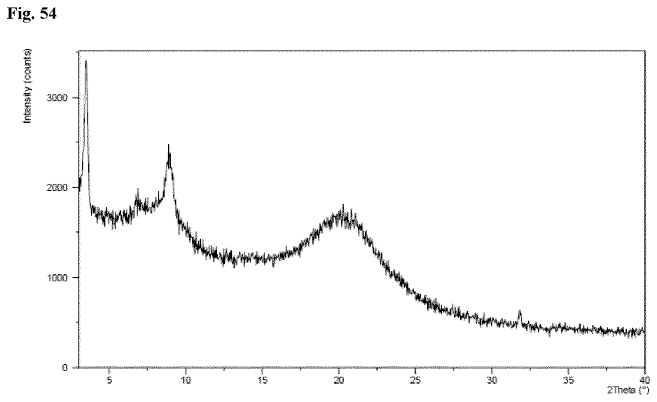

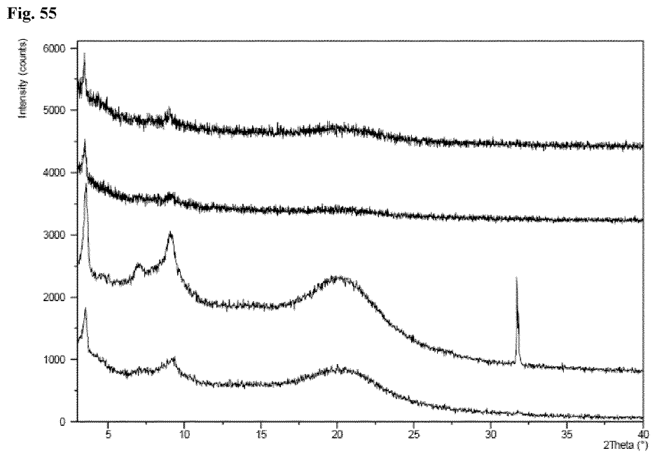

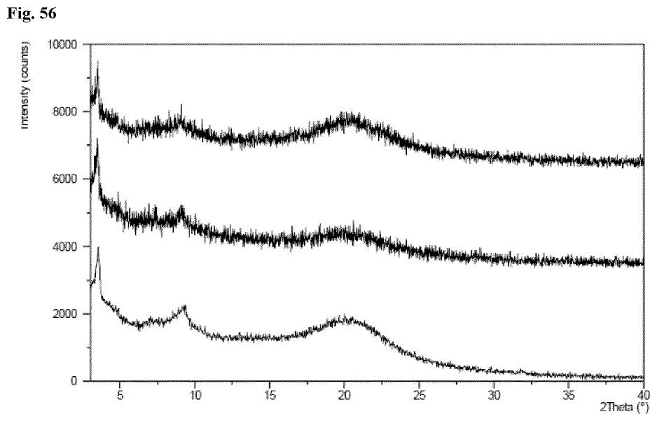

FIG. 54 is a plot of x-ray powder diffraction pattern taken of crystalline obicetrapib hemicalcium, and FIG. 55 is a plot of x-ray powder diffraction patterns taken of crystalline obicetrapib hemicalcium under stress conditions. In FIG. 55, there are four diffraction patterns shown based on stability study set forth in Example 11.27. Pattern 1 is an x-ray powder diffraction pattern of a sample of amorphous obicetrapib hemicalcium. Pattern 2 is the x-ray powder diffraction pattern of a sample of crystalline obicetrapib hemicalcium. In pattern 3, the sample of crystalline obicetrapib hemicalcium was exposed to 70° C. at 75% relative humidity for one day. As can be seen from pattern 3, the x-ray powder diffraction pattern shows the near total loss of crystallinity in that day. After 7 days under the same conditions, the result remains the same as seen in pattern 4. A similar experiment was performed on amorphous obicetrapib hemicalcium shown in FIG. 56. Pattern 1 was taken before the sample was placed on stability. Exposing that material to the same 70° C. and 75% relative humidity conditions did not trigger a crystallization and the material remained amorphous after 7 days (pattern 2) and 14 days (pattern 3). Thus, these experiments suggest, contrary to what one would expect, that the amorphous form of obicetrapib hemicalcium is more stable than crystalline obicetrapib hemicalcium.

In some embodiments of the disclosure, provided herein is stable amorphous obicetrapib hemicalcium. In these embodiments, the amorphous obicetrapib hemicalcium is more physically stable than crystalline obicetrapib hemicalcium under typical pharmaceutical use and processing conditions.

While not wishing to be bound by theory, it is possible that the kinetics here are such that the amorphous phase is kinetically stabilized with respect to the thermodynamically more stable crystalline phase at least under pharmaceutically relevant processing and use conditions. The result of this stability profile is that amorphous obicetrapib hemicalcium is more suitable for pharmaceutical development and use than the corresponding crystalline phases. Despite being more physically resilient, amorphous obicetrapib hemicalcium is more soluble than the highly insoluble crystalline obicetrapib hemicalcium.

Solubility is especially challenging with obicetrapib. At 20° C., for example, the solubility of obicetrapib has been measured to be substantially less than 0.1 mg/mL in water. It would be desirable to have a solid form of obicetrapib that would deliver a larger amount of obicetrapib.

While solubility is a thermodynamic quantity of a material, one can measure the kinetic solubility of a material without necessarily reaching thermodynamic equilibrium. Such measurements provide the solubility under metastable conditions and provide information, for example, of the amount of material undergoing dissolution as a function of time.

The amorphous form has a higher kinetic solubility and dissolution rate than the crystalline form (and by extension obicetrapib itself). Both crystalline and amorphous obicetrapib hemicalcium kinetic solubility determinations were made in biorelevant media at different pHs, namely at about 5.0 (FeSSIF conditions) and at pH of about 6.5 (FaSSIF) conditions as set forth in Example 11.28.

Table W shows the measured solubility of two different batches of amorphous obicetrapib hemicalcium versus crystalline obicetrapib hemicalcium over the course of 2 hours in FeSSIF media at 37° C. In both cases, the amorphous obicetrapib hemicalcium had a higher concentration in solution than the corresponding crystalline material for all time points measured. The concentrations in Table W are those of obicetrapib (i.e., the free acid).

TABLE W
Kinetic Solubility of Crystalline and Amorphous Obicetrapib
Hemicalciumin FeSSIF (pH 5.0) at 37° C.

| | Concentration in Solution (mg/ml) | | | | |
|---|---|---|---|---|---|
| | 15 mins | 30 mins | 60 mins | 90 mins | 120 mins |
| Amorphous | 0.520 | 0.661 | 0.675 | 0.696 | 0.675 |
| obicetrapib | | | | | |
| hemicalcium | | | | | |
| Batch #1 | | | | | |
| Amorphous | 0.502 | 0.618 | 0.628 | 0.658 | 0.642 |
| obicetrapib | | | | | |
| hemicalcium | | | | | |
| Batch #2 | | | | | |
| Crystalline | 0.085 | 0.169 | 0.369 | 0.453 | 0.529 |
| obicetrapib | | | | | |
| hemicalcium | | | | | |

Table X shows a similar experiment at 37° C. but in FaSSIF media at a pH of 6.5. As with Table W, in both batches, the amorphous obicetrapib hemicalcium had a higher concentration in solution than the corresponding crystalline material for all time points measured. The concentrations in Table X are those of obicetrapib (i.e., the free acid).

TABLE X
Kinetic Solubility of Crystalline and Amorphous Obicetrapib
Hemicalcium in FaSSIF (pH 6.5) at 37° C.

| | Concentration in Solution (mg/ml) | | | | |
|---|---|---|---|---|---|
| | 15 mins | 30 mins | 60 mins | 90 mins | 120 mins |
| Amorphous | 0.060 | 0.099 | 0.163 | 0.201 | 0.244 |
| obicetrapib | | | | | |
| hemicalcium | | | | | |
| Batch #1 | | | | | |
| Amorphous | 0.090 | 0.14 | 0.229 | 0.293 | 0.387 |
| obicetrapib | | | | | |
| hemicalcium | | | | | |
| Batch #2 | | | | | |
| Crystalline | 0.030 | 0.048 | 0.084 | 0.107 | 0.144 |
| obicetrapib | | | | | |
| hemicalcium | | | | | |

Because amorphous obicetrapib hemicalcium dissolves faster than the corresponding crystalline phase, more drug is available for immediate use and potentially higher bioavailability in the amorphous phase than in the crystalline phase.

Amorphous obicetrapib hemicalcium is also advantageous because, unlike many amorphous organic compounds, it does not readily pick up moisture. When exposed to relative humidities approaching 90%, moisture uptake has been measured to be typically less than about 5% for example. This lack of hygroscopicity is favorable because it does not require any special handling or storage conditions. Other drawbacks commonly associated with manufacturing and using amorphous materials are similarly not present. For example, amorphous materials are often challenging to make chemically pure. Here, however, amorphous obicetrapib hemicalcium can be made routinely with chemical purities of 99.9% or higher.

In some embodiments of the disclosure, there is provided substantially pure amorphous obicetrapib hemicalcium. In these and other embodiments, the chemical purity of substantially pure amorphous obicetrapib hemicalcium is 99.9% or greater.

In many aspects of the disclosure, there is provided a method of preparing an amorphous calcium salt of obicetrapib, such as amorphous obicetrapib hemicalcium, wherein the method comprises: treating obicetrapib with an acid to form a salt, solvate, or composition; isolating the resulting salt, solvate or composition; and treating that salt, solvate, or composition with a calcium source to create an amorphous obicetrapib calcium salt, such as amorphous obicetrapib hemicalcium. The resulting salt can then be isolated.

Examples of calcium sources include calcium salts such as halogenated calcium salts and soluble calcium salts. In many embodiments, the calcium source is calcium chloride.

The preparation of an amorphous salt of obicetrapib calcium such as amorphous obicetrapib hemicalcium has been found to occur when there is an intermediate salt, solvate or composition (such composition comprising the corresponding acid used to make a salt). Treating obicetrapib directly with a calcium base such as calcium hydroxide has not been found to be a viable way of making an amorphous salt of obicetrapib calcium due to either low solubility, the weakness of the bases available or both. Rather, it has been found that by deploying an intermediate salt, such as a sodium salt, the preparation of amorphous obicetrapib hemicalcium is viable. However, even with a sodium salt, it is preferable for purity and yield purposes to utilize an additional salt or salt-type exchange (such as with the use of a composition or solvate rather than an actual salt) in connection with the sodium salt of obicetrapib. In particular, the use of the salt, solvate, or composition enables the production of a highly pure amorphous calcium salt of obicetrapib such as amorphous obicetrapib hemicalcium.

Exemplary salts that may be made as an intermediate include those from a sulfonate (e.g., besylate, tosylate, napsylate, camsylate, esylate, edisylate, or mesylate), a sulfate (e.g., methylsulfate), a halogen (e.g., chloride, iodide, or bromide), acetate, aspartate, benzoate, bicarbonate, bitartrate, carbonate, citrate, decanoate, fumarate, gluceptate, gluconate, glutamate, glycolate, hexanoate, hydroxynaphthoate, isethionate, lactate, lactobionate, malate, maleate, mandelate, mucate, nitrate, octanoate, oleate, pamoate, pantothenate, phosphate, polygalacturonate, propionate, salicylate, stearate, succinate, tartrate, or a teoclate. When the intermediate is a solvate or a composition, then the corresponding acids may be used or present. In addition, when a solvate, the intermediate may further include a solvent such as an organic solvent or water, in which case the solvate would be a hydrate. One such organic solvent is CPME (cyclopentyl methyl ether).

In some embodiments, the intermediate is a solvate of an acid. In these and other embodiments, the intermediate is a solvate of an acid and an organic solvent. In some particular embodiments, the intermediate is a solvate comprising an acid and a solvent. In some of these embodiments, the acid is hydrochloric acid and a solvent is CPME.

In many aspects of the disclosure, the disclosure includes methods for preparing amorphous obicetrapib calcium salts, such as amorphous obicetrapib hemicalcium. The disclosure further includes amorphous obicetrapib calcium salts, including amorphous obicetrapib hemicalcium, so prepared. In one such preparation, an intermediate referred to herein as crystalline obicetrapib HCl is used in the processes for preparing amorphous obicetrapib calcium, such as amorphous obicetrapib hemicalcium.

In many aspects of the disclosure, amorphous obicetrapib hemicalcium is prepared via a chemical synthesis where an intermediate is used denoted by Formula (IH):

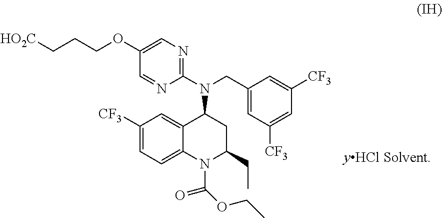 [SMILES: CCOC(=O)N1c2ccc(C(F)(F)F)cc2[C@@H](N(Cc2cc(C(F)(F)F)cc(C(F)(F)F)c2)c2ncc(OCCCC(=O)O)cn2)C[C@H]1CC]

Where y varies such that the mass percent of HCl varies from 0.01% to 8% by weight and is believed to further include an associated organic solvent such as by way of a solvate. In some embodiments, y varies from 0.002 to 1.5. In some embodiments, y varies from 0.3 to 1. In some embodiments, y varies from 0.4 to 0.6, including between 0.5 and 0.6. In some embodiments, Formula (IH), as a solvate, is isolated in its crystalline form. In many embodiments, the solvent is CPME. Other solvents which may form solvates include toluene and heptane.

Obicetrapib HCl as typically prepared herein is crystalline. Further, the term crystalline obicetrapib HCl may include CPME as a solvate when CPME is used in the preparation of crystalline obicetrapib HCl. In Formula (IH), the solvate is of an organic solvent and in many embodiments, that solvent is CPME. In some embodiments, the disclosure provides for compositions comprising crystalline obicetrapib HCl.

Formula (IH) is referred to as obicetrapib HCl and when crystalline, it is referred to as crystalline obicetrapib HCl.

Without being bound by theory, it is believed that crystalline HCl obicetrapib is a mixed salt solvate. It has been found that when CPME is used to deliver HCl in the reaction to create Formula (IH), the chloride content of Formula (IH) ranges between about 2.5% and 3.0% by weight which is below what one would expect for a neutral salt-namely about 4.8% by weight.

In many embodiments, when CPME is so used, it is found in the material when crystallized. When CPME is used in the reaction to deliver dry HCl and is thus found in the crystallized material, the resulting crystalline Formula (IH) material is referred to as crystalline obicetrapib HCl, those x-ray powder diffraction pattern are seen in FIG. 66. An advantage of using crystalline obicetrapib HCl as an intermediate is that the resulting amorphous obicetrapib hemicalcium has a chemical purity which is routinely 99.9% pure or greater. Chemical purity is the quantitative representation of whether other chemical entities other than the compound being measured are present. For example, a chemical purity of 99.9% amorphous obicetrapib hemicalcium means that not more than 0.1% of the compounds in a sample of amorphous obicetrapib hemicalcium are other entities. Physical purity refers to the amount of other solid forms of the same compound present which, in the case of amorphous obicetrapib hemicalcium, the other solid form being crystalline obicetrapib hemicalcium. The disclosure herein provides for amorphous obicetrapib hemicalcium which is physically pure meaning it is free or substantially free of crystalline obicetrapib hemicalcium. Unless otherwise stated herein, the purity measurements provided herein are measurements of chemical purity.

HCl obicetrapib, as used herein, is not limited to crystalline obicetrapib HCl. Indeed, upon desolvation, crystalline obicetrapib HCl may become amorphous.

Upon stress, crystalline obicetrapib HCl loses its crystallinity. In FIG. 66, pattern 2 reflects crystalline obicetrapib HCl subject to a mild drying treatment whereby surface solvent was removed and it can be seen that this compound is crystalline. By comparison, the sample whose x-ray powder diffraction was measured in pattern 1 was subject to a stronger drying treatment at 48 hours at 55° C. at a pressure of 2 mbar. As is apparent, this drying changed the material from crystalline to amorphous, likely due to loss of HCl and a desolvation of CPME. ¹H-NMR spectroscopy, for example, was used to show the presence of CPME in the top pattern, but was substantially absent in the lower, amorphous pattern. The amorphous pattern, therefore, represents HCl obicetrapib which is not crystalline obicetrapib. It may be obicetrapib, but is believed to have HCl associated with the obicetrapib as a solvate and thus is HCl obicetrapib, but with a lower chloride content than typically found in the ranges found for crystalline obicetrapib HCl. In some embodiments, that chloride content is less than 0.1% by weight such as between about 0.01% and 0.1% by weight.

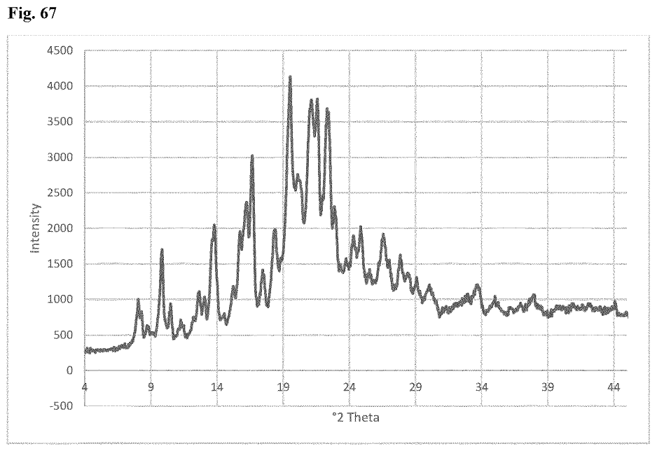

Crystalline obicetrapib HCl may be characterized by an x-ray powder diffraction pattern comprising a peak at about 9.8°2θ. In some embodiments, crystalline obicetrapib HCl may be characterized by an x-ray powder diffraction pattern comprising one or more peaks at about 8.1°2θ, about 9.8°2θ, about 13.8°2θ, about 16.7°2θ, or about 19.5°2θ. Table Y provides illustrative peaks which may be present in crystalline obicetrapib HCl. In some embodiments, crystalline obicetrapib HCl may be characterized by an x-ray powder diffraction pattern substantially the same as that in FIG. 67, although it is believed that the material analyzed in FIG. 67 was measured in such a way that a peak between about 4.3°2θ and about 4.7°2θ was not measured.

TABLE Y

| °2θ | Intensity |
|---|---|
| 8.1 | 950 |
| 9.8 | 1650 |
| 13.8 | 2000 |
| 16.7 | 2900 |
| 19.5 | 4100 |
| 21.1 | 3700 |
| 21.6 | 3800 |
| 22.4 | 3600 |
| 24.9 | 1950 |
| 26.6 | 1850 |

Another intermediate used in the preparation of obicetrapib is that of Formula (VI)

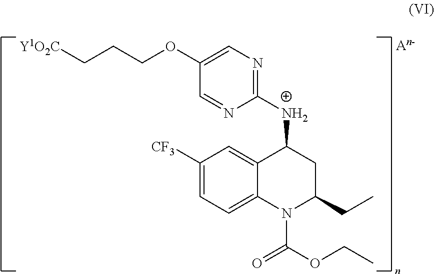

wherein Y¹ is a protecting group (e.g., as described herein); Aⁿ⁻ is an anion; and n is an integer from 1-3.

In one embodiment, the compound of Formula (VI) is a mesylate salt where n is 1, Y¹ is t-butyl, and has the structure of Compound 1D:

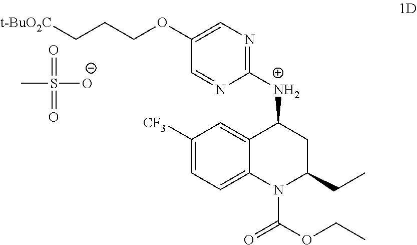

A ¹H-NMR spectrum of Compound 1D (in solution) can be found in FIG. 69. Crystalline Compound 1D may be characterized by an x-ray powder diffraction pattern comprising one or more peaks at about 5.2°2θ or about 9.1°2θ. In some embodiments, crystalline Compound 1D may be characterized by an x-ray powder diffraction pattern comprising one or more peaks at about 5.2°2θ, about 9.1°2θ, about 15.9°2θ, about 16.5°2θ, about 17.2°2θ, about 18.6°2θ, and about 19.2°2θ. Table Z provides illustrative peaks which may be present in crystalline Compound 1D (with the peak at about 5.2°2θ not measured due to instrument limitations in reflection mode). In some embodiments, crystalline Compound 1D may be characterized by an x-ray powder diffraction pattern substantially the same as FIG. 68.

TABLE Z

| °2θ | Intensity |
|---|---|
| 9.1 | 1530 |
| 15.9 | 1470 |
| 16.5 | 1700 |
| 17.2 | 1680 |
| 18.6 | 7331 |
| 19.2 | 3400 |
| 20.6 | 1870 |
| 21.6 | 2440 |
| 22.5 | 1800 |
| 23.5 | 2580 |
| 25.7 | 1400 |
| 27.2 | 1370 |

Crystalline compounds such as crystalline Compound 1D and a crystalline obicetrapib HCl, for example, may be characterized by x-ray powder diffraction. An x-ray powder diffraction pattern is an x-y graph with °2θ (diffraction angle) on the x-axis and intensity on the y-axis. The peaks are usually represented and referred to by their position on the x-axis rather than the intensity of peaks on the y-axis because peak intensity can be particularly sensitive to sample orientation (see Pharmaceutical Analysis, Lee & Web, pp. 255-257 (2003)). Thus, intensity is not typically used to characterize solid forms. The data from x-ray powder diffraction may be used in multiple ways to characterize crystalline forms. For example, the entire x-ray powder diffraction pattern output from a diffractometer may be used to characterize a crystalline obicetrapib HCl compound or a crystalline Compound 1D. A smaller subset of such data, however, may also be, and typically is, suitable for characterizing such compounds. For example, a collection of one or more peaks from such a pattern may be used to so characterize these compounds. When the phrase “one or more peaks” of a list of peaks from an x-ray powder diffraction pattern are provided, what is generally meant is that any combination of the peaks listed may be used for characterization. Further, the fact that other peaks are present in the x-ray powder diffraction pattern, generally does not negate or otherwise limit that characterization.

In addition to the variability in peak intensity, there may also be variability in the position of peaks on the x-axis. This variability can, however, typically be accounted for when reporting the positions of peaks for purposes of characterization. Such variability in the position of peaks along the x-axis may derive from several sources (e.g., sample preparation, particle size, moisture content, solvent content, instrument parameters, data analysis software, and sample orientation). For example, samples of the same crystalline material prepared under different conditions may yield slightly different diffractograms, and different x-ray instruments may operate using different parameters and these may lead to slightly different diffraction patterns from the same crystalline solid. Due to such sources of variability, it is common to recite x-ray diffraction peaks using the word “about” prior to the peak value in °2θ. For purposes of data reported herein, that value is generally ±0.2°2θ are intended to be reported with such a variability whenever disclosed herein whether the word “about” is present or not. Variability may, in some instances, be higher depending on instrumentation conditions including how well instruments are maintained.

In some embodiments, crystalline Compound 1D may be further characterized by an x-ray powder diffraction pattern substantially the same as the x-ray powder pattern as that of FIG. 68.

In many aspects of the disclosure, there is provided a method of preparing an amorphous calcium salt of obicetrapib, such as amorphous obicetrapib hemicalcium, wherein the method comprises:
i. treating obicetrapib with HCl to obtain crystalline obicetrapib HCl;
ii. isolating crystalline obicetrapib HCl;
iii. preparing an amorphous calcium salt of obicetrapib, such as amorphous obicetrapib hemicalcium, from the crystalline obicetrapib HCl isolated in step (ii); and
iv. isolating an amorphous calcium salt of obicetrapib, such as amorphous obicetrapib hemicalcium.

In other aspects of the disclosure, there is provided a method of preparing obicetrapib wherein the method comprises:
(a) preparing a compound of Formula (IV), by coupling a compound of Formula (II) or a salt thereof, with a compound of Formula (III);

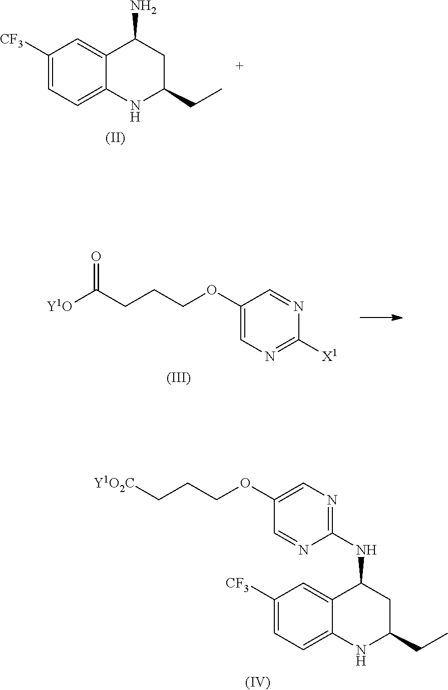

where X₁ is a leaving group and Y¹ is a protecting group;
(b) preparing a carbamate of Formula (V) from the compound of Formula (IV) and isolating as a solid salt form of Formula (VI):

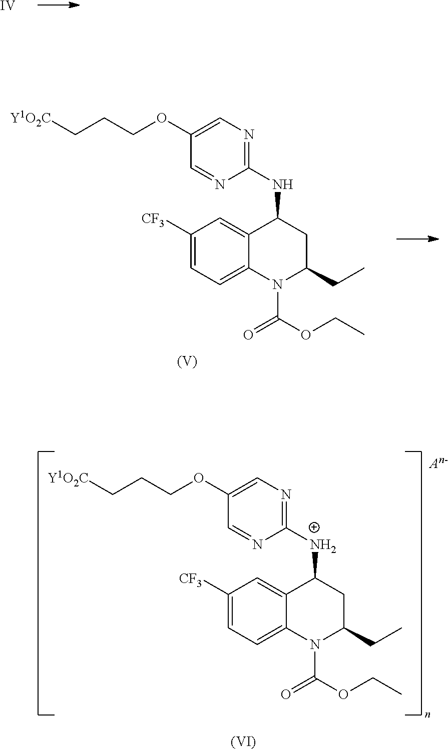

where Y¹ is a protecting group, Aⁿ⁻ is an anion and wherein n is an integer from 1-3;
(c) optionally desalting the compound of Formula (VI) and alkylating with a compound of Formula (VII) to provide a compound of Formula (VIII):

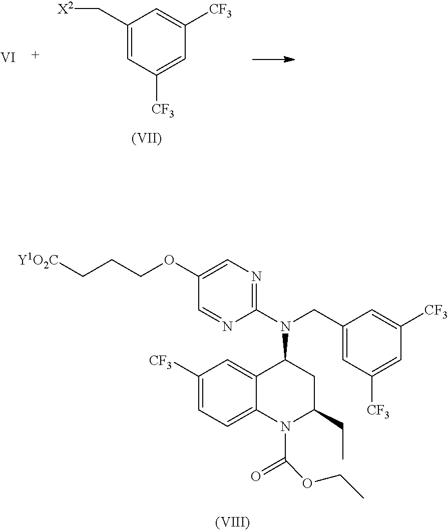

where, X₂ is a leaving group, Y¹ is a protecting group; and
(d) converting the compound of Formula (VIII) to obicetrapib, wherein the reaction steps (a)-(d) are performed in an organic solvent, compounds (IV), (V) and (VIII) are optionally not isolated from the organic solvent, and wherein the process does not need to comprise chromatography.

The reactions in steps (a)-(d) of the subject method are performed in a solvent, and intermediate compounds of Formulae (IV), (V) and (VIII) do not need to be isolated from their respective solvents if they are to be processed further to end products. This means that any solvent swap between reaction steps (x) and (x+1) takes places by evaporating at least part of the solvent used in step (x) and by gradually adding the solvent of step (x+1), such that the compound remains in solution during the solvent swap. The intermediate compound of Formula (VI) may be isolated from the solvent as a salt in solid form, such that it can be washed to remove impurities. This isolation step ensures sufficient purity of downstream products. The subject process does not need to comprise purification steps using chromatography, such as column chromatography to achieve the chemical purity levels described herein.

Method of Preparing an Amorphous Calcium Salt Such as Amorphous Obicetrapib Hemicalcium)—Steps (i)-(ii) from Aspects (i)-(iv)

In some embodiments of the method of preparing an amorphous calcium salt of obicetrapib such as amorphous obicetrapib hemicalcium, the method includes step (i), treating obicetrapib with HCl in an organic solvent to obtain crystalline obicetrapib HCl.

In some embodiments, crystalline obicetrapib HCl has a purity of 98% or more, such as 98.5% or more, 99% or more, 99.5% or more, or even more.

In some embodiments, the HCl in step (i) is in a suitable solvent. Such solvent may be an aqueous solvent or an organic solvent. In some embodiments, the organic solvent used in step (i) comprises a mixture of a solvent and an anti-solvent. In some embodiments, the solvent is selected from methanol, ethanol, isopropanol, acetic acid, acetonitrile, acetone, methyl isobutyl ketone, isopropyl acetate, tetrahydrofuran, methyl t-butyl ether, cyclopentyl methyl ether, N-methyl-2-pyrrolidone, dimethyl sulfoxide, dimethylformamide, 2-methyl-tetrahydrofuran, dichloromethane, 1,4-dioxane, 1,2-diflurobenzene, toluene, hexafluoroisopropanol, and water. In some embodiments, the anti-solvent is selected from n-heptane, n-hexane, n-pentane, and cyclohexane.

In some embodiments, the HCl has sufficient solubility in the anti-solvent such that it can be used as a suitable solvent. In some embodiments, the organic solvent used in step (i) comprises a mixture of cyclopentyl methyl ether and n-heptane. In some embodiments, the organic solvent used in step (i) further comprises toluene. In some embodiments, toluene is the majority component of the organic solvent.

In some embodiments, step (i) comprises providing obicetrapib in a mixture of cyclopentyl methyl ether and n-heptane, raising the temperature to between 35° C. and 40° C. under agitation, adding dry HCl in cyclopentyl methyl ether and raising the temperature again to between 50° C. and 55° C., then adding further n-heptane as an anti-solvent. At this point, a small portion of the reaction mixture can optionally be extracted, cooled to a temperature of between 10° C. and 15° C., to obtain a slurry of crystals of crystalline obicetrapib HCl in a mixture of cyclopentyl methyl ether and n-heptane (referred to herein as a “seed crystal slurry”). Optionally, all or a portion of the seed crystal slurry of crystalline obicetrapib HCl can then be added back to the reaction mixture. The seeds assist with nucleation but are not required. The resulting reaction mixture is then cooled to a temperature between 5° C. and 15° C. (such as from 10° C. to 15° C.), followed by crystallizing the crystalline obicetrapib HCl from the system under agitation. In some embodiments, the crystalline obicetrapib HCl is crystallized over a period of 12 hours or more, with subsequent filtration (e.g., through a filter dryer), one or more optional washing steps, such as with a mixture of cyclopentyl methyl ether and n-heptane, and drying. In some cases, a wet filter cake of crystalline obicetrapib HCl is dried in vacuo in steps using temperatures of 25° C.-30° C., 30° C.-40° C., 40° C.-50° C. then 50° C.-55° C., such as 25° C., 35° C., 46° C., and 54° C.

In some embodiments, the method of preparing crystalline obicetrapib HCl comprises the addition of seed crystals (e.g., as a seed crystal slurry). The seed crystals of an HCl compound can be formed as a slurry by following step (i) as set out above and after addition of dry HCl in cyclopentyl methyl ether and anti-solvent n-heptane, extracting a small portion of the reaction mixture, cooling to a temperature between 10° C. and 15° C., to provide a slurry of crystals of crystalline obicetrapib HCl in cyclopentyl methyl ether and n-heptane.

Accordingly, in one embodiment, step (i) comprises providing crystalline obicetrapib HCl in a mixture of cyclopentyl methyl ether and n-heptane, raising the temperature to between 35° C. and 45° C. under agitation, adding dry HCl in cyclopentyl methyl ether and raising the temperature again to between 50° C. and 55° C., addition of further n-heptane as anti-solvent, and the optional addition of seed crystals of an HCl compound (e.g., as a seed crystal slurry prepared as described herein), cooling to a temperature between 5° C. and 15° C. (such as from 10° C. to 15° C.), followed by crystallizing the crystalline obicetrapib HCl from the system under agitation. In some embodiments, the crystalline obicetrapib HCl is crystallized over a period of 12 hours or more, with subsequent filtration, one or more optional washing steps, such as with a mixture of cyclopentyl methyl ether and n-heptane, and drying. In some embodiments, crystalline obicetrapib HCl is dried in vacuo. In some embodiments, crystalline obicetrapib HCl is subjected to drying in a vacuum drying cabinet at 25 mbar pressure and at a temperature of 55° C. for 10 hours or more. In some embodiments, after the drying procedure, the crystalline obicetrapib HCl includes less than 0.1 weight percent residual cyclopentyl methyl ether.

In some embodiments, step (i) comprises providing the solution of obicetrapib in cyclopentyl methyl ether with a concentration between 30 and 40 weight percent, such as from 33 to 37 weight percent, based on the weight of the solution, less than 1 weight percent of the first organic solvent used in step (d) (such as toluene), less than 1 weight percent of n-heptane based on weight of solution, addition of n-heptane, raising the temperature to 35° C. to 45° C. under agitation, adding dry HCl in cyclopentyl methyl ether and raising the temperature again to 50° C. to 55° C., addition of further n-heptane as anti-solvent, optional addition of seed crystals of a crystalline obicetrapib HCl (e.g., as a seed crystal slurry prepared as described herein), cooling to a temperature between 10° C. and 15° C., followed by crystallizing crystalline obicetrapib HCl from the system under agitation, such as during a period of at least 12 hours, with subsequent filtration, one or more washing steps with a mixture of cyclopentyl methyl ether and n-heptane, and drying, such as in vacuo. In some embodiments, the amount of toluene is substantially greater.

In some embodiments the crystalline obicetrapib HCl from step (i) is isolated in step (ii). In some embodiments, the isolated crystalline obicetrapib HCl has a purity of 98% or more, such as 98.5% or more, 99% or more, 99.5% or more, 99.7%, or even more.

Another embodiment of the disclosure concerns the crystalline obicetrapib HCl, obtained by or obtainable by the process as defined herein.

Still another embodiment of the disclosure is directed to HCl obicetrapib, including crystalline obicetrapib HCl.

In some embodiments, the crystalline obicetrapib HCl is stored at controlled room temperature and under a nitrogen atmosphere and is protected from moisture to prevent the formation of an amorphous solid such as from desolvation.

Method of Preparing an Amorphous Calcium Salt of Obicetrapib, Such as Amorphous Obicetrapib Hemicalcium—Steps (iii)-(iv) from Aspects (i)-(iv)

In some embodiments of the method of preparing an amorphous calcium salt of obicetrapib, such as amorphous obicetrapib hemicalcium, the method includes step (iii)-(iv), preparing an amorphous calcium salt of obicetrapib from crystalline obicetrapib HCl isolated in step (ii), and isolating an amorphous calcium salt of obicetrapib, such as amorphous obicetrapib hemicalcium.

In some embodiments of the method of isolating an amorphous calcium salt of obicetrapib according to step (iv), the amorphous calcium salt of obicetrapib is in the form of amorphous obicetrapib hemicalcium:

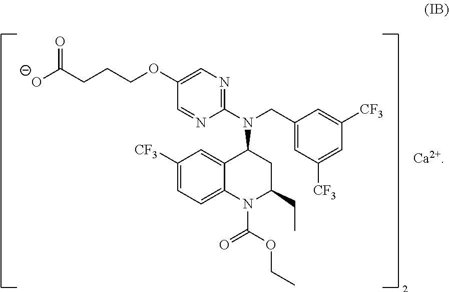

In some embodiments of the method of preparing obicetrapib, step (iii) includes the following steps:
(iii-1) converting crystalline obicetrapib HCl of step (ii) to provide obicetrapib in an organic solvent;
(iii-2) treating obicetrapib in the organic solvent with aqueous sodium hydroxide to form a sodium salt of obicetrapib; and
(iii-3) treating the sodium salt of obicetrapib with aqueous calcium chloride to form amorphous obicetrapib hemicalcium;

wherein the compounds in steps (iii-1) and (iii-2) are not isolated.

Accordingly, in some embodiments step, (iii-1) comprises the following steps:
(aa) providing crystalline obicetrapib HCl, as isolated in step (ii);
(bb) dissolving crystalline obicetrapib HCl in a mixture of water and isopropyl acetate under agitation. In some embodiments, step (bb) is conducted at a temperature between 15° C. and 25° C.;
(cc) allowing phase separation and subjecting the resulting organic phase to one or more subsequent washing steps with water, wherein each washing step is followed by separating off the aqueous phase, resulting in a washed organic phase; and
(dd) performing two or more distillations on the washed organic phase resulting from step (cc) at a temperature of 50° C. or lower (such as 30° C. or lower), with intermediate additions of ethanol, to obtain a solution of obicetrapib in ethanol.

In some embodiments step, (iii-2) comprises the following steps:
(ee) adding an aqueous NaOH solution to the solution obtained in step (dd) and agitating the resulting mixture, such as at a temperature between 20° C. and 25° C. for at least 4 hours, to obtain a solution of the sodium salt of obicetrapib; and
(ff) optionally filtering the solution obtained in step (ee).

In some embodiments step, (iii-3) comprises the following steps:
(gg) preparing a CaCl₂) solution by adding deionized water to CaCl₂) under agitation, followed by adding ethyl acetate as a co-solvent, and stirring the resulting mixture for 10 to 30 minutes;
(hh) cooling the CaCl₂) solution obtained in step (gg) to a temperature from 8° C. to 12° C. and adding via a filter to the solution obtained in step (ff) or (ee) under agitation at said temperature;
(ii) stirring the slurry resulting from step (hh) for about 1 to about 10 hours. In some embodiments of step (ii), the stirring is conducted at a temperature between 8° C. and 12° C.;
(jj) isolating the solids from the slurry obtained in step (ii) by filtration. In some embodiments of step (jj), the isolating is conducted at a temperature between 8° C. and 12° C.;
(kk) washing the filtration residue obtained in step (jj) with water in one or more washing steps. In some embodiments of step (kk), the washing is conducted at a temperature between 8° C. and 12° C.; and
(ll) drying the washed residue obtained in step (kk), such as in vacuo at a temperature from 40° C. to 50° C. for more than 16 hours (such as 50 hours, 100 hours, 150 hours, or 200 hours, or even more), to obtain amorphous obicetrapib hemicalcium (also sometimes referred to herein as compound 3).

In some embodiments, amorphous obicetrapib hemicalcium is submitted to a subsequent reworking procedure. In some embodiments, amorphous obicetrapib hemicalcium is further reworked by dissolving in ethanol (such as twice the weight of ethanol relative to amorphous obicetrapib hemicalcium) at a temperature of 25° C. to 50° C., followed by cooling to 10° C. to 15° C., followed by filtering into a mixture of aqueous calcium chloride solution and ethyl acetate, also cooled to 10° C. to 15° C., followed by filtering, washing with water and drying in vacuo at 45° C. or less for 20 hours or more.

In some embodiments of step (iv), amorphous obicetrapib hemicalcium is isolated with a purity of 95% or more, such as a purity of 95.5% or more, 96% or more, 96.5% or more, 97% or more, 97.5% or more, 98% or more, 98.5% or more, 99% or more, 99.1% or more, 99.2% or more, 99.3% or more, 99.4% or more, 99.5% or more, 99.6% or more, 99.7% or more, 99.8% or more, or 99.9% or more.

In some embodiments, amorphous obicetrapib hemicalcium is subjected to a milling process. In some embodiments, the milling process is adapted (e.g., parameters such as feed rate, venturi pressure and mill pressure are adapted) to allow production of micronized amorphous obicetrapib hemicalcium.

Method of Preparing Obicetrapib—Step (a) from Aspects (a)-(d)

In step (a) of the process for preparing obicetrapib according to the present disclosure, the compound of Formula (II), or a salt thereof, is coupled with a compound of Formula (III) to provide a compound of Formula (IV) (where X¹ is a leaving group and Y¹ is protecting group, e.g., as described herein).

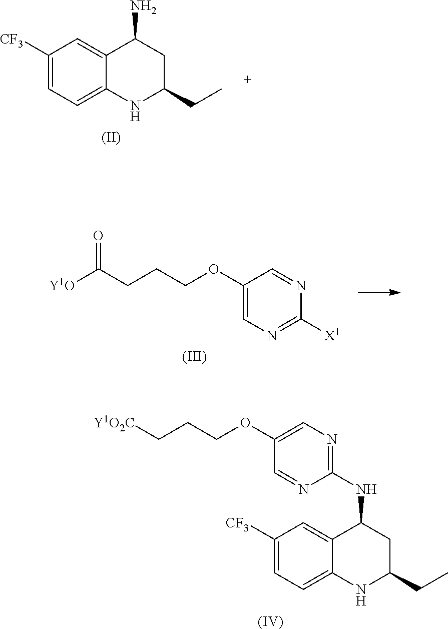

Step (a) of the subject method, starts with a compound of Formula (II) (2R,4S)-4-amino-2-ethyl-6-trifluoromethyl-3,4-dihydro-2H-quinoline), or a salt thereof:

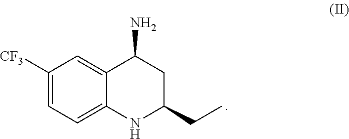 [SMILES: CC[C@@H]1C[C@H](N)c2cc(C(F)(F)F)ccc2N1]

The compound of Formula (II) can for example be obtained using a process as disclosed in WO2016/024858A1 or in WO2007/116922A1, both of which are incorporated herein by reference in their entirety. In some embodiments the compound of Formula (II) can be obtained from a corresponding salt that is stable and can be obtained in pure and solid form. The solid form can be amorphous or crystalline. In some embodiments, the compound of Formula (II) is obtained from a corresponding crystalline salt.

In some embodiments, the compound of Formula (II) provided in step (a) is a salt of the Formula (IIA) or (IIB):

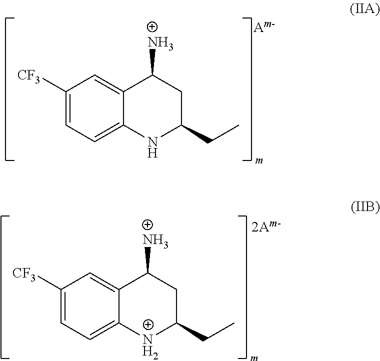

wherein A^{m-} is an anion and n is an integer from 1-3.

In some embodiments, the compound of Formula (II) provided in step (a) is a salt of Formula (IIA). In some embodiments, the compound of Formula (IIA) is used directly in the coupling reaction with the compound of Formula (III) without performing a desalting step.

In some embodiments, the compound of Formula (II) provided in step (a) is a salt of Formula (IIB). In some embodiments, the compound of Formula (IIB) is used directly in the coupling reaction with the compound of Formula (III) without performing a desalting step.

In some embodiments, the compound of Formula (II) in step (a) is obtained from a salt of Formula (IIA) or (IIB). In some embodiments, the following steps are carried out before the coupling reaction of step (a):
(pre-a1) providing a compound of Formula (IIA) or (IIB):

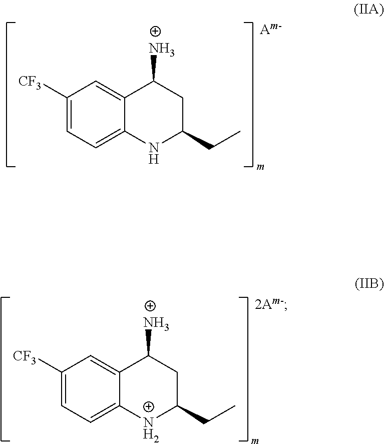

and
(pre-a2) desalting the compound of Formula (IIA) or (IIB) to obtain the compound of Formula (II),

wherein the reaction in step (pre-a2) is performed in an organic solvent, the compound of Formula (II) is not isolated from the organic solvent, and the process does not comprise chromatography.

In some embodiments, the compound of Formula (II) in step (a) is obtained from a salt of Formula (IIA). In some embodiments, the compound of Formula (II) in step (a) is obtained from a salt of Formula (IIB).

In some embodiments the salts of Formula (IIA) or (IIB), are chosen from salts with an anion A^{m-} selected from a sulfonate (e.g., besylate, tosylate, napsylate, camsylate, esylate, edisylate, or mesylate), a sulfate (e.g., methylsulfate), a halogen (e.g., chloride, iodide, or bromide), acetate, aspartate, benzoate, bicarbonate, bitartrate, carbonate, citrate, decanoate, fumarate, gluceptate, gluconate, glutamate, glycolate, hexanoate, hydroxynaphthoate, isethionate, lactate, lactobionate, malate, maleate, mandelate, mucate, nitrate, octanoate, oleate, pamoate, pantothenate, phosphate, polygalacturonate, propionate, salicylate, stearate, succinate, tartrate, and teoclate.

In some embodiments the salts of Formula (IIA) or (IIB), are chosen from salts with an anion A^{m-} selected from chloride, bromide, bitartrate, a sulfate, and a sulfonate.

In some embodiments the salts of Formula (IIA) or (IIB), are chosen from salts with an anion A^{m-} selected from chloride, bromide, bitartrate, and mesylate.

In some embodiments of the salts of Formula (IIA) or (IIB), m is 1.

In some embodiments the salt is of Formula (IIA), and the anion A^{m-} is mesylate, where m is 1. The mesylate (MSA) salt (also referred to herein as compound 1A, shown below) can be obtained via a process as disclosed in WO2016/024858A1 or in WO2007/116922A1, the disclosures of which are incorporated herein by reference in their entirety.

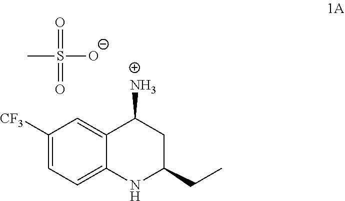

In some embodiments, the desalting of a compound of Formula (IIA) or (IIB) in step (pre-a2) is performed in a mixture of an aqueous sodium hydroxide solution and an organic solvent chosen from toluene, dichloromethane, cyclopentyl methyl ether, isopropyl ether, t-butyl methyl ether, ethyl acetate, isopropyl acetate, methyl ethyl ketone, methyl isobutyl ketone, chlorobenzene and combinations thereof, followed by heating the mixture then cooling the mixture, and allowing the system to phase separate, and separating off the aqueous phase. In some embodiments, the solvent is toluene. In some embodiments the reaction mixture is heated to a temperature between 45° C. and 60° C., then cooled to a temperature between 15° C. and 40° C.

In some embodiments, the organic phase obtained after separating off the aqueous phase is subjected to one or more aqueous washing steps wherein each aqueous washing step is followed by separating off the aqueous phase, such as one or more washing steps with an aqueous sodium chloride solution, followed by separating off the aqueous phase, and subsequently one or more washing steps with deionized water, again followed by separating off the aqueous phase. The resulting washed organic phase is then optionally subjected to distillation to reduce the water content to below 1000 ppm, based on the weight of the solution. Alternatively, in some embodiments, a small amount of water remains in the organic phase with the compound of Formula (II) and the subsequent coupling with a compound of Formula (III) proceeds in the presence of this small amount of water.

In some embodiments the desalting reaction in step (pre-2a) is performed on the mesylate salt (Compound 1A) in a mixture of an aqueous sodium hydroxide solution and toluene, at a temperature between 45° C. and 60° C., followed by cooling the mixture to a temperature between 15° C. and 25° C., allowing the system to phase separate, and separating off the aqueous phase. The toluene phase obtained after separating off the aqueous phase is then optionally subjected to one or more washing steps with an aqueous sodium chloride solution, followed by separating off the aqueous phase, and subsequently one or more washing steps with deionized water, again followed by separating off the aqueous phase, after which the resulting washed toluene phase is subjected to distillation at a temperature between 50° C. and 65° C. under reduced pressure to reduce the water content to below 1000 ppm, based on the weight of the total amount of the solution. Alternatively, a small amount of water remains in the toluene with the compound of Formula (II) and the subsequent coupling reaction with a compound of Formula (III) proceeds in the presence of this small amount of water.

As outlined above, in step (a) the compound of Formula (II), or a salt thereof (e.g., compound of Formula (IIA) or (IIB), such as the mesylate salt 1A), is coupled with a compound of Formula (III) to provide a compound of Formula (IV). In some embodiments, this process is carried out in an organic solvent.

The coupling partner of Formula (III) in step (a) includes a leaving group (X¹). It will be understood that any convenient leaving group may find use in the present disclosure for X¹. In some embodiments, the leaving group (X¹) in the compound of Formula (III) is selected from a halogen, a carbamate, and a substituted sulfonyloxy group. In some embodiments, the leaving group (X¹) in the compound of Formula (III) is a sulfonyloxy group selected from a methanesulfonyloxy, p-toluenesulfonyloxy or a trifluoromethanesulfonyloxy group. In some embodiments, the leaving group (X¹) is a carbamate. In some embodiments, the leaving group (X¹) is a halogen. In certain embodiments, the halogen is chloride. The coupling partner of Formula (III) in step (a) also includes a protecting group (Y¹). The term “protecting group” refers to any group which when bound to a functional group such as a carboxylic acid moiety of the compounds (including intermediates thereof) prevents reactions from occurring at the functional group and which protecting group can be removed by conventional chemical or enzymatic steps to reestablish the functional group e.g., the carboxylic acid moiety. The particular removable protecting group employed is not critical and examples of carboxylic acid protecting groups include conventional substituents such as t-butyl esters, methyl esters, ethyl esters, benzyl esters, allyl esters, 1,1-diethylallyl esters, 2,2,2-trifluro ethyl esters, phenyl esters, 4-methoxybenzyl esters, silyl esters, ortho esters, esters of 2,6-disubstituted phenols (e.g., 2,6-dimethylphenol) and any other groups that can be introduced chemically onto a carboxylic acid group or like functionality and later selectively removed either by chemical or enzymatic methods in mild conditions compatible with the nature of the product. It will be understood that any convenient protecting group (e.g., ester group) for a carboxylic acid moiety may find use in the present disclosure for Y¹, and the selection of appropriate protecting groups can be readily determined by one skilled in the art. Suitable groups for that purpose are discussed in standard textbooks in the field of chemistry, such as Protective Groups in Organic Synthesis, 4^{th} Ed., by T. W. Greene and P. G. M. Wuts (John Wiley & Sons, New York, 1999), in Protecting Group Chemistry, 1st Ed., by Jeremy Robertson (Oxford University Press, 2000); and in March's Advanced Organic chemistry: Reactions Mechanisms, and Structure, 8th Ed., by Michael B. Smith (Wiley-Interscience Publication, 2001). In some embodiments, the protecting group (Y¹) is selected from an alkyl group, a substituted alkyl group, an aryl group, a substituted aryl group, an allyl group, a substituted allyl group, and a silyl group. In some embodiments, the protecting group (Y¹) is selected from t-butyl, methyl, ethyl, benzyl, allyl, substituted allyl, 2,2,2-trifluro ethyl, phenyl, 4-methoxybenzyl ester, a 2,6-disubstituted phenol, and a silyl group. In some embodiments, the protecting group (Y¹) is a t-butyl group. In some embodiments, the compound of Formula (III) is of the structure 1B below:

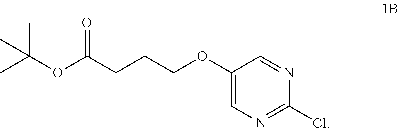 [SMILES: CC(C)(C)OC(=O)CCCOc1cnc(Cl)nc1]

In some embodiments of the coupling reaction of step (a), the solvent is selected from toluene, t-butanol, 1,4-dioxane, xylene, N-methyl-2-pyrrolidone, dimethylformamide, water, tetrahydrofuran, and combinations thereof. In some embodiments, the solvent is a mixture of organic solvent toluene and organic co-solvent t-butanol.

If steps (pre-a1) and (pre-a2) are performed before step (a), the compound of Formula (II) is already present in the required solvent, because the same organic solvents are used in steps (pre-a2) and (a) or because of a solvent swap in step (pre-a2). If need be, more organic solvent and for example an organic co-solvent can be added in step (a). As will be appreciated by the skilled person, an organic co-solvent can also be added during a solvent swap in step (pre-a2). In some embodiments, steps (pre-a1) and (pre-a2) are performed before step (a) and the compound of Formula (II) is present in toluene.

The coupling reaction in step (a) typically is a catalyzed reaction. In some embodiments, the reaction is a palladium-catalyzed coupling reaction in the presence of a base. Suitable examples of palladium catalysts are for example tris(dibenzylideneacetone) dipalladium and Pd(II) acetate. Suitable bases include organic bases (e.g., sodium t-butoxide, and potassium t-butoxide) and inorganic bases (e.g., K₃PO₄, K₃PO₄·H₂O, sodium carbonate, potassium carbonate, cesium carbonate, LiHMDS, NaHMDS, KOH, and NaOH).

In many embodiments, anhydrous K₃PO₄ is used as a base. In many such embodiments, the particle size distribution is such that 90% of the particles are smaller than between about 140 and about 307 microns including between about 140 and about 170 microns, including about 160 and about 290 microns, and about 180 and about 220 microns, and about 200 and about 210 microns. In some embodiments, 90% of the particles are less than 205 microns.

In these and other embodiments, 50% of the particles are between about 35 and about 173 microns or smaller, including between about 35 and about 40 microns.

In these and other embodiments, 10% of the particles between about 7 and about 74 microns including between about 7 and about 10 microns.

In some embodiments, the compound of Formula (II) is reacted in step (a) with a compound of Formula (III) in a solvent (e.g., an organic solvent), using a palladium catalyst, a base. In some embodiments, the reaction mixture further includes a ligand.

In some embodiments, the compound of Formula (IIA) or (IIB) is reacted in step (a) with a compound of Formula (III) in a solvent (e.g., an organic solvent), using a palladium catalyst, a base. In some embodiments, the reaction mixture further includes a ligand.

In some embodiments, the desalted compound of Formula (II) is reacted in step (a) with a compound of Formula (III) in the solvent (e.g., an organic solvent), using Pd(II) acetate, either (S)-BINAP [(S)-2,2′-bis(diphenylphosphino)-1,1′-binaphthyl] or rac-BINAP as a ligand. In some embodiments, (S)-BINAP is used as the ligand, and a base selected from sodium t-butoxide, potassium t-butoxide, anhydrous K₃PO₄, K₃PO₄·H₂O, sodium carbonate, potassium carbonate, cesium carbonate, LiHMDS, NaHMDS, KOH and NaOH.

In some embodiments, a salt of Formula (IIA) or (IIB) is reacted in step (a) with a compound of Formula (III) in the solvent (e.g., an organic solvent), using Pd(II) acetate, either (S)-BINAP [(S)-2,2′-bis(diphenylphosphino)-1,1′-binaphthyl], (R)-BINAP [(S)-2,2′-bis(diphenylphosphino)-1,1′-binaphthyl], or rac-BINAP as a ligand. In some embodiments, (S)-BINAP is used as the ligand, and a base selected from sodium t-butoxide, potassium t-butoxide, anhydrous K₃PO₄, K₃PO₄·H₂O, sodium carbonate, potassium carbonate, cesium carbonate, LiHMDS, NaHMDS, KOH and NaOH. In some embodiments, the salt of Formula (IIA) is the mesylate salt, Compound 1A.

In some embodiments, the reaction in step (a) is performed at a temperature from 70° C. and 80° C., optionally under a nitrogen atmosphere, for 2 or more hours.

In some embodiments, the compound of Formula (II) or salt of Formula (IIA) is reacted in step (a) with a compound of Formula (III) wherein X¹ is Cl and Y¹ is t-butyl, in a mixture of organic solvent toluene and organic co-solvent t-butanol, using Pd(II) acetate as catalyst, (S)-BINAP as a ligand, and anhydrous K₃PO₄ or K₃PO₄·H₂O as a base, at a temperature between 70° C. and 80° C., under a nitrogen atmosphere, for 2 or more hours.

In some embodiments, the one or more aqueous washing steps comprise one or more washing steps with water, preferably deionized water, followed by separating off the aqueous phase, subsequently one or more washing steps with an aqueous HCl solution, followed by separating off the aqueous phase, subsequently one or more washing steps with an aqueous sodium chloride solution, followed by separating off the aqueous phase, and finally one or more washing steps with again deionized water, followed by separating off the aqueous phase.

If t-butanol is used as an organic co-solvent in step (a), this organic co-solvent is removed from the organic phase during the washing steps.

If step (a) is performed in an organic solvent different from the solvent used in step (b), the organic solvent used in step (a) is swapped in step (a) with the organic solvent applied in step (b), such that the compound of Formula (IV) remains in solution.

In some embodiments wherein the (organic) solvents used in steps (a) and (b) are different, at least part of the (organic) solvent used in step (a) is evaporated, such as by using distillation at reduced pressure, and the organic solvent of step (b) is added, such that the compound of Formula (IV) remains in solution during the solvent swap. This process can be performed by continuously evaporating the (organic) solvent used in step (a) and by continuously adding the organic solvent of step (b), for example until the amount of the (organic) solvent used in step (a), based on the total amount of solvent, is below a certain threshold value. Alternatively, this process can be performed batch-wise in more than one steps of evaporating part of the (organic) solvent used in step (a) and subsequently adding part of the organic solvent used in step (b), for example until the amount of the (organic) solvent used in step (a), based on the total amount of solvent, is below a certain threshold value.

In some embodiments, the solvent used in step (a) is a mixture of organic solvent toluene and organic co-solvent t-butanol. The t-butanol is removed from the organic phase comprising the compound of Formula (IV) during the washing steps.

In some embodiments of step (a), the remaining organic solvent toluene is swapped with acetonitrile by distilling off in two or more steps, at a temperature between 50° C. and 65° C. under reduced pressure, part of the toluene with intermediate addition of acetonitrile, in an amount to obtain a solvent mixture with less than about 20 weight percent toluene, based on the combined weight of the solvents, such that the compound of Formula (IV) remains in solution. In some embodiments of the compound of Formula (IV), Y¹ is t-butyl.

Method of Preparing Obicetrapib—Step (b) from Aspects (a)-(d)

In step (b) of the method for preparing a compound of Formula (I) according to the disclosure, the compound of Formula (IV) is converted to the carbamate of Formula (V) in an organic solvent, and subsequently isolated as a solid salt of Formula (VI) (where Y¹ is a protecting group, e.g., as described herein).

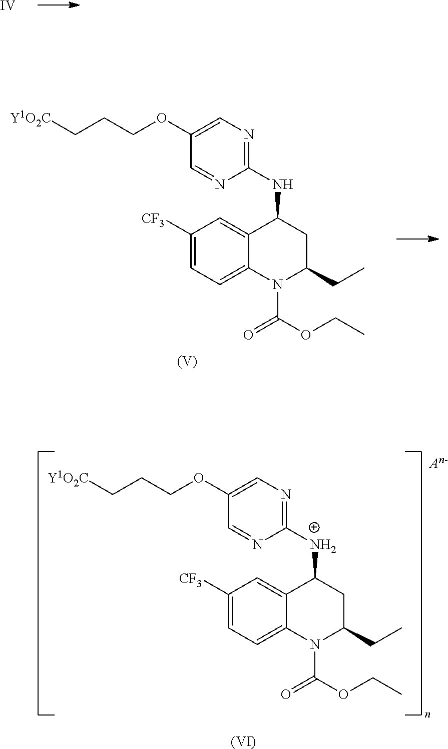

In some embodiments, the organic solvent used in step (b) is selected from acetonitrile, chlorobenzene, toluene, N-methyl-2-pyrrolidone, xylene, 1,4-dioxane, ethyl acetate, isopropyl acetate, methyl ethyl ketone, methyl isobutyl ketone, dichloromethane, t-butyl methyl ether, and combinations thereof. In some embodiments, the organic solvent is acetonitrile or a mixture of chlorobenzene and dichloromethane.

As explained hereinbefore, the compound of Formula (IV) is already provided in step (a) in the organic solvent used in step (b), either because the same organic solvents are used in steps (a) and (b) or because of a solvent swap in step (a). In some embodiments of the compounds of Formulae (IV), (V) and (VI), Y¹ is t-butyl.

In some embodiments, the organic solvent used in step (b) is a mixture of acetonitrile toluene, with less than about 20 weight percent toluene, based on the combined weight of the organic solvents.

In some embodiments, the conversion of the compound of Formula (IV) to the corresponding carbamate with Formula (V) in step (b) is performed in acetonitrile with less than about 20 weight percent toluene, based on the combined weight of the organic solvents, with an excess ethyl chloroformate in the presence of pyridine, at a temperature between 10° C. and 20° C.

If step (b) is performed in an organic solvent different from the organic solvent used in step (c), the organic solvent used in step (b) is swapped in step (b) with the organic solvent applied in step (c), such that the compound of Formula (V) remains in solution.

In some embodiments where the organic solvents used in steps (b) and (c) are different, at least part of the organic solvent used in step (b) is evaporated, such as by distillation at reduced pressure, and the organic solvent of step (c) is added, such that the compound of Formula (V) remains in solution during the organic solvent swap. This process can be performed by continuously evaporating the organic solvent used in step (b) and by continuously adding the organic solvent of step (c), for example until the amount of the organic solvent used in step (b), based on the total amount of organic solvent, is below a certain threshold value. Alternatively, this process can be performed batch-wise in more than one steps of evaporating part of the organic solvent used in step (b) and subsequently adding part of the organic solvent used in step (c), for example until the amount of the organic solvent used in step (b), based on the total amount of organic solvent, is below a certain threshold value.

The resulting mixture is preferably subjected to one or more treatments with an aqueous sodium chloride and/or HCl solution, followed by separating off the aqueous phase, and subsequently to one or more treatments with an aqueous bicarbonate solution, followed by separating off the aqueous phase.

In some embodiments, the conversion of the compound of Formula (IV) to the corresponding carbamate with Formula (V) in step (b) is performed in acetonitrile with an excess of ethyl chloroformate in the presence of pyridine, at a temperature between 10° C. and 20° C., and this solvent is swapped in step (b) with isopropyl acetate by distilling off in two or more steps, at a temperature of 60° C. or less under reduced pressure, part of the acetonitrile with intermediate addition of isopropyl acetate, in an amount to obtain a solution of the compound of Formula (V) in isopropyl acetate, wherein the solution may be subjected to one or more treatments with an aqueous NaCl/HCl solution, followed by separating off the aqueous phase, and subsequently to one or more treatments with an aqueous bicarbonate solution, followed by separating off the aqueous phase.

Next, the compound of Formula (V) dissolved in an organic solvent is converted to a corresponding salt according to Formula (VI), wherein Aⁿ⁻ is an anion and n is an integer from 1-3. The solid form of the salt according to Formula (VI) is then isolated as a solid form.

In some embodiments, the salt of Formula (VI) is chosen from salts with an anion A11-selected from a sulfonate (e.g., besylate, tosylate, napsylate, camsylate, esylate, edisylate and mesylate), a sulfate (e.g., methylsulfate), a halogen, acetate, aspartate, benzoate, bicarbonate, bitartrate, carbonate, citrate, decanoate, fumarate, gluceptate, gluconate, glutamate, glycolate, hexanoate, hydroxynaphthoate, isethionate, lactate, lactobionate, malate, maleate, mandelate, mucate, nitrate, octanoate, oleate, pamoate, pantothenate, phosphate, polygalacturonate, propionate, salicylate, stearate, succinate, tartrate, and teoclate.

In some embodiments, the salt of Formula (VI) is chosen from salts with an anion Aⁿ⁻ selected from chloride, bromide, bitartrate, a sulfate, and a sulfonate.

In some embodiments, the salt of Formula (VI) is chosen from salt with an anion Aⁿ⁻ selected from chloride, bromide, bitartrate, and mesylate.

In some embodiments, the salt from of Formula (VI) is the mesylate salt including the crystalline mesylate salt thereof, Compound 1D:

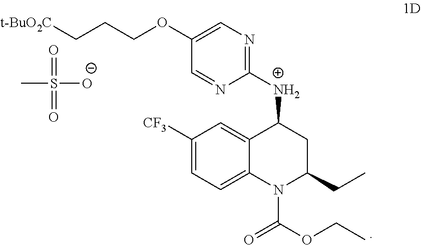

In some embodiments, of the salt of Formula (VI), n is 1.

The organic solvent used in the conversion of Formula (V) to (VI) is not particularly limited, but in some embodiments is selected from cyclopentyl methyl ether, isopropyl ether, t-butyl methyl ether, ethyl acetate, isopropyl acetate, and combinations thereof. In some embodiments, isopropyl acetate or a mixture comprising dichloromethane, n-heptane and isopropyl alcohol, such as a mixture of chlorobenzene, dichloromethane, n-heptane and isopropyl alcohol is used. It is noted that, the compound of Formula (V) is already provided in organic solvent owing to the solvent swap described herein before.

Accordingly, in some embodiments, the organic solvent used in the conversion of compound of Formula (V) to its corresponding salt of Formula (VI) selected from cyclopentyl methyl ether, isopropyl ether, t-butyl methyl ether, ethyl acetate, isopropyl acetate, and combinations thereof, with less than about 20 weight percent toluene and less than about 7 weight percent acetonitrile, based on the combined weight of the solvents. In some embodiments, the solvent is a mixture of isopropyl acetate, toluene and acetonitrile, with less than about 20 weight percent toluene and less than about 7 weight percent acetonitrile, based on the combined weight of the solvents.

In some embodiments, it is preferred to add an organic co-solvent different from the organic solvent already used in step (b). Exemplary organic co-solvents are selected from cyclopentyl methyl ether, isopropyl ether, t-butyl methyl ether, ethyl acetate, isopropyl acetate, and combinations thereof, such as methyl t-butyl ether. As will be appreciated by the skilled person, the need and advantages of using an organic co-solvent depend on the particular organic solvent already used in step (b). In certain cases, the use of a co-solvent can be dispensed with.

In some embodiments, the organic solvent for the conversion of a compound of Formula (V) to its corresponding salt of Formula (VI) comprises isopropyl acetate and methyl t-butyl ether as an organic co-solvent.

Subsequently, an acid is added to form the salts of Formula (VI) defined supra. In some embodiments the acid is selected from ditartartic acid, sulfuric acids, sulfonic acids, hydrogen bromide and hydrogen chloride. In some embodiments, the acid is methanesulfonic acid. In embodiments wherein the salt of Formula (VI) can be obtained in crystalline form, part of the acid needed to form the salt of Formula (VI) can be added before the crystallization and part during the crystallization.

The solid form of the salt of Formula (VI) is isolated by crystallization if the salt of Formula (VI) can be obtained in crystalline form, filtration, one or more optional washing steps of the filtration residue, and drying.

In some embodiments, the compound of Formula (V) is converted to the corresponding mesylate salt according to Formula (VI) with methanesulfonic acid in an organic solvent mixture of isopropyl acetate and methyl t-butyl ether with less than about 20 weight percent toluene and less than 7 weight percent acetonitrile, based on the combined weight of the organic solvents, followed by crystallizing the mesylate salt according to Compound 1D from the organic solvent, with subsequent filtration, one or more optional washing steps of the filtration residue, and drying.

In some embodiments wherein the salt according to Formula (VI) can be obtained in crystalline form, crystallization is induced by adding seed crystals of the salt according to Formula (VI).

In some embodiments, wherein the salt according to Formula (VI) can be obtained in crystalline form, crystallizing the salt according to Formula (VI) and obtaining the crystalline form of the salt according to Formula (VI) is performed by adding the acid needed to form the salts, by agitating the resulting mixture for more than 60 minutes at a temperature from 20° C. to 25° C., by allowing crystallization under agitation at a temperature between 15° C. and 25° C. for more than 120 minutes, followed by subjecting the resulting slurry to vacuum filtration, wherein the filtration residue is washed one or more times with the same organic solvent that is used to crystallize the salt according to Formula (VI) from, and by vacuum drying the crystalline form of the salt according to Formula (VI).

In an embodiment, the invention concerns the salt according to Formula (VI), wherein Aⁿ⁻ is an anion, wherein n is an integer from 1-3. In some embodiments, the compound is the crystalline mesylate (MSA) salt of Formula (VI) (e.g., Compound 1D as described herein).

In some embodiments, crystallizing the mesylate salt of Formula (VI) from an organic solvent mixture of isopropyl acetate and methyl t-butyl ether and obtaining the crystalline form of the mesylate salt according to Compound 1D is performed by adding methanesulfonic acid needed to form the salt, agitating the resulting mixture for more than 60 minutes at a temperature between 15° C. and 25° C. (e.g., 20° C.), then allowing crystallization under agitation at a temperature between 15° C. and 25° C. for more than 120 minutes. The resulting slurry is subjected to vacuum filtration, wherein the filtration residue is washed one or more times with a mixture of isopropyl acetate and methyl t-butyl ether, and dried under vacuum to provide a crystalline form of the mesylate salt according to Compound 1D.

In some embodiments, the compound of Formula (VI) is obtained in a yield of at least 70%, based on the number of moles of the compound of Formula (II). In some embodiments, the compound of Formula (VI) is obtained with a purity of 99% or more, such as a purity of 99.1% or more, 99.2% or more, 99.3% or more, 99.5% or more, or even more.

Method of Preparing Obicetrapib—Step (c) from Aspects (a)-(d)

In step (c) of the process according to the present disclosure, the isolated salt of Formula (VI), or the desalted derivative thereof (e.g., the compound according to Formula (V)), is alkylated with a compound of Formula (VII) to provide a compound of Formula (VIII):

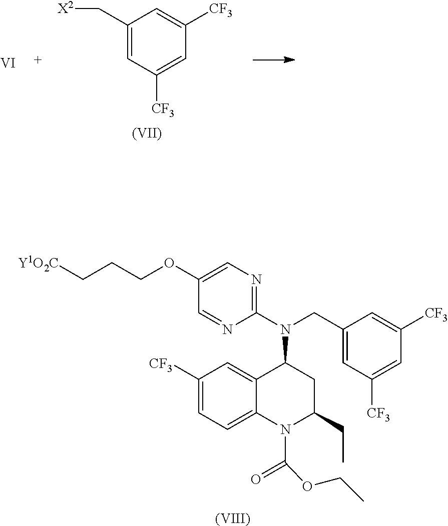

where, X² is a leaving group and Y¹ is a protecting group (e.g., as described herein).

In some embodiments of step (c), the isolated solid form of the salt according to Formula (VI), such as a crystalline form of the salt according to Formula (VI) (such as the crystalline mesylate salt, Compound 1D), is reacted directly with a compound of Formula (VII) in an organic solvent, to form a compound of Formula (VIII) (i.e., without a desalting step).

In some embodiments of step (c), the isolated solid form of the salt according to Formula (VI), such as a crystalline form of the salt according to Formula (VI) (such as the crystalline mesylate salt, Compound 1D), is desalted and reacted with a compound of Formula (VII) in an organic solvent, to form a compound of Formula (VIII). Desalting the compound of Formula (VI) results in a compound according to Formula (V).

When the compound of Formula (VI) is subjected to a desalting step, the desalting process and the subsequent reaction with a compound of Formula (V) are performed in the same organic solvent. In some embodiments, the organic solvent is selected from xylene, n-hexane, toluene, heptanes (mix of isomers), n-heptane, dichloromethane, chlorobenzene, and combinations thereof. In some embodiments, the organic solvent is toluene or n-heptane.

In some embodiments, step (c) is carried out in the presence of a base. In some embodiments, step (c) is carried out in the presence of a solid-liquid phase-transfer catalyst.

In some embodiments, the base is selected from alkali metal hydrides, alkali metal hydroxides, alkali earth metal hydroxides, alkali metal alkoxides, alkali metal carbonates, alkali metal bicarbonates and amines. In some embodiments, the base is chosen from alkali metal alkoxides. In some embodiments, the base is sodium t-pentoxide or a mixture of sodium t-butoxide, and potassium t-butoxide.

In some embodiments, the solid-liquid phase-transfer catalyst is selected from t-butylammonium hydrogensulfate, tetra-n-butylammonium bromide, tetra-n-butylammonium iodide, a crown ether, and combinations thereof. In some embodiments, the catalyst is t-butylammonium hydrogensulfate.

In some embodiments, the reaction of the compound of Formula (V) or (VI) with the compound of Formula (VII) is performed at a temperature between 0° C. and 25° C. (such as from 5° C. to 20° C.).

The coupling partner of Formula (VII) in step (c) includes a leaving group X². It will be understood that any convenient leaving group may find use in the present disclosure for X². In some embodiments, the leaving group X² in the compound of Formula (VII) is selected from a halogen, and a substituted sulfonyloxy group. In some embodiments, the leaving group X² in the compound of Formula (VII) is a substituted sulfonyloxy group selected from a methanesulfonyloxy, p-toluenesulfonyloxy or a trifluoromethanesulfonyloxy group. In some embodiments, the leaving group X² is a halogen. In certain embodiments, the halogen is bromide. In some embodiments, the compound of Formula (VII) is of the structure 1E below.

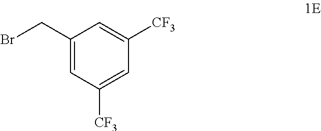 [SMILES: FC(F)(F)c1cc(CBr)cc(C(F)(F)F)c1]

In some embodiments, the desalting of the compound of Formula (VI) and the subsequent reaction with a compound of Formula (VII) in step (c) is performed in toluene as an organic solvent in the presence of a base and a catalyst at a temperature from 5° C. to 25° C. In some embodiments, the desalting of the compound of Formula (VI) and the subsequent reaction with a compound of Formula (VII) in step (c) is performed in toluene as an organic solvent in the presence of sodium t-pentoxide as a base and t-butylammonium hydrogensulfate as a catalyst at a temperature between 5° C. and 25° C. under agitation for about 1 to 8 hours. In some embodiments of the compound of Formula (VI), Y¹ is t-butyl.

In some embodiments, the alkylation of a compound of Formula (VI) (i.e., without an additional desalting step) with a compound of Formula (VII) in step (c) is performed in toluene as an organic solvent in the presence of a base and a catalyst at a temperature from 5° C. to 25° C. In some embodiments, the alkylation of a compound of Formula (VI) with a compound of Formula (VII) in step (c) is performed in toluene as an organic solvent in the presence of sodium t-pentoxide as a base and t-butylammonium hydrogensulfate as a catalyst at a temperature between 5° C. and 25° C. under agitation for about 1 to 8 hours.

In some embodiments, step (c) includes providing crystalline 1D, desalting this compound and reacting the desalted compound with a compound of Formula (VII) wherein X² is Br in toluene as an organic solvent in the presence of sodium t-pentoxide as a base and t-butylammonium hydrogensulfate as a catalyst, at a temperature between 5° C. and 25° C. under agitation for about 1 to 8 hours.

In some embodiments, step (c) includes reacting crystalline 1D with a compound of Formula (VII) wherein X² is Br in toluene as an organic solvent in the presence of sodium t-pentoxide as a base and t-butylammonium hydrogensulfate as a catalyst, at a temperature between 5° C. and 25° C. under agitation for about 1 to 8 hours.

In some embodiments of step (c), the base is the last reagent added to the reaction mixture. Without being bound to any particular theory, the inventors have discovered that by adding the base as the last reagent, the number of equivalents of both the base and the compound of Formula (VII) used in the reaction mixture can be reduced. A reduction in the number of equivalents of the compound of Formula (VII) can in turn reduce the risk of carryover of Formula (VII) related impurities to the final product.

Accordingly, step (c) results in the production of a compound of Formula (VIII) in an organic solvent. In some embodiments of the compound of Formula (VIII), Y¹ is t-butyl. In some embodiments, this reaction mixture is subjected in step (c) to one or more aqueous washing steps to remove impurities, followed by separating off the aqueous phase, and optionally one or more filtration steps, to obtain a washed reaction mixture comprising the compound of Formula (VIII) in the organic solvent. In some embodiments, the reaction mixture comprising the compound of Formula (VIII) in the organic solvent is concentrated by distilling off part of the organic phase to obtain a concentrated reaction mixture comprising the compound of Formula (VIII) in the organic solvent. In some embodiments, the organic solvent comprises from 30 to 40 weight percent of the compound of Formula (VIII) based on the weight of the reaction mixture. In some embodiments, the organic solvent comprises 34 to 37 weight percent of the compound of Formula (VIII) based on the weight of the reaction mixture.

The one or more aqueous washing steps, the optionally one or more filtration steps, and the concentration step are preferably combined such that a washed and concentrated reaction mixture comprising the compound of Formula (VIII) in the organic solvent is obtained. In some cases, the organic solvent includes from 30 to 40 weight percent of the compound of Formula (VIII). In some embodiments, the organic solvent includes from 34 to 37 weight percent of the compound of Formula (VIII) based on the weight of the reaction mixture.

In some embodiments, the one or more aqueous washing steps comprise one or more washing steps with an aqueous acetic acid solution.

In some embodiments, the reaction mixture comprising the compound of Formula (VIII) in toluene as an organic solvent is subjected in step (c) to one or more aqueous washing steps with an aqueous acetic acid solution followed by separating off the aqueous phase, and subsequently by distilling off part of the toluene, typically at a temperature from 75° C. to 90° C. under reduced pressure, to obtain a washed and concentrated reaction mixture comprising the compound of Formula (VIII) in toluene with from 30 to 40 weight percent of the compound of Formula (VIII) based on the weight of the reaction mixture. In some embodiments, the concentrated mixture includes from 34 to 37 weight percent of the compound of Formula (VIII) based on the weight of the reaction mixture.

If step (c) is performed in an organic solvent different from the organic solvent used in step (d), the organic solvent used in step (c) is swapped in step (c) with the organic solvent applied in step (d) such that the compound of Formula (VIII) remains in solution.

In some embodiments, wherein the organic solvents used in steps (c) and (d) are different, at least part of the organic solvent used in step (c) is evaporated, preferably using distillation at reduced pressure, and the organic solvent of step (d) is added, such that the compound of Formula (VIII) remains in solution during the organic solvent swap. This process can be performed by continuously evaporating the organic solvent used in step (c) and by continuously adding the organic solvent of step (d), for example until the amount of the organic solvent used in step (c), based on the total amount of organic solvent, is below a certain threshold value. Alternatively, this process can be performed batch-wise in more than one steps of evaporating part of the organic solvent used in step (c) and subsequently adding part of the organic solvent used in step (d), for example until the amount of the organic solvent used in step (c), based on the total amount of organic solvent, is below a certain threshold value.

Method of Preparing a Compound of Formula (I)—Step (d) from Aspects (a)-(d)

In step (d) of the process according to the present disclosure, the compound of Formula (VIII) is converted to obicetrapib in a first organic solvent (where Y¹ is a protecting group, e.g., as described herein).

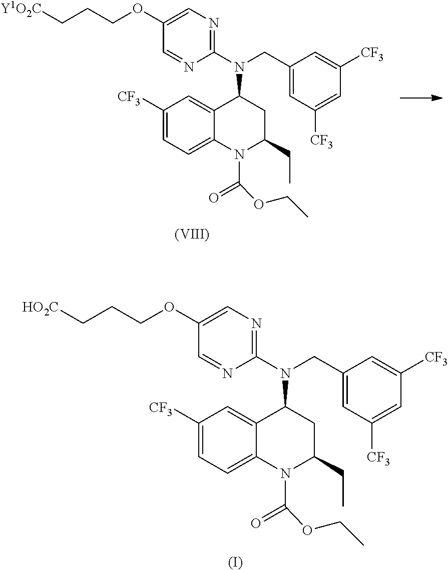

The selection of the first organic solvent used in step (d) is not particularly limited. In some embodiments, the first organic solvent is not an ether or an ester. In some embodiments, the first organic solvent is toluene or a mixture of n-heptane and acetic acid. As explained hereinbefore, the compound of Formula (VIII) is already provided in step (c) in the first solvent used in step (d), either because the same organic solvents are used in steps (c) and (d) or because of a solvent swap in step (c).

Accordingly, in some embodiments, the first organic solvent as defined hereinbefore with from 30 to 40 weight percent of the compound of Formula (VIII), such as from 34 to 37 weight percent, based on the weight of the reaction mixture, is provided in step (d).

In some embodiments, toluene as a first organic solvent with from 30 to 40 weight percent of the compound of Formula (VIII), such as from 34 to 37 weight percent, based on the weight of the reaction mixture, is provided in step (d).

Any convenient protecting group for a carboxylic acid, such as an ester moiety, may find use as Y¹ in the compound of Formula (VIII). As disclosed herein, the selection of an appropriate protecting group for a carboxylic acid can be readily determined by one skilled in the art. In some embodiments of Formula (VIII), the protecting group (Y¹) is selected from an alkyl group, a substituted alkyl group, an aryl group, a substituted aryl group, an allyl group, a substituted allyl group, and a silyl group. In some embodiments of Formula (VIII), the protecting group (Y) is selected from t-butyl, methyl, ethyl, benzyl, allyl, substituted allyl, 2,2,2-trifluro ethyl, phenyl, 4-methoxybenzyl ester, a 2,6-disubstituted phenol, and a silyl group. In some embodiments of the compound of Formula (VIII), the protecting group Y¹ is t-butyl. In some embodiments, the conversion of the compound of Formula (VIII) to obicetrapib is performed by contacting the compound of Formula (VIII) in the first organic solvent, such as toluene or a mixture of n-heptane and acetic acid, with acetic acid (AcOH) and dry HCl under agitation. In some embodiments, the reaction mixture is heated to a temperature between 40° C. and 55° C. and the resulting mixture is maintained at this temperature under agitation for at least 3 hours.

Obicetrapib can be isolated from the resulting mixture using techniques known to the skilled person.

In some embodiments, the resulting mixture comprising obicetrapib, is subjected in step (d) to one or more aqueous washing steps. In some embodiments, the one or more aqueous washing steps in step (d) are performed as follows:
(AA) the reaction mixture comprising obicetrapib is cooled to a temperature between 15° C. and 25° C., and subsequently a mixture of n-heptane, acetonitrile and water is added followed by agitating the resulting mixture for more than 15 minutes at this temperature;
(BB) the system obtained in step (AA) is allowed to phase separate into an organic phase and an aqueous phase and both phases are separated;
(CC) a mixture of n-heptane, acetonitrile, toluene and water is added to the aqueous phase obtained in step (BB), followed by agitating the resulting system for more than 15 minutes at a temperature between 15° C. and 25° C.;
(DD) the system obtained in step (CC) is allowed to phase separate into an organic phase and an aqueous phase and both phases are separated;
(EE) the organic phase obtained in step (BB) and the organic phase obtained in step (DD) are combined, water is added, and the resulting system is agitated for more than 15 minutes at a temperature between 15° C. and 25° C.;
(FF) the system obtained in step (EE) is allowed to phase separate into an organic phase and an aqueous phase and both phases are separated;
(GG) water is added to the organic phase obtained in step (FF) and the resulting system is agitated for more than 15 minutes at a temperature between 15° C. and 25° C.;
(HH) the system obtained in step (GG) is allowed to phase separate into an organic phase and an aqueous phase and both phases are separated;
(II) an aqueous solution of sodium citrate tribasic dihydrate is added to the organic phase obtained in step (HH) followed by agitating the resulting mixture for more than 15 minutes at a temperature between 15° C. and 25° C.;
(JJ) the system obtained in step (II) is allowed to phase separate into an organic phase and an aqueous phase and both phases are separated;
(KK) water is added to the organic phase obtained in step (JJ) and the resulting system is agitated for more than 15 minutes at a temperature between 15° C. and 25° C.; and
(LL) the system obtained in step (KK) is allowed to phase separate into an organic phase and an aqueous phase and both phases are separated.

Steps (AA) to (LL) in this embodiment result in a washed compound of Formula (I) in an organic solvent mixture comprising n-heptane, acetonitrile and the first organic solvent. In some embodiments the first solvent is toluene.

In some embodiments, wherein the first organic solvent does not already mainly consist of cyclopentyl methyl ether, the organic solvent mixture is swapped in a subsequent step (MM) with CPME such that obicetrapib remains in solution.

Hence, in some embodiments, step (LL) is followed by step (MM) wherein at least part of the solvents in the organic solvent mixture obtained in step (LL) is evaporated, such as by distillation at reduced pressure, and wherein cyclopentyl methyl ether is added, such that obicetrapib remains in solution during the solvent swap. In some embodiments, the process results in a solution of obicetrapib in cyclopentyl methyl ether with a concentration between 30 and 40 weight percent based on the weight of the solution. In some embodiments, the concentration of obicetrapib in cyclopentyl methyl ether is from 33 and 37 weight percent, based on the weight of the solution, less than 1 weight percent of the first organic solvent, and less than 1 weight percent of n-heptane based on the weight of the solution.

This process can be performed by continuously evaporating the solvents in the organic solvent mixture obtained in step (LL) and by continuously adding cyclopentyl methyl ether, for example until the amount of specific solvents in the organic solvent mixture, based on the total amount of organic solvents, is below a certain threshold value. Alternatively, this process can be performed batch-wise in more than one steps of evaporating part of the solvents in the organic solvent mixture obtained in step (LL) and by subsequently adding cyclopentyl methyl ether, for example until the amount of specific solvents in the organic solvent mixture, based on the total amount of solvent, is below a certain threshold value.

In some embodiments, the first organic solvent is toluene, step (LL) is followed by step (MM) wherein at least part of the n-heptane, acetonitrile and toluene in the organic solvent mixture obtained in step (LL) is evaporated, such as by distillation at a temperature of 45° C. or lower and at reduced pressure (in-vacuo), with intermediate additions of cyclopentyl methyl ether, such that obicetrapib remains in solution during the solvent swap, resulting in a solution of obicetrapib in cyclopentyl methyl ether with a concentration between 30 and 40 weight percent. In some embodiments, the concentration of obicetrapib in cyclopentyl methyl is from 33 to 37 weight percent based on the weight of the solution, with less than 0.5 weight percent of toluene, less than 0.5 weight percent of acetonitrile and less than 2.7 weight percent of n-heptane.

Method of Preparing Crystalline obicetrapib HCl—Steps (e)-(f) in addition to Aspects (a)-(d)

In some embodiments of the subject method, step (d) is followed by step (e)-(f), wherein obicetrapib is treated with HCl such as in a suitable solvent. Such solvent may be an aqueous solvent or an organic solvent. In some embodiments, the use of an organic solvent provides crystalline obicetrapib HCl.

In some embodiments, the organic solvent used in step (e) comprises a mixture of a solvent and an anti-solvent. In some embodiments, the solvent is selected from methanol, ethanol, isopropanol, acetic acid, acetonitrile, acetone, methyl isobutyl ketone, isopropyl acetate, tetrahydrofuran, methyl t-butyl ether, cyclopentyl methyl ether, N-methyl-2-pyrrolidone, dimethyl sulfoxide, dimethylformamide, 2-methyl-tetrahydrofuran, dichloromethane, 1,4-dioxane, 1,2-diflurobenzene, toluene, hexafluoroisopropanol, and water. In some embodiments, the anti-solvent is selected from n-heptane, n-hexane, n-pentane, and cyclohexane. In some embodiments, the HCl has sufficient solubility in the anti-solvent such that it can be used as a suitable solvent. In some embodiments, the organic solvent used in step (e) comprises a mixture of cyclopentyl methyl ether and n-heptane. In some embodiments, the organic solvent used in step (e) further comprises toluene.

In some embodiments, step (e) comprises providing obicetrapib in a mixture of cyclopentyl methyl ether and n-heptane, raising the temperature to between 35° C. and 40° C. under agitation, adding dry HCl in cyclopentyl methyl ether and raising the temperature again to between 50° C. and 55° C., then adding further n-heptane as an anti-solvent. At this point, a small portion of the reaction mixture can be extracted, cooled to a temperature of between 10° C. and 15° C., to obtain a slurry of crystals of crystalline obicetrapib HCl in a mixture cyclopentyl methyl ether and n-heptane (referred to herein as a “seed crystal slurry”). Optionally, all or a portion of the seed crystal slurry of crystalline obicetrapib HCl can then be added as seed crystals back to the reaction mixture. The seeds assist with nucleation but are not required and thus the process described herein can be done without seeding. The resulting reaction mixture is then cooled to a temperature between 5° C. and 15° C. (such as from 10° C. to 15° C.), followed by crystallizing the crystalline obicetrapib HCl from the system under agitation. In some embodiments, the crystalline obicetrapib HCl is crystallized over a period of 12 hours or more, with subsequent filtration (e.g., through a filter dryer), one or more optional washing steps, such as with a mixture of cyclopentyl methyl ether and n-heptane, and drying. In some cases, a wet filter cake of crystalline obicetrapib HCl is dried in vacuo in steps using temperatures of 25° C.-30° C., 30° C.-40° C., 40° C.-50° C. then 50° C.-55° C., such as 25° C., 35° C., 46° C., and 54° C.

Accordingly, in some embodiments, the method of preparing crystalline obicetrapib HCl comprises the addition of seed crystals (e.g., as a seed crystal slurry). The seed crystals of crystalline obicetrapib HCl can be formed as a slurry by following step (i) as set out above and after addition of dry HCl in cyclopentyl methyl ether and anti-solvent n-heptane, extracting a small portion of the reaction mixture, cooling to a temperature between 10° C. and 15° C., to provide a slurry of crystals of crystalline obicetrapib HCl in cyclopentyl methyl ether and n-heptane.

In some embodiments, the organic solvent used in step (e) comprises a mixture of cyclopentyl methyl ether and n-heptane. Accordingly, in one embodiment, step (e) comprises providing obicetrapib in a mixture of cyclopentyl methyl ether and n-heptane, raising the temperature to 35° C.-45° C. under agitation, adding dry HCl in cyclopentyl methyl ether and raising the temperature again to 50° C.-55° C., addition of further n-heptane as anti-solvent, the optional addition of seed crystals of crystalline obicetrapib HCl (e.g., as a seed crystal slurry prepared as described herein), cooling to a temperature between 5° C. and 15° C. (such as from 10° C. to 15° C.), followed by crystallizing the crystalline obicetrapib HCl from the system under agitation. In some embodiments, the crystalline obicetrapib HCl is crystallized over a period of at least 12 hours, with subsequent filtration, one or more optional washing steps, such as with a mixture of cyclopentyl methyl ether and n-heptane, and drying. In some embodiments, the crystalline obicetrapib HCl is dried in vacuo. In some embodiments, the crystalline obicetrapib HCl is subjected to drying in a vacuum drying cabinet at 25 mbar pressure and at a temperature of 55° C. for 10 hours or more. In some embodiments, after the drying procedure, the crystalline obicetrapib HCl includes less than 0.1 weight percent residual cyclopentyl methyl ether.

In some embodiments described hereinbefore, step (MM) of step (d) results in a solution of obicetrapib in cyclopentyl methyl ether with a concentration between 30 and 40 weight percent, such as from 33 to 37 weight percent, based on the weight of the solution, less than 1 weight percent of the first organic solvent used in step (d), and less than 1 weight percent of n-heptane. In some embodiments described hereinbefore, step (MM) of step (d) results in a solution of obicetrapib in cyclopentyl methyl ether with a concentration between 30 and 40 weight percent, such as from 33 to 37 weight percent, based on the weight of the solution, less than 1 weight percent of toluene, and less than 1 weight percent of n-heptane. These solutions can, after addition of n-heptane, advantageously be used in step (e). As will be appreciated by the skilled person, the n-heptane can also be added in step (d).

Accordingly, in some embodiments, step (e) comprises providing the solution of obicetrapib in cyclopentyl methyl ether with a concentration between 30 and 40 weight percent, such as from 33 to 37 weight percent, based on the weight of the solution, less than 1 weight percent of the first organic solvent used in step (d) (such as toluene), and less than 1 weight percent of n-heptane, addition of n-heptane, raising the temperature to 35° C. to 45° C. under agitation, adding dry HCl in cyclopentyl methyl ether and raising the temperature again to 50° C. to 55° C., addition of further n-heptane as anti-solvent, the optional addition of seed crystals of crystalline obicetrapib HCl (e.g., as a seed crystal slurry prepared as described herein), cooling to a temperature between 5° C. and 15° C. (such as from 10° C. to 15° C.), followed by crystallizing the crystalline obicetrapib HCl from the system under agitation, such as during a period of at least 12 hours, with subsequent filtration, one or more washing steps with a mixture of cyclopentyl methyl ether and n-heptane, and drying. In some cases, a wet filter cake of crystalline obicetrapib HCl is dried in vacuo in steps using temperatures of 25° C.-30° C., 30° C.-40° C., 40° C.-50° C. then 50° C.-55° C., such as 25° C., 35° C., 46° C., and 54° C.

In some embodiments, step (f) comprises the following steps:
(aa) providing crystalline obicetrapib HCl;
(bb) dissolving the crystalline obicetrapib HCl in ethanol under agitation. In some embodiments at a temperature between 15° C. and 25° C.;
(cc) adding an aqueous NaOH solution to the solution obtained in step (bb) and agitating the resulting mixture, such as at a temperature from 20° C. to 25° C. for at least 4 hours, to obtain a solution of the sodium salt obicetrapib;
(dd) optionally filtering the solution obtained in step (cc);
(ee) preparing a CaCl2 solution by adding deionized water to CaCl2 under agitation, followed by adding ethyl acetate as a co-solvent, and stirring the resulting mixture for 10 to 30 minutes;
(ff) cooling the CaCl2 solution obtained in step (ee) to a temperature between 8° C. and 12° C. and adding via a filter to the solution obtained in step (dd) (or (cc)) under agitation at said temperature;
(gg) stirring the slurry resulting from step (ff) for about 1 to about 10 hours. In some embodiments the slurry is stirred at a temperature between 8° C. and 12° C.;
(hh) isolating the solids from the slurry obtained in step (gg) by filtration. In some embodiments the isolating is conducted at a temperature between 8° C. and 12° C.;
(ii) washing the filtration residue obtained in step (hh) with water in one or more washing steps. In some embodiments the washing is conducted at a temperature between 8° C. and 12° C.; and
(jj) drying the washed residue obtained in step (ii), such as in vacuo at a temperature between 40° C. and 50° C. for more than 16 hours (such as 200 hours or more), to obtain amorphous obicetrapib hemicalcium.

In some embodiments of the subject method, crystalline obicetrapib HCl is isolated in step (f) with a purity of 98% or more, such as 98.5% or more, 99% or more 99.5% or more, or even more.

Another embodiment of the disclosure concerns the crystalline obicetrapib HCl obtained by or obtainable by the process as defined herein.

Still another embodiment of the disclosure is directed to crystalline obicetrapib HCl. In some embodiments, crystalline obicetrapib HCl is stored at controlled room temperature and under a nitrogen atmosphere and is protected from moisture to prevent the formation of an amorphous solid, because crystalline obicetrapib HCl including crystalline obicetrapib HCl is hygroscopic.

Method of Preparing Amorphous Obicetrapib Hemicalcium Steps (g)-(h) in Addition to Aspects (a) to (f)

In some embodiments of the subject method, step (f) is followed by steps (g)-(h), wherein the crystalline obicetrapib HCl is converted to amorphous obicetrapib hemicalcium (Formula IB):

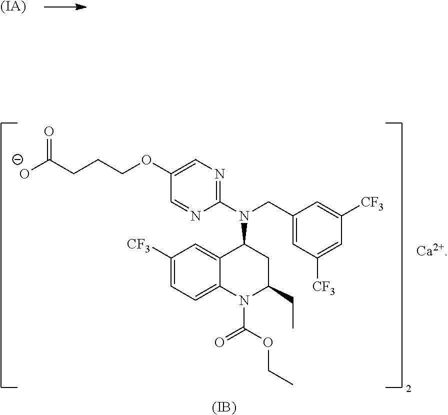

In some embodiments step (g), the preparation of amorphous obicetrapib hemicalcium includes steps (g1)-(g3) as set out below:
(g1) converting crystalline obicetrapib HCl of step (f) to obicetrapib in an organic solvent;
(g2) treating obicetrapib in the organic solvent with aqueous sodium hydroxide to form a sodium salt of obicetrapib; and
(g3) treating the sodium salt of obicetrapib with aqueous calcium chloride to form amorphous obicetrapib hemicalcium;
wherein the compounds in steps (g1) and (g2) are not isolated.

Accordingly, in some embodiments step, (g1) comprises the following steps:
(aa) providing crystalline obicetrapib HCl as defined or obtained in step (f);
(bb) dissolving crystalline obicetrapib HCl in a mixture of water and isopropyl acetate under agitation. In some embodiments, step (bb) is conducted at a temperature between 15° C. and 25° C.;
(cc) allowing phase separation and subjecting the resulting organic phase to one or more subsequent washing steps with water, wherein each washing step is followed by separating off the aqueous phase, resulting in a washed organic phase; and
(dd) performing two or more distillations on the washed organic phase resulting from step (cc) at a temperature of 50° C. or lower (such as 30° C. or lower), with intermediate additions of ethanol, to obtain a solution of the compound of obicetrapib in ethanol. In some embodiments step, (g2) comprises the following steps:
(ee) adding an aqueous NaOH solution to the solution obtained in step (dd) and agitating the resulting mixture, such as at a temperature between 20° C. and 25° C. for at least 4 hours, to obtain a solution of the sodium salt of obicetrapib; and
(ff) optionally filtering the solution obtained in step (ee).

In some embodiments step, (g3) comprises the following steps:
(gg) preparing a CaCl2 solution by adding deionized water to CaCl2 under agitation, followed by adding ethyl acetate as a co-solvent, and stirring the resulting mixture for 10 to 30 minutes;
(hh) cooling the CaCl2 solution obtained in step (gg) to a temperature from 8° C. to 12° C. and adding via a filter to the solution obtained in step (ff) or (ee) under agitation at said temperature;
(ii) stirring the slurry resulting from step (hh) for about 1 to 10 hours. In some embodiments of step (ii), the stirring is conducted at a temperature between 8° C. and 12° C.;
(jj) isolating the solids from the slurry obtained in step (ii) by filtration. In some embodiments of step (jj), the isolating is conducted at a temperature between 8° C. and 12° C.;
(kk) washing the filtration residue obtained in step (jj) with water in one or more washing steps. In some embodiments of step (kk), the washing is conducted at a temperature between 8° C. and 12° C.; and
(ll) drying the washed residue obtained in step (kk), such as in vacuo at a temperature from 40° C. to 50° C. for more than 16 hours (such as 50 hours, 100 hours, 150 hours, or 200 hours, or even more), to obtain the amorphous obicetrapib hemicalcium (also sometimes referred to herein as compound 3).

In some embodiments, step (g) comprises the following steps:
(aa) providing crystalline obicetrapib HCl, as defined or obtained in step (f);
(bb) dissolving crystalline obicetrapib HCl in ethanol under agitation. In some embodiments at a temperature between 15° C. and 25° C.;
(cc) adding an aqueous NaOH solution to the solution obtained in step (bb) and agitating the resulting mixture, such as at a temperature from 20° C. to 25° C. for at least 4 hours, to obtain a solution of the sodium salt of obicetrapib;
(dd) optionally filtering the solution obtained in step (cc);
(ee) preparing a CaCl2 solution by adding deionized water to CaCl2 under agitation, followed by adding ethyl acetate as a co-solvent, and stirring the resulting mixture for 10 to 30 minutes;
(ff) cooling the CaCl2 solution obtained in step (ee) to a temperature between 8° C. and 12° C. and adding via a filter to the solution obtained in step (dd) or (cc) under agitation at said temperature;
(gg) stirring the slurry resulting from step (ff) for about 1 to 10 hours. In some embodiments the slurry is stirred at a temperature between 8° C. and 12° C.;
(hh) isolating the solids from the slurry obtained in step (gg) by filtration. In some embodiments the isolating is conducted at a temperature between 8° C. and 12° C.;
(ii) washing the filtration residue obtained in step (hh) with water in one or more washing steps. In some embodiments the washing is conducted at a temperature between 8° C. and 12° C.; and
(jj) drying the washed residue obtained in step (ii), such as in vacuo at a temperature between 40° C. and 50° C. for more than 16 hours (such as 50 hours, 100 hours, 150 hours, or 200 hours, or even more), to obtain the amorphous hemicalcium-salt of Formula (IB).

In some embodiments, amorphous obicetrapib hemicalcium is stored sealed at a temperature of less than 30° C. and protected from light.

In some embodiments, amorphous obicetrapib hemicalcium is submitted to a subsequent reworking procedure. In some embodiments, amorphous obicetrapib hemicalcium is further reworked by dissolving in ethanol (such as twice the weight of ethanol relative to amorphous obicetrapib hemicalcium at a temperature of 25° C. to 50° C., followed by cooling to 10° C. to 15° C., followed by filtering into a mixture of aqueous calcium chloride solution and ethyl acetate, also cooled to 10° C. to 15° C., followed by filtering, washing with water and drying in vacuo at 45° C. or less for 20 hours or more.

In many embodiments of the disclosure, amorphous obicetrapib hemicalcium is processed to achieve a particle size distribution. In many embodiments such processing is by milling. Examples of milling include hammer milling, ball milling, and jet milling. In other embodiments, spray drying may be used to achieve a particle size distribution. Thus, in some embodiments, of the disclosure, spray-dried amorphous obicetrapib hemicalcium is provided. An example of jet-milled amorphous obicetrapib hemicalcium is provided in Example 11.14.

In many embodiments of the disclosure, unmilled amorphous obicetrapib hemicalcium is provided. In many embodiments of the disclosure, milled amorphous obicetrapib hemicalcium is provided.

In many embodiments, the particle size distribution of amorphous obicetrapib hemicalcium is such that 90% of the particles have a diameter of about 15 microns or less. In these and other embodiments, 90% of the particles have a diameter of about 14 microns or less, 13 microns or less, 12 microns or less, 11 microns or less, 10 microns or less, 9 microns or less, 8 microns or less, 7 microns or less, 6 microns or less, 5 microns or less, 4 microns or less, or 3 microns or less.

In some embodiments, 90% of the particles have a diameter between about 6 microns and 15 microns.

In these and other embodiments, the particle size distribution of amorphous obicetrapib hemicalcium is such that 50% of the particles have a diameter of about 5 microns or less, such as, for example, 4 microns or less or 3 microns or less.

In these and other embodiments, the particle size distribution of amorphous obicetrapib hemicalcium is such that 10% of the particles have a diameter of about 2 microns or less.

Amorphous obicetrapib hemicalcium of the disclosure can be made with high chemical purity according to the processes of the disclosure. Such levels of purity include greater than 98.0% pure such as greater than 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% or more. The highest level of purities such as greater than 99.8% or 99.9% pure are more readily achieved with processes where crystalline obicetrapib HCl is used as an intermediate.

As summarized above, also provided herein are amorphous calcium salts of obicetrapib including amorphous obicetrapib hemicalcium. New intermediates for use in the synthesis of obicetrapib and salts thereof are also provided.

Thus, the subject method has been described by reference to certain embodiments discussed above. It will be recognized that these embodiments are susceptible to various modifications and alternative forms well known to those of skill in the art.

In certain preferred embodiments of the invention, obicetrapib, as contained in the present pharmaceutical compositions, as used in the present methods, as contained in the unit dosage forms (comprised in the pharmaceutical kit), etc., is a salt form of obicetrapib, more particularly a salt as described by one or more of the non-limiting clauses that follow:

Clause 1. An amorphous calcium salt of obicetrapib.
Clause 2. Amorphous obicetrapib hemicalcium.
Clause 3. Stable amorphous obicetrapib hemicalcium.
Clause 4. Substantially pure amorphous obicetrapib hemicalcium.
Clause 5. The amorphous obicetrapib hemicalcium salt of clauses 2-4, substantially free of any crystalline salt of obicetrapib hemicalcium.
Clause 6. The amorphous obicetrapib hemicalcium of clauses 2-5, having an x-ray powder diffraction pattern substantially the same as that of FIG. 49.
Clause 7. The amorphous obicetrapib hemicalcium of clauses 2-5, having an x-ray powder diffraction pattern comprising one or more x-ray powder diffraction peaks at about 3.4°2θ, about 7.0°2θ, and about 9.2°2θ.
Clause 8. The amorphous obicetrapib hemicalcium of clauses 2-7, wherein the amorphous obicetrapib hemicalcium does not birefringe.
Clause 9. The amorphous obicetrapib hemicalcium of clauses 2-8, having a glass transition temperature at a value between about 107° C. and about 112° C.
Clause 10. The amorphous obicetrapib hemicalcium of clause 9, wherein the glass transition temperature is measured with modulated differential scanning calorimetry.
Clause 11. The amorphous obicetrapib hemicalcium of clause 10, wherein the measurement with modulated differential scanning calorimetry uses a sample pan which is open.
Clause 12. The amorphous obicetrapib hemicalcium of clause 11, wherein the opening is a pinhole.
Clause 13. The amorphous obicetrapib hemicalcium of clauses 8-12, wherein the glass transition temperature is at a value between about 110° C. and about 112° C.
Clause 14. The amorphous obicetrapib hemicalcium of clauses 2-13, having a glass transition temperature of less than about 100° C. when measured by differential scanning calorimetry using a closed sample pan.
Clause 15. The amorphous obicetrapib hemicalcium of clause 14, having a glass transition temperature at a value between about 70° C. and about 92° C. when measured by differential scanning calorimetry using a closed sample pan.
Clause 16. The amorphous obicetrapib hemicalcium of clauses 2-15, having a loss in weight of less than about 1% when heated to about 200° C.
Clause 17. The amorphous obicetrapib hemicalcium of clause 16, wherein the weight loss is between about 0.8% and about 0.95%.
Clause 18. The amorphous obicetrapib hemicalcium of clause 17, wherein the weight loss is between about 0.84% and about 0.92%.
Clause 19. The amorphous obicetrapib hemicalcium of clauses 2-18, having a water content of less than about 5%.
Clause 20. The amorphous obicetrapib hemicalcium of clause 19, having a water content of less than about 4%.
Clause 21. The amorphous obicetrapib hemicalcium of clause 20, having a water content of less than about 3%.
Clause 22. The amorphous obicetrapib hemicalcium of clause 19, having a water content of between about 0.5% and about 1.5%.
Clause 23. The amorphous obicetrapib hemicalcium of clauses 2-22, in a bulk form or formulated composition having a particle size distribution wherein about 90% of the particles have a diameter of about 15 microns or less.
Clause 24. The amorphous obicetrapib hemicalcium of clause 23, wherein about 90% of the particles have a diameter of between about 6 microns and about 15 microns.
Clause 25. The amorphous obicetrapib hemicalcium of clause 24, having a particle size distribution wherein about 90% or more of the particles have a diameter of about 14 microns or less.
Clause 26. The amorphous obicetrapib hemicalcium of clause 25, having a particle size distribution wherein about 90% or more of the particles have a diameter of about 13 microns or less.
Clause 27. The amorphous obicetrapib hemicalcium of clause 26, having a particle size distribution wherein about 90% or more of the particles have a diameter of about 12 microns or less.
Clause 28. The amorphous obicetrapib hemicalcium of clause 27, having a particle size distribution wherein about 90% or more of the particles have a diameter of about 11 microns or less.
Clause 29. The amorphous obicetrapib hemicalcium of clause 28, having a particle size distribution wherein about 90% or more of the particles have a diameter of about 10 microns or less.
Clause 30. The amorphous obicetrapib hemicalcium of clause 29, having a particle size distribution wherein about 90% or more of the particles have a diameter of about 9 microns or less.
Clause 31. The amorphous obicetrapib hemicalcium of clause 30, having a particle size distribution wherein about 90% or more of the particles have a diameter of about 8 microns or less.
Clause 32. The amorphous obicetrapib hemicalcium of clause 31, having a particle size distribution wherein about 90% or more of the particles have a diameter of about 7 microns or less.
Clause 33. The amorphous obicetrapib hemicalcium of clause 32, having a particle size distribution wherein about 90% or more of the particles have a diameter of about 6 microns or less.
Clause 34. The amorphous obicetrapib hemicalcium of clause 33, having a particle size distribution wherein about 90% or more of the particles have a diameter of about 5 microns or less.
Clause 35. The amorphous obicetrapib hemicalcium of clause 34, having a particle size distribution wherein about 90% or more of the particles have a diameter of about 4 microns or less.
Clause 36. The amorphous obicetrapib hemicalcium of clause 35, having a particle size distribution wherein about 90% or more of the particles have a diameter of about 3 microns or less.
Clause 37. The amorphous obicetrapib hemicalcium of clauses 2-36, in a bulk form or formulated composition having a particle size distribution wherein about 50% of the particles have a diameter of about 5 microns or less.
Clause 38. The amorphous obicetrapib hemicalcium of clause 37, having a particle size distribution wherein about 50% of the particles have a diameter of about 4 microns or less.
Clause 39. The amorphous obicetrapib hemicalcium of clause 38, having a particle size distribution wherein about 50% of the particles have a diameter of about 3 microns or less.
Clause 40. The amorphous obicetrapib hemicalcium of clauses 2-39, in a bulk form or formulated composition having a particle size distribution wherein about 10% of the particles have a diameter of about 2 microns or less.
Clause 41. The amorphous obicetrapib hemicalcium of clauses 2-40, having a chemical purity of at least 98.0%.
Clause 42. The amorphous obicetrapib hemicalcium of clause 41, having a chemical purity of at least 99.0%.
Clause 43. The amorphous obicetrapib hemicalcium of clause 42, having a chemical purity of at least 99.5%.
Clause 44. The amorphous obicetrapib hemicalcium of clause 43, having a chemical purity of at least 99.6%.
Clause 45. The amorphous obicetrapib hemicalcium of clause 44, having a chemical purity of at least 99.7%.
Clause 46. The amorphous obicetrapib hemicalcium of clause 45, having a chemical purity of at least 99.8%.
Clause 47. The amorphous obicetrapib hemicalcium of clause 46, having a chemical purity of at least 99.9%.
Clause 48. The amorphous obicetrapib hemicalcium of clauses 2-47, having a solid-state ¹³C-NMR spectrum substantially the same as that of FIG. 65.
Clause 49. The amorphous obicetrapib hemicalcium of clauses 2-48, having a solid-state ¹³C-NMR spectrum where no peak is present at about 22.1 ppm.
Clause 50. The amorphous obicetrapib hemicalcium of clauses 2-49, having a solid-state ¹³C-NMR spectrum where no peak is present at about 29.5 ppm.
Clause 51. Unmilled amorphous obicetrapib hemicalcium.
Clause 52. Milled amorphous obicetrapib hemicalcium.
Clause 53. The amorphous obicetrapib hemicalcium of clauses 2-50, wherein the amorphous obicetrapib hemicalcium has been milled.
Clause 54. The amorphous obicetrapib hemicalcium of clauses 2-50 or 53, wherein the amorphous obicetrapib hemicalcium has been jet milled.
Clause 55. The amorphous obicetrapib hemicalcium of clauses 2-50 or 53-54 wherein the amorphous obicetrapib hemicalcium has been spray dried.
Clause 56. Amorphous obicetrapib hemicalcium prepared by a synthetic process wherein an intermediate in the process comprises crystalline obicetrapib HCl.
Clause 57. The amorphous obicetrapib hemicalcium of clauses 2-56, wherein the amorphous obicetrapib hemicalcium is prepared by a synthetic process wherein an intermediate in the process comprises crystalline obicetrapib HCl.

### Clause 58. Obicetrapib HCl.

Clause 59. Crystalline obicetrapib HCl.
Clause 60. An amorphous HCl obicetrapib compound.
Clause 61. A solvate of the HCl obicetrapib of clauses 58-60.
Clause 62. HCl obicetrapib of clauses 58-61, wherein the weight percent of HCl is between about 0.01% and about 8%.
Clause 63. A composition comprising crystalline obicetrapib HCl of any one of clauses 58-62.
Clause 64. The crystalline obicetrapib HCl of clause 58-60 or 62-63, wherein the crystalline obicetrapib HCl is a solvate.
Clause 65. The crystalline obicetrapib HCl of clause 64, wherein the solvate comprises obicetrapib and hydrochloric acid.
Clause 66. The crystalline obicetrapib HCl of clause 65, wherein the solvate comprises an organic solvent.
Clause 67. The crystalline obicetrapib HCl of clause 66, where the solvate comprises a solvent wherein the solubility is sufficient to dissolve sufficient HCl so as to deliver sufficient HCl to create crystalline obicetrapib HCl.
Clause 68. The solvate of any one of clauses 61, or 64-67, wherein the solvent of the solvate is selected from methanol, ethanol, isopropanol, acetic acid, acetonitrile, acetone, methyl isobutyl ketone, isopropyl acetate, tetrahydrofuran, methyl t-butyl ether, cyclopentyl methyl ether (CPME), N-methyl-2-pyrrolidone, dimethyl sulfoxide, dimethylformamide, 2-methyl-tetrahydrofuran, dichloromethane, 1,4-dioxane, 1,2-diflurobenzene, toluene, and hexafluoroisopropanol.
Clause 69. The crystalline obicetrapib HCl of clause 68, wherein the solvent is CPME.
Clause 70. The crystalline obicetrapib HCl of any one of clauses 58-59 or 61-69, having an x-ray powder diffraction pattern substantially the same as that in FIG. 67.
Clause 71. The crystalline obicetrapib HCl of any one of clauses 58-59 or 61-69, having an x-ray powder diffraction pattern comprising a peak at about 9.8°2θ.
Clause 72. The crystalline obicetrapib HCl of any one of clauses 58-59, 61-69 or 71, having an x-ray powder diffraction pattern comprising one or more peaks at about 8.1°2θ, about 9.8°2θ, about 13.8°2θ, about 16.7°2θ, and about 19.5°2θ.
Clause 73. A salt according to Formula (VI):

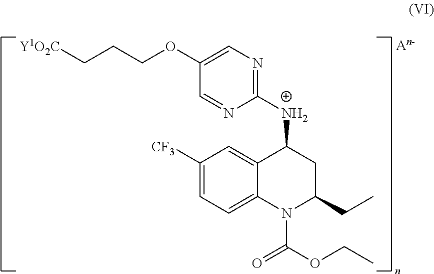

wherein Y¹ is a protecting group, An− is an anion; and n is an integer from 1-3.
Clause 74. The salt according to clause 73, wherein the compound is a mesylate salt of the following structure (Compound 1D):

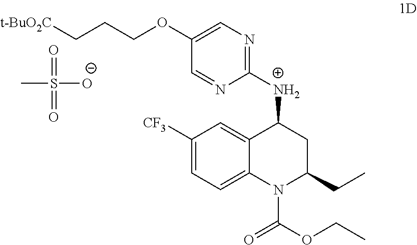

Clause 75. The crystalline mesylate salt of Compound 1D of clause 74.
Clause 76. The crystalline mesylate salt of Compound 1D of clause 75, having a powder diffraction pattern substantially the same as any of the four x-ray powder patterns set forth in FIG. 68.
Clause 77. The crystalline mesylate salt of Compound 1D of clause 75, having an x-ray powder diffraction pattern comprising one or more peaks at about 5.2°2θ and about 9.1°2θ.
Clause 78. The crystalline mesylate salt of Compound 1D of clauses 75-77 having an x-ray powder diffraction pattern comprising one or more peaks at about 9.1°2θ, about 15.9°2θ, about 16.5°2θ, about 17.2°2θ, about 18.6°2θ, and about 19.2°2θ.

In certain preferred embodiments of the invention, obicetrapib, as contained in the present pharmaceutical compositions, as used in the present methods, as contained in the unit dosage forms (comprised in the pharmaceutical kit), etc., is a salt form of obicetrapib, more particularly a salt that can be prepared using methods as described by one or more of the non-limiting clauses that follow:

Clause 79. A method of preparing obicetrapib, wherein the method comprises:
(a) preparing a compound of Formula (IV), by coupling a compound of Formula (II) or a salt thereof, with a compound of Formula (III):

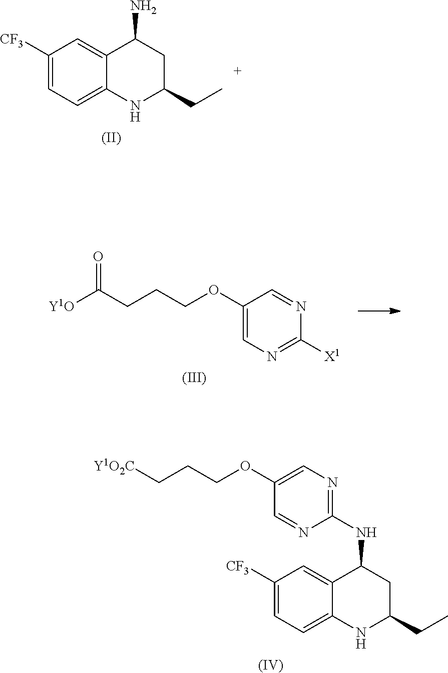

where, X¹ is a leaving group and Y¹ is a protecting group;
(b) preparing a carbamate of Formula (V) from the compound of Formula (IV) and isolating as a solid salt form of Formula (VI):

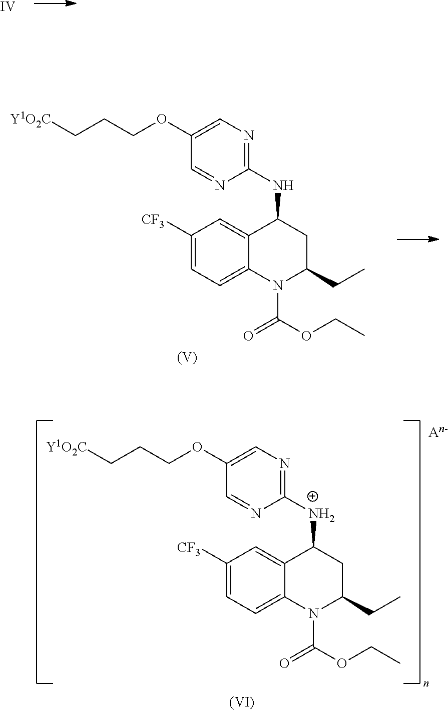

where Y¹ is a protecting group, Aⁿ⁻ is an anion and n is an integer from 1-3;
(c) optionally desalting the compound of Formula (VI) and alkylating with a compound of Formula (VII) to provide a compound of Formula (VIII):

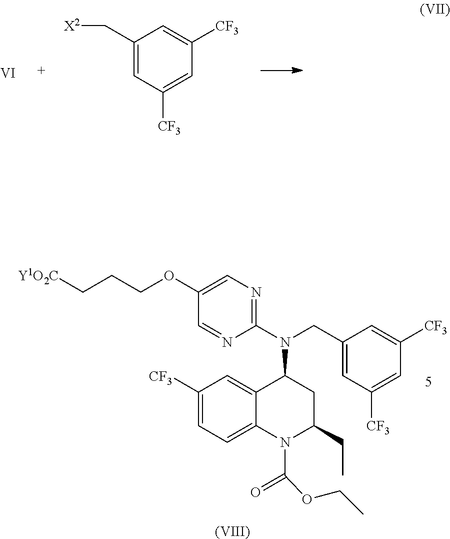

where X² is a leaving group and Y¹ is a protecting group; and
(d) converting the compound of Formula (VIII) to obicetrapib, wherein the reaction steps (a)-(d) are performed in an organic solvent, compounds (IV), (V), and (VIII) are optionally not isolated from the organic solvent, and wherein the process does not require chromatography.
   Clause 80. The method according to clause 79, wherein the compound of Formula (II) in step (a) is obtained by applying the following steps before step (a):
(pre-a1) providing a compound of Formula (IIA) or (IIB):

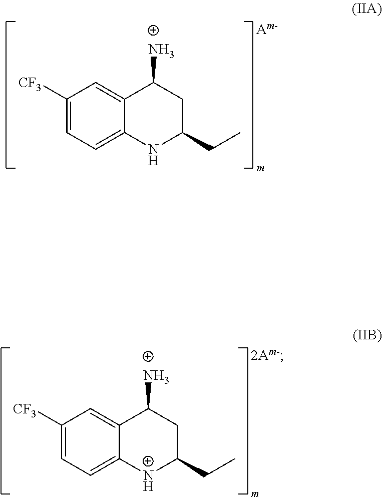

and
(pre-a2) desalting the compound of Formula (IIA) or (IIB) to obtain the compound of Formula (II);
wherein the reaction in step (pre-a2) is performed in an organic solvent and the compound of Formula (II) is optionally not isolated from the organic solvent, and the process does not require chromatography.
   Clause 81. The method according to clause 80, wherein the salt of Formula (IIA) or (IIB) is chosen from salts with an anion Am− selected from a sulfonate, a sulfate, a halogen, acetate, aspartate, benzoate, bicarbonate, bitartrate, carbonate, citrate, decanoate, fumarate, gluceptate, gluconate, glutamate, glycolate, hexanoate, hydroxynaphthoate, isethionate, lactate, lactobionate, malate, maleate, mandelate, mucate, nitrate, octanoate, oleate, pamoate, pantothenate, phosphate, polygalacturonate, propionate, salicylate, stearate, succinate, tartrate, and teoclate; wherein the sulfonate may be a besylate, tosylate, napsylate, camsylate, esylate, edisylate, or mesylate; the sulfate may be a methylsulfate; and the halogen may be a chloride, iodide, or bromide.
   Clause 82. The method of clause 81, wherein the salt with an anion A^{m-} is selected from chloride, bromide, bitartrate, a sulfate, and a sulfonate.
   Clause 83. The method of clause 82, wherein the salt with an anion A^{m-} is selected from chloride, bromide, bitartrate, and mesylate.
   Clause 84. The method of any one of clauses 79-83, wherein Y¹ in the compounds of Formulae (III)-(VI) and (VIII) is selected from an alkyl group, a substituted alkyl group, an aryl group, a substituted aryl group, an allyl group, a substituted allyl group and a silyl group.
   Clause 85. The method of clause 84, wherein Y¹ in the compounds of Formulae (III)-(VI) and (VIII) is selected from t-butyl, methyl, ethyl, benzyl, allyl, substituted allyl, 2,2,2-trifluroethyl, phenyl, 4-methoxybenzyl ester, a 2,6-disubstituted phenol, and a silyl group.
   Clause 86. The method of clause 85, wherein Y¹ in the compounds of Formulae (III)-(VI) and (VIII) is t-butyl.
   Clause 87. The method of any one of clauses 79-86, wherein the salt of Formula (VI) is chosen from salts with an anion Aⁿ⁻ selected from a sulfonate, a sulfate, a halogen, acetate, aspartate, benzoate, bicarbonate, bitartrate, carbonate, citrate, decanoate, fumarate, gluceptate, gluconate, glutamate, glycolate, hexanoate, hydroxynaphthoate, isethionate, lactate, lactobionate, malate, maleate, mandelate, mucate, nitrate, octanoate, oleate, pamoate, pantothenate, phosphate, polygalacturonate, propionate, salicylate, stearate, succinate, tartrate, and teoclate; wherein the sulfonate may be a besylate, tosylate, napsylate, camsylate, esylate, edisylate, or mesylate; the sulfate may be a methylsulfate; and the halogen may be a chloride, iodide, or bromide.
   Clause 88. The method of clause 87, wherein the salt with an anion Aⁿ⁻ is selected from chloride, bromide, bitartrate, a sulfate, and a sulfonate.
   Clause 89. The method of clause 87, wherein the salt with an anion Aⁿ⁻ is selected from chloride, bromide, bitartrate, and mesylate.
   Clause 90. The method of clause 87, wherein the salt form of Formula (VI) is the mesylate salt, Compound 1D:

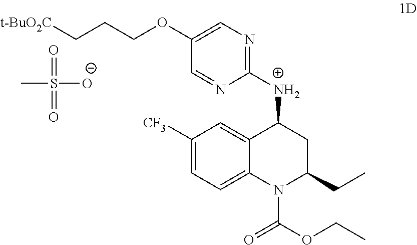

Clause 91. The method of clause 90, wherein the mesylate salt is crystalline.
Clause 92. The method of any one of clauses 79-91, wherein X¹ in the compound of Formula (III) is selected from a halogen, a carbamate, and a substituted sulfonyloxy group.
Clause 93. The method of clause 92, wherein X¹ in the compound of Formula (III) is a halogen.
Clause 94. The method of clause 93, wherein the halogen is chloride.
Clause 95. The method of any one of clauses 79-94, wherein X² in the compound of Formula (VII) is selected from a halogen and a substituted sulfonyloxy group.
Clause 96. The method of clause 95, wherein X² in the compound of Formula (III) is a halogen.
Clause 97. The method of clause 96, wherein the halogen is bromide.
Clause 98. A method of preparing an amorphous hemicalcium salt of obicetrapib wherein the method comprises:
(i) treating obicetrapib with HCl to obtain a crystalline obicetrapib HCl compound;
(ii) isolating the crystalline obicetrapib HCl compound;
(iii) preparing an amorphous hemicalcium salt of obicetrapib from the crystalline obicetrapib HCl compound isolated in step (ii); and
(iv) isolating an amorphous hemicalcium salt of obicetrapib.
   Clause 99. The method of clause 98, wherein the isolated crystalline obicetrapib HCl compound in step (ii) comprises a compound of Formula (IH):

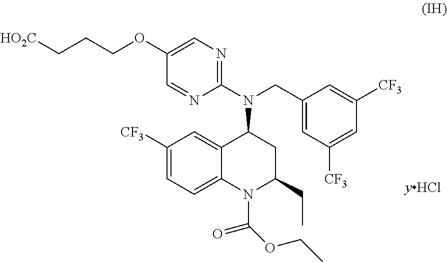 [SMILES: CCOC(=O)N1c2ccc(C(F)(F)F)cc2[C@@H](N(Cc2cc(C(F)(F)F)cc(C(F)(F)F)c2)c2ncc(OCCCC(=O)O)cn2)C[C@H]1CC]

wherein y varies from 0.002 to 1.5.
Clause 100. The method according to clauses 98 or 99, wherein the preparation of the amorphous hemicalcium salt of Formula (I) in step (iii) comprises the following steps:
(iii-1) converting the crystalline obicetrapib HCl compound of step (ii) to provide obicetrapib in one or more suitable solvents selected from organic solvents and aqueous solvents;
(iii-2) treating obicetrapib in the organic solvent with aqueous sodium hydroxide to form a sodium salt of obicetrapib; and
(iii-3) treating the sodium salt of obicetrapib with aqueous calcium chloride to form the amorphous hemicalcium salt of obicetrapib;
   wherein the compounds in steps (iii-1) and (iii-2) are optionally not isolated.
   Clause 101. The method according to clauses any one of clauses 98-100, wherein the amorphous hemicalcium salt of obicetrapib is amorphous obicetrapib hemicalcium.
   Clause 102. The method of any one of clauses 98-101, wherein the amorphous calcium salt of obicetrapib is isolated with a chemical purity of at least 99%.
   Clause 103. The method of clause 102, wherein the amorphous calcium salt of obicetrapib is isolated with a purity of at least 99.1%.
   Clause 104. The method of clause 102, wherein the amorphous calcium salt of obicetrapib is isolated with a purity of at least 99.2%.
   Clause 105. The method of clause 102, wherein the amorphous calcium salt of obicetrapib is isolated with a purity of at least 99.3%.
   Clause 106. The method of clause 102, wherein the amorphous calcium salt of obicetrapib is isolated with a purity of at least 99.4%.
   Clause 107. The method of clause 102, wherein the amorphous calcium salt of obicetrapib is isolated with a purity of at least 99.5%.
   Clause 108. The method of clause 102, wherein the amorphous calcium salt of obicetrapib is isolated with a purity of at least 99.6%.
   Clause 109. The method of clause 102, wherein the amorphous calcium salt of obicetrapib is isolated with a purity of at least 99.7%.
   Clause 110. The method of clause 102, wherein the amorphous calcium salt of obicetrapib is isolated with a purity of at least 99.8%.
   Clause 111. The method of clause 102, wherein the amorphous calcium salt of obicetrapib is isolated with a purity of at least 99.9%.
   Clause 112. The method according to clauses any of clauses 102-111, wherein the amorphous calcium salt of obicetrapib is amorphous obicetrapib hemicalcium.
   Clause 113. A pharmaceutical composition comprising an amorphous salt of obicetrapib calcium of any one of clauses 1-57 and one or more pharmaceutically acceptable carriers.
   Clause 114. The pharmaceutical composition of clause 113, wherein the amorphous salt of obicetrapib calcium is amorphous obicetrapib hemicalcium.
   Clause 115. A method of treating a subject suffering from or having an increased risk of developing a cardiovascular disease, the method comprising administering a therapeutically effective amount of a pharmaceutical composition according to clauses 113 or 114 to the subject.
   Clause 116. An amorphous calcium salt of obicetrapib prepared according to the processes of any one of clauses 79-112.
   Clause 117. The amorphous calcium salt of clause 116, which is amorphous obicetrapib hemicalcium.
   Clause 118. A method of making an amorphous obicetrapib calcium salt comprising treating obicetrapib with an acid to form a salt, solvate composition, or combination thereof; isolating the salt, solvate, composition, or combination thereof; treating the salt, solvate, composition, or combination thereof with a calcium source to make an amorphous obicetrapib hemicalcium salt.
   Clause 119. The method of clause 118, wherein the calcium source is calcium chloride.
   Clause 120. A salt, solvate, composition or combination thereof, comprising obicetrapib and a free acid.
   Clause 121. The salt of clause 120.
   Clause 122. The solvate of clause 120.
   Clause 123. The composition of clause 120.
   Clause 124. The salt, solvate, composition, or combination thereof of clause 120, wherein the free acid is selected from a sulfonic acid, a sulfuric acid, a halogenated acid, acetic acid, aspartic acid, benzoic acid, bicarbonic acid, bitartaric acid, carbonic acid, citric acid, decanoic acid, fumaric acid, gluceptic acid, gluconic acid, glutamic acid, glycolic acid, hexanoic acid, hydroxynaphthoic acid, isethionic acid, lactic acid, lactobionic acid, malic acid, maleic acid, mandelic acid, mucic acid, nitric acid, octanoic acid, oleic acid, pamoic acid, pantothenic acid, phosphic acid, polygalacturonic acid, propionic acid, salicylic acid, stearic acid, succinic acid, tartric acid, and a teoclic acid; wherein the sulfonic acid may be a benzene sulfonic acid, toluene sulfonic acid, naphthalene sulfonic acid, ethane disulfonic acid, or methanesulfonic acid; the sulfuric acid a methyl sulfuric acid; and the halogenated acid may be HCl, HBr, or HI.
   Clause 125. The method of clause 118, wherein the calcium source is a halogenated calcium salt.
   Clause 126. The method of clause 118, wherein the calcium source is a soluble calcium salt.
   Clause 127. The method of clause 118, wherein the calcium source is a calcium salt.

The present disclosure may further be a method as described by one or more of the preceding non-limiting clauses.

### EXAMPLES

### Analytical and Physical Characterization Methods:

The methods used throughout the study are summarised in Table A. The specific parameters and conditions for analytical and physical assessments used for each non-limiting example is described in relevant section of such example.

TABLE A

| Test | Title |
|---|---|
| XRPD | Transmission method for phase identification using X-Ray Powder Diffraction |
| PSD | Method for particle size distribution by laser diffraction dry powder dispersion |
| TBD | standard test method for tapped bulk density analysis using drop height density tester |
| KF | determination of water content by indirect (coulometric) karl fischer titration |
| Discriminating Dissolution | discriminating dissolution method for obicetrapib at ph 6.8 and 50 rpm paddle speed. |
| Content Uniformity | determination of content uniformity of obicetrapib and ezetimibe by hplc-UV. |
| Assay and related | identity, assay and impurities/related substances profile determination of obicetrapib by hplc-UV. |
| substances/impurities for | |
| obicetrapib | |
| Assay and impurities/related | identity, assay and impurities/related substances profile determination of ezetimibe by hplc-UV |
| substances for ezetimibe | |
| Visual Appearance | determination of appearance using the Munsell colour system |

### XRPD

The XRPD analyses were run in transmission mode on an X'pert Pro/Empyrean X-Ray Diffractometer (PANalytical) equipped with an X'Celerator detector using a standard XRPD Aptuit method. The data were evaluated using the Highscore Plus software. The instrumental parameters used are listed in table B below.

TABLE B

| Instrumental Parameter | Value |
|---|---|
| 2-theta range | 2-45° |
| Step size [°2-theta] | 0.0167 |
| Time per step [sec] | 59.690 sec |
| Scan Mode | Continuous |
| Sample Movement | Spinning. 1.0 sec rotation time |
| Wavelength [nm] | Cu Kα₁ = 1.54060 Kα₂ = 1.54443 |
| X-ray Mirror | Inc. Beam Cu W/Si focusing MPD. Acceptance Angle 0.8° C. Length 55.3 mm |
| Slits Divergence/Antiscatter/Mask | Slit Fixed 1/2°/Slit Fixed 1/2°/10 mm Inc Beam Mask |
| Temperature/RH | Room temperature. Lab RH |
| Fixed Slits | Soller slits 0.02 rad on Incident and Diffracted beam |
| Detector type | X'Celerator (active length 2.122°) |
| Scanning mode | Transmission |
| Sample holder | Transmission sample holder. Samples are mounted as a thick film. to minimise preferred |
| | orientation effects between Mylar film) using a tin plated stainless steel spacer to afford a |
| | compact that is 1 mm thick and up to 1 cm in diameter. |
| Configuration | Transmission |
| Generator voltage/current | 40 Kv/40 mA |

### Particle Size Distribution (PSD)

The PSD analyses were run on a Sympatec Helos laser diffraction instrument equipped with the RODOS/M for dispersion and the ASPIROS or VIBRI for sample delivery. The powder dispersion is achieved by the use of compressed air and through a gun that uses the Venturi effect. PSD method details are listed in table C.

TABLE C

| Instrumental Parameter | Value |
|---|---|
| Software | Sympatec Windox 5 Version 5.1.2.0 |
| Dispersing Accessory | Vibri |
| Optical concentration | 2-10% |
| Lens | R6 |
| Product details | Density: 1.0000 g/cm3 |
| | Shape factor 1.000 |
| | Calculation mode: HRLD |
| Trigger Condition | Reference measurement: duration 10 s/single |
| | Time base: 100 ms |
| | Measurement: standard mode |
| | Start 0.000 s opt. concentration ≥1.0% |
| | Stop after 5.000 s opt. concentration ≤1.0% |
| | Trigger timeout 20 s |
| Disperser Condition | RODOS |
| | Disperser Type: OASISDRY |
| | Injector: 4 mm |
| | Primary pressure 0.5 bar |
| Feeder | Vibri 70% 0.5 mm gap |
| Vacuum | Nilfisk/delay 3 s from start of extraction to start of measurement |

### Discriminating Dissolution Method pH 6.8 (Obicetrapib)

TABLE D

| Instrumental Parameter | Value |
|---|---|
| Apparatus | USP apparatus II |
| Dissolution medium | Phosphate buffer solution pH 6.8 + 0.2% w/v of Polysorbate 80 |
| Dissolution medium volume [mL] | 900 |
| Dissolution medium Temperature [° C.] | 37 ± 0.5 |
| Rotation Speed [rpm] | 50-Infinity point 250 rpm |
| Sampling Time [min] | 5; 10; 15; 30; 45; 60 and 70 (70 as Infinity point) |
| Sampling Volume [mL] | 1 |
| Separative technique (*) | PVDF 0.45 μm membrane |
| Filter Pre-wetting volume [mL] | 9 (sampling 10 mL and discarding the first 9 mL back to the vessel) |
| Detection | HPLC-UV |

### Discriminating Dissolution Method pH 4.5 (Ezetimibe)

TABLE E

| Instrumental Parameter | Value |
|---|---|
| Apparatus | USP apparatus II |
| Dissolution medium | Acetate buffer solution pH 4.5 + 0.45% w/v of SLS |
| Dissolution medium volume [mL] | 500 |
| Dissolution medium Temperature [° C.] | 37 ± 0.5 |
| Rotation Speed [rpm] | 50-Infinity point 250 rpm |
| Sampling Time [min] | 15; 30; 45; 60 and 70 (70′ as Infinity point). |
| Sampling Volume [mL] | 1 |
| Separative technique (*) | ww/PTFE 0.45 um membrane |
| Filter Pre-wetting volume [mL] | 9 (sampling 10 mL and discarding the first 9 mL back to the vessel) |
| Detection | HPLC-UV |

### QC Dissolution Method pH 6.8 (Obicetrapib)

TABLE F

| Instrumental Parameter | Value |
|---|---|
| Apparatus | USP apparatus II |
| Dissolution medium | Phosphate buffer solution pH 6.8 + 0.2% w/v of Polysorbate 80 |
| Dissolution medium volume [mL] | 1000 |
| Dissolution medium Temperature [° C.] | 37 ± 0.5 |
| Rotation Speed [rpm] | 75-Infinity point 250 rpm |
| Sampling Time [min] | 15; 30; 45; 60 and 70 (70′ as Infinity point at 250 rpm) |
| Sampling Volume [mL] | 1 |
| Separative technique (*) | PVDF 0.45 μm membrane |
| Filter Pre-wetting volume [mL] | 9 (sampling 10 mL and discarding the first 9 mL back to the vessel) |
| Detection | HPLC-UV |

### QC Dissolution Method pH 4.5 (Ezetimibe)

TABLE G

| Instrumental Parameter | Value |
|---|---|
| Apparatus | USP apparatus II |
| Dissolution medium | Acetate buffer solution pH 4.5 + 0.45% w/v of SLS |
| Dissolution medium volume [mL] | 500 |
| Dissolution medium Temperature [° C.] | 37 ± 0.5 |
| Rotation Speed [rpm] | 75-Infinity point 250 rpm |
| Sampling Time [min] | 15; 30; 45; 60 and 70 (70′ as Infinity point). |
| Sampling Volume [mL] | 1 |
| Separative technique (*) | ww/PTFE 0.45 |
| Filter Pre-wetting volume [mL] | 9 (sampling 10 mL and discarding the first 9 mL back to the vessel) |
| Detection | HPLC-UV |

### Assay and Impurities/Related Substances (Obicetrapib)

TABLE H

| Intrumental Parameters | Value | | | |
|---|---|---|---|---|
| Column | Kinetex PFP, 100 mm × 4.6 mm (particle size: 2.6 μm) | | | |
| Mobile Phase | A): 70/30% v/v Water/Methanol + TFA 0.025% | | | |
| | B): 30/70% v/v Methanol/Acetonitrile + TFA 0.025% | | | |
| | Time | Flow Rate | Mobile Phase (A) | Mobile Phase (B) |
| | (min) | [mL/min] | | |
| Gradient Program | 0.0 | 1.0 | 70 | 30 |
| | 5.0 | | 50 | 50 |
| | 8.0 | | 50 | 50 |
| | 30.0 | | 3 | 97 |
| | 30.1 | | 70 | 30 |
| | 35.0 | | 70 | 30 |
| Column Temperature [° C.] | 35 | | | |
| Auto Sampler Temperature [° C.] | R.T. | | | |
| Detector | UV, MWD or DAD (DAD for release ID) | | | |
| Detector Wavelength [nm] | 245 | | | |
| Injection Volume [μL] | 10 | | | |
| Diluent (Standard, sensitivity and | Water/Acetonitrile, 50/50% v/v | | | |
| resolution check solutions) | | | | |

| | |
|---|---|
| Diluent (Sample solutions) | Diluent A: Water 100% |
| | Diluent B: Acetonitrile 100% |
| Run Time [min] | 30 (5 minutes next injection delay) |
| Typical Retention Time [min] | 16.7 |

### Assay and Impurities/Related Substances (Ezetimibe)

TABLE I

| Intrumental Parameters | Value | | | |
|---|---|---|---|---|
| | Kinetex PFP F5, 150 mm × 4.6 mm (particle size: 5.0 μm) | | | |
| | A): Water/Methanol 90/10% v/v | | | |
| | B): Acetonitrile/Methanol 90/10% v/v | | | |
| Gradient Program | Time | Flow Rate | Mobile Phase (A) | Mobile Phase (B) |
| | (min) | [mL/min] | | |
| | 0.0 | 2.0 | 70 | 30 |
| | 37.0 | | 70 | 30 |
| | 60.0 | | 44.4 | 55.6 |
| | 70.0 | | 44.4 | 55.6 |
| | 80.0 | | 11.1 | 88.9 |
| | 90.0 | | 11.1 | 88.9 |
| | 90.1 | | 70 | 30 |
| | 100.0 | | 70 | 30 |
| Column Temperature [° C.] | 25 | | | |
| Auto Sampler Temperature [° C.] | R.T. | | | |
| Detector | 0-5 min: 215 nm and 5-100 min: 248 nm | | | |
| Detector Wavelength [nm] | UV, MWD or DAD (DAD for release ID if required) | | | |
| Injection Volume [μL] | 60 | | | |
| Diluent (Standard, sensitivity and | Water/Acetonitrile/Methanol 63/27/10% v/v with 0.1% of acetic acid | | | |
| identification check solutions) | | | | |

| | |
|---|---|
| Diluent (Sample solutions) | Diluent A: Water with 0.1% of acetic acid |
| | Diluent B: Acetonitrile 1 with 0.1% of acetic acid |
| | Diluent C: Methanol with 0.1% of acetic acid |
| Run Time [min] | 100.0 |
| Typical Retention Time [min] | 21.0 |

### Example 1: Fixed Dose Combination Tablet of 10 mg Ezetimibe, 5 mg Obicetrapib (Small Scale Batch ~500 g)

### High Shear Granulation and Fluid Bed Drying

Four prototype formulations were assessed. The excipients contained in the granule were plastic filler (Avicel PH101), brittle filler (Pharmatose 200M), binder (Kollidon 30), disintegrant (glycolys) and surfactant (Kolliphor SLS fine). In the preliminary four trials (granule batches A4459/05/01, A4459/05/02, A4459/05/03 and A4459/05/04) the quantity of the plastic filler and the brittle filler was assessed at high or low level and two high shear granulation processing conditions were tested. In the last two trials (granule batches A4459/07/01 and A4459/08/01) the formulations were prepared at a high level of lactose and a lower impeller speed (as per processing condition 2). The composition and method of the addition of the excipients was amended as detailed in Table 1.

The materials were dispensed at the target weight and ezetimibe, obicetrapib and the intra-granular excipients were manually sieved and transferred into a granulation bowl. The granulation solution was prepared by solubilising the required excipients in water.

The small-scale granules were dried using a STREA fluid bed granulator and the material was fluidised in the bowl by adjusting the air volume as required and until the LOD of the dried granule was equal or lower than the initial LOD. The inlet air temperature, product temperature, exhaust temperature as well as the air flow volume were registered throughout drying. Following drying, the granules were tested for granule homogeneity of APIs, LOD, sieve analysis, TBD and XRPD.

### Preparation of the Final Blend and Tableting

The final blends were prepared by weighing accurately the required amount of extra-granular excipients. Then, the excipients (with the exception of Magnesium Stearate (MgSt)) were manually sieved, added with the granule to a bin of suitable volume and blended using a Pharmatech mixer. The MgSt was sieved separately and added to the bin. For the compression, a single punch compression machine (specifically, the EK0 tableting machine) was used to generate the compression profile and manufacture tablets with 150.0 mg target weight. Based on the information collected for the compression profile, a small-scale tablet manufacture was performed. These tablets were tested for appearance, assay and impurities content, discriminating dissolution, ezetimibe USP tablet dissolution method, content uniformity, water content by KF and XRPD. All the intermediates of production and the uncoated tablets were stored in double LDPE bags closed with a cable tie and transferred into a sealed aluminium bag with silica.

TABLE 1
Composition (% w/w) of granule and tablet of small scale 10 mg ezetimibe and
5 mg obicetrapib trials

| | Formulation prototype | | | | | | | |
|---|---|---|---|---|---|---|---|---|
| | 1 | | 2 | | 3 | | 4 | |
| | Processing condition | | | | | | | |
| | Condition 1 | Condition 2 | Condition 1 | Condition 2 | Condition 2 | | Condition 2 | |
| | Granule Batch number | | | | | | | |
| | A4459/05/01 | A4459/05/02 | A4459/05/03 | A4459/05/04 | A4459/07/01 | | A4459/08/01 | |
| | Tablet Batch number | | | | | | | |
| | A4459/05/05 | A4459/05/06 | A4459/05/07 | A4459/05/08 | A4459/07/02 | | A4459/08/02 | |
| | % (w/w) | % (w/w) | % (w/w) | % (w/w) | % (w/w) | % (w/w) | % (w/w) | % (w/w) |
| Component | granule | tablet | granule | tablet | granule | tablet | granule | tablet |
| Ezetimibe | 7.168 | 6.666 | 7.168 | 6.666 | 7.017 | 6.666 | 7.073 | 6.666 |
| Obicetrapib | 3.678 | 3.421 | 3.678 | 3.421 | 3.601 | 3.421 | 3.630 | 3.421 |
| (salt) | | | | | | | | |
| Avicel PH | 26.826 | 24.948 | 53.651 | 49.895 | 20.968 | 19.920 | 21.775 | 20.717 |
| 101 | | | | | | | | |
| Pharmatose | 53.651 | 49.895 | 26.826 | 24.948 | 57.887 | 54.993 | 60.095 | 57.196 |
| 200M | | | | | | | | |
| Kollidon 30 | 5.018 (out) | 4.667 | 5.018 (out) | 4.667 | 4.211 | 4.000 | 1.061 (in) | 1.000 |
| | | | | | (in) | | | |
| Glycolys | 3.301 | 3.070 | 3.301 | 3.070 | 4.211 | 4.000 | 4.244 | 4.000 |
| Kolliphor | 0.358 (out) | 0.333 | 0.358 (out) | 0.333 | 2.105 | 2.000 | 2.122 (out) | 2.000 |
| SLS fine | | | | | (out) | | | |
| MilliQ water | 30.000 | N/A | 30.000 | N/A | 20.000 | N/A | 20.000 | N/A |
| * | | | | | | | | |
| Total granule | 100.000 | 93.000 | 100.000 | 93.000 | 100.000 | 95.000 | 100.000 | 95.000 |
| Avicel PH | N/A | 3.000 | N/A | 3.000 | N/A | N/A | N/A | N/A |
| 101 | | | | | | | | |
| Glycolys | N/A | 2.500 | N/A | 2.500 | N/A | 4.000 | N/A | 4.000 |
| Aerosil 200 | N/A | 0.750 | N/A | 0.750 | N/A | N/A | | N/A |
| Ligamed MF- | N/A | 0.750 | N/A | 0.750 | N/A | 1.000 | N/A | 1.000 |
| 2-V | | | | | | | | |
| Total tablet | N/A | 100.000 | N/A | 100.000 | N/A | 100.000 | N/A | 100.000 |

### Results:

### Small-Scale Trials to Develop 10 mg Ezetimibe, 5 mg Obicetrapib Tablets

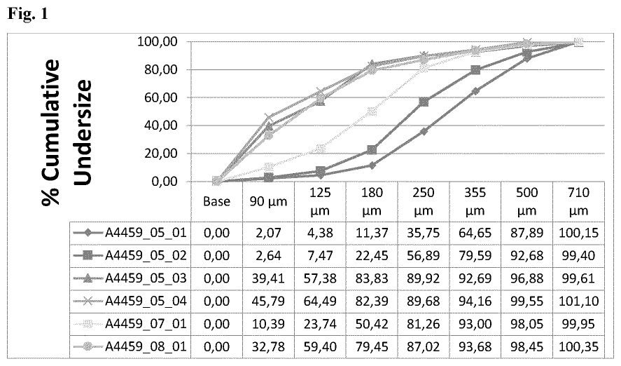

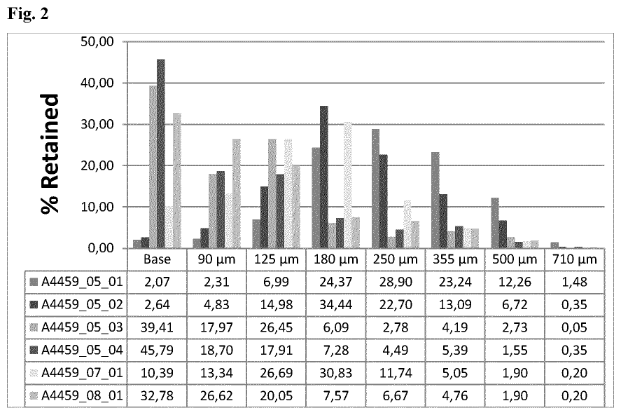

The manufactures of the granule for the small-scale batches were conducted successfully. During granulation the energy consumption increased upon addition of the granulation solution and, after drying, the LOD of the granule was lower than the initial LOD (Table 2). The granules A4459/05/01 and A4459/05/02 presented coarser particles in comparison to granule batches A4459/05/03 and A4459/05/04. This was linked to a higher level of lactose in the formulation rather than to the parameters of granulation (process condition 1 vs process condition 2). As the quantity of the binder and water for granulation was reduced and the level of surfactant increased, the granule batches A4459/07/01 and A4459/08/01 (that were manufactured with a higher level of lactose), presented particles with a larger portion of fines in comparison to batches A4459/05/01 and A4459/05/02 (FIG. 1 and FIG. 2). Briefly, the tablets with higher content of microcrystalline cellulose (batches A4459/05/07 and A4459/05/08) showed faster disintegration time, lower friability and higher hardness values than those containing a higher quantity of lactose (batches A4459/05/05 and A4459/05/06). Overall, these tablet batches presented a suitable appearance. The tablet batches A4459/07/02 and A4459/08/02 (containing high level of lactose) presented faster time of disintegration and suitable dissolution profile of both drug substances. However, the hardness and friability of the tablets could not be improved to a level considered acceptable due to capping and failure of the friability test. The tablet hardness was lower compared to that obtained with the previous trials (batch A4459/05/05 and A4459/05/06).

TABLE 2
LOD of 10 mg ezetimibe, 5 mg obicetrapib small-scale trials

| | A4459/05/01 | A4459/05/02 | A4459/05/03 | A4459/05/04 | A4459/07/01 | A4459/08/01 |
|---|---|---|---|---|---|---|
| | Prototype | Prototype | Prototype | Prototype | Prototype | Prototype |
| | 1 | 1 | 2 | 2 | 3 | 4 |
| | condition | condition | condition | condition | condition | condition |
| LOD(%) | 1 | 2 | 1 | 2 | 2 | 2 |
| Blend for granulation (prior to spraying) | 3.62% | 2.84% | 4.00% | 3.49% | 2.79% | 2.17% |
| Granule following spraying (prior to drying) | 16.79% | 26.78% | 26.42% | 27.68% | 20.06% | 20.35% |
| Granule following drying | 2.26% | 2.33% | 2.70% | 3.07% | 1.97% | 1.14% |
| Granule following milling | 3.82% | 2.58% | 2.70% | 2.51% | 2.07% | 1.55% |
| Granule following additional drying | 2.95% | N/A | N/A | N/A | N/A | N/A |

### Granules Characterization

### Chemical Characterization Analysis

The granules were tested for homogeneity of both obicetrapib and ezetimibe, results are reported in Table 3. Both APIs were homogenously dispersed in the granule with maximum RSD % values obtained for batch A4459/05/03, nonetheless within the typical acceptable range for granule homogeneity.

TABLE 3
Granule uniformity of 500 g batch scale prototypes

| | A4459/05/01 | | A4459/05/02 | | A4459/05/03 | |
|---|---|---|---|---|---|---|
| | Prototype 1 | | Prototype 1 | | Prototype 2 | |
| | condition 1 | | condition 2 | | condition 1 | |
| | Ezetimibe | Obicetrapib | Ezetimibe | Obicetrapib | Ezetimibe | Obicetrapib |
| | (% | (% | (% | (% | (% | (% |
| | Claim) | claim) | Claim) | claim) | Claim) | claim) |
| Sam#1 | 101.9 | 97.9 | 102.5 | 98.5 | 99.4 | 98.1 |
| Sam#2 | 101.1 | 97.6 | 103.3 | 99.6 | 101.8 | 98.8 |
| Sam#3 | 103.0 | 98.8 | 103.4 | 99.2 | 103.7 | 100.9 |
| Sam#4 | 101.7 | 97.9 | 103.6 | 99.7 | 101.6 | 98.9 |
| Sam#5 | 102.6 | 98.8 | 103.1 | 98.8 | 101.6 | 98.6 |
| Sam#6 | 103.3 | 99.1 | 103.9 | 98.6 | 101.8 | 98.7 |
| Mean | 102.3 | 98.4 | 103.1 | 99.1 | 101.7 | 99.0 |
| RSD | 0.82 | 0.66 | 0.40 | 0.55 | 1.32 | 0.99 |
| % | | | | | | |
| | A4459/05/04 | | A4459/07/01 | | A4459/08/01 | |
| | Prototype 2 | | Prototype 3 | | Prototype 4 | |
| | condition 2 | | condition 2 | | condition 2 | |
| | Ezetimibe | Obicetrapib | Ezetimibe | Obicetrapib | Ezetimibe | Obicetrapib |
| | (% | (% | (% | (% | (% | (% |
| | Claim) | claim) | Claim) | claim) | Claim) | claim) |
| Sam#1 | 102.2 | 98.3 | 102.4 | 98.5 | 103.0 | 101.8 |
| Sam#2 | 101.7 | 97.9 | 102.1 | 98.1 | 99.5 | 97.9 |
| Sam#3 | 103.1 | 98.5 | 101.4 | 97.4 | 101.8 | 100.3 |
| Sam#4 | 102.9 | 98.6 | 99.3 | 95.2 | 99.5 | 98.2 |
| Sam#5 | 102.3 | 98.0 | 98.9 | 94.7 | 100.6 | 99.3 |
| Sam#6 | 102.3 | 98.5 | 102.6 | 98.5 | 101.7 | 99.6 |
| Mean | 102.8 | 98.3 | 101.1 | 97.1 | 101.0 | 99.5 |
| RSD | 0.53 | 0.31 | 1.6 | 1.7 | 1.4 | 1.4 |
| % | | | | | | |

### Physical Properties Characterization

As reported in Table 4, a small amount of ezetimibe (EZE) hydrate was found in all the wet granules samples. However, during the drying process the formed EZE hydrate converts back to EZE anhydrous with exception of batch 05/01 in which small trace of the hydrate polymorphic form appears to be still present.

TABLE 4
XRPD data summary small scale 10 mg ezetimibe and
5 mg obicetrapib blends and wet and dry granules

| Sample | Batch ID | XRPD |
|---|---|---|
| Prototype 1 condition 1 Blend | A4459/05/01 | OBI + EZE Anhydrous |
| Prototype 1 condition 1 Wet Granules | | OBI + EZE Anhydrous + some EZE Hydrate |
| Prototype 1 condition 1 Dry Granules | | OBI + EZE Anhydrous + small EZE Hydrate |
| Prototype 1 condition 2 Blend | A4459/05/02 | OBI + EZE Anhydrous |
| Prototype 1 condition 2 Wet Granules | | OBI + EZE Anhydrous + small EZE Hydrate |
| Prototype 1 condition 2 Dry Granules | | OBI + EZE Anhydrous |
| Prototype 2 condition 1 Blend | A4459/05/03 | OBI + EZE Anhydrous |
| Prototype 2 condition 1 Wet Granules | | OBI + EZE Anhydrous + small EZE Hydrate |
| Prototype 2 condition 1 Dry Granules | | OBI + EZE Anhydrous |
| Prototype 2 condition 2 Blend | A4459/05/04 | OBI + EZE Anhydrous |
| Prototype 2 condition 2 Wet Granules | | OBI + EZE Anhydrous + small EZE Hydrate |
| Prototype 2 condition 2 Dry Granules | | OBI + EZE Anhydrous |

### Tablets Characterization

### Chemical Characterization Analysis

Results for the characterization of the small scale batch are reported in Table 5. Results for assay and impurities met the expectation and the impurity profile was consistent with both input APIs. All the prototypes were also found with an homogenous APIs content as the content uniformity results was with AV values significantly lower than the pharmacopeial requirement of AV. Water content results were found to be in range from 4.5 and 5.0%, and no defects were observed on the appearance.

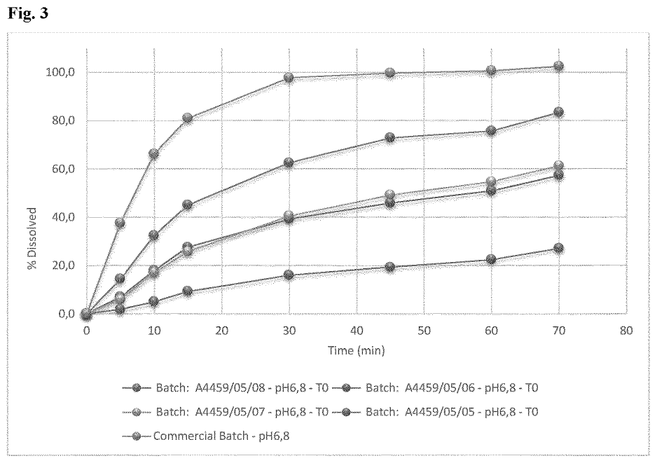

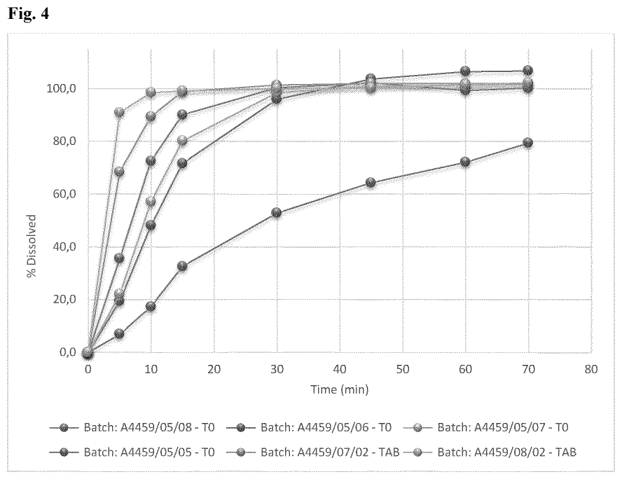

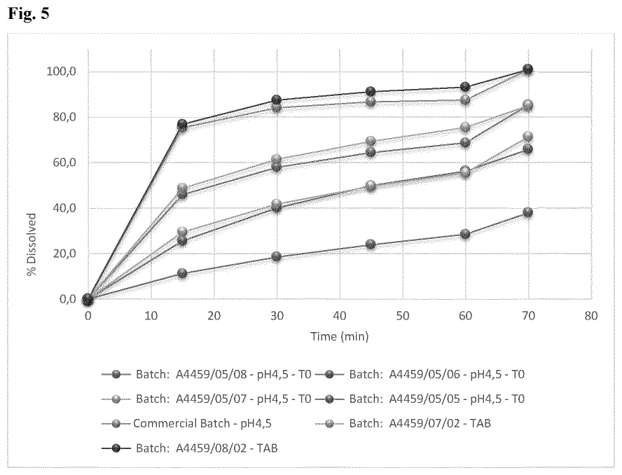

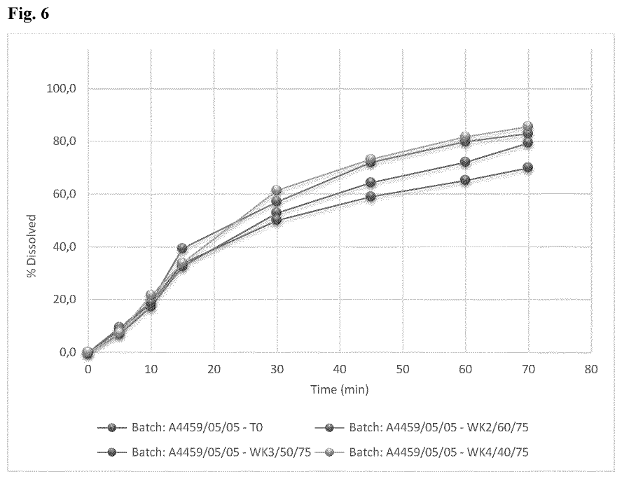

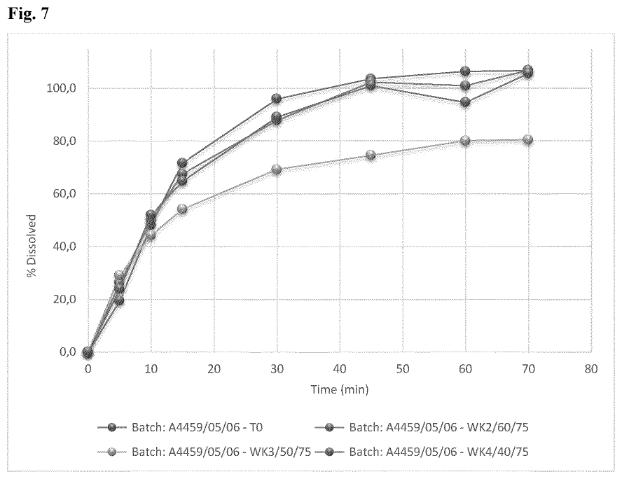

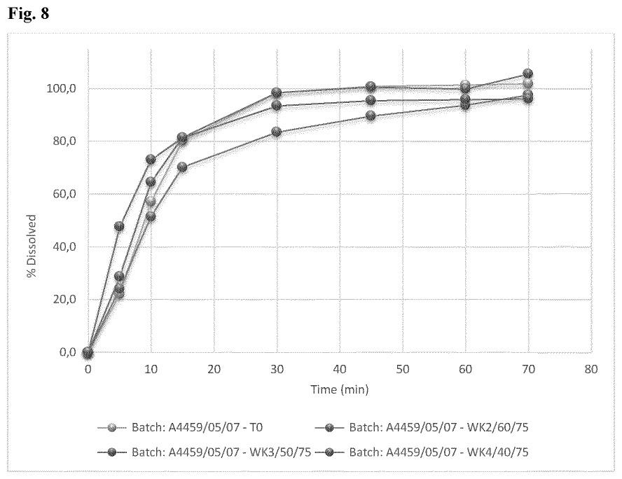

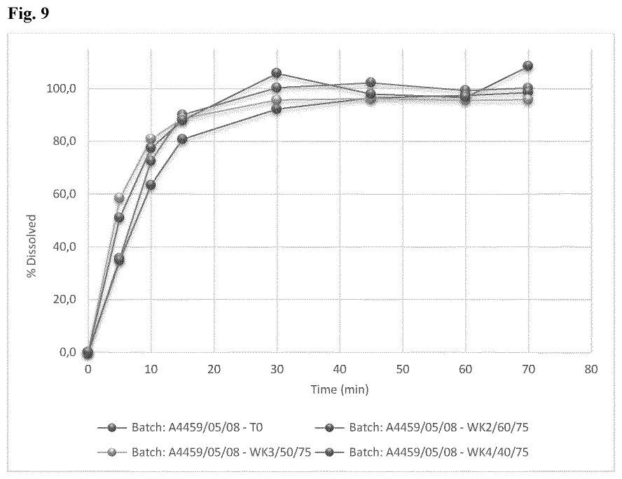

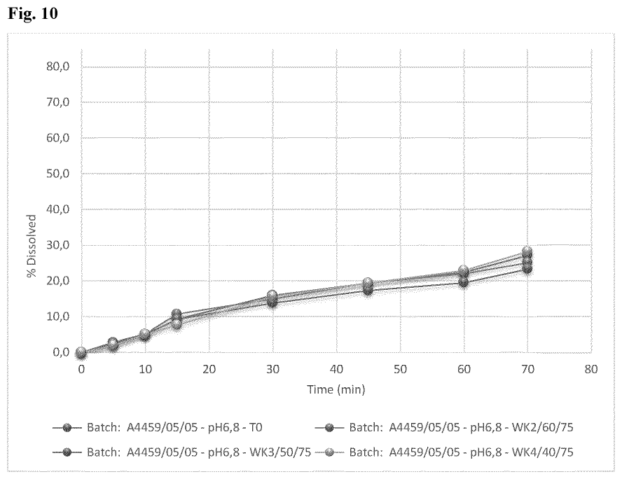

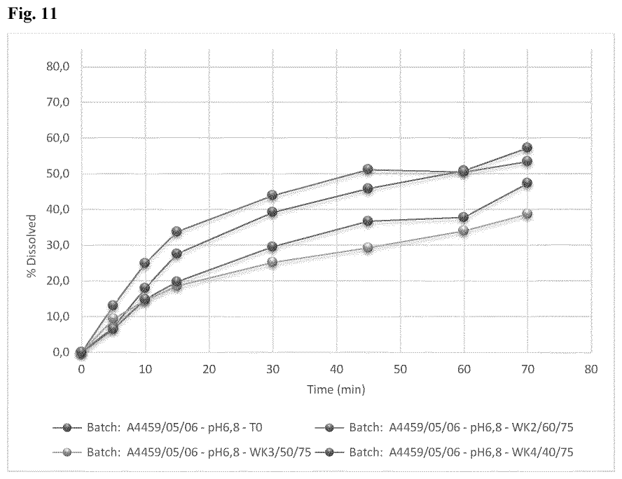

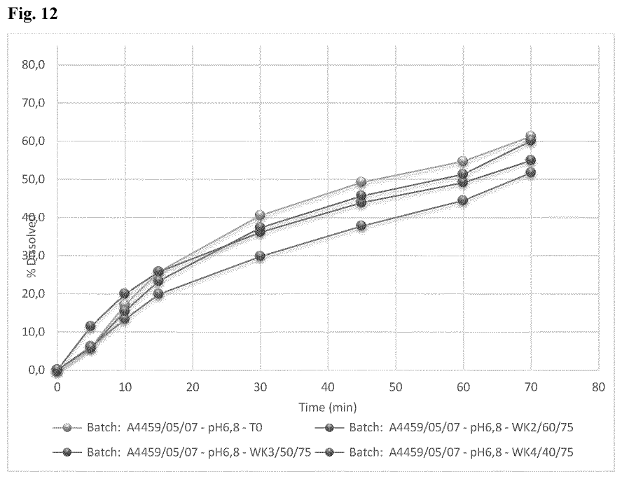

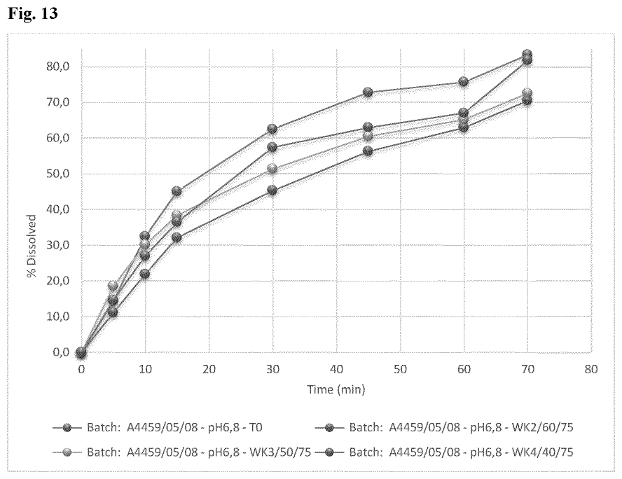

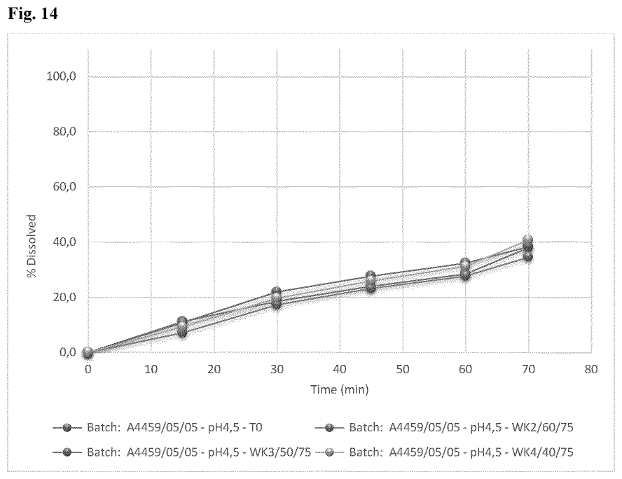

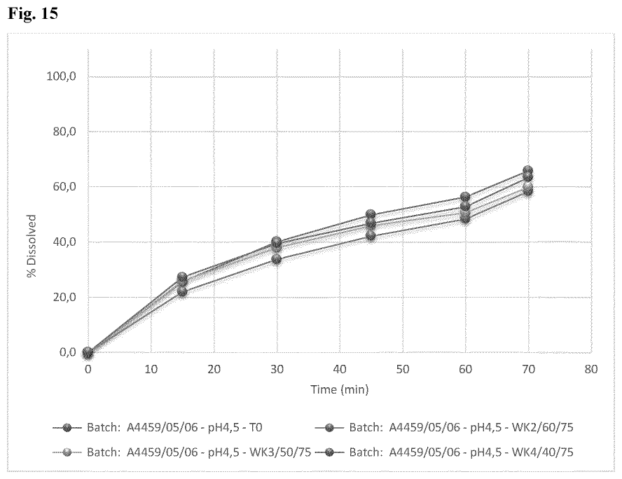

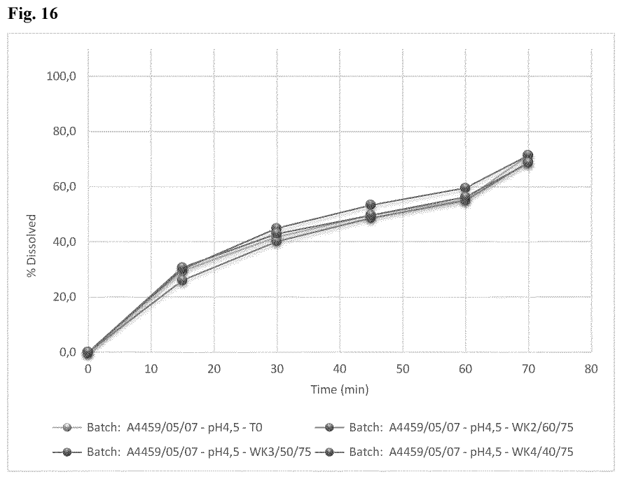

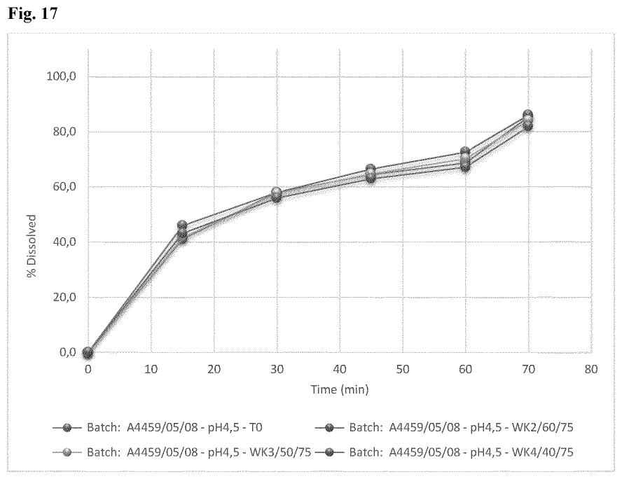

The dissolution profiles for Obicetrapib showed for Prototypes A4459/05/08, A4459/05/06 and A4459/05/07 a similar trend in dissolution with Prototype A4459/05/08 (high amount of Avicel & low impeller speed) dissolving rapidly in the range 5-15 minutes. Prototype A4459/05/05 (high Lactose & high impeller speed) dissolved significantly slower. Dissolution results in USP ezetimibe method pH 4.5 were consistent with what observed in pH 6.8. For prototypes 3 and 4 batches A4459/07/02 (4% Binder) and A4459/08/02 (1% binder) a significant improvement was observed in dissolution characterization which shows for prototype 4 a profile consistent with the reference commercial ezetimibe tablet. Dissolution profiles are presented in FIG. 3, FIG. 4 and FIG. 5. Assay, content uniformity and impurities profile showed no significant differences across the four formulations for both obicetrapib and ezetimibe.

TABLE 5
Results of the analytical characterization of small scale 10 mg ezetimibe and 5 mg
obicetrapib

| | | Manufacturing lot number | | | | | |
|---|---|---|---|---|---|---|---|
| | | A4459/05/05 | A4459/05/06 | A4459/05/07 | A4459/05/08 | A4459/07/02 | A4459/08/02 |
| | | Prototype and Process condition | | | | | |
| | | 1-high | 2-low | 1-high | 2-low | | |
| | | impeller | impeller | impeller | impeller | | |
| | | speed & 3 | speed & 1 | speed & 3 | speed & 1 | Prototype 3 | Prototype 4 |
| | | min wet | min wet | min wet | min wet | condition 2 | condition 2 |
| | | massing | massing | massing | massing | (4% Binder) | (1% Binder) |
| | | MCC:Lactose Ratio | | | | | |
| | | 24.9:49.9 | 24.9:49.9 | 49.9:24.9 | 49.9:24.9 | N/A | N/A |
| Test | Method | Result | Result | Result | Result | Result | Result |
| Appearance | Visual | White round | White round | White round | White round | White round | White round |
| | | coated | coated | coated | coated | coated | coated |
| | | tablets | tablets | tablets | tablets | tablets | tablets |
| Assay | HPLC | | | | | | |
| Obicetrapib | 2730 | 100.2 | 101.5 | 99.5 | 98.5 | 100.5 | 101.9 |
| (% claim) | | | | | | | |
| Ezetimibe (% claim) | 2731 | 102.3 | 98.4 | 99.2 | 101.4 | 102.8 | 101.2 |
| Impurities | HPLC | | | | | | |
| IMP_RRT0.92 (% | 2730 | N.A. | N.A. | N.A. | N.A. | 0.05 | N.A. |
| claim) | | | | | | | |
| IMP RRT0.94 (% | 2730 | 0.05 | 0.05 | 0.05 | 0.05 | 0.07 | 0.07 |
| claim) | | | | | | | |
| FROL(% claim) | 2730 | 0.05 | 0.05 | 0.05 | 0.05 | 0.07 | 0.07 |
| MONO-BN | 2730 | 0.17 | 0.17 | 0.17 | 0.17 | 0.18 | 0.18 |
| FREE(% claim) | | | | | | | |
| Total impurities of | 2730 | 0.27 | 0.27 | 0.27 | 0.26 | 0.37 | 0.32 |
| Obicetrapib ≥ | | | | | | | |
| 0.05% | | | | | | | |
| EzetimibeKetone | 2731 | N.D. | N.D. | N.D. | 0.05 | N.D. | N.D. |
| (% a/a) | | | | | | | |
| Ezetimibe | 2731 | N.D. | N.D. | N.D. | 0.07 | N.D. | 0.05 |
| tetrahydropyran | | | | | | | |
| analog (% a/a) | | | | | | | |
| Total impurities of | 2731 | 0.06 | 0.06 | 0.07 | N.D. | N.D. | 0.05 |
| Ezetimbe ≥ 0.05% | | | | | | | |
| Uniformity of | USP <905> | Average % | Average % | Average % | Average % | Average % | Average % |
| dosage units | Ph. Eur. 2.9.40 | Claim: 98.5 | Claim: 98.3 | Claim: 98.4 | Claim: 97.2 | Claim: 99.2 | Claim: 100.7 |
| Obicetrapib | or | Range % | Range % | Range % | Range % | Range % | Range % |
| | JP 6.02 by | Claim: | Claim: | Claim: (97.9- | Claim: | Claim: | Claim: |
| | UNIC/ | (96.7-99.7) | (97.4-99.7) | 99.1) | (96.6-97.7) | (96.7-100.7) | (99.6-101.9) |
| | 1190 | AV = 2.3 | AV = 1.8 | AV = 0.9 | AV= 2.1 | AV =4.0 | AV = 1.9 |
| Ezetimibe | | Average % | Average % | Average % | Average % | Average % | Average % |
| | | Claim: 101.2 | Claim: 100.7 | Claim: 99.2 | Claim: 99.4 | Claim: 103.3 | Claim: 102.1 |
| | | Range % | Range % | Range % | Range % | Range % | Range % |
| | | Claim: | Claim: | Claim: | Claim: | Claim: | Claim: |
| | | (99.5-102.3) | (100.1- | (99.0-99.8) | (98.8-99.8) | (101.3- | (100.6- |
| | | AV = 2.2 | 102.1) | AV = 0.5 | AV = 0.9 | 105.0) | 103.1) |
| | | | AV = 1.4 | | | AV = 5.8 | AV = 2.9 |
| Dissolution | | | | | | | |
| Obicetrapib | | | | | | | |
| 5 min Average | Discriminating | 7 | 20 | 22 | 36 | 68 | 91 |
| (Min Max) | method at pH | (6-8) | (15-25) | (20-26) | (29-41) | (64-72) | (77-95) |
| 10 min Average | 6.8, 50 rpm | 17 | 49 | 57 | 73 | 89 | 98 |
| (Min Max) | paddle speed | (16-19) | (40-57) | (52-61) | (65-77) | (73-98) | (97-99) |
| 15 min Average | and 900 mL | 33 | 73 | 80 | 91 | 95 | 95 |
| (Min Max) | vessel volume | (31-35) | (70-76) | (79-82) | (89-93) | (79-99) | (77-101) |
| 30 min Average | | 53 | 97 | 98 | 101 | 100 | 100 |
| (Min Max) | | (51-56) | (96-99) | (97-100) | (98-105) | (92-103) | (99-101) |
| 45 min Average | | 64 | 105 | 101 | 103 | 102 | 100 |
| (Min Max) | | (63-68) | (104-106) | (99-103) | (100-107) | (101-103) | (99-102) |
| 60 min Average | | 72 | 108 | 101 | 100 | 96 | 98 |
| (Min Max) | | (70-73) | (106-110) | (100-103) | (97-11) | (70-102) | (80-105) |
| 70 min Average | | 79 | 108 | 101 | 101 | 102 | 102 |
| (Min Max) | | (78-81) | (107-109) | (100-103) | (98-105) | (101-102) | (100-105) |
| Dissolution | | | | | | | |
| Ezetimibe | | | | | | | |
| 5 min Average | Discriminating | 2 | 7 | 6 | 15 | — | — |
| (Min Max) | method at pH | (2-2) | (5-9) | (5-7) | (12-17) | | |
| 10 min Average | 6.80.2% w/v | 5 | 18 | 17 | 33 | — | — |
| (Min Max) | of Tween 80, | (4-6) | (15-22) | (16-18) | (28-35) | | |
| 15 min Average | 50 rpm paddle | 9 | 28 | 26 | 45 | — | — |
| (Min Max) | speed and 900 | (8-10) | (27-29) | (25-27) | (43-48) | | |
| 30 min Average | mL vessel | 16 | 40 | 40 | 63 | — | — |
| (Min Max) | volume | (15-18) | (39-42) | (39-41) | (61-64) | | |
| 45 min Average | | 19 | 47 | 49 | 73 | — | — |
| (Min Max) | | (19-21) | (45-50) | (47-50) | (70-77) | | |
| 60 min Average | | 22 | 52 | 55 | 76 | — | — |
| (Min Max) | | (21-23) | (51-54) | (53-56) | (73-84) | | |
| 70 min Average | | 27 | 58 | 61 | 84 | — | — |
| (Min Max) | | (26-27) | (57-61) | (60-62) | (82-88) | | |
| Dissolution | | | | | | | |
| Ezetimibe (n = 3) | | | | | | | |
| 15 min Average | Dissolution | 11 | 26 | 29 | 46 | 47 | 78 |
| (Min Max) | method based | (11-12) | (25-26) | (28-31) | (42-49) | (46-52) | (73-80) |
| 30 min Average | on USP | 19 | 41 | 42 | 59 | 61 | 87 |
| (Min Max) | method | (18-19) | (40-41) | (41-43) | (55-61) | (59-65) | (83-90) |
| 45 min Average | conditions at | 24 | 51 | 49 | 65 | 69 | 91 |
| (Min Max) | pH 4.5, 50 | (23-25) | (50-51) | (49-50) | (62-67) | (67-72) | (86-94) |
| 60 min Average | rpm paddle | 29 | 57 | 55 | 69 | 75 | 93 |
| (Min Max) | speed and 500 | (27-29) | (57-58) | (55-55) | (67-71) | (73-78) | (88-96) |
| 70 min Average | mL vessel | 38 | 67 | 71 | 86 | 85 | 101 |
| (Min Max) | volume | (36-40) | (66-68) | (71-71) | (85-87) | (82-87) | (100-102) |
| Water content | KF | 5.0% w/w | 4.5% w/w | 4.5% w/w | 4.4% w/w | 6.1% w/w | 5.4% w/w |

### Stress Stability

Prototypes 1 and 2 with different process conditions were evaluated in a stress stability study with the following design

TABLE 6
Stress stability study design

| Storage Conditions | | Time (weeks) | | | |
|---|---|---|---|---|---|
| T/RH | Type | 0 | 2 | 3 | 4 |
| 25° C./60% RH | Long-term | T | (T) | — | (T) |
| 40° C./75% RH uncovered | Stress (T/H) | | (T) | — | T |
| 40° C./75% RH closed | Accelerated | | (T) | — | (T) |
| 50° C./75% RH uncovered | Stress (T/H) | | (T) | T | |
| 60° C./75% RH closed | Stress (T) | | T | (T) | |

Key:
T = Tested for appearance, assay & related substances, discriminating dissolution, water content by KF, and form check by XRPD
(T) = Optional testing

Results are reported in Table 7, Table 8 and Table 9.

TABLE 7
Result for Appearance, assay and water content of small scale 10 mg ezetimibe
and 5 mg obicetrapib

| | | | | Assay | Assay | Water |
|---|---|---|---|---|---|---|
| | Storage conditions | Time point | Appearance | Obicetrapib | Ezetimibe | Content |
| Batch # | (° C./% RH) | (weeks) | Visual inspection | (% claim) | (% ClaimP | (% w/w) |
| A4459/05/05 | Initial | | White round tablets | 100.2 | 101.4 | 4.99 |
| MCC:Lactose = 24.9:49.9 | 60° C./75% RH | 2 weeks | Off-white round | 99.3 | 101.7 | 5.25 |
| | Packaged | | tablets | | | |
| Process condition 1-high | 50° C./75% RH exposed | 3 weeks | Consistent with | 99.8 | 100.5 | 6.40 |
| | | | T = 2 w | | | |
| impeller speed & 3 min wet | 40° C./75% RH exposed | 4 weeks | Consistent with | 99.7 | 102.7 | 7.04 |
| massing | | | T = 2 w | | | |
| A4459/05/06 | Initial | | White round tablets | 101.5 | 102.3 | 4.53 |
| MCC:Lactose = 24.9:49.9 | 60° C./75% RH | 2 weeks | Off-white round | 101.6 | 103.2 | 5.02 |
| | Packaged | | tablets | | | |
| Process condition 2-low | 50° C./75% RH exposed | 3 weeks | Consistent with | 102.1 | 101.8 | 6.51 |
| | | | T = 2 w | | | |
| impeller speed & | 40° C./75% RH exposed | 4 weeks | Consistent with | 101.1 | 103.2 | 6.86 |
| 1 min wet | | | T = 2 w | | | |
| massing | | | | | | |
| A4459/05/07 | Initial | | White round tablets | 99.5 | 98.4 | 4.50 |
| MCC:Lactose = 49.9:24.9 | 60° C./75% RH | 2 weeks | Off-white round | 98.8 | 99.2 | 4.95 |
| | Packaged | | tablets | | | |
| Process condition 1-high | 50° C./75% RH exposed | 3 weeks | Consistent with | 98.9 | 98.7 | 7.01 |
| | | | T = 2 w | | | |
| impeller speed & 3 min wet | 40° C./75% RH exposed | 4 weeks | Consistent with | 98.5 | 100.4 | 7.48 |
| massing | | | T = 2 w | | | |
| A4459/05/08 | Initial | | White round tablets | 98.5 | 99.2 | 4.41 |
| MCC:Lactose = 49.9:24.9 | 60° C./75% RH | 2 weeks | Off-white round | 98.3 | 101.1 | 4.91 |
| | Packaged | | tablets | | | |
| Process condition2-low | 50° C./75% RH exposed | 3 weeks | Consistent with | 99.0 | 99.5 | 7.07 |
| | | | T = 2 w | | | |
| impeller speed & | 40° C./75% RH exposed | 4 weeks | Consistent with | 98.4 | 101.3 | 7.43 |
| 1 min wet | | | T = 2 w | | | |
| massing | | | | | | |

TABLE 8
Impurities profile of small scale 10 mg ezetimibe and 5 mg obicetrapib

| | Storage | | Obicetrapib related impurities | | | | | | Ezetimibe related impurities | | |
|---|---|---|---|---|---|---|---|---|---|---|---|
| | conditions | Time | | | | | MONO- | Total | | | Total |
| | (° C./% | point | IMP_ | IMP_ | IMP_ | | BN | Impurities | IMP_ | IMP_ | Impurities |
| Batch # | RH) | (weeks) | RRT0.47 | RRT0.50 | RRT0.94 | FROL | FREE | (≥0.05%) | RRT1.50 | RRT2.08 | (≥0.05%) |
| A4459/05/ | Initial | | N.D. | N.D. | 0.05 | 0.05 | 0.17 | 0.27 | N.D. | N.D. | <0.05% |
| 05 | | | | | | | | | | | |
| MCC: | 60° C./75% | 2 | N.D. | N.D. | 0.07 | 0.06 | 0.16 | 0.29 | 0.53 | 0.38 | 0.91 |
| Lactose = | RH | weeks | | | | | | | | | |
| 24.9:49.9 | Packaged | | | | | | | | | | |
| Process | 50° C./75% | 3 | 0.05 | 0.06 | 0.07 | 0.06 | 0.17 | 0.40 | 1.25 | 0.10 | 1.35 |
| condition 1- | RH | weeks | | | | | | | | | |
| high | exposed | | | | | | | | | | |
| impeller | | | | | | | | | | | |
| speed & 3 | | | | | | | | | | | |
| min wet | | | | | | | | | | | |
| massing | | | | | | | | | | | |
| | 40° C./75% | 4 | N.D. | N.D. | 0.08 | 0.06 | 0.17 | 0.31 | 0.26 | 0.08 | 0.33 |
| | RH | weeks | | | | | | | | | |
| | exposed | | | | | | | | | | |
| A4459/05/ | Initial | | N.D. | N.D. | 0.05 | 0.05 | 0.17 | 0.27 | N.D. | N.D. | <0.05% |
| 06 | | | | | | | | | | | |
| MCC: | 60° C./75% | 2 | N.D. | N.D. | 0.07 | 0.06 | 0.17 | 0.31 | 0.06 | 0.20 | 0.26 |
| Lactose = | RH | weeks | | | | | | | | | |
| 24.9:49.9 | Packaged | | | | | | | | | | |
| Process | 50° C./75% | 3 | <0.05% | 0.06 | 0.07 | 0.06 | 0.17 | 0.35 | 1.19 | 0.10 | 1.29 |
| condition 2- | RH | weeks | | | | | | | | | |
| low | exposed | | | | | | | | | | |
| impeller | | | | | | | | | | | |
| speed & 1 | | | | | | | | | | | |
| min wet | | | | | | | | | | | |
| massing | | | | | | | | | | | |
| | 40° C./75% | 4 | N.D. | N.D. | 0.08 | 0.06 | 0.16 | 0.31 | 0.23 | 0.09 | 0.32 |
| | RH | weeks | | | | | | | | | |
| | exposed | | | | | | | | | | |
| A4459/05/ | Initial | | N.D. | N.D. | 0.05 | 0.05 | 0.17 | 0.27 | N.D. | N.D. | <0.05% |
| 07 | | | | | | | | | | | |
| MCC: | 60° C./75% | 2 | N.D. | N.D. | 0.07 | 0.06 | 0.16 | 0.29 | 0.05 | 0.17 | 0.23 |
| Lactose = | RH | weeks | | | | | | | | | |
| 49.9:24.9 | Packaged | | | | | | | | | | |
| Process | 50° C./75% | 3 | N.D | N.D. | 0.05 | 0.05 | 0.17 | 0.24 | 0.53 | 0.10 | 0.63 |
| condition 1- | RH | weeks | | | | | | | | | |
| high | exposed | | | | | | | | | | |
| impeller | | | | | | | | | | | |
| speed & 3 | | | | | | | | | | | |
| min wet | | | | | | | | | | | |
| massing | | | | | | | | | | | |
| | 40° C./75% | 4 | N.D. | N.D. | 0.09 | 0.06 | 0.16 | 0.31 | 0.08 | 0.08 | 0.16 |
| | RH | weeks | | | | | | | | | |
| | exposed | | | | | | | | | | |
| A4459/05/ | Initial | | N.D. | N.D. | 0.05 | 0.05 | 0.17 | 0.26 | N.D. | N.D. | <0.05% |
| 08 | | | | | | | | | | | |
| MCC: | 60° C./75% | 2 | N.D. | N.D. | 0.07 | 0.06 | 0.16 | 0.30 | N.D. | 0.15 | 0.15 |
| Lactose = | RH | weeks | | | | | | | | | |
| 49.9:24.9 | Packaged | | | | | | | | | | |
| Process | 50° C./75% | 3 | N.D | N.D. | 0.07 | 0.06 | 0.16 | 0.30 | 0.50 | 0.10 | 0.60 |
| condition2- | RH | weeks | | | | | | | | | |
| low | exposed | | | | | | | | | | |
| impeller | | | | | | | | | | | |
| speed & 1 | | | | | | | | | | | |
| min wet | | | | | | | | | | | |
| massing | | | | | | | | | | | |
| | 40° C./75% | 4 | N.D. | N.D. | 0.08 | 0.06 | 0.16 | 0.30 | 0.08 | 0.09 | 0.18 |
| | RH | weeks | | | | | | | | | |
| | exposed | | | | | | | | | | |

TABLE 9
Results of the dissolution characterization of small scale 10 mg ezetimibe and
5 mg obicetrapib stress stability

| | | Manufacturing lot number | | | | | | | |
|---|---|---|---|---|---|---|---|---|---|
| | | A4459/05/05 | | | | A4459/05/06 | | | |
| | | Process condition | | | | | | | |
| | | 1-high impeller speed & 3 min wet massing | | | | 2-low impeller speed & 1 min wet massing | | | |
| | | Time point and storage conditions | | | | | | | |
| | | | 2 weeks- | 3 weeks- | | | 2 weeks- | 3 weeks- | |
| | | | 60° C./ | 50° C./ | 4 weeks- | | 60° C./ | 50° C./ | 4 weeks- |
| | | | 75% RH | 75% RH | 40° C./75% | | 75% RH | 75% RH | 40° C./75% |
| | | Initial | Packaged | exposed | RH packed | Initial | Packaged | exposed | RH packed |
| Test | | Result | Result | Result | Result | Result | Result | Result | Result |
| Obicetrapib | Method | (n = 6) | (n = 3) | (n = 3) | (n = 3) | (n = 6) | (n = 3) | (n = 3) | (n = 3) |
| 5 min | Discriminating | 7 | 9 | 9 | 7 | 20 | 26 | 30 | 25 |
| Average | method at | (6- | (9-10) | (7-10) | (6-9) | (15- | (26-27) | (29-31) | (23-28) |
| (Min Max) | pH 6.80.2 | 8) | | | | 25) | | | |
| 10 min | % w/v of | 17 | 19 | 19 | 22 | 49 | 51 | 45 | 54 |
| Average | rpm paddle | (16- | (18-20) | (17-21) | (20-24) | (40- | (48-52) | (45-46) | (52-55) |
| (Min Max) | speed and 900 | 19) | | | | 57) | | | |
| 15 min | mL vessel | 33 | 40 | 34 | 35 | 73 | 68 | 56 | 68 |
| Average | volume | (31- | (38-41) | (32-36) | (31-38) | (70- | (67-70) | (55-56) | (63-72) |
| (Min Max) | | 35) | | | | 76) | | | |
| 30 min | | 53 | 58 | 51 | 63 | 97 | 89 | 71 | 93 |
| Average | | (51- | (56-59) | (51-51) | (61-65) | (96 | (89-89) | (70-73) | (86-103) |
| (Min Max) | | 56) | | | | 99) | | | |
| 45 min | | 64 | 73 | 60 | 75 | 105 | 104 | 77 | 105 |
| Average | | (63- | (70-75) | (60-61) | (73-77) | (104- | (103- | (76-77) | (101-112) |
| (Min Max) | | 68 | | | | 106) | 105) | | |
| 60 min | | 72 | 81 | 66 | 84 | 108 | 102 | 82 | 99 |
| Average | | (70- | (78-83) | (66-67) | (83-85) | (106- | (98- | (81-85) | (97-102) |
| (Min Max) | | 73) | | | | 110) | 107) | | |
| 70 min | | 79 | 84 | 71 | 88 | 108 | 108 | 83 | 110 |
| Average | | (78- | (82-85) | (71-72) | (87-89) | (107- | (97- | (81-84) | (106-117) |
| (Min Max) | | 81) | | | | 109) | 118) | | |
| | | Time point and storage conditions | | | | | | | |
| | | | 2 weeks- | 3 weeks- | | | 2 weeks- | 3 weeks- | 4 weeks- |
| | | | 60° C./ | 50° C./ | 4 weeks- | | 60° C./ | 50° C./ | 40° C./ |
| | | | 75% RH | 75% RH | 40° C./75% | | 75% RH | 75% RH | 75% RH |
| | | Initial | Packaged | exposed | RH packed | Initial | Packaged | exposed | packed |
| Test | | Result | Result | Result | Result | Result | Result | Result | Result |
| Ezetimibe | Method | (n = 6) | (n = 3) | (n = 3) | (n = 3) | (n = 6) | (n = 3) | (n = 3) | (n = 3) |
| 5 min | Discriminating | 2 | 3 | 3 | 2 | 7 | 13 | 10 | 7 |
| Average | method at | (2- | (3-3) | (2-3) | (1-2) | (5 | (13-14) | (9-10) | (6-8) |
| (Min Max) | pH 6.80.2 | 2) | | | | 9) | | | |
| 10 min | % w/v of | 5 | 5 | 5 | 5 | 18 | 25 | 15 | 15 |
| Average | Tween 80, 50 | (4- | (5-5) | (4-6) | (5-6) | (15- | (24-26) | (14-16) | (14-16) |
| (Min Max) | rpm paddle | 6) | | | | 22) | | | |
| 15 min | speed and 900 | | | | | | | | |
| Average | mL vessel | 9 | 11 | 9 | 8 | 28 | 34 | 19 | 21 |
| (Min Max) | volume | (8- | (10-11) | (8-10) | (7-9) | (27- | (33-35) | (19-20) | (20-22) |
| 30 min | | 10 | | | | 29) | | | |
| Average | | | | | | | | | |
| (Min Max) | | 16 | 15 | 14 | 16 | 40 | 45 | 26 | 31 |
| 45 min | | (15- | (15-15) | (13-15) | (15-17) | (39- | (44-45) | (26-26) | (28-34) |
| Average | | 18) | | | | 42) | | | |
| (Min Max) | | | | | | | | | |
| 60 min | | 19 | 20 | 18 | 20 | 47 | 52 | 30 | 38 |
| Average | | (19- | (19-20) | (17-18) | (19-21) | (45- | (51-53) | (30-30) | (37-40) |
| (Min Max) | | 21) | | | | 50) | | | |
| 70 min | | 22 | 22 | 20 | 24 | 52 | 51 | 35 | 39 |
| Average | | (21- | (22-23) | (20-20) | (23-25) | (51- | (49-54) | (35-35) | (39-40) |
| (Min Max) | | 23) | | | | 54) | | | |
| | | 27 | 25 | 24 | 29 | 58 | 54 | 40 | 49 |
| | | (26- | (25-26) | (23-24) | (27-30) | (57- | (49-59) | (38-41) | (47-52) |
| | | 27) | | | | 61) | | | |
| | | Prototype | | | | | | | |
| | | | 2 weeks- | 3 weeks- | 4 weeks- | | 2 weeks- | 3 weeks- | 4 weeks- |
| | | | 60° C./ | 50° C./ | 40° C./ | | 60° C./ | 50° C./ | 40° C./ |
| | | | 75% RH | 75% RH | 75% RH | | 75% RH | 75% RH | 75% RH |
| | | Initial | Packaged | exposed | packed | Initial | Packaged | exposed | packed |
| Ezetimibe | | (n = 3) | (n = 3) | (n = 3) | (n = 3) | (n = 6) | (n = 3) | (n = 3) | (n = 3) |
| 15 min | Dissolution | 11 | 7 | 11 | 9 | 26 | 22 | 27 | 29 |
| Average | method based | (11- | (7-7) | (9-13) | (8-10) | | (22-23) | (27-27) | (28-29) |
| (Min Max) | on USP | 12) | | | | (25- | | | |
| 30 min | method | | | | | 26) | | | |
| Average | conditions at | 19 | 17 | 22 | 20 | 41 | 34 | 39 | 41 |
| (Min Max) | pH 4.5, 50 | (18- | (17-18) | (21-24) | (19-21) | (40- | (33-35) | (39-40) | (40-42) |
| 45 min | rpm paddle | 19) | | | | 41) | | | |
| Average | speed and 500 | 24 | 23 | 28 | 27 | 51 | 43 | 48 | 49 |
| (Min Max) | mL vessel | (23- | (23-24) | (27-30) | (25-28) | (50- | (42-44) | (47-50) | (48-50) |
| 60 min | volume | 25) | | | | 51) | | | |
| Average | | 29 | 28 | 33 | 32 | 57 | 49 | 53 | 55 |
| (Min Max) | | (27- | (27-29) | (31-35) | (30-34) | (57- | (48-50) | (52-53) | (55-56) |
| 70 min | | 29) | | | | 58) | | | |
| Average | | 38 | 35 | 39 | 42 | 67 | 59 | 62 | 67 |
| (Min Max) | | (36- | (34-36) | (38-40) | (40-44) | (66- | (59-60) | (62-63) | (67-67) |
| | | 40) | | | | 68) | | | |
| | | Manufacturing lot number | | | | | | | |
| | | A4459/05/07 | | | | A4459/05/08 | | | |
| | | Process condition | | | | | | | |
| | | 1-high impeller speed & 3 min wet massing | | | | 2-low impeller speed & 1 min wet massing | | | |
| | | Time point and storage conditions | | | | | | | |
| | | | 2 weeks- | 3 weeks- | 4 weeks- | | 2 weeks- | 3 weeks- | 4 weeks- |
| | | | 60° C./ | 50° C./ | 40° C./ | | 60° C./ | 50° C./ | 40° C./ |
| | | | 75% RH | 75% RH | 75% RH | | 75% RH | 75% RH | 75% RH |
| | | Initial | Packaged | exposed | packed | Initial | Packaged | exposed | packed |
| Test | | Result | Result | Result | Result | Result | Result | Result | Result |
| Obicetrapib | Method | (n = 6) | (n = 3) | (n = 3) | (n = 3) | (n = 6) | (n = 3) | (n = 3) | (n = 3) |
| 5 min | Discriminating | 22 | 24 | 49 | 30 | 36 | 35 | 61 | 54 |
| Average | method at | (20- | (24-25) | (47-51) | (27-32) | (29- | (35-36) | (56-65) | (46-61) |
| (Min Max) | pH 6.80.2 | 26) | | | | 41) | | | |
| 10 min | % w/v of | 57 | 52 | 75 | 67 | 73 | 65 | 85 | 81 |
| Average | Tween 80, 50 | (52- | (48-55) | (73-78) | (62-71) | (65 | (58-70) | (82-86) | (78-86) |
| (Min Max) | rpm paddle | 61) | | | | 77) | | | |
| 15 min | speed and 900 | 80 | 71 | 84 | 84 | 91 | 82 | 93 | 92 |
| Average | mL vessel | (79- | (67-73) | (83-85) | (83-89) | (89 | (80-84) | (90-95) | (91-94) |
| (Min Max) | volume | 82) | | | | 93) | | | |
| 30 min | | 98 | 84 | 96 | 102 | 101 | 94 | 100 | 111 |
| Average | | (97- | (80-86) | (96-97) | (100-105) | (98- | (94-95) | (99- | (103-117) |
| (Min Max) | | 100) | | | | 105) | | 102) | |
| 45 min | | 101 | 90 | 98 | 104 | 103 | 98 | 101 | 103 |
| Average | | (99- | (89-91) | (97-99) | (96-111) | (100- | (97-99) | (100- | (99-109) |
| (Min Max) | | 103) | | | | 107) | | 102) | |
| 60 min | | 101 | 95 | 99 | 103 | 100 | 99 | 100 | 102 |
| Average | | (100- | (94-95) | (98-99) | (98-107) | (97- | (98- | (99- | (99-104) |
| (Min Max) | | 103) | | | | 110) | 100) | 101) | |
| 70 min | | 101 | 99 | 99 | 109 | 101 | 101 | 100 | 114 |
| Average | | (100- | (98- | (98-99) | (106-113) | (98- | (99- | (100- | (112-117) |
| (Min Max) | | 103) | 100) | | | 105) | 102) | 101) | |
| | | Time point and storage conditions | | | | | | | |
| | | | 2 weeks- | 3 weeks- | 4 weeks- | | 2 weeks- | 3 weeks- | 4 weeks- |
| | | | 60° C./ | 50° C./ | 40° C./ | | 60° C./ | 50° C./ | 40° C./ |
| | | | 75% RH | 75% RH | 75% RH | | 75% RH | 75% RH | 75% RH |
| | | Initial | Packaged | exposed | packed | Initial | Packaged | exposed | packed |
| Test | | Result | Result | Result | Result | Result | Result | Result | Result |
| Ezetimibe | Method | (n = 6) | (n = 3) | (n = 3) | (n = 3) | (n = 6) | (n = 3) | (n = 3) | (n = 3) |
| 5 min | Discriminating | 6 | 6 | 12 | 6 | 15 | 11 | 19 | 15 |
| Average | method at | (5- | (6-7) | (11-13) | (5-7) | (12 | (11-11) | (18-21) | (13-18) |
| (Min Max) | pH 6.80.2 | 7) | | | | 17) | | | |
| 10 min | % w/v of | 17 | 13 | 21 | 16 | 33 | 22 | 31 | 28 |
| Average | Tween 80, 50 | (16- | (12-15) | (20-22) | (15-17) | (28- | (20-24) | (29-33) | (26-29) |
| (Min Max) | rpm paddle | 18) | | | | 35) | | | |
| 15 min | speed and 900 | 26 | 20 | 26 | 24 | 45 | 33 | 40 | 38 |
| Average | mL vessel | (25- | (19-21) | (26-28) | (24-24) | (43- | (31-34) | (37-43) | (38-40) |
| (Min Max) | volume | 27) | | | | 48) | | | |
| 30 min | | 40 | 30 | 37 | 39 | 63 | 46 | 54 | 60 |
| Average | | (39- | (28-31) | (36-38) | (37-40) | (61- | (46-47) | (52-56) | (56-63) |
| (Min Max) | | 41) | | | | 64) | | | |
| 45 min | | 49 | 38 | 45 | 47 | 73 | 57 | 63 | 66 |
| Average | | (47- | (36-39) | (44-47) | (43-51) | (70- | (56-59) | (62-65) | (64-69) |
| (Min Max) | | 50) | | | | 77) | | | |
| 60 min | | 55 | 45 | 51 | 53 | 76 | 64 | 68 | 71 |
| Average | | (53- | (44-45) | (50-52) | (51-55) | (73 | (63-66) | (67-60) | (67-74) |
| (Min Max) | | 56) | | | | 84) | | | |
| 70 min | | 61 | 52 | 57 | 62 | 84 | 72 | 76 | 86 |
| Average | | (60- | (52-53) | (56-58) | (60-65) | (82- | (71-73) | (75-78) | (84-88) |
| (Min Max) | | 62) | | | | 88) | | | |
| | | Manufacturing lot number | | | | | | | |
| | | A4459/05/07 | | | | A4459/05/08 | | | |
| | | Process condition | | | | | | | |
| | | 1-high impeller speed & 3 min wet massing | | | | 2-low impeller speed & 1 min wet massing | | | |
| | | Prototype | | | | | | | |
| | | | 2 weeks- | 3 weeks- | 4 weeks- | | 2 weeks- | 3 weeks- | 4 weeks- |
| | | | 60° C./ | 50° C./ | 40° C./ | | 60° C./ | 50° C./ | 40° C./ |
| | | | 75% RH | 75% RH | 75% RH | | 75% RH | 75% RH | 75% RH |
| | | Initial | Packaged | exposed | packed | Initial | Packaged | exposed | packed |
| Ezetimibe | | (n=3) | (n = 3) | (n = 3) | (n = 3) | (n = 3) | (n = 3) | (n = 3) | (n = 3) |
| 15 min | Dissolution | 29 | 26 | 31 | 32 | 46 | 42 | 43 | 45 |
| Average | method based | (28- | (24-27) | (29-33) | (28-34) | (42- | (37-45) | (43-44) | (42-47) |
| (Min Max) | on USP | 31) | | | | 49) | | | |
| 30 min | method | 42 | 40 | 46 | 44 | 59 | 59 | 60 | 59 |
| Average | conditions at | (41- | (39-41) | (44-49) | (41-46) | (55- | (55-61) | (59-60) | (57-59) |
| (Min Max) | pH 4.5, 50 | 43) | | | | 61) | | | |
| 45 min | rpm paddle | 49 | 49 | 54 | 51 | 65 | 67 | 67 | 66 |
| Average | speed and 500 | (49- | (47-50) | (52-58) | (49-54) | (62- | (65-70) | (66-68) | (65-66) |
| (Min Max) | mL vessel | 50) | | | | 67) | | | |
| 60 min | volume | 55 | 55 | 61 | 58 | 69 | 74 | 73 | 70 |
| Average | | (55- | (53-56) | (58-65) | (55-60) | (67- | (71-76) | (72-73) | (70-70) |
| (Min Max) | | 55) | | | | 71) | | | |
| 70 min | | 71 | 69 | 73 | 71 | 86 | 87 | 87 | 86 |
| Average | | (71- | (67-70) | (72-75) | (69-72) | (85- | (85-88) | (87-88) | (86-86) |
| (Min Max) | | 71) | | | | 87) | | | |

### Physical Properties Characterization

With exception of prototype tablet A4459/05/05 in which the small presence of EZE hydrate is observed in the initial time point sample and in all the samples placed on stability, for the other tablets prototypes a small amount of the EZE hydrate form appears at the 3WK and 4WK time points. XRPD data are summarized in Table 10.

TABLE 10
XRPD data summary of small scale 10 mg ezetimibe and 5 mg obicetrapib tablets stress stability

| Sample | Batch ID | XRPD |
|---|---|---|
| Prototype 1 | A4459/05/05 Initial | OBI + EZE Anhydrous + small EZE Hydrate |
| condition 1 Tablet | A4459/05/05 2 WK@60/75 pack | OBI + EZE Anhydrous + small EZE Hydrate |
| | A4459/05/05 3 WK@50/75 exposed | OBI + EZE Anhydrous + small EZE Hydrate |
| | A4459/05/05 4 WK@40/75 exposed | OBI + EZE Anhydrous + small EZE Hydrate |
| Prototype 1 | A4459/05/06 Initial | OBI + EZE Anhydrous |
| condition 2 Tablet | A4459/05/06 2 WK@60/75 pack | OBI + EZE Anhydrous |
| | A4459/05/06 3 WK@50/75 exposed | OBI + EZE Anhydrous + small EZE Hydrate |
| | A4459/05/06 4 WK@40/75 exposed | OBI + EZE Anhydrous + small EZE Hydrate |
| Prototype 2 | A4459/05/07 Initial | OBI + EZE Anhydrous |
| condition 1 Tablet | A4459/05/07 2 WK@60/75 pack | OBI + EZE Anhydrous |
| | A4459/05/07 3 WK@50/75 exposed | OBI + EZE Anhydrous + small EZE Hydrate |
| | A4459/05/07 4 WK@40/75 exposed | OBI + EZE Anhydrous + small EZE Hydrate |
| Prototype | A4459/05/08 Initial | OBI + EZE Anhydrous |
| condition 2 Tablet | A4459/05/08 2 WK@60/75 pack | OBI + EZE Anhydrous |
| | A4459/05/08 3 WK@50/75 exposed | OBI + EZE Anhydrous + small EZE Hydrate |
| | A4459/05/08 4 WK@40/75 exposed | OBI + EZE Anhydrous + small EZE Hydrate |

### Example 2: Fixed Dose Combination for 10 mg Ezetimibe and 10 mg Obicetrapib Tablets (Small Scale Batch ~500 g)

The details of the prototype formulations manufactured in this set of experiments are summarised in Table 11 and Table 12. A key amendment in the formulation composition was the increment of dose strength of obicetrapib (free acid) from 5.0 mg to 10.0 mg.

### High Shear Granulation and Fluid Bed Drying

These trials were executed as small-scale batches (500 g batch size) according to process condition 2. However, batch A4459/16/02 (known as “prototype C scale-up”) was executed at 2 Kg batch size scale.

The powders were sieved manually, loaded into the granulation bowl and mixed for 5 mins. The granulation solution was sprayed at the required spray rate and wet massing was conducted prior to drying the material in a fluid bed drier. The inlet air temperature and the air volume was adjusted as required to fluidise the granule that was dried until its LOD was equal or lower than the initial LOD. The granules were characterised as for content uniformity of APIs, LOD (soon after milling), sieve analysis, TBD and XRPD. The granule batch A4459/13/01 (prototype A) and batch A4459/16/02 (prototype C scale-up) were divided in two aliquots to manufacture the final blends required to generate the 150 mg tablet and the 200 mg tablet.

### Preparation of the Final Blend, Tableting and Coating

The final blend was prepared by weighing accurately the extra-granular excipients to manufacture tablets with the required composition. The excipients were manually sieved and a bin of suitable volume was used for mixing. The lubricant (MgSt) was sieved separately, added to the bowl and mixed. A single punch compression machine was used to generate a compression profile and manufacture a small-scale batch of tablets. The friability, disintegration time, hardness, appearance and thickness of the tablets was monitored throughout processing. The tablets were tested for discriminating dissolution, ezetimibe USP tablet dissolution method and XRPD.

Three selected tablet batches (prototype B, prototype C scale-up and prototype C scale-up 200) were coated using a 20% w/w Opadry AMB II white aqueous suspension. The coating process parameters as well as the weight gain of the tablets were monitored throughout processing. The coated tablets were tested for XRPD, discriminating dissolution for obicetrapib, ezetimibe USP tablet dissolution method and ezetimibe USP tablet dissolution method with 75 rpm paddle speed.

All the intermediates of production and the final drug product were stored in double LDPE bags closed with cable ties and transferred into a thermosealed aluminium bag containing silica.

TABLE 11
Composition (% w/w) of granule and tablet of small scale 10 mg ezetimibe 10
mg obicetrapib

| | | | Prototype B | | | |
|---|---|---|---|---|---|---|
| | Prototype A | | | | Coated | Prototype C |
| | Granule | Tablet | Granule | Tablet | tablet | Granule |
| | A4459/ | A4459/ | A4459/ | A4459/ | A4459/ | A4459/ |
| Component | 13/01 | 14/01 | 13/02 | 14/02 | 19/01 | 13/03 |
| Ezetimibe | 6.981 | 6.667 | 6.981 | 6.667 | 6.667 | 6.981 |
| Obicetrapib | 7.162 | 6.840 | 7.162 | 6.840 | 6.840 | 7.162 |
| Avicel PH | 20.935 | 19.993 | 21.982 | 20.993 | 20.993 | 21.982 |
| 101 | | | | | | |
| Pharmatose | 57.592 | 55.000 | 57.592 | 55.000 | 55.000 | 57.592 |
| Kollidon 30 | 1.047 (in)* | 1.000 (in)* | 1.047 | 1.000 | 1.000 (in)* | 1.047 |
| | | | (in)* | (in)* | | (in)* |
| Glycolys | 4.189 | 4.000 | 4.189 | 4.000 | 4.000 | 4.189 |
| Kolliphor SLS | 2.094 | 2.000 | 1.047 | 1.000 | 1.000 | 1.047 |
| MilliQ water* | 20.000+ | N/A | 20.000 + | N/A | N/A | 20.000 + |
| | 5.000 | | 5.000 | | | 5.000 |
| Total granule | 100.000 | 95.500 | 100.000 | 95.500 | 95.500 | 100.000 |
| Avicel PH | N/A | N/A | N/A | N/A | N/A | N/A |
| 101 | | | | | | |
| Pharmatose | N/A | N/A | N/A | N/A | N/A | N/A |
| Kollidon 30 | N/A | N/A | N/A | N/A | N/A | N/A |
| Glycolys | N/A | 4.000 | N/A | 4.000 | 4.000 | N/A |
| Ligamed | N/A | 0.500 | N/A | 0.500 | 0.500 | N/A |
| MF-2- [symbol] | | | | | | |
| Opadry | N/A | N/A | N/A | N/A | 3.000 | N/A |
| Total | 100.000 | 100.000 | 100.000 | 100.000 | 103.000 | 100.000 |
| | | Prototype | Prototype | | | |
| | | C | D1 | D2 | Prototype A200 | |
| | | Tablet | Granule | Granule | Granule | Tablet |
| | | A4459/ | A4459/ | A4459/ | A4459/ | A4459/ |
| | Component | 14/03 | 13/04 | 13/05 | 13/01 | 14/04 |
| | Ezetimibe | 6.667 | 6.981 | 6.981 | 6.981 | 5.000 |
| | Obicetrapib | 6.840 | 7.162 | 7.162 | 7.162 | 5.130 |
| | Avicel PH | 20.993 | 21.982 | 21.982 | 20.935 | 14.995 |
| | 101 | | | | | |
| | Pharmatose | 55.000 | 57.592 | 57.592 | 57.592 | 41.250 |
| | Kollidon 30 | 1.000 | 1.047 | 1.047 | 1.047 | 0.750 (in)* |
| | | (in)* | (out)* | (out)* | (in)* | |
| | Glycolys | 4.000 | 4.189 | 4.189 | 4.189 | 3.000 |
| | Kolliphor SLS | 1.000 | 1.047 | 1.047 | 2.094 | 1.500 |
| | MilliQ water* | N/A | 20.000 | 5.000 ** | 20.000 + | N/A |
| | | | | | 5.000 | |
| | Total granule | 95.500 | 100.000 | 100.000 | 100.000 | 71.625 |
| | Avicel PH | N/A | N/A | N/A | N/A | 10.437 |
| | 101 | | | | | |
| | Pharmatose | N/A | N/A | N/A | N/A | 10.438 |
| | Kollidon 30 | N/A | N/A | N/A | N/A | 3.000 |
| | Glycolys | 4.000 | N/A | N/A | N/A | 4.000 |
| | Ligamed | 0.500 | N/A | N/A | N/A | 0.500 |
| | MF-2- [symbol] | | | | | |
| | Opadry | N/A | N/A | N/A | N/A | N/A |
| | Total | 100.000 | 100.000 | 100.000 | 100.000 | 100.000 |

*Water does not appear in the tablet; in = material added as a dry powder, out = material solubilised in the water for granulation;
** Lower quantity added due to an issue with the equipment [symbol]
indicates data missing or illegible when filed

TABLE 12
Composition (% w/w) of granule and tablet of 10 mg ezetimibe and 10 mg
obicetrapib

| | Scale-up Prototype C | | | Prototype C (2% w/w | |
|---|---|---|---|---|---|
| | | | Coated | SLS) | |
| | Granule | Tablet | tablet | Granule | Tablet |
| Component | A4459/16/02 | A4459/16/03 | A4459/19/02 | A4459/17/01 | A4459/18/01 |
| Ezetimibe | 6.981 | 6.632 | 6.632 | 6.981 | 6.667 |
| Obicetrapib | 7.162 | 6.804 | 6.804 | 7.162 | 6.840 |
| Avicel PH 101 | 21.982 | 20.883 | 20.883 | 20.935 | 19.993 |
| Pharmatose | 57.592 | 54.712 | 54.712 | 57.592 | 55.000 |
| Kollidon 30 | 1.047 | 0.995 | 0.995 (in)* | 1.047 | 1.000 (in)* |
| Glycolys | 4.189 | 3.980 | 3.980 | 4.189 | 4.000 |
| Kolliphor SLS | 1.047 | 0.995 | 0.995 (in)* | 2.094 | 2.000 (in)* |
| MilliQ water * | 20.000 + | N/A | N/A | 20.000 + | N/A |
| | 5.000 | | | 5.000 | |
| Total granule | 100.000 | 95.001 | 95.001 | 100.000 | 95.500 |
| Avicel PH 101 | N/A | N/A | N/A | N/A | N/A |
| Pharmatose | N/A | N/A | N/A | N/A | N/A |
| Kollidon 30 | N/A | N/A | N/A | N/A | N/A |
| Glycolys | N/A | 4.000 | 4.000 | N/A | 4.000 |
| Ligamed MF- | N/A | 1.000 | 1.000 | N/A | 0.500 |
| 2- [symbol] | | | | | |
| Opadry | N/A | N/A | 3.000 | N/A | N/A |
| Total | 100.000 | 100.001 | 103.001 | 100.000 | 100.000 |
| | | | Prototype C scale-up 200 | | |
| | Prototype D | | | | Coated |
| | Granule | Tablet | Granule | Tablet | tablet |
| Component | A4459/17/02 | A4459/18/02 | A4459/16/02 | A4459/18/03 | A4459/19/03 |
| Ezetimibe | 6.981 | 6.667 | 6.981 | 5.000 | 5.000 |
| Obicetrapib | 7.162 | 6.840 | 7.162 | 5.130 | 5.130 |
| Avicel PH 101 | 21.982 | 20.993 | 21.982 | 15.745 | 15.745 |
| Pharmatose | 57.592 | 55.000 | 57.592 | 41.250 | 41.250 |
| Kollidon 30 | 1.047 (out)* | 1.000 | 1.047 (in)* | 0.750 | 0.750 (in)* |
| Glycolys | 4.189 | 4.000 | 4.189 | 3.000 | 3.000 |
| Kolliphor SLS | 1.047 (in)* | 1.000 | 1.047 (in)* | 0.750 | 0.750 (in)* |
| MilliQ water * | 20.000 + | N/A | 20.000 + | N/A | N/A |
| | 5.000 | | 5.000 | | |
| Total granule | 100.000 | 95.500 | 100.000 | 71.625 | 71.625 |
| Avicel PH 101 | N/A | N/A | N/A | 10.437 | 10.437 |
| Pharmatose | N/A | N/A | N/A | 10.438 | 10.438 |
| Kollidon 30 | N/A | N/A | N/A | 3.000 | 3.000 |
| Glycolys | N/A | 4.000 | N/A | 4.000 | 4.000 |
| Ligamed MF- | N/A | 0.500 | N/A | 0.500 | 0.500 |
| 2- [symbol] | | | | | |
| Opadry | N/A | N/A | N/A | N/A | 3.000 |
| Total | 100.000 | 100.000 | 100.000 | 100.000 | 103.000 |

* Water does not appear in the tablet; in = material added as a dry powder, out = material solubilised in the water for granulation [symbol]
indicates data missing or illegible when filed

### Results:

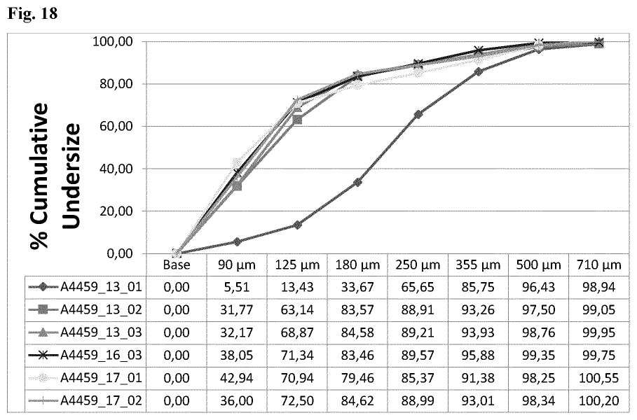

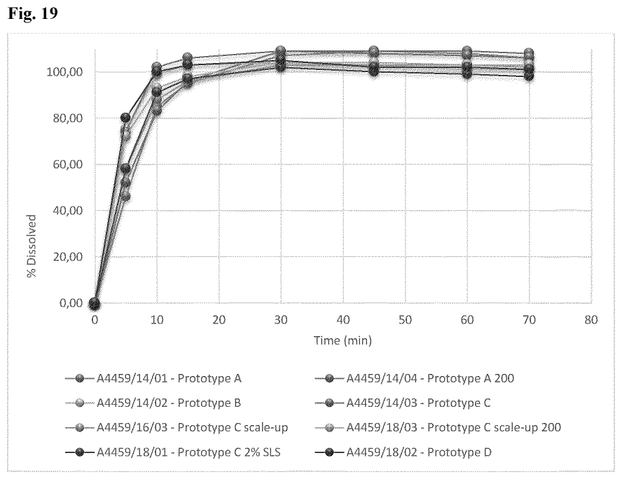

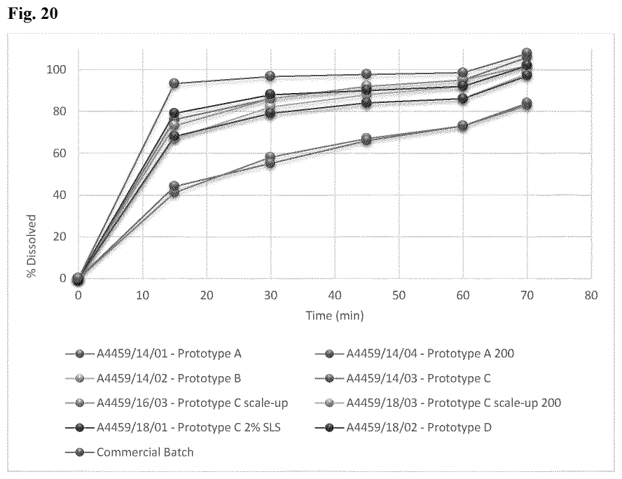

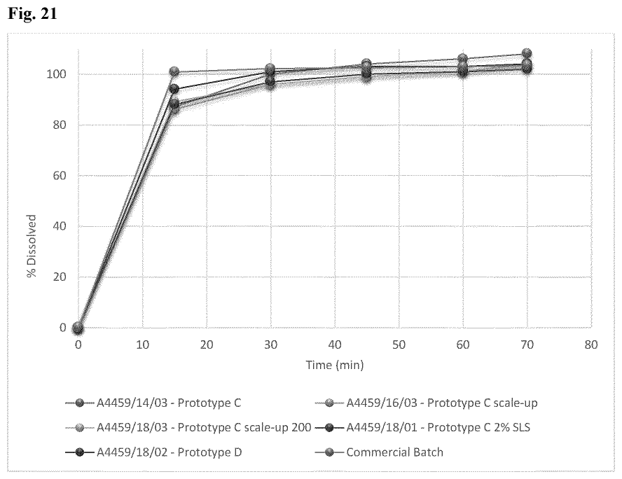

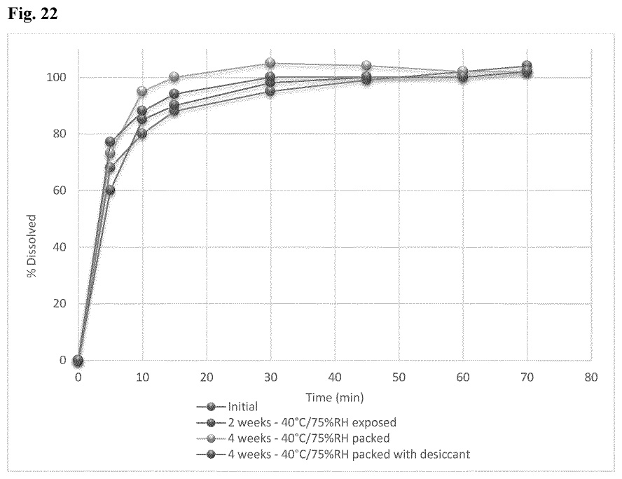

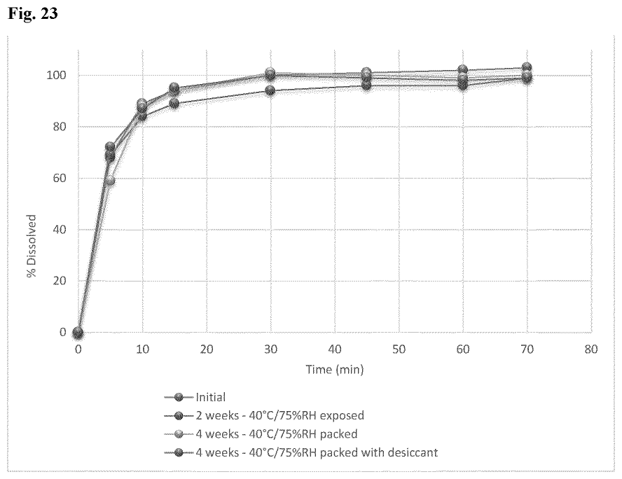

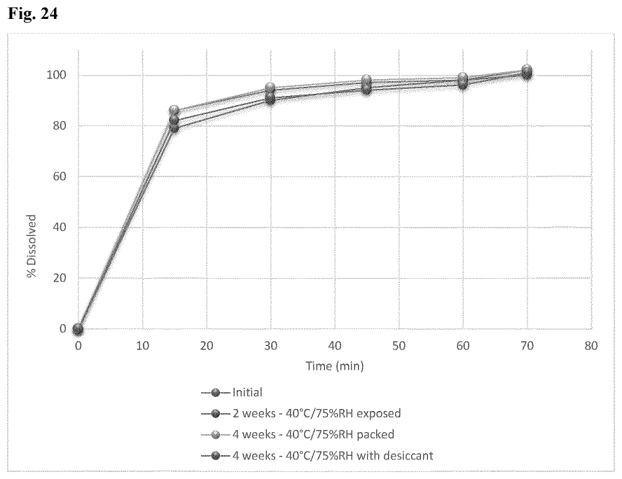

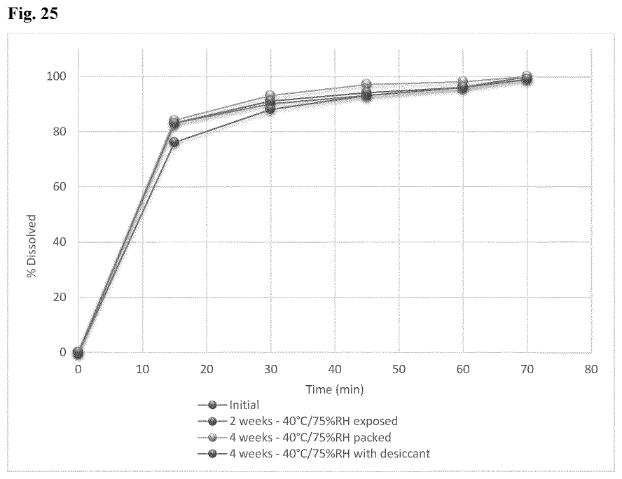

These granulation trials were conducted successfully. Overall, the granules presented a PSD similar to that of batch A4459/08/01 (prototype 4) and showed a relatively large quantity of fine particles (FIG. 18). In comparison to batch A4459/08/02 (prototype 4, condition 2), the tablet batches presented comparable time of disintegration and higher hardness and lower friability values for similar compression forces. The tablets did not present any critical defects (e.g., capping, lamination). The coated tablets presented a smooth and white surface without any visual cosmetic defects upon close inspection.

### Granules Characterization

### Chemical Characterization Analysis

The granules were tested for homogeneity of both obicetrapib and ezetimibe, results are reported in Table 13. Analysis were performed on n=6 except for scale up batch performed with n=10.

### Physical Properties Characterization

XRPD data of the development prototypes are summarized in Table 14. EZE hydrate can be observed in samples before the granulation process or during the granulation. However, the amount of EZE hydrate detected appears always to be very limited.

### Tablets Characterization

### Chemical Characterization Analysis

Results for the characterization of the small scale batches are reported in Table 15. Prototypes were tested for dissolution. The dissolution results for obicetrapib showed similar profiles for all the prototypes tested with small differences deemed to be analytical variability. For ezetimibe most promising results were obtained for prototypes D and C which obtained promising results with prototypes C meeting the USP specification of Q-80+5 at 30 minutes, on three vessel. This most promising prototypes were also characterized with USP dissolution method conditions for ezetimibe at the higher paddle speed of 75 rpm. This was due since it was highlighted that the USP method, developed for a lighter tablet comparing to the developed fixed dose combination, appeared to be overdiscriminating for tablets with target weight up to 200 mg. The results showed profiles consistent with that currently commercialized formulation.

### Physical Properties Characterization

All the tablets of small scale prototypes produced presented small amount of EZE hydrate with exception of the Prototype C and C scale up 200 mg batches as reported in Table 16.

TABLE 13
Granule uniformity of 10 mg ezetimibe and 10 mg (free acid)
obicetrapib development prototypes

| | A4459/13/01 | | A4459/13/02 | | A4459/13/03 | |
|---|---|---|---|---|---|---|
| | Prototype A | | Prototype B | | Prototype C | |
| | Ezetimibe | Obicetrapib | Ezetimibe | Obicetrapib | Ezetimibe | Obicetrapib |
| | (% | (% | (% | (% | (% | (% |
| | Claim) | claim) | Claim) | claim) | Claim) | claim) |
| Sam# | 105.6 | 100.1 | 101.2 | 97.8 | 104.0 | 99.1 |
| 1 | | | | | | |
| Sam# | 103.2 | 97.9 | 100.4 | 96.1 | 105.3 | 100.3 |
| 2 | | | | | | |
| Sam# | 106.2 | 100.8 | 99.6 | 95.5 | 105.2 | 100.5 |
| 3 | | | | | | |
| Sam# | 100.3 | 94.5 | 97.3 | 92.6 | 106.4 | 101.5 |
| 4 | | | | | | |
| Sam# | 102.1 | 96.4 | 97.9 | 93.5 | 104.9 | 99.9 |
| 5 | | | | | | |
| Sam# | 105.1 | 99.4 | 99.7 | 95.8 | 105.0 | 100.1 |
| 6 | | | | | | |
| Sam# | — | — | — | — | — | — |
| 7 | | | | | | |
| Sam# | — | — | — | — | — | — |
| 8 | | | | | | |
| Sam# | — | — | — | — | — | — |
| 9 | | | | | | |
| Sam# | — | — | — | — | — | — |
| 10 | | | | | | |
| Mean | 103.8 | 98.2 | 99.3 | 95.2 | 105.1 | 100.2 |
| RSD | 2.2 | 2.47 | 1.5 | 2.0 | 0.7 | 0.8 |
| % | | | | | | |
| | A4459/16/02 | | A4459/17/01 | | | |
| | Prototype C | | Prototype C2% | | A4459/17/02 | |
| | Scale-up | | SLS | | Prototype D | |
| | Ezetimibe | Obicetrapib | Ezetimibe | Obicetrapib | Ezetimibe | Obicetrapib |
| | (% | (% | (% | (% | (% | (% |
| | Claim) | claim) | Claim) | claim) | Claim) | claim) |
| Sam# | 102.3 | 98.4 | 106.6 | 102.1 | 103.6 | 98.6 |
| 1 | | | | | | |
| Sam# | 102.8 | 100.2 | 106.2 | 101.4 | 103.8 | 99.0 |
| 2 | | | | | | |
| Sam# | 103.2 | 98.9 | 104.3 | 100.1 | 104.4 | 99.5 |
| 3 | | | | | | |
| Sam# | 101.8 | 98.0 | 102.1 | 98.0 | 105.1 | 100.0 |
| 4 | | | | | | |
| Sam# | 102.2 | 98.6 | 99.6 | 95.7 | 103.4 | 98.3 |
| 5 | | | | | | |
| Sam# | 101.6 | 97.9 | 100.0 | 96.3 | 105.4 | 100.7 |
| 6 | | | | | | |
| Sam# | 100.8 | 97.5 | — | — | — | — |
| 7 | | | | | | |
| Sam# | 101.1 | 97.2 | — | — | — | — |
| 8 | | | | | | |
| Sam# | 100.8 | 97.0 | — | — | — | — |
| 9 | | | | | | |
| Sam# | 100.2 | 96.7 | — | — | — | — |
| 10 | | | | | | |
| Mean | 101.7 | 98.0 | 103.1 | 98.9 | 104.3 | 99.4 |
| RSD | 0.9 | 98.4 | 3.0 | 2.7 | 0.8 | 0.9 |
| % | | | | | | |

TABLE 14
XRPD data summary 10 mg ezetimibe and 10 mg (free
acid) obicetrapib development prototypes

| Sample | Batch ID | XRPD |
|---|---|---|
| Prototype A Blend | A4459/13/01 | OBI + EZE Anhydrous |
| Prototype A Wet Granules | | OBI + EZE Anhydrous + some EZE Hydrate |
| Prototype A Dry Granules | | OBI + EZE Anhydrous + some EZE Hydrate |
| Prototype B Blend | A4459/13/02 | OBI + EZE Anhydrous |
| Prototype B Wet Granules | | OBI + EZE Anhydrous + small EZE Hydrate |
| Prototype B Dry Granules | | OBI + EZE Anhydrous + small EZE Hydrate |
| Prototype C Blend | A4459/13/03 | OBI + EZE Anhydrous |
| Prototype C Wet Granules | | OBI + EZE Anhydrous + small EZE Hydrate |
| Prototype C Dry Granules | | OBI + EZE Anhydrous |
| Prototype C Scale-up Blend | A4459/16/02 | OBI + EZE Anhydrous + small EZE Hydrate |
| Prototype C Scale-up Wet Granules | | OBI + EZE Anhydrous + small EZE Hydrate |
| Prototype C Scale-up Dry Granules | | OBI + EZE Anhydrous |
| Prototype C 2% SLS Blend | A4459/17/01 | OBI + EZE Anhydrous |
| Prototype C 2% SLS Wet Granules | | OBI + EZE Anhydrous + small EZE Hydrate |
| Prototype C 2% SLS Dry Granules | | OBI + EZE Anhydrous + small EZE Hydrate |
| Prototype D Blend | A4459/17/02 | OBI + EZE Anhydrous + small EZE Hydrate |
| Prototype D Wet Granules | | OBI + EZE Anhydrous + small EZE Hydrate |
| Prototype D Dry Granules | | OBI + EZE Anhydrous |

TABLE 15
Results of the dissolution characterization of small scale 10 mg ezetimibe and
10 mg obicetrapib (free acid)

| | | Manufacturing lot number | | | | | | | |
|---|---|---|---|---|---|---|---|---|---|
| | | A4459/14/ | A4459/14/ | A4459/14/ | A4459/14/ | A4459/16/ | A4459/18/ | A4459/18/ | A4459/18/ |
| | | 01 | 04 | 02 | 03 | 03 | 03 | 01 | 02 |
| | | Prototype | | | | | | | |
| | | | | | | Prototype | Prototype | Prototype | |
| | | Prototype | Prototype | Prototype | Prototype | C scale- | C scale- | C 2% | Prototype |
| | | A | A 200 | B | C | up | up 200 | SLS | D |
| Test | Method | Result | Result | Result | Result | Result | Result | Result | Result |
| Dissolution | | | | | | | | | |
| Obicetrapib | | | | | | | | | |
| 5 min | Discriminating | 46 | 52 | 75 | 74 | 58 | 72 | 58 | 80 |
| Average | method | (43-50) | (47-54) | (68-81) | (71- | (53-63) | (70-73) | (51-64) | (77- |
| (Min Max) | at pH | | | | 75) | | | | 86) |
| 10 min | 6.80.2 | 85 | 83 | 99 | 102 | 88 | 93 | 91 | 100 |
| Average | % w/v of | (83-88) | (82-84) | (96-101) | (101- | (85-90) | (93-93) | (87-93) | (98-104) |
| (Min Max) | Tween 80, | | | | 102) | | | | |
| 15 min | 50 rpm | 95 | 96 | 103 | 106 | 96 | 98 | 97 | 103 |
| Average | paddle | (95-96) | (95-97) | (102- | (105- | (95-97) | (97-100) | (94-100) | (101- |
| (Min Max) | speed and | | | 104) | 108) | | | | 106) |
| 30 min | 900 mL | 107 | 109 | 104 | 109 | 103 | 104 | 102 | 105 |
| Average | vessel | (107- | (107- | (103- | (107- | (102- | (102- | (101- | (101- |
| (Min Max) | volume | 108) | 110) | 105) | 111) | 105) | 105) | 104) | 108) |
| 45 min | | 109 | 109 | 103 | 108 | 103 | 104 | 100 | 102 |
| Average | | (108- | (109- | (100- | (106 | (102- | (102- | (99-102) | (101- |
| (Min Max) | | 109) | 110) | 104) | 109) | 104) | 105) | | 104) |
| 60 min | | 108 | 109 | 101 | 107 | 103 | 103 | 99 | 102 |
| Average | | (107- | (108- | (100- | (105- | (103- | (103- | (98-100) | (100- |
| (Min Max) | | 108) | 110) | 103) | 108) | 103) | 104) | | 104) |
| 70 min | | 106 | 108 | 101 | 106 | 102 | 103 | 98 | 101 |
| Average | | (106- | (107- | (99-103) | (105- | (102- | (103- | (97-99) | (100- |
| (Min Max) | | 107) | 108) | | 107) | 103) | 104) | | 103) |
| Dissolution | | | | | | | | | |
| Ezetimibe | | | | | | | | | |
| (n = 3) | | | | | | | | | |
| 15 min | Dissolution | 41 | 44 | 67 | 76 | 73 | 68 | 68 | 79 |
| Average | method | (40-43) | (41-45) | (65-69) | (75-76) | (70-76) | (65-71) | (66-70) | (74- |
| (Min Max) | based on | | | | | | | | 86) |
| 30 min | USP | 58 | 55 | 82 | 86 | 86 | 79 | 79 | 88 |
| Average | method | (55-60) | (52-57) | (80-84) | (85-88) | (84-87) | (79-81) | (77-80) | (84- |
| (Min Max) | conditions | | | | | | | | 94) |
| 45 min | at pH 4.5, | 67 | 66 | 88 | 92 | 92 | 84 | 84 | 90 |
| Average | 50 rpm | (64-69) | (62-67) | (86-89) | (90-94) | (92-93) | (82-86) | (82-84) | (87- |
| (Min Max) | paddle | | | | | | | | 95) |
| 60 min | speed and | 73 | 73 | 92 | 95 | 95 | 86 | 86 | 92 |
| Average | 500 mL | (71-76) | (69-77) | (90-94) | (93-97) | (93-97) | (84-88) | (85-88) | (90- |
| (Min Max) | vessel | | | | | | | | 96) |
| 70 min | volume | 83 | 84 | 102 | 106 | 102 | 98 | 97 | 102 |
| Average | | (80-86) | (80-87) | (101- | (105- | (102- | (98-98) | (96-98) | (100- |
| (Min Max) | | | | 103) | 106) | 102) | | | 104) |
| Dissolution | | | | | | | | | |
| Ezetimibe | | | | | | | | | |
| (n = 3) | | | | | | | | | |
| 15 min | Dissolution | — | — | — | 87 | 86 | 89 | 88 | 94 |
| Average | method | | | | (85-89) | (84-88) | (88-89) | (86-90) | (93- |
| (Min Max) | based on | | | | | | | | 96) |
| 30 min | USP | — | — | — | 100 | 96 | 96 | 97 | 101 |
| Average | method | | | | (98-101) | (93-98) | (95-97) | (96-99) | (99- |
| (Min Max) | conditions | | | | | | | | 103) |
| 45 min | at pH 4.5, | — | — | — | 104 | 99 | 99 | 100 | 103 |
| Average | 75 rpm | | | | (102- | (97-101) | (99-99) | (99-101) | (101- |
| (Min Max) | paddle | | | | 106) | | | | 105) |
| 60 min | speed and | — | — | — | 106 | 101 | 101 | 101 | 103 |
| Average | 500 mL | | | | (105- | (99-102) | (100- | (100- | (101- |
| (Min Max) | vessel | | | | 108) | | 101) | 102) | 105) |
| 70 min | volume | — | — | — | 108 | 104 | 103 | 102 | 104 |
| Average | | | | | (106- | (102- | (102- | (101- | (101- |
| (Min Max) | | | | | 109) | 106) | 103) | 104) | 105) |

TABLE 16
XRPD data summary of small scale tablets prototype batches

| Sample | Batch ID | XRPD |
|---|---|---|
| Tablet Prototype A | A4459/14/01 | OBI + EZE Anhydrous + small EZE Hydrate |
| Tablet Prototype A 200 mg | A4459/14/04 | OBI + EZE Anhydrous + small EZE Hydrate |
| Tablet Prototype B | A4459/14/02 | OBI + EZE Anhydrous + small EZE Hydrate |
| Tablet Prototype C | A4459/14/03 | OBI + EZE Anhydrous |
| Tablet Prototype C scale up | A4459/16/03 | OBI + EZE Anhydrous |
| Tablet Prototype C scale up 200 mg | A4459/18/03 | OBI + EZE Anhydrous + small EZE Hydrate |
| Tablet Prototype C 2% SLS | A4459/18/01 | OBI + EZE Anhydrous + small EZE Hydrate |
| Tablet Prototype D | A4459/18/02 | OBI + EZE Anhydrous + small EZE Hydrate |

### Stress Stability

Based on the process tableting parameters and dissolution data, the following tablet prototypes were selected to assess the feasibility of the coating process:
A4459/16/03 (150 mg/tab, prototype C scale-up)
A4459/18/03 (200 mg/tab prototype C scale-up/200 mg),
   and subsequently set down for stability a stress stability study with the following design. Results are reported in Table 17 and Table 18.

TABLE 17
Stress stability study design

| Storage Conditions | | Time (weeks) | | | |
|---|---|---|---|---|---|
| T/RH | Type | 0 | 2 | 3 | 4 |
| 25° C./60% RH | Long-term | T | (T) | — | (T) |
| 40° C./75% RH uncovered | Stress (T) | | T | — | T |
| 40° C./75% RH closed | Accelerated | | (T) | — | (T) |
| 40° C./75% RH closed with desiccant | Stress (T) | | (T) | — | (T) |

Key:
T = Tested for appearance, assay & related substances, Content Uniformity (only at initial) discriminating dissolution, water content by KF, and form check by XRPD
(T) = Optional testing

TABLE 18
Results for assay, water content and visual appearance of small scale 10 mg ezetimibe and
10 mg obicetrapib (free acid) prototype C 200 and prototype C scale-up stress stability

| | | Assay of | Assay of | Water | |
|---|---|---|---|---|---|
| Prototype | | Ezetimibe | Obicetrapib | content | Appearance |
| No. | Time point - Storage conditions | (% Claim) | (% Claim) | (% w/w) | (visual) |
| Prototype C | Initial | 99.1 | 101.2 | 5.1 | White round tablet |
| 200 BN | 2 weeks - 40° C./75% RH exposed | 99.4 | 100.7 | 7.3 | No tested |
| A4459/19/03 | 4 weeks - 40° C./75% RH exposed | 99.6 | 100.8 | 7.1 | White round tablet |
| | 4 weeks - 40° C./75% RH packed | 100.3 | 100.5 | 4.3 | No tested |
| | 4 weeks - 40° C./75% RH packed | 100.6 | 100.6 | 3.7 | White round tablet |
| | With dessiccant | | | | |
| Prototype C | Initial | 97.3 | 98.4 | 4.9 | |
| scale up | 2 weeks - 40° C./75% RH exposed | 97.6 | 98.8 | 6.6 | White round tablet |
| BN | 4 weeks - 40° C./75% RH exposed | 97.5 | 98.6 | 6.8 | No tested |
| A4459/19/02 | 4 weeks - 40° C./75% RH packed | 98.4 | 98.4 | 4.3 | White round tablet |
| | 4 weeks - 40° C./75% RH packed | 98.3 | 98.3 | 3.6 | No tested |
| | With dessiccant | | | | |

TABLE 19
Results for impurities profile of small scale 10 mg ezetimibe and 10 mg
obicetrapib (free acid) prototype C 200 and prototype C scale-up stress stability

| | | Obicetrapib related impurities | | | | Ezetimibe related impurities | | | |
|---|---|---|---|---|---|---|---|---|---|
| | | IMP_ | | MONO- | | IMP_ | | | Total |
| | Time point- | RRT | FROL | BN | Total | RRT | EZETIMIBE | EZETIMIBE | impurities |
| | Storage | 0.94 | (% | FREE | impurities | 0.79 | CYCLIC | KETONE | (≥0.05) |
| Batch | conditions | (% a/a) | a/a) | (% a/a) | (% a/a) | (% a/a) | (% a/a) | (% a/a) | (% a/a) |
| Prototype C | Initial | 0.08 | 0.07 | 0.18 | 0.33 | 0.05 | N.D. | 0.05 | 0.10 |
| 200 | 2 weeks- | 0.08 | 0.07 | 0.16 | 0.31 | 0.05 | 0.22 | 0.07 | 0.33 |
| BN | 40° C./75% | | | | | | | | |
| A4459/19/ | RH exposed | | | | | | | | |
| 03 | 4 weeks- | 0.08 | 0.06 | 0.16 | 0.31 | 0.05 | 0.52 | 0.08 | 0.65 |
| | 40° C./75% | | | | | | | | |
| | RH exposed | | | | | | | | |
| | 4 weeks- | 0.06 | 0.05 | 0.17 | 0.28 | 0.04 | N.D | 0.05 | 0.05 |
| | 40° C./75% | | | | | | | | |
| | RH packed | | | | | | | | |
| | 4 weeks- | 0.06 | 0.05 | 0.17 | 0.28 | 0.04 | N.D | 0.05 | 0.05 |
| | 40° C./75% | | | | | | | | |
| | RH packed | | | | | | | | |
| | With | | | | | | | | |
| | desiccant | | | | | | | | |
| Prototype C | Initial | 0.09 | 0.07 | 0.18 | 0.33 | 0.05 | N.D. | 0.05 | 0.10 |
| scale up | 2 weeks- | 0.08 | 0.07 | 0.16 | 0.31 | 0.05 | 0.09 | 0.06 | 0.19 |
| BN | 40° C./75% | | | | | | | | |
| A4459/19/ | RH | | | | | | | | |
| 02 | exposed | | | | | | | | |
| | 4 weeks- | 0.06 | 0.06 | 0.17 | 0.29 | 0.05 | 0.22 | 0.06 | 0.32 |
| | 40° C./75% | | | | | | | | |
| | RH exposed | | | | | | | | |
| | 4 weeks- | 0.06 | 0.05 | 0.17 | 0.28 | 0.05 | N.D. | 0.05 | 0.10 |
| | 40° C./75% | | | | | | | | |
| | rH packed | | | | | | | | |
| | 4 weeks- | 0.06 | 0.05 | 0.17 | 0.28 | 0.05 | N.D. | 0.05 | 0.10 |
| | 40° C./75% | | | | | | | | |
| | RH packed | | | | | | | | |
| | With | | | | | | | | |
| | desiccant | | | | | | | | |

N.D. = Not Detected

TABLE 20
Results of the dissolution characterization of small scale 10 mg ezetimibe and 10
mg obicetrapib (free acid) prototype C 200 and prototype C scale-up stress stability

| | | Manufacturing lot number | | | | | | | |
|---|---|---|---|---|---|---|---|---|---|
| | | Prototype C 200 BN A4459/19/03 | | | | Prototype C scale up BN A4459/19/02 | | | |
| | | Prototype | | | | | | | |
| | | | | | 4 weeks- | | | | 4 weeks- |
| | | | 2 weeks- | | 40° C./75% | | 2 weeks- | | 40° C./75% |
| | | | 40° C./75% | 4 weeks- | RH packed | | 40° C./75% | 4 weeks- | RH packed |
| | | | RH | 40° C./75% | with | | RH | 40° C./75% | with |
| | | Initial | exposed | RH packed | desiccant | Initial | exposed | RH packed | desiccant |
| Test | | Result | Result | Result | Result | Result | Result | Result | Result |
| Obicetrapib | Method | (n = 6) | (n = 6) | (n = 6) | (n = 6) | (n = 6) | (n = 6) | (n = 6) | (n = 6) |
| 5 min | Discriminating | 68 | 60 | 73 | 77 | 68 | 69 | 59 | 72 |
| Average | method | (67- | (55-64) | (67-76) | (70-81) | (65- | (66-72) | (35-69) | (69-76) |
| (Min Max) | at pH 6.80.2 | 71) | | | | 73) | | | |
| 10 min | % w/v of | 80 | 85 | 95 | 88 | 89 | 84 | 88 | 87 |
| Average | Tween 80, | (78- | (83-87) | (93-97) | (83-91) | (86- | (81-87) | (81-92) | (84-91) |
| (Min Max) | 50 rpm | 83) | | | | 93) | | | |
| 15 min | paddle | 88 | 90 | 100 | 94 | 94 | 89 | 94 | 95 |
| Average | speed and | (84- | (89-91) | (98-102) | (90-97) | (91- | (87-93) | (91-97) | (92-98) |
| (Min Max) | 900 mL | 90) | | | | 98) | | | |
| 30 min | vessel | 95 | 98 | 105 | 100 | 100 | 94 | 101 | 100 |
| Average | volume | (93- | (96-99) | (102-110) | (97-103) | (95- | (91-99) | (96-106) | (96-103) |
| (Min Max) | | 96) | | | | 105) | | | |
| 45 min | | 99 | 100 | 104 | 100 | 101 | 96 | 100 | 99 |
| Average | | (97- | (98-101) | (101-107) | (97-103) | (97- | (93-101) | (96-105) | (96-102) |
| (Min Max) | | 101) | | | | 105) | | | |
| 60 min | | 102 | 100 | 102 | 100 | 102 | 96 | 99 | 98 |
| Average | | (100- | (98-102) | (101-104) | (97-102) | (97- | (94-100) | (96-103) | (96-101) |
| (Min Max) | | 104) | | | | 104) | | | |
| 70 min | | 104 | 102 | 102 | 102 | 103 | 99 | 100 | 99 |
| Average | | (102- | (100-103) | (101-104) | (101-103) | (99- | (98-101) | (98-103) | (97-101) |
| (Min Max) | | 107) | | | | 106) | | | |
| | | Prototype | | | | | | | |
| | | | 2 weeks- | | 4 weeks- | | 2 weeks- | | 4 weeks- |
| | | | 40° C./75% | 4 weeks- | 40° C./75% | | 40° C./75% | 4 weeks- | 40° C./75% |
| | | Initial | RH | 40° C./75% | RH with | Initial | RH | 40° C./75% | RH with |
| Ezetimibe | | (n = 3) | exposed | RH packed | desiccant | (n = 3) | exposed | RH packed | desiccant |
| 15 min | Dissolution | 69 | | | | 68 | | | |
| Average | method | (59- | | | | (63- | | | |
| (Min Max) | based on | 75) | | | | 71) | | | |
| 30 min | USP method | 78 | | | | 74 | | | |
| Average | conditions at | (76- | | | | (68- | | | |
| (Min Max) | pH 4.5, 50 | 81) | | | | 77) | | | |
| 45 min | rpm paddle | 81 | | | | 76 | | | |
| Average | speed and | (79- | | | | (69- | | | |
| (Min Max) | 500 mL | 83) | | | | 79) | | | |
| 60 min | vessel | 82 | | | | 77 | | | |
| Average | volume | (80- | | | | (70- | | | |
| (Min Max) | | 84) | | | | 80) | | | |
| 70 min | | 96 | | | | 92 | | | |
| Average | | (94- | | | | (90- | | | |
| (Min Max) | | 100) | | | | 93) | | | |
| Ezetimibe | | (n = 3) | (n = 6) | (n = 6) | (n = 6) | (n = 3) | (n = 6) | (n = 6) | (n = 6) |
| 15 min | Dissolution | 86 | 79 | 86 | 82 | 83 | 76 | 84 | 83 |
| Average | method | (84- | (78-81) | (84-87) | (80-84) | (80- | (73-78) | (83-86) | (81-87) |
| (Min Max) | based on | 89) | | | | 85) | | | |
| 30 min | USP method | 94 | 90 | 95 | 91 | 90 | 88 | 93 | 91 |
| Average | conditions at | (92- | (89-91) | (94-95) | (87-93) | (87- | (84-90) | (92-95) | (89-94) |
| (Min Max) | pH 4.5, 75 | 97) | | | | 93) | | | |
| 45 min | rpm paddle | 97 | 95 | 98 | 94 | 93 | 93 | 97 | 94 |
| Average | speed and | (95- | (94-96) | (97-99) | (90-97) | (89- | (90-95) | (96-97) | (92-96) |
| (Min Max) | 500 mL | 99) | | | | 96) | | | |
| 60 min | vessel | 98 | 98 | 99 | 96 | 96 | 96 | 98 | 96 |
| Average | volume | (96- | (97-98) | (98-101) | (92-99) | (91- | (93-98) | (95-99) | (94-99) |
| (Min Max) | (n = 3) | 101) | | | | 99) | | | |
| 70 min | | 102 | 100 | 102 | 101 | 99 | 100 | 100 | 99 |
| Average | | (101- | (100-100) | (101-103) | (98-102) | (96- | (97-101) | (100-101) | (98-101) |
| (Min Max) | | 103) | | | | 102) | | | |

### Physical Properties Characterization

XRPD data of samples placed on stress stability are summarized in Table 21. Both prototype tablets present ezetimibe hydrate when exposed at 40° C./75% RH condition at 2WK, however once packaged the polymorphic conversion does not happen up to 4WK storage.

TABLE 21
XRPD data summary of prototype C 200 and scale up placed on stress stability

| Sample | Batch ID | XRPD |
|---|---|---|
| Tablet Prototype C scale up Initial | A4459/19/02 | OBI + EZE Anhydrous |
| 2 WK @40/75 exposed | | OBI + EZE Anhydrous + small EZE Hydrate |
| 4 WK 40/75 exposed | | OBI + EZE Anhydrous + small EZE Hydrate |
| 4 WK 40/75 packaged | | OBI + EZE Anhydrous |
| 4 WK 40/75 packaged with desiccant | | OBI + EZE Anhydrous |
| Tablet Prototype C 200 mg Initial | A4459/19/03 | OBI + EZE Anhydrous |
| 2 WK @40/75 exposed | | OBI + EZE Anhydrous + small EZE Hydrate |
| 4 WK 40/75 exposed | | OBI + EZE Anhydrous + small EZE Hydrate |
| 4 WK 40/75 packaged | | OBI + EZE Anhydrous |
| 4 WK 40/75 packaged with desiccant | | OBI + EZE Anhydrous |

### Example 3: Fixed Dose Combination of 10 mg Ezetimibe and 10 mg Obicetrapib by Co-Granulation of Drug Substances/Active Ingredients (FDC1) (Small Scale Batch ~500 g)

### High Shear Granulation, Drying, Preparation of Final Blend and Tableting

Three compositions (Composition 1 batch A4459/20/02, Composition 2 batch A4459/20/03 and Composition 3 batch A4459/20/04) were prepared as summarised in Table 22. The prototype formulation composition selected for these compositions was that of “prototype C” (e.g. granule batch A4459/13/03). The preparation and characterization (LOD and XRPD) of the granules are described in the previous sections (small-scale manufactures). The granules were tested for content uniformity, LOD, sieve analysis, TBD and XRPD. The blend for tableting and the compression profile and manufacture of a small batch of tablets at 150 mg tablet weight was performed as described in the previous example. The tablets were tested for content uniformity, XRPD, dissolution and water content by KF. All the intermediates of production and the final drug product were stored as described in the previous sections.

TABLE 22
Composition (% w/w) of granule and tablet for the FDC1 composition

| | Composition #1 | | Composition #2 | | Composition #3 | |
|---|---|---|---|---|---|---|
| | A4459/20/02 | A4459/23/02 | A4459/20/03 | A4459/23/03 | A4459/20/04 | A4459/23/04 |
| | Granule | Tablet | Granule | Tablet | Granule | Tablet |
| Component | (%, w/w) | (%, w/w) | (%, w/w) | (%, w/w) | (%, w/w) | (%, w/w) |

Intra-granular

| | | | | | | |
|---|---|---|---|---|---|---|
| Ezetimibe | 6.981 | 6.667 | 6.981 | 6.667 | 6.981 | 6.667 |
| Obicetrapib | 7.162 | 6.840 | 7.162 | 6.840 | 7.162 | 6.840 |
| Avicel PH 101 | 21.982 | 20.993 | 21.982 | 20.993 | 21.982 | 20.993 |
| Pharmatose 200 M | 57.592 | 55.000 | 57.592 | 55.000 | 57.592 | 55.000 |
| Kollidon 30 | 1.047 | 1.000 | 1.047 | 1.000 | 1.047 | 1.000 |
| Glycolys | 4.189 | 4.000 | 4.189 | 4.000 | 4.189 | 4.000 |
| Kolliphor SLS | 1.047 | 1.000 | 1.047 | 1.000 | 1.047 | 1.000 |
| MilliQ water* | 20.000 + 5.000 | N/A | 20.000 + 5.000 | N/A | up to visual | N/A |
| | | | | | (30.000) | |
| Total Granule | 100.000 | 95.500 | 100.000 | 95.500 | 100.000 | 95.500 |

Extra-granular

| | | | | | | |
|---|---|---|---|---|---|---|
| Glycolys | N/A | 4.000 | N/A | 4.000 | N/A | 4.000 |
| Ligamed MF-2-V | N/A | 0.500 | N/A | 0.500 | N/A | 0.500 |
| Total tablet | N/A | 100.000 | N/A | 100.000 | N/A | 100.000 |

*Water does not appear in the final product. All excipients for granulation added as a dry powder

### Results

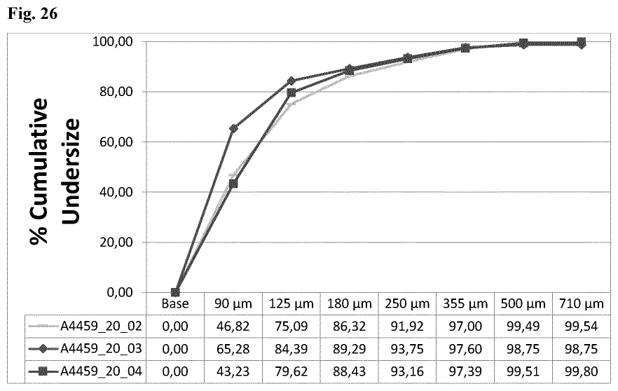

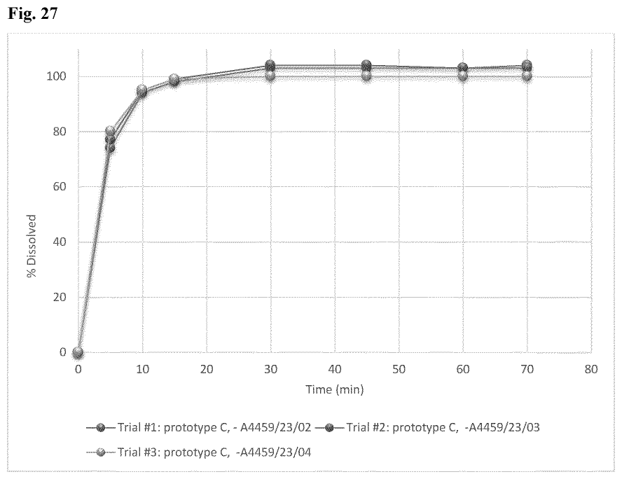

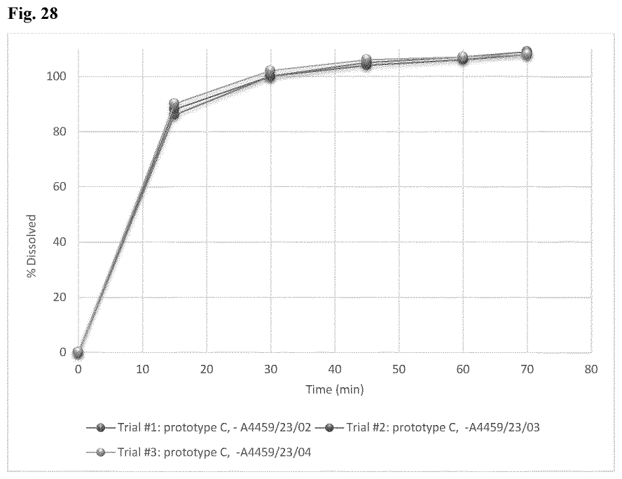

The high shear granulation was conducted successfully. The drying step was conducted without any issues and, after 15 minutes drying the LOD of the granules was lower than the initial LOD. In general, the and the granules presented a relatively large quantity of fine particles despite the increase of the impeller speed (composition 1) (FIG. 26), the time of wet massing (composition 2) or the quantity of granulating agent (composition 3). The tablet friability, time of disintegration, thickness and hardness were found to be similar among these tablet batches.

### Granules Chemical Characterization

The granules were tested for homogeneity, and obicetrapib and ezetimibe were found to be homogeneously dispersed.

### Physical Properties Characterization

XRPD data of the Blend/granules prototypes FDC1 approach are summarized in Table 23. Eze hydrate appears only in the wet granules samples. All the three prototypes present similar flowability.

### Tablets Chemical Characterization

Results of the chemical characterization of the FDC 1 tablets are reported in Table 24. The results of the analytical characterization did not show any significant differences between the three compositions.

### Physical Properties Characterization

XRPD data of the Tablets from FDC1 compositions are summarized in Table 25. There is no presence of Eze hydrate in all samples.

TABLE 23
XRPD data summary of granules from FDC1 compositions

| Sample | Batch ID | XRPD |
|---|---|---|
| Prototype 1 Blend before granulation | A4459/20/02 | OBI + EZE Anhydrous |
| Prototype 1 Wet Granules | | OBI + EZE Anhydrous + small EZE Hydrate |
| Prototype 1 Final dry Granules | | OBI + EZE Anhydrous |
| Prototype 2 Blend before granulation | A4459/20/03 | OBI + EZE Anhydrous |
| Prototype 2 Wet Granules | | OBI + EZE Anhydrous + small EZE Hydrate |
| Prototype 2 Final dry Granules | | OBI + EZE Anhydrous |
| Prototype 3 Blend before granulation | A4459/20/04 | OBI + EZE Anhydrous |
| Prototype 3 Wet Granules | | OBI + EZE Anhydrous + small EZE Hydrate |
| Prototype 3 Final dry Granules | | OBI + EZE Anhydrous |

TABLE 24
Results of the chemical characterization of FDC1 compositions

| | | A4459/23/02 | | A4459/23/03 | | A4459/23/04 | |
|---|---|---|---|---|---|---|---|
| Manufacturing lot number | | Composition #1: | | Composition #2: | | Composition #3: | |
| Prototype | | prototype C, | | prototype C, | | prototype C, | |
| Test | Method | Result | | Result | | Result | |
| Assay | HPLC | | | | | | |
| Obicetrapib (% claim) | 2730 | 100.1 | | 97.6 | | 99.2 | |
| Ezetimibe (% claim) | 2731 | 99.9 | | 99.7 | | 99.7 | |
| Impurities | HPLC | | | | | | |
| IMP_RRT0.94 (% claim) | 2730 | 0.06 | | 0.06 | | 0.06 | |
| FROL(% claim) | 2730 | 0.05 | | 0.06 | | 0.05 | |
| MONO-BN FREE(% claim) | 2730 | 0.17 | | 0.16 | | 0.17 | |
| Total impurities of Obicetrapib ≥0.05% | 2730 | 0.28 | | 0.28 | | 0.28 | |
| EzetimibeKetone (% a/a) | 2731 | <QL (0.05%) | | <QL (0.05%) | | <QL (0.05%) | |
| Ezetimibe tetrahydropyran analog (% a/a) | 2731 | N.D. | | N.D. | | N.D. | |
| Total impurities of Ezetimbe ≥0.05% | 2731 | <QL (0.05%) | | <QL (0.05%) | | <QL (0.05%) | |
| Uniformity of dosage units | USP <905> | | | | | | |
| Obicetrapib | Ph. Eur. 2.9.40 or | Average % Claim: | | Average % Claim: | | Average % Claim: | |
| | JP 6.02 by UNIC/ | 99.7 | | 98.5 | | 99.3 | |
| | 1190 | Range % Claim: | | Range % Claim: | | Range % Claim: | |
| | | (94.0-102.4) | | (97.1 -99.2) | | (97.6-100.5) | |
| | | AV = 5.5 | | AV = 1.8 | | AV = 2.3 | |
| Ezetimibe | | Average % Claim: | | Average % Claim: | | Average % Claim: | |
| | | 102.6 | | 101.5 | | 101.6 | |
| | | Range % Claim: | | Range % Claim: | | Range % Claim: | |
| | | (96.8-105.6) | | (100.3-102.2) | | (100.0-103.1) | |
| | | AV = 7.0 | | AV = 1.5 | | AV = 2.5 | |
| Dissolution Obicetrapib | | | | | | | |
| 5 min Average (Min Max) | Discriminating | 74 | (72-76) | 77 | (74-79) | 80 | (77-83) |
| 10 min Average (Min Max) | method at pH 6.80.2% | 94 | (90-97) | 95 | (92-97) | 95 | (94-96) |
| 15 min Average (Min Max) | w/v of Tween 80, | 98 | (95-102) | 99 | (98-101) | 99 | (98-99) |
| 30 min Average (Min Max) | 50 rpm paddle speed | 103 | (99-106) | 104 | (103-106) | 100 | (102-106) |
| 45 min Average (Min Max) | and 900 mL vessel | 103 | (100-105) | 104 | (103-107) | 100 | (102-101) |
| 60 min Average (Min Max) | volume | 103 | (101-107) | 103 | (102-105) | 100 | (100-100) |
| 70 min Average (Min Max) | | 104 | (102-108) | 103 | (102-104) | 100 | (99-101) |
| Dissolution Ezetimibe | | | | | | | |
| 15 min Average (Min Max) | Dissolution method | 86 | (83-88) | 88 | (86-89) | 90 | (86-92) |
| 30 min Average (Min Max) | based on USP method | 100 | (97-102) | 100 | (98-102) | 102 | (99-104) |
| 45 min Average (Min Max) | conditions at pH 4.5, | 105 | (102-107) | 104 | (102-105) | 106 | (104-107) |
| 60 min Average (Min Max) | 75 rpm paddle speed | 107 | (105-109) | 106 | (105-107) | 107 | (106-109) |
| 70 min Average (Min Max) | and 500 mL vessel | 109 | (107-111) | 108 | (107-108) | 108 | (107-109) |
| | volume | | | | | | |

| | | | | |
|---|---|---|---|---|
| Water content | KF | 4.6% w/w | 4.6% w/w | 4.6% w/w |

TABLE 25
XRPD data summary of granules of FDC1 compositions

| Sample | Batch ID | XRPD |
|---|---|---|
| Composition 1 Tablet | A4459/23/02 | OBI + EZE Anhydrous |
| Composition 2 Tablet | A4459/23/03 | OBI + EZE Anhydrous |
| Composition 3 Tablet | A4459/23/04 | OBI + EZE Anhydrous |

### Example 4: Fixed Dose Combination of 10 mg Ezetimibe and 10 mg Obicetrapib by Granulation of Ezetimibe and Addition of Obicetrapib in the Extra-Granule (FDC2)

### High Shear Granulation and Drying

Three compositions were prepared as summarised in Table 26. The excipients contained in the granule were the same of those used for the manufacture of the granule for the FDC1 approach. The formulation composition of these granules reflected that of FDC1 granule “prototype C”. The method of high shear granulation, granule drying, and milling was described in the previous sections. The granules were tested for content uniformity (ezetimibe only), sieve analysis, TBD and XRPD.

### Preparation of Final Blend, Tableting and Coating

The components of the extra-granular formulation are listed below:
Obicetrapib
Plastic filler (Avicel PH 200)
Brittle filler (Pearlitol 200 SD)
Disintegrant (Glycolys)
Glydant (Aerosil 200)
Lubricant (Ligamed MF-2-V)

The final blend was prepared by weighing accurately and sieving the extra-granular components (excipients and API). The excipients and the granule were loaded in a bin of suitable volume and blended using a Pharmatech mixer. Then, the lubricant (MgSt) was added to the bin and mixed.

For the generation of a compression profile and the manufacture of a small batch of tablets, a single punch compression machine (EK0) equipped with an 9.0 mm round punch (R=11) was used. The target tablet weight was 230 mg and, throughout the process, the tablet friability, disintegration time, hardness, appearance end thickness was monitored as well as the individual tablet weight and the tablet weight of ten tablets.

The tablets were tested for content uniformity (stratified sample: start, middle and end of production), XRPD, dissolution and water content by KF.

The tablets were coated using a 20% w/w Opadry AMB II white aqueous suspension at the required target weight increase (target weight increase 3% w/w, limits 2% w/w-4% w/w). The coating suspension and the method of coating was described in the previous section. The coating parameters as well as the weight gain of the tablets was monitored throughout processing. The coated tablets were tested for XRPD, dissolution, appearance, content uniformity and water content by KF.

All the intermediates of production and the final drug product were stored in double LDPE bags with silica and transferred into an aluminium bag that was thermosealed.

TABLE 26
Composition (% w/w) of granule, tablet and coated tablet of FDC2 compositions

| | Prototype 1 | | | Prototype 2 | | | Prototype 3 | | |
|---|---|---|---|---|---|---|---|---|---|
| | A4459/ | A4459/ | A4459/ | A4459/ | A4459/ | A4459/ | A4459/ | A4459/ | A4459/ |
| | 20/01 | 23/01 | 27/01 | 25/01 | 26/01 | 27/02 | 25/02 | 26/02 | 27/03 |
| | Granule | Tablet | Tablet coated | Granule | Tablet | Tablet coated | Granule | Tablet | Tablet coated |
| Component | (%, w/w) | (%, w/w) | (%, w/w) | (%, w/w) | (%, w/w) | (%, w/w) | (%, w/w) | (%, w/w) | (%, w/w) |

Intra-granule

| | | | | | | | | | |
|---|---|---|---|---|---|---|---|---|---|
| Ezetimibe | 7.692 | 4.348 | 4.348 | 7.692 | 4.348 | 4.348 | 7.692 | 4.348 | 4.348 |
| Avicel PH 101 | 23.614 | 13.347 | 13.347 | 23.614 | 13.347 | 13.347 | 23.614 | 13.347 | 13.347 |
| Pharmatose | 61.771 | 34.914 | 34.914 | 61.771 | 34.914 | 34.914 | 61.771 | 34.914 | 34.914 |
| 200 M | | | | | | | | | |
| Kollidon 30 | 1.154 | 0.652 | 0.652 | 1.154 | 0.652 | 0.652 | 1.154 | 0.652 | 0.652 |
| Glycolys | 4.615 | 2.608 | 2.608 | 4.615 | 2.608 | 2.608 | 4.615 | 2.608 | 2.608 |
| Kolliphor SLS | 1.154 | 0.652 | 0.652 | 1.154 | 0.652 | 0.652 | 1.154 | 0.652 | 0.652 |
| MilliQ water* | 20 + 5 | N/A | N/A | 20 + 5 | N/A | N/A | 20 + 5 | N/A | N/A |
| Total Granule | 100.000 | 56.521 | 56.521 | 100.000 | 56.521 | 56.521 | 100.000 | 56.521 | 56.521 |

Extra-granule

| | | | | | | | | | |
|---|---|---|---|---|---|---|---|---|---|
| Obicetrapib | N/A | 4.461 | 4.461 | N/A | 4.461 | 4.461 | N/A | 4.461 | 4.461 |
| Avicel PH 200 | N/A | 25.974 | 25.974 | N/A | 23.118 | 23.118 | N/A | 22.757 | 22.757 |
| Pearlitol 200 SD | N/A | 10.000 | 10.000 | N/A | 8.900 | 8.900 | N/A | 8.761 | 8.761 |
| Glycolys | N/A | 2.174 | 2.174 | N/A | 5.000 | 5.000 | N/A | 5.000 | 5.000 |
| Aerosil 200 | N/A | 0.435 | 0.435 | N/A | 1.000 | 1.000 | N/A | 1.000 | 1.000 |
| Ligamed MF-2-V | N/A | 0.435 | 0.435 | N/A | 1.000 | 1.000 | N/A | 1.500 | 1.500 |
| Opadry AMB II | N/A | N/A | 3.000 | N/A | N/A | 3.000 | N/A | N/A | 3.000 |
| Total tablet | N/A | 100.000 | 103.000 | N/A | 100.000 | 103.00 | N/A | 100.000 | 103.00 |

*Water does not appear in the final product. All excipients for granulation added as a dry powder

### Results

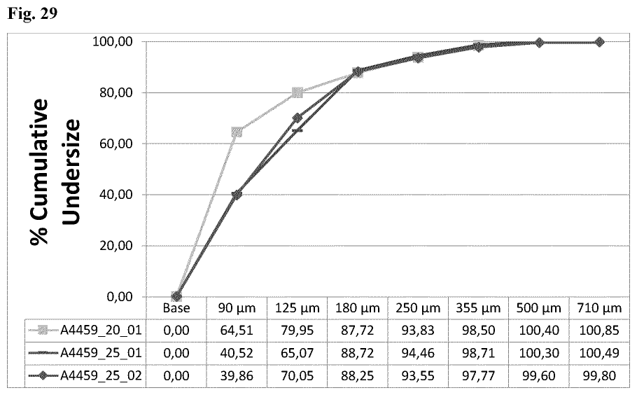

The high shear granulation of the FDC2 compositions was conducted successfully. The drying step was conducted without any issues and, after ca. 16 minutes drying, the LOD of the granules was lower than the initial LOD. The granules showed a relatively large quantity of fine particles (FIG. 29). The values of disintegration time and thickness were similar among FDC2 tablet batches.

### Granules Chemical Characterization

A4459/20/01 blend was tested for homogeneity of both obicetrapib and ezetimibe, and was found to be homogeneously dispersed. Results for other two granules were not collected.

### Tablets Chemical Characterization

Chemical characterization of the protoype 1 of FDC 2 tablets results are reported in Table 27 and Table 28. The results of the analytical characterization did not show any significant differences between the three prototypes of FDC2.

### Physical Properties Characterization

XRPD data of the Blend/granules from FDC2 compositions are summarized in Table 29. XRPD data of the tablets from FDC2 approach are summarized in Table 30. There was a presence of small amount of Eze hydrate in prototype 1.

TABLE 27
Results of the analytical characterization of FDC2 - Uncoated

| Manufacturing lot number | | A4459/23/01 | | A4459/26/01 | | A4459/26/02 | |
|---|---|---|---|---|---|---|---|
| Prototype | | Prototype 1 | | Prototype 2 | | Prototype 3 | |
| Test | Method | Result | | Result | | Result | |
| Assay | HPLC | | | | | | |
| Obicetrapib (% claim) | 2730 | 97.6 | | 96.8 | | 95.6 | |
| Ezetimibe (% claim) | 2731 | 102.1 | | 102.7 | | 101.0 | |
| Impurities | HPLC | | | | | | |
| IMP_RRT0.94 (% claim) | 2730 | 0.06 | | 0.06 | | 0.05 | |
| FROL(% claim) | 2730 | 0.05 | | 0.05 | | 0.05 | |
| MONO-BN FREE(% claim) | 2730 | 0.19 | | 0.16 | | 0.16 | |
| Total impurities of | 2730 | 0.31 | | 0.27 | | 0.26 | |
| Obicetrapib ≥0.05% | | | | | | | |
| Ezetimibe Ketone (% a/a) | 2731 | 0.05 | | 0.05 | | 0.05 | |
| Ezetimibe tetrahydropyran | 2731 | N.D. | | N.D. | | N.D. | |
| analog (% a/a) | | | | | | | |
| Total impurities of | 2731 | 0.05 | | 0.05 | | 0.05 | |
| Ezetimbe ≥0.05% | | | | | | | |
| Uniformity of dosage units | USP <905> | Average % Claim: | | Average % Claim: | | Average % Claim: | |
| Obicetrapib | Ph. Eur. 2.9.40 or | 97.6 | | 96.7 | | 96.4 | |
| | JP 6.02 by UNIC/ | Range % Claim: | | Range % Claim: | | Range % Claim: | |
| | 1190 | (95.5-100.3) | | (96.1-97.4) | | (95.2-98.2) | |
| | | AV = 4.9 | | AV = 2.9 | | AV = 4.7 | |
| Ezetimibe | | Average % Claim: | | Average % Claim: | | Average % Claim: | |
| | | 101.7 | | 102.3 | | 101.1 | |
| | | Range % Claim: | | Range % Claim: | | Range % Claim: | |
| | | (100.6-103.1) | | (101.0-103.2) | | (99.3-103.1) | |
| | | AV = 2.2 | | AV = 2.6 | | AV = 2.9 | |
| Dissolution Obicetrapib | | | | | | | |
| 5 min Average (Min Max) | Discriminating method | 56 | (51-62) | 56 | (51-61) | 55 | (50-57) |
| 10 min Average (Min Max) | at pH 6.80.2% w/v of | 80 | (73-86) | 78 | (74-79) | 77 | (76-78) |
| 15 min Average (Min Max) | Tween 80, 50 rpm | 86 | (77-92) | 86 | (84-89) | 83 | (82-86) |
| 30 min Average (Min Max) | paddle speed and 900 | 89 | (81-97) | 91 | (89-92) | 91 | (87-93) |
| 45 min Average (Min Max) | mL vessel volume | 89 | (80-96) | 91 | (88-92) | 91 | (88-93) |
| 60 min Average (Min Max) | | 88 | (80-95) | 91 | (89-92) | 90 | (88-92) |
| 70 min Average (Min Max) | | 98 | (95-100) | 97 | (96-89) | 96 | (95-97) |
| Dissolution Ezetimibe | | | | | | | |
| 15 min Average (Min Max) | Dissolution method | 68 | (61-71) | — | | — | |
| 30 min Average (Min Max) | based on USP method | 76 | (70-79) | — | | — | |
| 45 min Average (Min Max) | conditions at pH 4.5, 50 | 79 | (74-81) | — | | — | |
| 60 min Average (Min Max) | rpm paddle speed and | 80 | (75-83) | — | | — | |
| 70 min Average (Min Max) | 500 mL vessel volume | 101 | (100-101) | — | | — | |
| Dissolution Ezetimibe | | | | | | | |
| 15 min Average (Min Max) | Dissolution method | 88 | (85-94) | 85 | (81-88) | 82 | (76-88) |
| 30 min Average (Min Max) | based on USP method | 94 | (92-101) | 94 | (90-96) | 90 | (84-97) |
| 45 min Average (Min Max) | conditions at pH 4.5, 75 | 97 | (94-103) | 97 | (93-100) | 93 | (87-101) |
| 60 min Average (Min Max) | rpm paddle speed and | 98 | (95-104) | 98 | (94-101) | 94 | (88-102) |
| 70 min Average (Min Max) | 500 mL vessel volume | 103 | (102-105) | 102 | (101-104) | 101 | (99-103) |

| | | | | |
|---|---|---|---|---|
| Water content | KF | 4.4% w/w | 4.3% w/w | 4.0% w/w |

TABLE 28
Results of the analytical characterization of FDC2- Coated

| | | A4459/27/01 | | A4459/27/02 | | A4459/27/03 | |
|---|---|---|---|---|---|---|---|
| Manufacturing lot number | | Prototype 1 - | | Prototype 2 | | Prototype 3 | |
| Prototype | | Coated | | Coated | | Coated | |
| Test | Method | Result | | Result | | Result | |
| Assay | HPLC | | | | | | |
| Obicetrapib (% claim) | 2730 | 95.9 | | 96.4 | | 95.0 | |
| Ezetimibe (% claim) | 2731 | 102.1 | | 102.6 | | 101.0 | |
| Impurities | HPLC | | | | | | |
| IMP_RRT0.94 (% claim) | 2730 | 0.06 | | 0.06 | | 0.06 | |
| FROL(% claim) | 2730 | 0.05 | | 0.05 | | 0.05 | |
| MONO-BN FREE(% claim) | 2730 | 0.16 | | 0.16 | | 0.16 | |
| Total impurities of Obicetrapib ≥0.05% | 2730 | 0.27 | | 0.27 | | 0.27 | |
| EzetimibeKetone (% a/a) | 2731 | <QL (0.05%) | | <QL (0.05%) | | <QL (0.05%) | |
| Ezetimibe tetrahydropyran analog (% a/a) | 2731 | N.D. | | N.D. | | N.D. | |
| Total impurities of Ezetimbe ≥0.05% | 2731 | <QL (0.05%) | | <QL (0.05%) | | <QL (0.05%) | |
| Uniformity of dosage units | USP <905> | Average % Claim: | | Average % Claim: | | Average % Claim: | |
| Obicetrapib | Ph. Eur. 2.9.40 or | 97.0 | | 96.4 | | 95.2 | |
| | JP 6.02 by UNIC/ | Range % Claim: | | Range % Claim: | | Range % Claim: | |
| | 1190 | (94.0-99.1) | | (95.4-97.8) | | (93.2-96.1) | |
| | | AV = 6.1 | | AV = 4.0 | | AV = 5.5 | |
| Ezetimibe | | Average % Claim: | | Average % Claim: | | Average % Claim: | |
| | | 101.2 | | 101.9 | | 101.0 | |
| | | Range % Claim: | | Range % Claim: | | Range % Claim: | |
| | | (99.6-102.5) | | (100.7-102.9) | | (99.6-102.3) | |
| | | AV = 2.2 | | AV = 2.2 | | AV = 1.7 | |
| Dissolution Obicetrapib | | | | | | | |
| 5 min Average (Min Max) | Discriminating | 58 | (43-66) | 59 | (51-63) | 57 | (52-61) |
| 10 min Average (Min Max) | method at pH 6.8 + | 83 | (76-86) | 79 | (77-83) | 85 | (82-87) |
| 15 min Average (Min Max) | 0.2% w/v of Tween | 88 | (84-91) | 84 | (82-90) | 90 | (87-93) |
| 30 min Average (Min Max) | 80, 50 rpm paddle | 94 | (91-96) | 90 | (87-97) | 96 | (92-101) |
| 45 min Average (Min Max) | speed and 900 mL | 95 | (91-99) | 90 | (88-98) | 95 | (91-102) |
| 60 min Average (Min Max) | vessel volume | 95 | (91-102) | 90 | (90-98) | 94 | (91-100) |
| 70 min Average (Min Max) | | 103 | (100-104) | 97 | (95-98) | 98 | (97-99) |
| Dissolution Obicetrapib | | | | | | | |
| 15 min Average (Min Max) | Discriminating | 89 | (87-91) | 90 | (88-92) | 90 | (87-93) |
| 30 min Average (Min Max) | method at pH 6.8 + | 95 | (94-96) | 95 | (94-97) | 95 | (92-97) |
| 45 min Average (Min Max) | 0.2% w/v of Tween | 96 | (95-98) | 96 | (95-97) | 96 | (94-97) |
| 60 min Average (Min Max) | 80, 75 rpm paddle | 97 | (96-98) | 96 | (95-97) | 96 | (94-97) |
| 70 min Average (Min Max) | speed and 1000 mL | 98 | (95-100) | 96 | (94-98) | 96 | (95-98) |
| | vessel volume | | | | | | |
| Dissolution Ezetimibe | | | | | | | |
| 15 min Average (Min Max) | Dissolution method | 82 | (76-89) | 84 | (78-90) | 85 | (76-88) |
| 30 min Average (Min Max) | based on USP method | 92 | (85-101) | 92 | (87-97) | 92 | (86-95) |
| 45 min Average (Min Max) | conditions at pH 4.5, | 94 | (87-102) | 95 | (90-100) | 95 | (89-98) |
| 60 min Average (Min Max) | 75 rpm paddle speed | 95 | (88-103) | 96 | (92-101) | 96 | (90-99) |
| 70 min Average (Min Max) | and 500 mL vessel | 102 | (101-104) | 103 | (102-104) | 102 | (102-103) |
| | volume | | | | | | |

| | | | | |
|---|---|---|---|---|
| Water content | KF | 4.0% w/w | 4.1% w/w | 4.0% w/w |

TABLE 29
XRPD data summary of granules from FDC2 compositions

| Sample | Batch ID | XRPD |
|---|---|---|
| Prototype 1 Final | A4459/20/01 | OBI + EZE Anhydrous + |
| dry Granules | | small amount of EZE hydrate |
| Prototype 2 Final | A4459/25/01 | OBI + EZE Anhydrous |
| dry Granules | | |
| Prototype 3 Final | A4459/25/02 | OBI + EZE Anhydrous |
| dry Granules | | |

TABLE 30
XRPD data summary of tablets from FDC2 compositions

| Sample | Batch ID | XRPD |
|---|---|---|
| Prototype 1 Tablet uncoated-coated | A4459/23/01/-A4459/27/01 | OBI + EZE Anhydrous + small amount of EZE hydrate |
| Prototype 2 Tablet uncoated-coated | A4459/26/01- A4459/27/02 | OBI + EZE Anhydrous |
| Prototype 3 Tablet uncoated-coated | A4459/26/02 - A4459/27/03 | OBI + EZE Anhydrous |

### Stress Stability

Prototype 2 coated tablet was selected for the stress stability study with the following design

TABLE 31
Stress stability study design

| Storage Conditions | | Time (weeks) | | | |
|---|---|---|---|---|---|
| T/RH | Type | 0 | 2 | 3 | 4 |
| 25° C./60% RH | Long-term | T | (T) | — | (T) |
| 40° C./75% RH uncovered | Stress (T) | | T | — | T |
| 40° C./75% RH closed | Accelerated | | (T) | — | (T) |

Key:
T = Tested for appearance, assay & related substances, discriminating dissolution, water content by KF, and form check by XRPD
(T) = Optional testing

Results are reported in Table 66, Table 67 and Table 68.

TABLE 32
Results for assay, water content and visual appearance
of prototype 2 FDC2 stress stability

| | Time point- | Assay of | Assay of | Water | |
|---|---|---|---|---|---|
| | Storage | Ezetimibe | Obicetrapib | content | Appearance |
| Prototype No. | conditions | (% Claim) | (% Claim) | (% w/w) | (visual) |
| Prototype 2 BN | Initial | 102.7 | 96.4 | 4.1 | White round tablet |
| A4459/28/02 | 2 weeks - 40° C./ | 101.2 | 95.8 | 4.6 | White round tablets |
| | 75% RH exposed | | | | |
| | 4 weeks - 40° C./ | 102.1 | 94.6 | 5.1 | White round tablet |
| | 75% RH exposed | | | | |
| | 4 weeks - 40° C./ | 101.4 | 96.0 | 3.1 | Not tested |
| | 75% RH packed | | | | |

TABLE 33
Results for impurities profile of prototype 2 FDC2 stress stability

| | | Obicetrapib related impurities | | | | Ezetimibe related impurities | | | | |
|---|---|---|---|---|---|---|---|---|---|---|
| | Time | | | MONO- | | | | | | Total |
| | point- | IMP_RRT | | BN | Total | IMP_RRT | IMP_RRT | EZETIMIBE | EZETIMIBE | impurities |
| | Storage | 0.94 | FROL | FREE | impurities | 0.73 | 0.79 | CYCLIC | KETONE | (≥0.05) |
| Batch | conditions | (% claim) | (% claim) | (% claim) | (% claim) | (% a/a) | (% a/a) | (% a/a) | (% a/a) | (% a/a) |
| Prototype | Initial | 0.06 | 0.05 | 0.16 | 0.27 | N.D. | 0.04 | N.D | 0.04 | < QL |
| 2 BN | 2 weeks- | 0.06 | 0.05 | 0.16 | 0.26 | N.D. | 0.05 | N.D. | 0.06 | 0.11 |
| | 40° C./75% | | | | | | | | | |
| | RH | | | | | | | | | |
| | exposed | | | | | | | | | |
| A4459/ | 4 weeks- | 0.05 | 0.05 | 0.16 | 0.26 | N.D. | 0.05 | 0.06 | 0.07 | 0.18 |
| 28/02 | 40° C./75% | | | | | | | | | |
| | RH | | | | | | | | | |
| | exposed | | | | | | | | | |
| | 4 weeks- | 0.06 | 0.06 | 0.16 | 0.28 | < QL | 0.03 | 0.05 | 0.07 | 0.12 |
| | 40° C./75% | | | | | (0.05%) | | | | |
| | RH packed | | | | | | | | | |

N.D. = Not Detected

TABLE 34
Results of the dissolution characterization of prototype 2 FDC2 stress stability

Manufacturing lot number
Prototype 2 BN A4459/28/02
Time point and storage conditions

| | | | 2 weeks - | 4 weeks - | |
|---|---|---|---|---|---|
| | | | 40° C./75% | 40° C./75% | |
| | | Initial | RH exposed | RH exposed | 4 weeks - |
| Test | | Result | Result | Result | 40° C./75% |
| Obicetrapib | Method | (n = 6) | (n = 6) | (n = 6) | RH packed |
| 5 min | Discriminating | 59 | 51 | 57 | 52 |
| Average | method at pH 6.80.2% | (51-63) | (45-53) | (55-65) | (47-57) |
| (Min Max) | w/v of Tween 80, | | | | |
| 10 min | 50 rpm paddle speed | 79 | 71 | 77 | 80 |
| Average | and 900 mL vessel | (77-83) | (68-74) | (74-81) | (78-83) |
| (Min Max) | volume | | | | |
| 15 min | | 84 | 78 | 83 | 88 |
| Averag | | (82-90) | (76-81) | (80-88) | (87-89) |
| (Min Max) | | | | | |
| 30 min | | 90 | 86 | 89 | 94 |
| Average | | (87-97) | (84-89) | (86-93) | (91-96) |
| (Min Max) | | | | | |
| 45 min | | 90 | 87 | 89 | 93 |
| Average | | (88-98) | (85-89) | (87-92) | (87-92) |
| (Min Max) | | | | | |
| 60 min | | 90 | 87 | 89 | 91 |
| Average | | (90-98) | (84-90) | (88-93) | (88-93) |
| (Min Max) | | | | | |
| 70 min | | 97 | 97 | 97 | 97 |
| Average | | (95-98) | (95-97) | (96-98) | (96-98) |
| (Min Max) | | | | | |

| | | Time point and storage conditions | | | |
|---|---|---|---|---|---|
| | | | 2 weeks - | 4 weeks - | 4 weeks - |
| | | | 40° C./75% | 40° C./75% | 40° C./75% |
| | | Initial | RH exposed | RH exposed | RH packed |
| Ezetimibe | | (n = 6) | (n = 6) | (n = 6) | (n = 6) |
| 15 min | Dissolution method | 84 | 69 | 72 | 78 |
| Average | based on USP method | (78-90) | (66-73) | (66-76) | (64-87) |
| (Min Max) | conditions at pH 4.5, | | | | |
| 30 min | 75 rpm paddle speed | 92 | 81 | 86 | 90 |
| Average | and 500 mL vessel | (87-97) | (77-84) | (81-90) | (79-98) |
| (Min Max) | volume (n = 3) | | | | |
| 45 min | | 95 | 86 | 92 | 94 |
| Average | | (90-100) | (83-89) | (87-96) | (85-101) |
| (Min Max) | | | | | |
| 60 min | | 96 | 89 | 96 | 96 |
| Average | | (92-101) | (86-91) | (90-101) | (88-102) |
| (Min Max) | | | | | |
| 70 min | | 103 | 99 | 102 | 103 |
| Average | | (102-104) | (97-100) | (100-104) | (101-106) |
| (Min Max) | | | | | |

### Example 5: Fixed Dose Combination of 10 mg Ezetimibe and 10 mg Obicetrapib by Granulation of Obicetrapib and Addition of Ezetimib in the Extra-Granule (FDC3)

Prototype compositions were prepared as summarised in Table 35. The method of granulation was as per process condition 2 as described above. The method of high shear granulation, granule drying, and milling was described in the previous sections. The granules were tested for content uniformity (obicetrapib only), sieve analysis, TBD and XRPD.

### Preparation of Final Blend, Tableting and Coating

The final blend was prepared by weighing accurately and sieving the extra-granular components (excipients and API). The excipients and the granule were loaded in a bin of suitable volume and blended using a Pharmatech mixer. Then, the lubricant (MgSt) was added to the bin and mixed.

For the generation of a compression profile and the manufacture of a small batch of tablets, a single punch compression machine was used. The target tablet weight was 230 mg and, throughout the process, the tablet friability, disintegration time, hardness, appearance end thickness was monitored as well as the individual tablet weight and the tablet weight of ten tablets. The tablets were tested for content uniformity (stratified sample: start, middle and end of production), XRPD, dissolution and water content by KF.

The tablets were coated using a 20% w/w Opadry AMB II white aqueous suspension at the required target weight increase. The coating suspension and the method of coating was described in the previous section. The coating parameters as well as the weight gain of the tablets was monitored throughout processing. The coated tablets were tested for XRPD, dissolution, appearance, content uniformity and water content by KF.

All the intermediates of production and the final drug product were stored in double LDPE bags with silica and transferred into an aluminium bag that was thermosealed.

TABLE 35
Composition (% w/w) of the granule, tablet
and coated tablet of the FDC3 composition

| | | | | |
|---|---|---|---|---|
| | Granule | Tablet | Tablet | Coated Tablet |
| Component | (%, w/w) | (%, w/w) | (mg) | (%, w/w) |

Intra-granule

| | | | | |
|---|---|---|---|---|
| Obicetrapib | 7.89 | 4.46 | 10.26 | 10.26 |
| Avicel PH 101 | 23.614 | 13.347 | 30.7 | 30.7 |
| Pharmatose 200 M | 61.573 | 34.802 | 80.04 | 80.04 |
| Kollidone 30 | 1.154 | 0.652 | 1.5 | 1.5 |
| Glycolys | 4.615 | 2.608 | 6 | 6 |
| Kolliphor SLS | 1.154 | 0.652 | 1.5 | 1.5 |
| Purified Water* | 30 | N/A | N/A | N/A |
| Total Granule | 100 | 56.521 | 130 | 130 |

Extra-granule

| | | | | |
|---|---|---|---|---|
| Ezetimib | N/A | 4.438 | 10 | 10 |
| Avicel PH 200 | N/A | 23.23 | 53.43 | 53.43 |
| Pearlitol 200 SD** | N/A | 8.9 | 20.47 | 20.47 |
| Glycolys | N/A | 5 | 11.5 | 11.5 |
| Aerosil 200** | N/A | 1 | 2.3 | 2.3 |
| Ligamed MF-2-V | N/A | 1 | 2.3 | 2.3 |
| Opadry AMB II | | N/A | N/A | 6.9 |
| Total Tablet | | 100 | 230 | 236.9 |

**or lactose
***optional

### Example 6: Fixed Dose Combination of 10 mg Ezetimibe and 10 mg Obicetrapib as a Bilayer Tablet by Individual Granulation of Obicetrapib and Ezetimib Followed by Compression (FDC4)

Prototype compositions were prepared as summarised in Table 36. The method of granulation of ezetimibe was same as described above for FDC1 and of obicetrapib was same as described above for FDC3. The method of high shear granulation, granule drying, and milling was same described in the previous section for FDC1. The granules were then fed via two hoppers into the compression machine. The first granule was used to fill the die followed by a light compression. The second granule was then filled followed by compression as per the method explained in previous examples. The individual granules were tested for content uniformity (obicetrapib or ezetimibe), sieve analysis, TBD and XRPD. The granules were compressed to form a tablet as per the methods described above. The tablets were tested for content uniformity (stratified sample: start, middle and end of production), XRPD, dissolution and water content by KF.

The tablets were coated using a 20% w/w Opadry AMB II white aqueous suspension at the required target weight increase. The coating suspension and the method of coating was described in the previous section. The coating parameters as well as the weight gain of the tablets was monitored throughout processing. The coated tablets were tested for XRPD, dissolution, appearance, content uniformity and water content by KF.

All the intermediates of production and the final drug product were stored in double LDPE bags with silica and transferred into an aluminium bag that was thermosealed.

TABLE 36
Composition (% w/w) of the granule, tablet and coated tablet of the FDC4 composition

| | FDC4 (obicetrapib granule) | | | | | FDC4 (ezetimibe granule) | | | | |
|---|---|---|---|---|---|---|---|---|---|---|
| | | | | | Final | | | | | Final |
| | | | Bi- | Coated | Bi-layer | | | Bi- | | Bi-layer |
| | Granule | Tablet | layer | Tablet | Tablet | Granule | Tablet | layer | Tablet | Tablet |
| Component | (%, w/w) | (%, w/w) | (mg) | (%, w/w) | (mg) | (%, w/w) | (%, w/w) | (mg) | (%, w/w) | (mg) |

Intra-granule

| | | | | | | |
|---|---|---|---|---|---|---|
| Ezetimibe | N/A | N/A | N/A | 7.692 | 10 | 10 |
| Obicetrapib | 7.89 | 10.26 | 10.26 | N/A | N/A | N/A |
| Avicel PH | 23.614 | 30.7 | 30.7 | 23.614 | 30.7 | 30.7 |
| 101 | | | | | | |
| Pharmatose | 61.573 | 80.04 | 80.04 | 61.771 | 80.3 | 80.3 |
| 200 M | | | | | | |
| Kollidon 30 | 1.154 | 1.5 | 1.5 | 1.154 | 1.5 | 1.5 |
| Glycolys | 4.615 | 6 | 6 | 4.615 | 6 | 6 |
| Kolliphor | 1.154 | 1.5 | 1.5 | 1.154 | 1.5 | 1.5 |
| SLS | | | | | | |
| Purified | 30 | N/A | N/A | 30 | N/A | N/A |
| Water* | | | | | | |
| Total | 100 | 130 | 130 | 100 | 130 | 130 |
| Granule | | | | | | |

Extra-granule for each bilayer

| Glycolys | N/A | 5.5 | | 5.5 | N/A | 5.5 | 5.5 |
|---|---|---|---|---|---|---|---|
| Aerosil 200 | N/A | N/A | | N/A | N/A | N/A | N/A |
| Ligamed | N/A | 1.4 | | 1.4 | N/A | 1.4 | 1.4 |
| MF-2-V | | | | | | | |
| Individual Bilayer | N/A | 136.9 | | | N/A | 136.9 | |
| Total Tablet | N/A | | | 273.8 | | | |
| Coating of 273.8 mg bi-layer tablets | | | | | | | |
| Opadry | | | | | | | |
| AMB II | N/A | N/A | 3 | 8.2 | | | |

*purified water is a granulating agent and does not feature in any of the final formulations

### Example 7: Scale-Up of FDC1 and FDC2 Compositions

### High Shear Granulation, Drying, Final Blend, Tableting and Coating

The API and excipients were dispensed accurately, sieved and added to the granulation bowl according to the approach detailed in the FDC1 and FDC2 formulation approaches above. The parameters of granulation for both the FDC1 and FDC2 approach were identical. The granules were tested for content uniformity (only for the FDC1 approach), LOD, sieve analysis, TBD and XRPD.

The final blend of the FDC1 and FDC2 compositions were prepared to manufacture tablets whose batch number and composition is detailed in Table 37. The components of the extra-granule were manually sieved and loaded in a bin of suitable volume. The granule was mixed with the extra-granular materials using a Pharmatech mixer. Then, the FDC2 blend was tested for content uniformity.

For the generation of a compression profile and for the tableting exercise the use of rotary press machines was assessed. The friability, disintegration time, hardness, thickness and appearance of the tablets as well as the individual tablet weight and the tablet weight of ten tablets was monitored during the tableting exercise. The tablets were tested for content uniformity (stratified samples: start, middle and end of production), XRPD, dissolution and water content by KF.

The tablets were coated using a 20% w/w Opadry AMB II white aqueous suspension at the required target weight increase (target weight increase 3% w/w, limits 2% w/w-4% w/w). The coating suspension and the method of coating was described in the previous section. The coating parameters as well as the weight gain of the tablets was monitored throughout processing. The coated tablets were tested for XRPD, dissolution, appearance, assay and impurities, and water content by KF.

All the intermediates of production and the final drug product were stored in double LDPE bags with silica and transferred into an aluminium bag that was thermosealed.

TABLE 37
Composition (% w/w) of granule, tablet and coated tablet of FDC1 and FDC2
scale-up trials

| | Scale-Up-FDC2 | | | Scale-Up FDC1 | | |
|---|---|---|---|---|---|---|
| | A4459/29/04 | A4459/29/05 | | A4459/29/01 | A4459/29/02 | |
| | Granule | Tablet | Coated tablet | Granule | Tablet | Coated tablet |
| Component | (%, w/w) | (%, w/w) | (%, w/w) | (%, w/w) | (%, w/w) | (%, w/w) |

Intra-granular

| | | | | | | |
|---|---|---|---|---|---|---|
| Ezetimibe | 7.692 | 4.348 | 4.348 | 7.018 | 6.667 | 6.667 |
| Obicetrapib | N/A | N/A | N/A | 7.200 | 6.840 | 6.840 |
| Avicel PH 101 | 23.614 | 13.347 | 13.347 | 21.950 | 20.853 | 20.853 |
| Pharmatose 200 | 61.771 | 34.914 | 34.914 | 57.516 | 54.640 | 54.640 |
| M | | | | | | |
| Kollidon 30 | 1.154 | 0.652 | 0.652 | 1.053 | 1.000 | 1.000 |
| Glycolys | 4.615 | 2.608 | 2.608 | 4.210 | 4.000 | 4.000 |
| Kolliphor SLS | 1.154 | 0.652 | 0.652 | 1.053 | 1.000 | 1.000 |
| Purified | 20.000 + | N/A | N/A | 20.000 + | N/A | N/A |
| Water* | 5.000 + 5.000 | | | 5.000 + 5.000 | | |
| Total Granule | 100.000 | 56.521 | 56.521 | 100.000 | 95.000 | 95.000 |

Extra-granular

| | | | | | | |
|---|---|---|---|---|---|---|
| Obicetrapib | N/A | 4.461 | 4.461 | N/A | N/A | N/A |
| Avicel PH 200 | N/A | 23.118 | 23.118 | N/A | N/A | N/A |
| Pearlitol 200 | N/A | 8.900 | 8.900 | N/A | N/A | N/A |
| SD | | | | | | |
| Glycolys | N/A | 5.000 | 5.000 | N/A | 4.000 | 4.000 |
| Aerosil 200 | N/A | 1.000 | 1.000 | N/A | N/A | N/A |
| Ligamed | N/A | 1.000 | 1.000 | N/A | 1.000 | 1.000 |
| MF-2-V | | | | | | |
| Opadry AMB II | N/A | N/A | 3.000 | N/A | N/A | 3.000 |
| Total Tablet | N/A | 100.000 | 103.000 | N/A | 100.000 | 103.000 |

*Water does not appear in the final product. The excipient used in the granulation were added as a dry powder

### Results

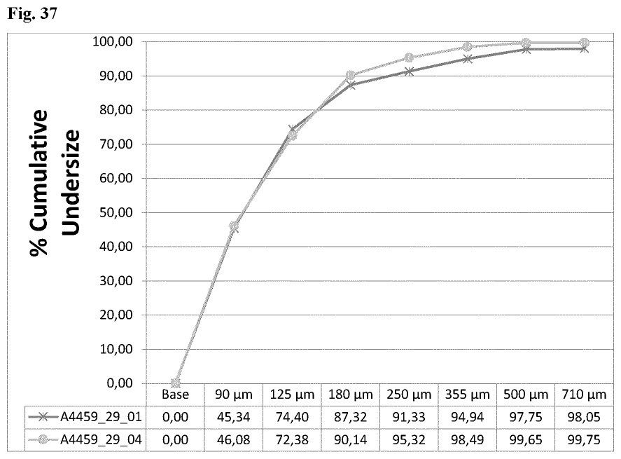

The high shear granulation of the scale-up batches was conducted successfully. The granules presented similar values of PSD by sieve analysis and a large quantity of fines (FIG. 37). These PSD values were comparable to those found in the previous trials (e.g. batch A4459/16/02 as a reference for the FDC1 scale-up batch and batch A4459/25/01 as a reference for the FDC2 scale-up batch). Despite this similarity of PSD data, the flowability of the scale-up batches improved in comparison to that of the reference batches according to the value of the Hauser ratio. These tablets presented disintegration time shorter than 5 mins and friability lower than 0.2%. The coating process was performed without any critical issues and the appearance of the coated tablets for both formulation approaches was suitable since the surface of the tablets was smooth.

### Granules Chemical Characterization

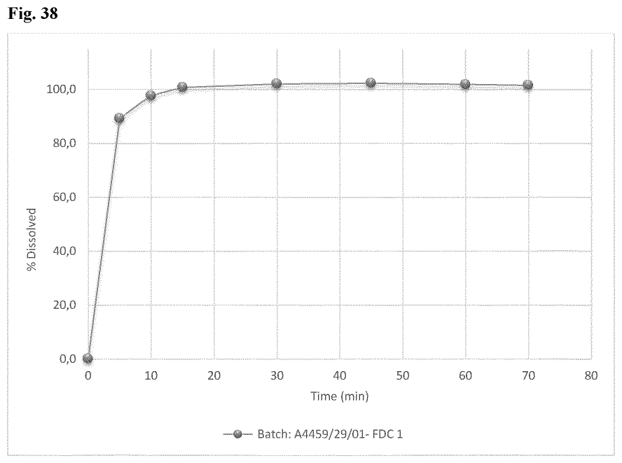

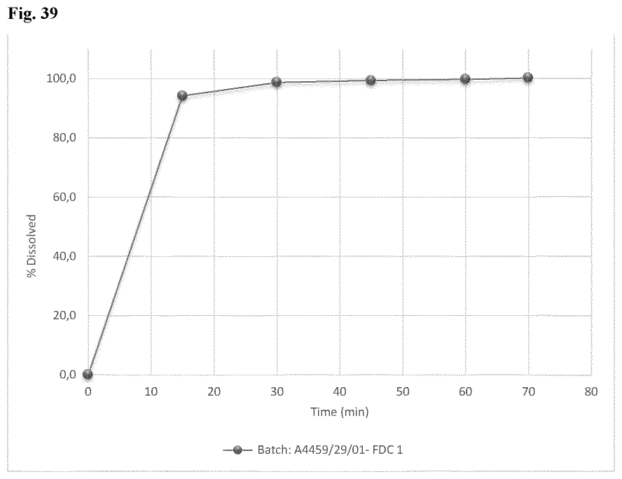

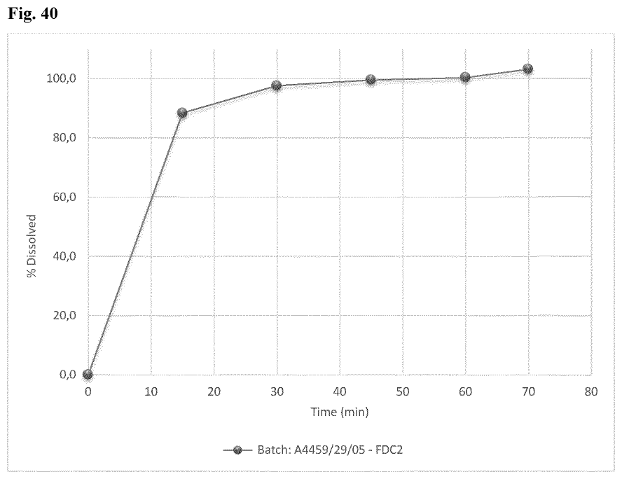

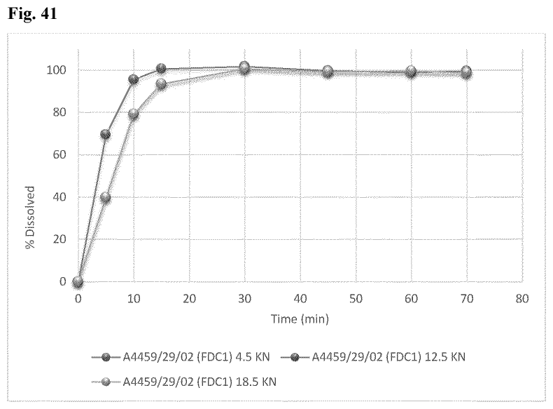

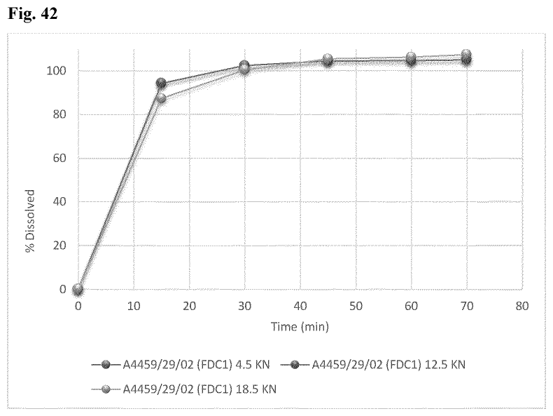

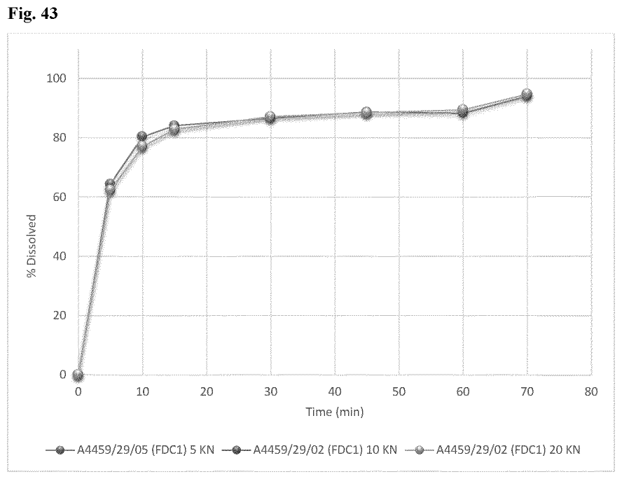

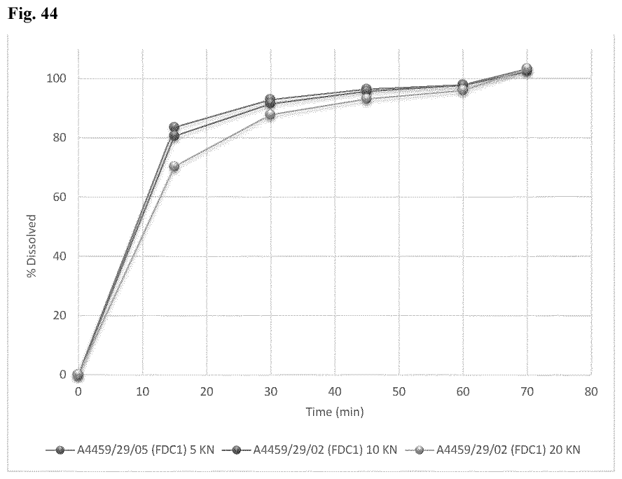

The granule and the final blend were tested for homogeneity of both obicetrapib and ezetimibe and were found to be homogeneously dispersed. Dissolution of both obicetrapib and ezetimibe and impurities profile of ezetimibe, results reported in Table 38, FIG. 38, FIG. 39 and FIG. 40.

### Tablets Chemical Characterization

Chemical characterization of the scale up batches uncoated tablets results are reported in Table 39. Dissolution results on uncoated tablets at different compression forces are reported in Table 40, Table 41, Table 42 and Table 43. Results for coated tablets are reported in Table 42.

### Physical Properties Characterization

XRPD data of the Granules/Tablets from Scale up batches are summarized in Table 43. All the batches tested showed the absence of EZE hydrate.

TABLE 38
Granule and final blend impurities profile
and dissolution result for scale up batches

| | | Manufacturing lot number | |
|---|---|---|---|
| | | A4459/29/01 | A4459/29/05 |
| | | Prototype | |
| | | FDC 1 | FDC 2 |
| | | (intra-Intra) | (intra-Extra) |
| Test | Method | Result | Result |
| Impurities | HPLC | | |
| IMP_RRT0.79 (% a/a) | 2731 | <QL (0.05%) | 0.05 |
| Ezetimibe□Ketone (% a/a) | 2731 | 0.05 | 0.05 |
| Ezetimibe tetrahydro- | 2731 | N.D. | N.D. |
| pyran analog (% a/a) | | | |
| Total impurities of | 2731 | 0.05 | 0.10 |
| Ezetimbe ≥0.05% | | | |
| Dissolution Obicetrapib | | | |
| 5 min Average | Discriminating method | 85 | — |
| (Min Max) | at pH 6.80.2% w/v of | (78-95) | |
| 10 min Average | Tween 80, 50 rpm | 93 | — |
| (Min Max) | paddle | (90-95) | |
| 15 min Average | speed and 900 mL | 96 | — |
| (Min Max) | vessel volume | (94-98) | |
| 30 min Average | | 97 | — |
| (Min Max) | | (95-99) | |
| 45 min Average | | 98 | — |
| (Min Max) | | (95-100) | |
| 60 min Average | | 97 | — |
| (Min Max) | | (94-99) | |
| 70 min Average | | 97 | — |
| (Min Max) | | (94-99) | |
| Dissolution Ezetimibe | | | |
| 15 min Average | Dissolution method | 89 | 90 |
| (Min Max) | based on USP method | (87-94) | (87-91) |
| 30 min Average | conditions at pH | 94 | 99 |
| (Min Max) | 4.5, 75 rpm paddle | (91-99) | (96-101) |
| 45 min Average | speed and 500 mL | 94 | 101 |
| (Min Max) | vessel volume | (91-100) | (98-103) |
| 60 min Average | | 95 | 102 |
| (Min Max) | | (92-100) | (99-105) |
| 70 min Average | | 95 | 105 |
| (Min Max) | | (93-100) | (102-106) |

TABLE 39
Results of the analytical characterization
of scale up batches - Uncoated

| | | Manufacturing lot number | |
|---|---|---|---|
| | | A4459/29/02 | A4459/29/05 |
| | | Prototype | |
| | | FDC 1 | FDC 2 |
| | | (intra-Intra) | (intra-Extra) |
| Test | Method | Result | Result |
| Uniformity of | USP <905> | Average % | Average % |
| dosage units | Ph. Eur. 2.9.40 | Claim: 99.3 | Claim: 97.0 |
| Obicetrapib | or JP 6.02 by | Range % Claim: | Range % Claim: |
| | UNIC/1190 | (98.0-100.1) | (94.0-99.1) |
| | | AV = 1.6 | AV = 6.1 |
| Ezetimibe | | Average % | Average % |
| | | Claim: 102.5 | Claim: 96.4 |
| | | Range % Claim: | Range % Claim: |
| | | (100.3-103.9) | (95.4-97.8) |
| | | AV = 3.2 | AV = 4.0 |
| Water content | KF | 4.6% w/w | 4.3% w/w |

TABLE 40
Ezetimibe dissolution results for scale-up batch A4459/29/02 (FDC1) at
different compression forces

| | | | | | | | | |
|---|---|---|---|---|---|---|---|---|
| Time (min) | Vessel 1 | Vessel 2 | Vessel 3 | Vessel 4 | Vessel 5 | Vessel 6 | Mean% Release | % RSD |

Tablet at 4.5 KN

| | | | | | | | | |
|---|---|---|---|---|---|---|---|---|
| 15 | 93.0 | 93.8 | 93.5 | 93.9 | 95.4 | 95.5 | 94.2 | 1.1 |
| 30 | 102.4 | 101.8 | 101.2 | 101.3 | 103.4 | 103.6 | 102.3 | 1.0 |
| 45 | 104.9 | 103.6 | 103.5 | 103.4 | 105.3 | 105.5 | 104.4 | 0.9 |
| 60 | 104.7 | 103.7 | 103.8 | 104.1 | 105.7 | 105.4 | 104.6 | 0.8 |
| 70 | 105.2 | 104.0 | 104.6 | 104.5 | 105.9 | 105.7 | 105.0 | 0.7 |

Tablet at 12.5 KN

| | | | | | | | | |
|---|---|---|---|---|---|---|---|---|
| 15 | 85.0 | 84.8 | 86.7 | 87.9 | 88.7 | 90.2 | 94.2 | 2.4 |
| 30 | 99.7 | 99.4 | 98.8 | 101.7 | 99.8 | 102.3 | 102.3 | 1.4 |
| 45 | 103.0 | 103.5 | 102.4 | 104.8 | 103.3 | 105.5 | 104.4 | 1.1 |
| 60 | 105.0 | 105.1 | 104.6 | 106.6 | 104.3 | 106.5 | 104.6 | 0.9 |
| 70 | 105.2 | 105.6 | 105.1 | 106.8 | 105.2 | 107.7 | 105.0 | 1.0 |

Tablet at 18.5 KN

| | | | | | | | | |
|---|---|---|---|---|---|---|---|---|
| 15 | 80.5 | 82.5 | 87.4 | 88.1 | 85.6 | 87.1 | 87.2 | 2.4 |
| 30 | 99.0 | 99.3 | 101.1 | 101.8 | 100.5 | 101.4 | 100.3 | 1.4 |
| 45 | 103.5 | 104.7 | 106.0 | 106.5 | 104.2 | 107.3 | 105.4 | 1.4 |
| 60 | 104.8 | 105.2 | 106.7 | 107.9 | 105.3 | 107.4 | 106.2 | 1.2 |
| 70 | 106.6 | 106.5 | 107.9 | 108.4 | 106.8 | 108.6 | 107.4 | 0.9 |

TABLE 41
Obicetrapib dissolution results for scale-up batch A4459/29/02 (FDC1) at
different compression forces

| | | | | | | | | |
|---|---|---|---|---|---|---|---|---|
| Time (min) | Vessel 1 | Vessel 2 | Vessel 3 | Vessel 4 | Vessel 5 | Vessel 6 | Mean% Release | % RSD |

Tablet at 4.5 KN

| | | | | | | | | |
|---|---|---|---|---|---|---|---|---|
| 5 | 60.8 | 61.0 | 73.4 | 74.6 | 72.6 | 73.5 | 69.3 | 9.4 |
| 10 | 94.1 | 96.0 | 98.1 | 97.3 | 91.5 | 95.4 | 95.4 | 2.5 |
| 15 | 101.0 | 103.0 | 102.6 | 100.5 | 96.8 | 99.0 | 100.5 | 2.3 |
| 30 | 102.3 | 103.3 | 103.6 | 102.6 | 98.5 | 100.2 | 101.7 | 1.9 |
| 45 | 99.1 | 101.1 | 101.2 | 100.9 | 97.8 | 98.2 | 99.7 | 1.5 |
| 60 | 99.7 | 99.6 | 100.0 | 98.8 | 97.1 | 97.6 | 98.8 | 1.2 |
| 70 | 104.6 | 96.9 | 100.1 | 99.8 | 98.4 | 97.4 | 99.5 | 2.8 |

Tablet at 12.5 KN

| | | | | | | | | |
|---|---|---|---|---|---|---|---|---|
| 5 | 31.7 | 33.0 | 41.0 | 43.4 | 45.6 | 43.4 | 39.7 | 14.8 |
| 10 | 74.2 | 76.8 | 78.9 | 81.6 | 83.1 | 80.1 | 79.1 | 4.1 |
| 15 | 95.3 | 90.8 | 93.0 | 94.0 | 94.0 | 93.5 | 93.4 | 1.6 |
| 30 | 103.5 | 98.8 | 104.1 | 99.2 | 99.4 | 98.6 | 100.6 | 2.5 |
| 45 | 100.5 | 98.7 | 101.7 | 98.0 | 97.7 | 98.8 | 99.2 | 1.6 |
| 60 | 100.5 | 100.1 | 101.1 | 96.9 | 98.9 | 99.3 | 99.5 | 1.5 |
| 70 | 97.9 | 97.7 | 100.9 | 98.0 | 98.6 | 98.7 | 98.7 | 1.2 |

Tablet at 12.5 KN

| | | | | | | | | |
|---|---|---|---|---|---|---|---|---|
| 5 | 31.7 | 33.0 | 41.0 | 43.4 | 45.6 | 43.4 | 39.7 | 14.8 |
| 10 | 74.2 | 76.8 | 78.9 | 81.6 | 83.1 | 80.1 | 79.1 | 4.1 |
| 15 | 95.3 | 90.8 | 93.0 | 94.0 | 94.0 | 93.5 | 93.4 | 1.6 |
| 30 | 103.5 | 98.8 | 104.1 | 99.2 | 99.4 | 98.6 | 100.6 | 2.5 |
| 45 | 100.5 | 98.7 | 101.7 | 98.0 | 97.7 | 98.8 | 99.2 | 1.6 |
| 60 | 100.5 | 100.1 | 101.1 | 96.9 | 98.9 | 99.3 | 99.5 | 1.5 |
| 70 | 97.9 | 97.7 | 100.9 | 98.0 | 98.6 | 98.7 | 98.7 | 1.2 |

TABLE 42
Ezetimibe dissolution results for scale-up batch A4459/29/05 (FDC2) at
different compression forces

| | | | | | | | | |
|---|---|---|---|---|---|---|---|---|
| Time (min) | Vessel 1 | Vessel 2 | Vessel 3 | Vessel 4 | Vessel 5 | Vessel 6 | Mean% Release | % RSD |

Tablet at 5 KN

| | | | | | | | | |
|---|---|---|---|---|---|---|---|---|
| 15 | 80.4 | 81.5 | 88.9 | 80.3 | 85.6 | 84.0 | 83.4 | 4.1 |
| 30 | 91.2 | 90.7 | 98.5 | 89.3 | 94.5 | 92.4 | 92.8 | 3.6 |
| 45 | 95.2 | 94.5 | 102.2 | 92.5 | 97.8 | 95.9 | 96.3 | 3.5 |
| 60 | 96.9 | 96.1 | 103.4 | 94.1 | 99.1 | 97.2 | 97.8 | 3.3 |
| 70 | 104.2 | 103.9 | 104.9 | 99.0 | 104.0 | 103.1 | 103.2 | 2.1 |

Tablet at 10 KN

| | | | | | | | | |
|---|---|---|---|---|---|---|---|---|
| 15 | 81.9 | 79.2 | 82.9 | 82.9 | 79.6 | 76.2 | 80.5 | 3.3 |
| 30 | 92.3 | 89.4 | 94.5 | 93.2 | 91.4 | 87.5 | 91.4 | 2.8 |
| 45 | 96.6 | 94.0 | 98.8 | 96.8 | 95.4 | 92.2 | 95.6 | 2.4 |
| 60 | 98.3 | 95.6 | 100.9 | 98.9 | 98.1 | 93.9 | 97.6 | 2.6 |
| 70 | 101.2 | 102.0 | 103.7 | 103.0 | 102.5 | 101.4 | 102.3 | 0.9 |

Tablet at 20 KN

| | | | | | | | | |
|---|---|---|---|---|---|---|---|---|
| 15 | 62.8 | 64.5 | 73.0 | 73.0 | 79.6 | 68.4 | 70.2 | 8.9 |
| 30 | 84.4 | 84.4 | 89.1 | 87.8 | 92.5 | 87.8 | 87.7 | 3.5 |
| 45 | 90.1 | 90.8 | 95.0 | 93.1 | 96.7 | 92.4 | 93.0 | 2.7 |
| 60 | 93.1 | 94.1 | 97.8 | 96.1 | 98.8 | 95.7 | 95.9 | 2.2 |
| 70 | 102.7 | 102.6 | 102.9 | 104.0 | 104.8 | 102.2 | 103.2 | 1.0 |

TABLE 43
Obicetrapib dissolution results for scale-up batch A4459/29/05 (FDC2) at
different compression forces

| | | | | | | | | |
|---|---|---|---|---|---|---|---|---|
| Time (min) | Vessel 1 | Vessel 2 | Vessel 3 | Vessel 4 | Vessel 5 | Vessel 6 | Mean% Release | % RSD |

Tablet at 5 KN

| | | | | | | | | |
|---|---|---|---|---|---|---|---|---|
| 5 | 57.7 | 60.1 | 64.5 | 64.5 | 65.1 | 74.2 | 64.3 | 8.8 |
| 10 | 78.4 | 77.5 | 80.1 | 78.4 | 79.2 | 88.2 | 80.3 | 4.9 |
| 15 | 83.7 | 82.0 | 83.3 | 81.9 | 82.7 | 90.6 | 84.0 | 3.9 |
| 30 | 86.7 | 84.4 | 86.3 | 83.5 | 85.6 | 92.7 | 86.5 | 3.8 |
| 45 | 88.1 | 85.5 | 89.7 | 83.8 | 92.2 | 92.5 | 88.6 | 4.0 |
| 60 | 88.2 | 85.8 | 89.8 | 84.1 | 87.3 | 93.9 | 88.2 | 3.9 |
| 70 | 94.8 | 93.9 | 93.9 | 90.5 | 94.5 | 96.2 | 94.0 | 2.0 |

Tablet at 10 KN

| | | | | | | | | |
|---|---|---|---|---|---|---|---|---|
| 5 | 56.5 | 56.6 | 62.2 | 62.0 | 66.0 | 70.2 | 62.2 | 8.6 |
| 10 | 75.1 | 75.0 | 75.8 | 76.1 | 78.4 | 81.9 | 77.0 | 3.4 |
| 15 | 81.7 | 79.2 | 80.6 | 80.7 | 87.3 | 86.8 | 82.7 | 4.2 |
| 30 | 87.9 | 85.1 | 84.2 | 84.2 | 92.4 | 88.9 | 87.1 | 3.7 |
| 45 | 88.1 | 87.7 | 85.9 | 85.3 | 93.8 | 89.4 | 88.4 | 3.5 |
| 60 | 89.5 | 88.7 | 86.4 | 86.5 | 94.6 | 90.5 | 89.4 | 3.4 |
| 70 | 94.5 | 94.4 | 94.1 | 94.4 | 95.3 | 95.5 | 94.7 | 0.6 |

Tablet at 20 KN

| | | | | | | | | |
|---|---|---|---|---|---|---|---|---|
| 5 | 56.5 | 56.6 | 62.2 | 62.0 | 66.0 | 70.2 | 62.2 | 8.6 |
| 10 | 75.1 | 75.0 | 75.8 | 76.1 | 78.4 | 81.9 | 77.0 | 3.4 |
| 15 | 81.7 | 79.2 | 80.6 | 80.7 | 87.3 | 86.8 | 82.7 | 4.2 |
| 30 | 87.9 | 85.1 | 84.2 | 84.2 | 92.4 | 88.9 | 87.1 | 3.7 |
| 45 | 88.1 | 87.7 | 85.9 | 85.3 | 93.8 | 89.4 | 88.4 | 3.5 |
| 60 | 89.5 | 88.7 | 86.4 | 86.5 | 94.6 | 90.5 | 89.4 | 3.4 |
| 70 | 94.5 | 94.4 | 94.1 | 94.4 | 95.3 | 95.5 | 94.7 | 0.6 |

TABLE 44
Results of the analytical characterization of scale
up batches 10 mg ezetimibe 10 mg obicetrapib - Coated

| | | Manufacturing lot number | |
|---|---|---|---|
| | | A4459/29/03 | A4459/29/06 |
| | | Prototype | |
| | | FDC 1 | FDC 2 |
| | | Coated | Coated |
| Test | Method | Result | Result |
| Appearance | Visual | White round | White round |
| | | coated tablets | coated tablets |
| Assay | HPLC | | |
| Obicetrapib (% claim) | 2730 | 98.3 | 96.9 |
| Ezetimibe (% claim) | 2731 | 101.3 | 101.6 |
| Impurities | HPLC | | — |
| IMP_RRT0.94 (% claim) | 2730 | 0.06 | 0.06 |
| FROL(% claim) | 2730 | 0.07 | 0.06 |
| MONO-BN FREE(% claim) | 2730 | 0.16 | 0.16 |
| Total impurities of | 2730 | 0.29 | 0.28 |
| Obicetrapib ≥0.05% | | | |
| IMP_RRT0.79 (% a/a) | 2731 | 0.05 | 0.05 |
| Ezetimibe□Ketone (% a/a) | 2731 | 0.05 | 0.05 |
| Ezetimibe tetrahydropyran | 2731 | N.D. | N.D. |
| analog (% a/a) | | | |
| Total impurities of | 2731 | 0.10 | 0.10 |
| Ezetimbe ≥0.05% | | | |
| Dissolution Obicetrapib | | | |
| 5 min Average | Discriminating | 53 | 60 |
| (Min Max) | method at pH 6.80.2% | (38-60) | (56-66) |
| 10 min Average | w/v of Tween 80, | 89 | 84 |
| (Min Max) | 50 rpm paddle speed | (84-93) | (80-90) |
| 15 min Average | and 900 mL vessel | 100 | 90 |
| (Min Max) | volume | (97-106) | (87-97) |
| 30 min Average | | 108 | 98 |
| (Min Max) | | (103-112) | (95-103) |
| 45 min Average | | 106 | 98 |
| (Min Max) | | (103-111) | (95-102) |
| 60 min Average | | 103 | 97 |
| (Min Max) | | (101-106) | (94-102) |
| 70 min Average | | 102 | 98 |
| (Min Max) | | (101-104) | (96-101) |
| Dissolution Ezetimibe | | | |
| 15 min Average | Dissolution method | 87 | 84 |
| (Min Max) | based on USP | (75-91) | (76-87) |
| 30 min Average | method conditions at | 100 | 94 |
| (Min Max) | pH 4.5, 75 rpm | (98-101) | (87-98) |
| 45 min Average | paddle speed and | 103 | 98 |
| (Min Max) | 500 mL vessel | (102-105) | (92-101) |
| 60 min Average | volume | 104 | 99 |
| (Min Max) | | (103-106) | (94-102) |
| 70 min Average | | 106 | 104 |
| (Min Max) | | (105-107) | (102-106) |
| Water content | KF | 4.5% w/w | 4.2% w/w |

TABLE 45
XRPD data summary of granules/tablets from scale up batch

| Sample | Batch ID | XRPD |
|---|---|---|
| FDC1 (INTRA-INTRA) Granules | A4459/29/01 | OBI + EZE Anhydrous |
| FDC1 (INTRA-INTRA) Tablet | A4459/29/02-A4459/29/03 | OBI + EZE Anhydrous |
| uncoated - coated | | |
| FDC2 (INTRA-EXTRA) Granules | A4459/29/04 | OBI + EZE Anhydrous |
| FDC2 (INTRA-EXTRA) Tablet | A4459/29/05-A4459/29/06 | OBI + EZE Anhydrous |
| uncoated - coated | | |

### Example 8: Technical Batches of FDC1 and FDC2 Compositions

The batch numbers and compositions of the granules, tablets and coated tablets of the technical manufactures of the FDC1 and FDC2 formulations are detailed in Table 49.

### High Shear Granulation and Drying

The components of the FDC1 granule (batch A4459/30/02) and FDC2 granule (batch A4459/30/01) were sieved manually and added to the granulation bowl. For both FDC1 and FDC2 compositions, the batch size of the granule was 2 kg, and, identical processing condition of granulation were used. The physical mixture was blended for 5 mins at 220 rpm and a LOD testing was executed. The granulating agent (purified water) was sprayed and, following spraying, 1 min wet massing was conducted. A sample for XRPD and LOD was taken. Then, the granule was dried using a fluid bed drier. The drying step was concluded as the LOD of the granule was equal or lower than the initial LOD or below 3% (w/w). The granules were tested for content uniformity (only for FDC1), PSD, LOD, sieve analysis, TBD and XRPD.

### Final Blend and Tableting

The method of preparation of the final blend was described in the previous section (scale-up batches). The FDC2 blend was tested for content uniformity.

For the generation of a compression profile and for the tableting campaign, a rotary press machine was used. To manufacture FDC1 tablets (150 mg target tablet weight), a 7.0 mm diameter punch was used whereas, for FDC2 tablets (230.0 mg tablet weight) an 8.5 mm diameter punch. The friability, disintegration time, hardness, thickness and appearance of the tablets as well as the individual tablet weight and the tablet weight of ten tablets was monitored during tableting. The tablets were tested for content uniformity (stratified sample: start, middle and end of production), XRPD, dissolution and water content by KF.

### Coating

The coating suspension was prepared at 20% (w/w) solid content by adding the required quantity of Opadry to water under stirring. The suspension was mixed for no less than 45 minutes and its visual homogeneity confirmed. Then, the spray rate of the coating suspension was measured. The coating suspension was kept under stirring all the time. The weight gain of the tablets was monitored throughout the manufacture and spraying was stopped as the required target gain (3% w/w, 2% w/w-4% w/w limits) was achieved. The coated tablets were visually inspected and tested for XRPD, dissolution, appearance, content uniformity, and water content by KF.

All the intermediates of production and the final drug product were stored in the stability chambers with following packaging:
60 mL High Density Polyethylene (HDPE) induction sealed and closed with child resistant cap. The fill count for tablets is 20;
60 mL High Density Polyethylene (HDPE) induction sealed with 2 g of desiccant canister and closed with child resistant cap. The fill count for tablets is 20.

TABLE 49
Composition (% w/w) of the granule, tablet and coated tablet of the FDC1 and
FDC2 technical batches

| | FDC2 (ezeetimibe intra-obicetrapib extra) | | | | | FDC1 (ezetimibe intra-obicetrapib intra) | | | | |
|---|---|---|---|---|---|---|---|---|---|---|
| | | A4459/ | | | | | A4459/ | | | |
| | A4459/ | 31/01 | | | | A4459/ | 31/02 | | | |
| | 30/01 | | | Coated | Coated | 30/02 | | | | Coated |
| | Granule | Tablet | Tablet | Tablet | tablet | Granule | Tablet | Tablet | Tablet | tablet |
| Component | (%, w/w) | (%, w/w) | (mg) | (%, w/w) | (mg) | (%, w/w) | (%, w/w) | (mg) | (%, w/w) | (mg) |

Intra-granule

| | | | | | | | | | | |
|---|---|---|---|---|---|---|---|---|---|---|
| Ezetimibe | 7.692 | 4.348 | 10.00 | 4.348 | 10.00 | 7.018 | 6.667 | 10.00 | 6.667 | 10.00 |
| Obicetrapib | N/A | N/A | N/A | N/A | N/A | 7.200 | 6.840 | 10.26 | 6.840 | 10.26 |
| Avicel PH 101 | 23.614 | 13.347 | 30.70 | 13.347 | 30.70 | 21.950 | 20.853 | 31.28 | 20.853 | 31.28 |
| Pharmatose | 61.771 | 34.914 | 80.30 | 34.914 | 80.30 | 57.516 | 54.640 | 81.96 | 54.640 | 81.96 |
| 200 M | | | | | | | | | | |
| Kollidon 30 | 1.154 | 0.652 | 1.50 | 0.652 | 1.50 | 1.053 | 1.000 | 1.50 | 1.000 | 1.50 |
| Glycolys | 4.615 | 2.608 | 6.00 | 2.608 | 6.00 | 4.210 | 4.000 | 6.00 | 4.000 | 6.00 |
| Kolliphor SLS | 1.154 | 0.652 | 1.50 | 0.652 | 1.50 | 1.053 | 1.000 | 1.50 | 1.000 | 1.50 |
| Purified Water | 30.000 | N/A | N/A | N/A | N/A | 30.000 | N/A | N/A | N/A | N/A |
| Total Granule | 100.000 | 56.521 | 130.00 | 56.521 | 130.00 | 100.000 | 95.000 | 142.50 | 95.000 | 142.50 |

Extra-granule

| | | | | | | | | | | |
|---|---|---|---|---|---|---|---|---|---|---|
| Obicetrapib | N/A | 4.461 | 10.26 | 4.461 | 10.26 | N/A | N/A | N/A | N/A | N/A |
| Avicel PH 200 | N/A | 23.118 | 53.17 | 23.118 | 53.17 | N/A | N/A | N/A | N/A | N/A |
| Pearlitol 200 SD | N/A | 8.900 | 20.47 | 8.900 | 20.47 | N/A | N/A | N/A | N/A | N/A |
| Glycolys | N/A | 5.000 | 11.50 | 5.000 | 11.50 | N/A | 4.000 | 6.00 | 4.000 | 6.00 |
| Aerosil 200 | N/A | 1.000 | 2.30 | 1.000 | 2.30 | N/A | N/A | N/A | N/A | N/A |
| Ligamed MF-2-V | N/A | 1.000 | 2.30 | 1.000 | 2.30 | N/A | 1.000 | 1.50 | 1.000 | 1.50 |
| Opadry AMB II | N/A | N/A | N/A | 3.000 | 6.90 | N/A | N/A | N/A | 3.000 | 4.50 |
| Total Tablet | N/A | 100.000 | 230.00 | 103.000 | 236.90 | N/A | 100.000 | 150.00 | 103.000 | 154.50 |

### Results

### Technical Batches of FDC1 and FDC2 Compositions

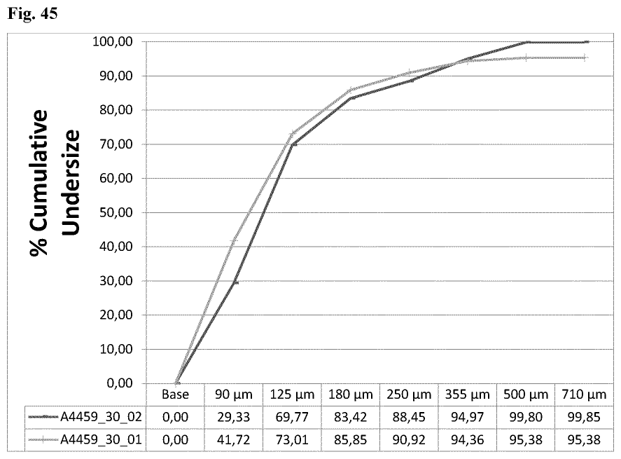

The high shear granulation of the technical batches was conducted successfully and no issues occurred during the process. The powder consumption was comparable to that observed in the scale-up batches. The drying process was executed within 45 mins as the LOD of the granule was lower than 3%. The granules showed similar PSD values and the quantity of fine particles was relatively large (e.g. the quantity of particles smaller than 125 μm was ca. 70%-73%) (FIG. 45). No relevant differences were observed in comparison to the scale-up batches. In comparison to the FDC2 scale-up batch (batch A4459/29/06), the FDC2 technical batch presented harder tablets for similar levels of compression force albeit, the time of disintegration between these batches was very similar for a given value of tablet hardness.

### Granules Chemical Characterization

The granule and the final blend were tested for homogeneity of both obicetrapib and ezetimibe, and were found to be homogeneously dispersed.

### Tablets Chemical Characterization

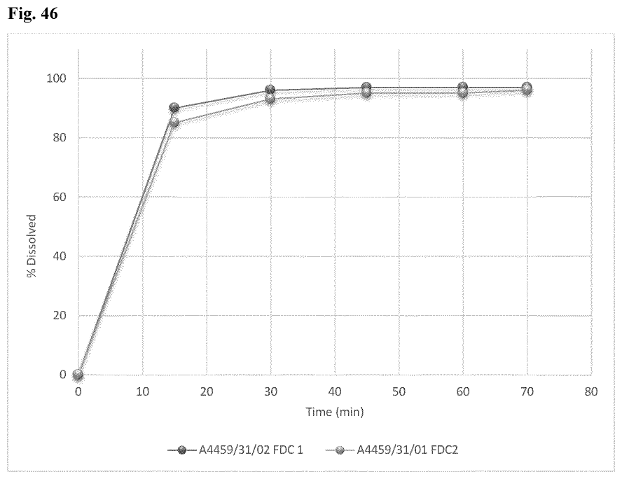

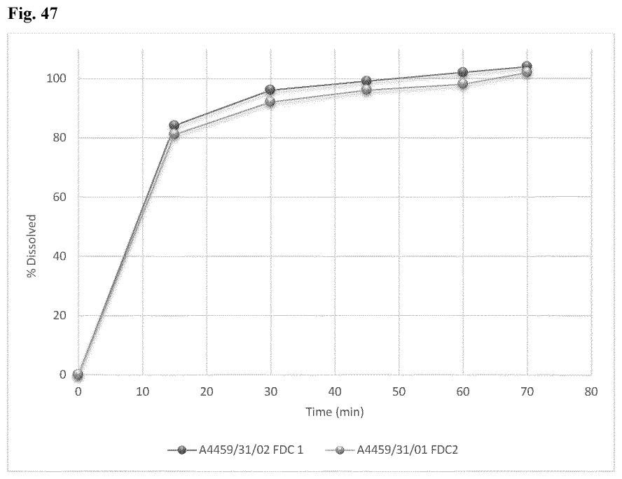

Chemical characterization of the technical batches coated tablets results are reported in Table 50. The tablets showed a suitable level of quality with the % claims within the typical acceptance criterion for clinical phases (i.e. 90.0-110.0%). The results of the content uniformity test met the pharmacopoeia requirement of AV<15.0. Dissolution met the proposed specification for Q-75% at 45 minutes (FIG. 46 and FIG. 47).

### Physical Properties Characterization

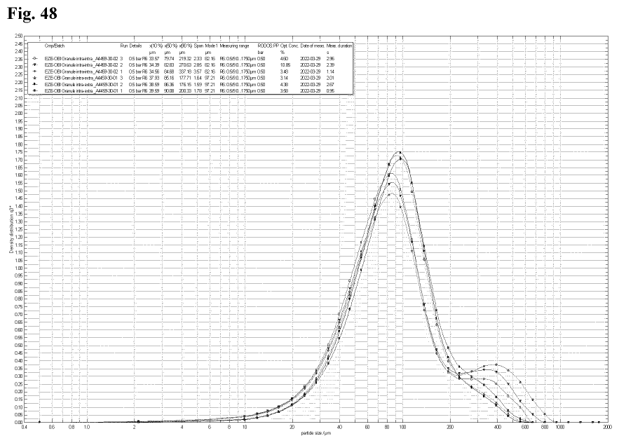

XRPD data of the Granules/Tablets from Technical Batches are summarized in Table 51. All the batches tested showed the absence of EZE hydrate with exception of the granules from the FDC2 formulation. However, the hydrate form disappears in the coated tablet. Both batches of Granules show similar flowability. PSD data of granules are reported in Table 52 and in FIG. 48. Batches show similar bi-modal curves.

### Stability Studies

A summary of physicochemical analysis of technical batches after three months of storage for FDC2 composition is provided in Table 53 (without desiccant) and Table 54 (with desiccant), and for FDC1 composition in Table 55 (without desiccant) and Table 56 (with desiccant).

TABLE 50
Results of the analytical characterization of technical batches - Coated

| | | Manufacturing lot number | |
|---|---|---|---|
| | | A4459/31/02 | A4459/31/01 |
| | | Prototype | |
| | | FDC 1 | FDC 2 |
| | | (intra-Intra) | (intra-Extra) |
| Test | Method | Result | Result |
| Appearance | Visual | White round | White round |
| | | coated tablet | coated tablet |
| Assay | HPLC | | |
| Obicetrapib (% claim) | 2730 | 96.8 | 94.3 |
| Ezetimibe (% claim) | 2731 | 100.8 | 100.7 |
| Impurities | HPLC | | |
| IMP_RRT0.93 (% claim) | 2730 | 0.07 | <QL (0.05%) |
| IMP_RRT0.94 (% claim) | 2730 | N.D | 0.05 |
| FROL(% claim) | 2730 | 0.11 | 0.06 |
| MONO-BN FREE(% claim) | 2730 | 0.15 | 0.16 |
| Total impurities of | 2730 | 0.33 | 0.26 |
| Obicetrapib ≥0.05% | | | |
| IMP_RRT0.73 (% a/a) | 2731 | N.D. | N.D. |
| IMP_RRT0.79 (% a/a) | 2731 | 0.05 | <QL (0.05%) |
| Ezetimibe□Ketone (% a/a) | 2731 | 0.05 | 0.05 |
| Ezetimibe tetrahydro- | 2731 | N.D. | N.D. |
| pyran analog (% a/a) | | | |
| Total impurities of | 2731 | 0.10 | 0.05 |
| Ezetimbe ≥ 0.05% | | | |
| Uniformity of | | | |
| dosage units | | | |
| Obicetrapib | USP <905> | Average % | Average % |
| | Ph. Eur. 2.9.40 or | Claim: 98.3 | Claim: 96.1 |
| | JP 6.02 by UNIC/ | Range % Claim: | Range % Claim: |
| | 1190 | (97.1-100.4) | (93.9-99.0) |
| | | AV = 2.4 | AV = 4.0 |
| Ezetimibe | | Average % | Average % |
| | | Claim: 100.8 | Claim: 99.2 |
| | | Range % Claim: | Range % Claim: |
| | | (99.3-101.5) | (97.7-101.5) |
| | | AV = 2.4 | AV = 2.7 |
| Dissolution Obicetrapib | | | |
| 15 min Average | Medium pH 6.8 + 0.2% | 90 | 85 |
| (Min Max) | w/v of Tween 80, 75 | (89-92) | (82-86) |
| 30 min Average | rpm paddle speed and | 96 | 93 |
| (Min Max) | 1000 mL vessel | (94-97) | (92-94) |
| 45 min Average | volume | 97 | 95 |
| (Min Max) | | (95-98) | (93-96) |
| 60 min Average | | 97 | 95 |
| (Min Max) | | (96-98) | (94-97) |
| 70 min Average | | 97 | 96 |
| (Min Max) | | (96-98) | (94-97) |
| Dissolution Ezetimibe | | | |
| 15 min Average | Dissolution method | 84 | 81 |
| (Min Max) | based on USP method | (82-87) | (72-83) |
| 30 min Average | conditions at pH 4.5, | 96 | 92 |
| (Min Max) | 75 rpm paddle speed | (93-99) | (88-94) |
| 45 min Average | and 500 mL vessel | 99 | 96 |
| (Min Max) | volume | (97-103) | (95-97) |
| 60 min Average | | 102 | 98 |
| (Min Max) | | (99-105) | (97-99) |
| 70 min Average | | 104 | 102 |
| (Min Max) | | (101-107) | (101-104) |
| Water content | KF | 4.0% w/w | 3.7% w/w |

TABLE 51
XRPD data summary of granules/tablets from technical batches

| Sample | Batch ID | XRPD |
|---|---|---|
| FDC2 (INTRA-EXTRA) | A4459/30/01 | OBI + EZE Anhydrous + |
| Granules | | small amount |
| | | EZE hydrate |
| FDC2 (INTRA-EXTRA) | A4459/31/01 | OBI + EZE Anhydrous |
| Tablet coated | | |
| FDC1 (INTRA-INTRA) | A4459/30/02 | OBI + EZE Anhydrous |
| Granules | | |
| FDC1 (INTRA-INTRA) | A4459/31/02 | OBI + EZE Anhydrous |
| Tablet coated | | |

TABLE 52
PSD data summary of granules from technical batches

| | Dv10 | Dv50 | Dv90 | |
|---|---|---|---|---|
| | (x10) | (x50) | (x90) | Span |
| Granules FDC2 A4459/30/01 | 38.70 | 87.20 | 184.73 | 1.67 |
| Granules FDC1 A4459/30/02 | 34.17 | 82.42 | 275.71 | 2.92 |

TABLE 53
Long-term and accelerated storage stability results for FDC2 technical batch
no. A4459/31/01 (without desiccant)

| | | | OBICETRAPIB | | ETHYL | | MONO- |
|---|---|---|---|---|---|---|---|
| | | | (TA-8995) | DIASTEREOISOMER | ESTER | FROL | BN FREE |
| | | | Amount | Amount | Amount | Amount | Amount |
| | | Appearan./ | HPLC/2730 | HPLC/2730 | HPLC/2730 | HPLC/2730 | HPLC/2730 |
| Storage | Time | Colour APP/1001 | (% claim) | (% claim) | (% claim) | (% claim) | (% claim) |
| Condition | (Months) | (Individual) | (Mean) | (Mean) | (Mean) | (Mean) | (Mean) |
| Specification | N/A | White to off- | 90.0-110.0% | NGT 0.3% claim | NGT 0.3% | NGT 0.3% | NGT 0.3% |
| | | white round | claim | | claim | claim | claim |
| | | coated tablet | | | | | |
| Initial | 0 | White round | 94.3 | <0.05 | <0.05 | 0.06 | 0.16 |
| | | coated tablet | | | | | |
| 25° C./60% | 1 | White round | 96.7 | <0.05 | <0.05 | 0.07 | 0.16 |
| RH | | coated tablet | | | | | |
| Packaged | 3 | White round | 95.5 | <0.05 | <0.05 | 0.07 | 0.16 |
| | | coated tablet | | | | | |
| 30° C./65% | 1 | White round | 95.6 | <0.05 | <0.05 | 0.07 | 0.16 |
| RH | | coated tablet | | | | | |
| Packaged | | | | | | | |
| 40° C./75% | 1 | White round | 95.5 | <0.05 | <0.05 | 0.07 | 0.16 |
| RH | | coated tablet | | | | | |
| Packaged | 3 | White round | 95.3 | <0.05 | <0.05 | 0.07 | 0.16 |
| | | coated tablet | | | | | |
| | | | | Total | | | |
| | | | | degradation | | | |
| | | | | products | | EZETIMIBE | EZETIMIBE |
| | | IMP_RRT0.93 | IMP_RRT0.94 | equal/greater | EZETIMIBE | CYCLIC | KETONE |
| | | Amount | Amount | than 0.05% | Amount | Amount | Amount |
| | | HPLC/2730 | HPLC/2730 | claim | HPLC/2731 | HPLC/2731 | HPLC/2731 |
| Storage | Time | (% claim) | (% claim) | (% claim) | (% claim) | (% claim) | (% claim) |
| Condition | (Months) | (Mean) | (Mean) | (Mean) | (Mean) | (Mean) | (Mean) |
| Specification | N/A | NGT 0.3% | NGT 0.3% | NGT 2.0% | 90.0-110.0% | NGT 0.5% | NGT 0.5% |
| | | claim | claim | claim | claim | claim | claim |
| Initial | 0 | <0.05 | 0.05 | 0.26 | 100.7 | <0.05 | 0.05 |
| 25° C./60% RH | 1 | <0.05 | <0.05 | 0.23 | 103.0 | <0.05 | 0.05 |
| Packaged | 3 | <0.05 | 0.06 | 0.29 | 103.2 | <0.05 | <0.05 |
| 30° C./65% RH | 1 | <0.05 | <0.05 | 0.23 | 102.2 | <0.05 | 0.05 |
| Packaged | | | | | | | |
| 40° C./75% RH | 1 | <0.05 | <0.05 | 0.23 | 101.6 | <0.05 | 0.06 |
| Packaged | 3 | <0.05 | 0.07 | 0.30 | 103.1 | <0.05 | 0.05 |

Table 53 (contd.)

| | | | | | Total | Time | Time |
|---|---|---|---|---|---|---|---|
| | | | | | degradation | point(min) 15 | point(min) 15 |
| | | IMP_RRT0.73 | IMP_RRT0.79 | IMP_RRT3.11 | products | OBICETRAPIB | OBICETRAPIB |
| | | Amount | Amount | Amount | greater/equal | (TA-8995) | (TA-8995) |
| | | HPLC/2731 | HPLC/2731 | HPLC/2731 | than 0.05% | DISS/1295 | DISS/1295 |
| Storage | Time | (% claim) | (%claim) | (% claim) | claim | (% claim) | (% claim) |
| Condition | (Months) | (Mean) | (Mean) | (Mean) | (Mean) | (Mean) | (Range) |
| Specification | N/A | NGT 0.5% | NGT 0.5% | NGT 0.5% | NGT 2.0% | Report result | Report result |
| | | claim | claim | claim | claim | | |
| Initial | 0 | <0.05 | <0.05 | | 0.05 | 85 | 82-86 |
| 25° C./60% | 1 | <0.05 | 0.05 | <0.05 | 0.10 | 85 | 80-91 |
| RH | 3 | <0.05 | 0.05 | <0.05 | 0.05 | 87 | 85-95 |
| Packaged | | | | | | | |
| 30° C./65% | 1 | <0.05 | 0.05 | <0.05 | 0.10 | 89 | 87-91 |
| RH | | | | | | | |
| Packaged | | | | | | | |
| 40° C./75% | 1 | <0.05 | 0.05 | <0.05 | 0.10 | 85 | 84-88 |
| RH | 3 | <0.05 | 0.05 | <0.05 | 0.10 | 85 | 83-88 |
| Packaged | | | | | | | |

Table 53 (contd.)

| | | Time | Time | Time | Time | Time | Time |
|---|---|---|---|---|---|---|---|
| | | point(min) 30 | point(min) 30 | point(min) 45 | point(min) 45 | point(min) 60 | point(min) 60 |
| | | OBICETRAPIB | OBICETRAPIB | OBICETRAPIB | OBICETRAPIB | OBICETRAPIB | OBICETRAPIB |
| | | (TA-8995) | (TA-8995) | (TA-8995) | (TA-8995) | (TA-8995) | (TA-8995) |
| | | DISS/1295 | DISS/1295 | DISS/1295 | DISS/1295 | DISS/1295 | DISS/1295 |
| Storage | Time | (% claim) | (% claim) | (% claim) | (% claim) | (% claim) | (% claim) |
| Condition | (Months) | (Mean) | (Range) | (Mean) | (Range) | (Mean) | (Range) |
| Specification | N/A | Report result | Report result | Complies with | Report result | Report result | Report result |
| | | | | EP, USP, JP | | | |
| | | | | where | | | |
| | | | | Q = 75% at 45 | | | |
| | | | | minutes | | | |
| Initial | 0 | 93 | 92-94 | 95 | 93-96 | 95 | 94-97 |
| 25° C./60% | 1 | 98 | 94-104 | 98 | 94-104 | 99 | 95-104 |
| RH | 3 | 96 | 94-98 | 98 | 96-99 | 97 | 96-100 |
| Packaged | | | | | | | |
| 30° C./65% | 1 | 99 | 97-101 | 99 | 97-101 | 100 | 97-102 |
| RH | | | | | | | |
| Packaged | | | | | | | |
| 40° C./75% | 1 | 95 | 93-98 | 96 | 94-99 | 97 | 95-100 |
| RH | | | | | | | |
| Packaged | 3 | 95 | 93-97 | 97 | 95-99 | 97 | 94-98 |
| | | Time | Time | Time | Time | Time | Time |
| | | point(min) 70 | point(min) 70 | point(min) | point(min) | point(min) | point(min) |
| | | OBICETRAPIB | OBICETRAPIB | 15 | 15 | 30 | 30 |
| | | (TA-8995) | (TA-8995) | EZETIMIBE | EZETIMIBE | EZETIMIBE | EZETIMIBE |
| | | DISS/1295 | DISS/1295 (% | DISS/1298 | DISS/1298 | DISS/1298 | DISS/1298 |
| Storage | Time | (% claim) | claim) | (% claim) | (% claim) | (% claim) | (% claim) |
| Condition | (Months) | (Mean) | (Range) | (Mean) | (Range) | (Mean) | (Range) |
| Specification | N/A | Report result | Report result | Report result | Report result | Report result | Report result |
| Initial | 0 | 96 | 94-97 | 81 | 72-83 | 92 | 88-94 |
| 25° C./60% | 1 | 99 | 96-104 | 79 | 65-87 | 95 | 87-101 |
| RH | 3 | 97 | 96-100 | 85 | 82-88 | 95 | 92-100 |
| Packaged | | | | | | | |
| 30° C./65% | 1 | 100 | 97-102 | 88 | 86-90 | 95 | 93-97 |
| RH | | | | | | | |
| Packaged | | | | | | | |
| 40° C./75% | 1 | 97 | 95-100 | 80 | 58-88 | 91 | 74-97 |
| RH | | | | | | | |
| Packaged | 3 | 97 | 94-99 | 80 | 75-84 | 93 | 90-94 |
| | | Time | Time | Time | | | Time |
| | | point(min) | point(min) | point(min) | Time | Time | point(min) |
| | | 45 | 45 | 60 | point(min) 60 | point(min) 70 | 70 |
| | | EZETIMIBE | EZETIMIBE | EZETIMIBE | EZETIMIBE | EZETIMIBE | EZETIMIBE |
| | | DISS/1298 | DISS/1298 | DISS/1298 | DISS/1298 | DISS/1298 | DISS/1298 |
| Storage | Time | (% claim) | (% claim) | (% claim) | (% claim) | (% claim) | (% claim) |
| Condition | (Months) | (Mean) | (Range) | (Mean) | (Range) | (Mean) | (Range) |
| Specification | N/A | Complies | Report result | Report result | Report result | Report result | Report result |
| | | with EP, USP, | | | | | |
| | | JP where | | | | | |
| | | Q = 75% at | | | | | |
| | | 45 minutes | | | | | |
| Initial | 0 | 96 | 95-97 | 98 | 97-99 | 102 | 101-104 |
| 25° C./60% RH | 1 | 99 | 94-105 | 99 | 96-103 | 106 | 103-108 |
| Packaged | 3 | 98 | 95-103 | 100 | 96-105 | 103 | 100-105 |
| 30°° C./65% RH | 1 | 98 | 96-101 | 99 | 97-102 | 103 | 100-105 |
| Packaged | | | | | | | |
| 40° C./75% RH | 1 | 94 | 79-101 | 97 | 82-102 | 104 | 101-106 |
| Packaged | 3 | 98 | 97-101 | 101 | 101-104 | 105 | 102-107 |

| | | Water content | Strength | Strength | Strength | Strength |
|---|---|---|---|---|---|---|
| | | UMID/1231 | RESR/1001 | RESR/1001 | RESR/1001 | RESR/1001 |
| Storage | Time | (% w/w) | (N) | (N) | (Kp) | (Kp) |
| Condition | (Months) | (Mean) | (Mean) | (Range) | (Mean) | (Range) |
| Specification | N/A | Report Results | Report Results | Report Results | Report Results | Report Results |
| Initial | 0 | 3.7 | 92 | 81-99 | 9.36 | 8.26-10.10 |
| 25° C./60% RH | 1 | 3.8 | 109 | 98-123 | 11.15 | 9.99-12.54 |
| Packaged | 3 | 4.1 | 116 | 104-124 | 11.81 | 10.61-12.64 |
| 30° C./65% RH | 1 | 4.0 | 109 | 97-128 | 11.09 | 9.89-13.05 |
| Packaged | | | | | | |
| 40° C./75% RH | 1 | 4.2 | 100 | 95-105 | 10.22 | 9.69-10.71 |
| Packaged | 3 | 4.8 | 93 | 84-107 | 9.45 | 8.57-10.91 |

N/A: Not Applicable

TABLE 54
Long-term and accelerated storage stability results for FDC1 technical batch
no. A4459/31/02 (without desiccant)

| | | | OBICETRAPIB | | ETHYL | | MONO- |
|---|---|---|---|---|---|---|---|
| | | | (TA-8995) | DIASTEREOISOM | ESTER | FROL | BN FREE |
| | | Appearan./ | Amount | ER Amount | Amount | Amount | Amount |
| | | Colour | HPLC/2730 | HPLC/2730 | HPLC/2730 | HPLC/2730 | HPLC/2730 |
| Storage | Time | APP/1001 | (% claim) | (% claim) | (% claim) | (% claim) | (% claim) |
| Condition | (Months) | (Individual) | (Mean) | (Mean) | (Mean) | (Mean) | (Mean) |
| Specification | N/A | White to off- | 90.0-110.0% | NGT 0.3% claim | NGT 0.3% | NGT 0.3% | NGT 0.3% |
| | | white round | claim | | claim | claim | claim |
| | | coated tablet | | | | | |
| Initial | 0 | White round | 96.7 | <0.05 | <0.05 | 0.11 | 0.15 |
| | | coated tablet | | | | | |
| 25° C./60% | 1 | White round | 97.7 | <0.05 | <0.05 | 0.11 | 0.16 |
| RH | | coated tablet | | | | | |
| Packaged | 3 | White round | 98.4 | <0.05 | <0.05 | 0.11 | 0.16 |
| | | coated tablet | | | | | |
| 30° C./65% | 1 | White round | 97.7 | <0.05 | <0.05 | 0.11 | 0.16 |
| RH | | coated tablet | | | | | |
| Packaged | | | | | | | |
| 40° C./75% | 1 | White round | 97.4 | <0.05 | <0.05 | 0.12 | 0.16 |
| RH | | coated tablet | | | | | |
| Packaged | 3 | White round | 98.2 | <0.05 | <0.05 | 0.11 | 0.16 |
| | | coated tablet | | | | | |
| | | | | Total | | | |
| | | | | degradation | | | |
| | | | | products | | EZETIMIBE | EZETIMIBE |
| | | IMP_RRT0.93 | IMP_RRT0.94 | equal/greater | EZETIMIBE | CYCLIC | KETONE |
| | | Amount | Amount | than 0.05% | Amount | Amount | Amount |
| | | HPLC/2730 | HPLC/2730 | claim | HPLC/2731 | HPLC/2731 | HPLC/2731 |
| Storage | Time | (% claim) | (% claim) | (% claim) | (% claim) | (% claim) | (% claim) |
| Condition | (Months) | (Mean) | (Mean) | (Mean) | (Mean) | (Mean) | (Mean) |
| Specification | N/A | NGT 0.3% | NGT 0.3% | NGT 2.0% | 90.0-110.0% | NGT 0.5% | NGT 0.5% |
| | | claim | claim | claim | claim | claim | claim |
| Initial | 0 | 0.07 | <0.05 | 0.33 | 100.8 | <0.05 | 0.05 |
| 25° C./60% RH | 1 | 0.07 | <0.05 | 0.35 | 102.6 | <0.05 | 0.05 |
| Packaged | 3 | 0.06 | <0.05 | 0.33 | 104.0 | <0.05 | <0.05 |
| 30° C./65% RH | 1 | 0.07 | <0.05 | 0.35 | 103.1 | <0.05 | 0.05 |
| Packaged | | | | | | | |
| 40° C./75% RH | 1 | 0.07 | <0.05 | 0.34 | 103.6 | <0.05 | 0.05 |
| Packaged | 3 | 0.07 | <0.05 | 0.34 | 104.3 | <0.05 | <0.05 |
| | | | | | Total | Time | Time |
| | | | | | degradation | point(min) 15 | point(min) 15 |
| | | IMP_RRT0.73 | IMP_RRT0.79 | IMP_RRT3.11 | products | OBICETRAPIB | OBICETRAPIB |
| | | Amount | Amount | Amount | greater/equal | (TA-8995) | (TA-8995) |
| | | HPLC/2731 | HPLC/2731 | HPLC/2731 | than 0.05% | DISS/1295 | DISS/1295 |
| Storage | Time | (% claim) | (% claim) | (% claim) | claim | (% claim) | (% claim) |
| Condition | (Months | (Mean) | (Mean) | (Mean) | (Mean) | (Mean) | (Range) |
| Specificatio | N/A | NGT 0.5% | NGT 0.5 | NGT 0.5% | NGT 2.0% | Report result | Report result |
| n | | claim | claim | claim | claim | | |
| Initial | 0 | <0.05 | 0.05 | — | 0.10 | 90 | 89-92 |
| 25° C./60% | 1 | <0.05 | 0.05 | <0.05 | 0.10 | 94 | 93-95 |
| RH | 3 | <0.05 | 0.05 | <0.05 | 0.05 | 91 | 90-95 |
| Packaged | | | | | | | |
| 30° C./65% | 1 | <0.05 | 0.05 | <0.05 | 0.10 | 94 | 93-95 |
| RH | | | | | | | |
| Packaged | | | | | | | |
| 40° C./75% | 1 | <0.05 | 0.05 | <0.05 | 0.10 | 93 | 91-94 |
| RH | 3 | <0.05 | 0.05 | <0.05 | 0.05 | 90 | 89-91 |
| Packaged | | | | | | | |

Table 54(contd.)

| | | Time | Time | Time | Time | Time | Time |
|---|---|---|---|---|---|---|---|
| | | point(min) 30 | point(min) 30 | point(min) 45 | point(min) 45 | point(min) 60 | point(min) 60 |
| | | OBICETRAPIB | OBICETRAPIB | OBICETRAPIB | OBICETRAPIB | OBICETRAPIB | OBICETRAPIB |
| | | (TA-8995) | (TA-8995) | (TA-8995) | (TA-8995) | (TA-8995) | (TA-8995) |
| | | DISS/1295 | DISS/1295 | DISS/1295 | DISS/1295 | DISS/1295 | DISS/1295 |
| Storage | Time | (% claim) | (% claim) | (% claim) | (% claim) | (% claim) | (% claim) |
| Condition | (Months) | (Mean) | (Range) | (Mean) | (Range) | (Mean) | (Range) |
| Specificati | N/A | Report results | Report results | Complies with | Report results | Report results | Report results |
| on | | | | EP, USP, JP | | | |
| | | | | where | | | |
| | | | | Q = 75% at 45 | | | |
| | | | | minutes | | | |
| Initial | 0 | 96 | 94-97 | 97 | 95-98 | 97 | 96-98 |
| 25° C./60% | 1 | 99 | 97-100 | 99 | 99-100 | 99 | 98-101 |
| RH | 3 | 100 | 97-102 | 101 | 97-108 | 99 | 97-102 |
| Packaged | | | | | | | |
| 30° C./65% | 1 | 100 | 97-103 | 100 | 98-101 | 100 | 98-101 |
| RH | | | | | | | |
| Packaged | | | | | | | |
| 40° C./75% | 1 | 99 | 98-100 | 100 | 98-101 | 100 | 98-101 |
| RH | 3 | 99 | 97-101 | 99 | 98-101 | 99 | 98-101 |
| Packaged | | | | | | | |
| | | Time | Time | Time | Time | Time | Time |
| | | point(min) 70 | point(min) 70 | point(min) | point(min) | point(min) | point(min) |
| | | OBICETRAPIB | OBICETRAPIB | 15 | 15 | 30 | 30 |
| | | (TA-8995) | (TA-8995) | EZETIMIBE | EZETIMIBE | EZETIMIBE | EZETIMIBE |
| | | DISS/1295 | DISS/1295 | DISS/1298 | DISS/1298 | DISS/1298 | DISS/1298 |
| Storage | Time | (% claim) | (% claim) | (% claim) | (% claim) | (% claim) | (% claim) |
| Condition | (Months) | (Mean) | (Range) | (Mean) | (Range) | (Mean) | (Range) |
| Specification | N/A | Report result | Report result | Report result | Report result | Report result | Report result |
| Initial | 0 | 97 | 96-98 | 84 | 82-87 | 96 | 93-99 |
| 25° C./60% | 1 | 99 | 98-101 | 85 | 73-89 | 98 | 97-100 |
| RH | 3 | 99 | 96-101 | 89 | 85-91 | 99 | 94-101 |
| Packaged | | | | | | | |
| 30° C./65% | 1 | 100 | 98-101 | 89 | 83-93 | 96 | 91-100 |
| RH | | | | | | | |
| Packaged | | | | | | | |
| 40° C./75% | 1 | 100 | 99-101 | 90 | 87-91 | 98 | 96-100 |
| RH | 3 | 99 | 98-101 | 86 | 81-90 | 98 | 92-102 |
| Packaged | | | | | | | |
| | | Time | Time | Time | Time | Time | Time |
| | | point(min) | point(min) | point(min) | point(min) | point(min) | point(min) |
| | | 45 | 45 | 60 | 60 | 70 | 70 |
| | | EZETIMIBE | EZETIMIBE | EZETIMIBE | EZETIMIBE | EZETIMIBE | EZETIMIBE |
| | | DISS/1298 | DISS/1298 | DISS/1298 | DISS/1298 | DISS/1298 | DISS/1298 |
| Storage | Time | (% claim) | (% claim) | (% claim) | (% claim) | (% claim) | (% claim) |
| Condition | (Months) | (Mean) | (Range) | (Mean) | (Range) | (Mean) | (Range) |
| Specification | N/A | Complies | Report result | Report result | Report result | Report result | Report result |
| | | with EP, USP, | | | | | |
| | | JP where | | | | | |
| | | Q = 75% at | | | | | |
| | | 45 minutes | | | | | |
| Initial | 0 | 99 | 97-103 | 102 | 99-105 | 104 | 101-107 |
| 25° C./60% RH | 1 | 104 | 103-105 | 105 | 104-107 | 107 | 105-109 |
| Packaged | 3 | 103 | 98-105 | 105 | 100-106 | 106 | 102-107 |
| 30° C./65% RH | 1 | 98 | 94-103 | 101 | 96-105 | 104 | 102-107 |
| Packaged | | | | | | | |
| 40° C./75% RH | 1 | 101 | 98-103 | 103 | 100-104 | 105 | 103-107 |
| Packaged | 3 | 102 | 96-107 | 104 | 98-108 | 106 | 102-109 |

| | | Water content | Strength | Strength | Strength | Strength |
|---|---|---|---|---|---|---|
| | | UMID/1231 | RESR/1001 | RESR/1001 | RESR/1001 | RESR/1001 |
| Storage | Time | (% w/w) | (N) | (N) | (Kp) | (Kp) |
| Condition | (Months) | (Mean) | (Mean) | (Range) | (Mean) | (Range) |
| Specification | NA | Report result | Report result | Report result | Report result | Report result |
| Initial | 0 | 4.0 | | | | |
| 25° C./60% RH | 1 | 4.2 | 74 | 70-76 | 7.58 | 7.14-7.75 |
| Packaged | 3 | 4.6 | 70 | 66-78 | 7.17 | 6.73-7.95 |
| 30° C./65% RH | 1 | 4.4 | 73 | 70-77 | 7.46 | 7.14-7.85 |
| Packaged | | | | | | |
| 40° C./75% RH | 1 | 4.8 | 65 | 61-69 | 6.62 | 6.22-7.04 |
| Packaged | 3 | 5.5 | 60 | 57-63 | 6.10 | 5.81-6.42 |

N/A: Not Applicable

TABLE 55
Long-term and accelerated storage condition results for FDC2 technical batch
no. A4459/31/01 (with desiccant)

| | | | OBICETRAPIB | | ETHYL | | MONO- |
|---|---|---|---|---|---|---|---|
| | | | (TA-8995) | DIASTEREOISOMER | ESTER | FROL | BN FREE |
| | | Appearan./ | Amount | Amount | Amount | Amount | Amount |
| | | Colour | HPLC/2730 | HPLC/2730 | HPLC/2730 | HPLC/2730 | HPLC/2730 |
| Storage | Time | APP/1001 | (% claim) | (% claim) | (% claim) | (% claim) | (% claim) |
| Condition | (Months) | (Individual) | (Mean) | (Mean) | (Mean) | (Mean) | (Mean) |
| Specification | | White to off- | 90.0-110.0% | NGT 0.3% claim | NGT 0.3% | NGT 0.3% | NGT 0.3% |
| | | white round | claim | | claim | claim | claim |
| | | coated tablet | | | | | |
| Initial | 0 | White round | 94.3 | <0.05 | <0.05 | 0.06 | 0.16 |
| | | coated tablet | | | | | |
| 25° C./60% | 3 | White round | 96.4 | <0.05 | <0.05 | 0.07 | 0.16 |
| RH | | coated tablet | | | | | |
| Packaged | | | | | | | |
| 40° C./75% | 3 | White round | 96.3 | <0.05 | <0.05 | 0.07 | 0.16 |
| RH | | coated tablet | | | | | |
| Packaged | | | | | | | |
| | | | | Total | | | |
| | | | | degradation | | | |
| | | | | products | | EZETIMIBE | EZETIMIBE |
| | | IMP_RRT0.93 | IMP_RRT0.94 | equal/greater | EZETIMIBE | CYCLIC | KETONE |
| | | Amount | Amount | than 0.05% | Amount | Amount | Amount |
| | | HPLC/2730 | HPLC/2730 | claim | HPLC/2731 | HPLC/2731 | HPLC/2731 |
| Storage | Time | (% claim) | (% claim) | (% claim) | (% claim) | (% claim) | (% claim) |
| Condition | (Months) | (Mean) | (Mean) | (Mean) | (Mean) | (Mean) | (Mean) |
| Specification | | NGT 0.3% | NGT 0.3% | NGT 2.0% | 90.0-110.0% | NGT 0.5% | NGT 0.5% |
| | | claim | claim | claim | claim | claim | claim |
| Initial | 0 | <0.05 | 0.05 | 0.26 | 100.7 | <0.05 | 0.05 |
| 25° C./60% RH | 3 | <0.05 | 0.07 | 0.30 | 103.5 | <0.05 | <0.05 |
| Packaged | | | | | | | |
| 40° C./75% RH | 3 | <0.05 | 0.06 | 0.29 | 104.1 | <0.05 | <0.05 |
| Packaged | | | | | | | |
| | | | | Total | Time | Time | Time |
| | | | | degradation | point(min) 15 | point(min) 15 | point(min) 30 |
| | | IMP_RRT0.73 | IMP_RRT0.79 | products | OBICETRAPIB | OBICETRAPIB | OBICETRAPIB |
| | | Amount | Amount | greater/equal | (TA-8995) | (TA-8995) | (TA-8995) |
| | | HPLC/2731 | HPLC/2731 | than | DISS/1295 | DISS/1295 | DISS/1295 |
| Storage | Time | (% claim) | (% claim) | 0.05% claim | (% claim) | (% claim) | (% claim) |
| Condition | (Months) | (Mean) | (Mean) | (Mean) | (Mean) | (Range) | (Mean) |
| Specificatio | | NGT 0.5% | NGT 0.5% | NGT 2.0% | Report Results | Report Results | Report Results |
| n | | claim | claim | claim | | | |
| Initial | 0 | <0.05 | <0.05 | 0.05 | 85 | 82-86 | 93 |
| 25° C./60% | 3 | <0.05 | 0.05 | 0.05 | 85 | 83-86 | 93 |
| RH | | | | | | | |
| Packaged | | | | | | | |
| 40° C./75% | 3 | <0.05 | 0.06 | 0.06 | 83 | 82-85 | 92 |
| RH | | | | | | | |
| Packaged | | | | | | | |
| | | Time | Time | Time | Time | Time | Time |
| | | point(min) 30 | point(min) 45 | point(min) 45 | point(min) 60 | point(min) 60 | point(min) 70 |
| | | OBICETRAPIB | OBICETRAPIB | OBICETRAPIB | OBICETRAPIB | OBICETRAPIB | OBICETRAPIB |
| | | (TA-8995) | (TA-8995) | (TA-8995) | (TA-8995) | (TA-8995) | (TA-8995) |
| | | DISS/1295 | DISS/1295 | DISS/1295 | DISS/1295 | DISS/1295 | DISS/1295 |
| Storage | Time | (% claim) | (% claim) | (% claim) | (% claim) | (% claim) | (% claim) |
| Condition | (Months) | (Range) | (Mean) | (Range) | (Mean) | (Range) | (Mean) |
| Specification | | Report Results | Complies with | Report Results | Report Results | Report Results | Report Results |
| | | | EP, USP, JP | | | | |
| | | | where | | | | |
| | | | Q = 75% at 45 | | | | |
| | | | minutes | | | | |
| Initial | 0 | 92-94 | 95 | 93-96 | 95 | 94-97 | 96 |
| 25° C./60% | 3 | 92-95 | 95 | 93-97 | 95 | 93-97 | 95 |
| RH | | | | | | | |
| Packaged | | | | | | | |
| 40° C./75% | 3 | 91-93 | 95 | 93-95 | 95 | 93-96 | 95 |
| RH | | | | | | | |
| Packaged | | | | | | | |

Table 55(contd.)

| | | Time | Time | Time | Time | Time | Time |
|---|---|---|---|---|---|---|---|
| | | point(min) 70 | point(min) | point(min) | point(min) | point(min) | point(min) |
| | | OBICETRAPIB | 15 | 15 | 30 | 30 | 45 |
| | | (TA-8995) | EZETIMIBE | EZETIMIBE | EZETIMIBE | EZETIMIBE | EZETIMIBE |
| | | DISS/1295 | DISS/1298 | DISS/1298 | DISS/1298 | DISS/1298 | DISS/1298 |
| Storage | Time | (% claim) | (% claim) | (% claim) | (% claim) | (% claim) | (% claim) |
| Condition | (Months) | (Range) | (Mean) | (Range) | (Mean) | (Range) | (Mean) |
| Specification | | Report Results | Report | Report | Report | Report | Complies |
| | | | Results | Results | Results | Results | with EP, USP, |
| | | | | | | | JP where |
| | | | | | | | Q = 75% at 45 |
| | | | | | | | minutes |
| Initial | 0 | 94-97 | 81 | 72-83 | 92 | 88-94 | 96 |
| 25° C./60% RH | 3 | 93-97 | 81 | 60-87 | 95 | 85-99 | 97 |
| Packaged | | | | | | | |
| 40° C./75% RH | 3 | 93-96 | 86 | 83-88 | 94 | 91-96 | 96 |
| Packaged | | | | | | | |
| | | Time | Time | Time | Time | Time | |
| | | point(min) | point(min) | point(min) | point(min) | point(min) | |
| | | 45 | 60 | 60 | 70 | 70 | Water |
| | | EZETIMIBE | EZETIMIBE | EZETIMIBE | EZETIMIBE | EZETIMIBE | content |
| | | DISS/1298 | DISS/1298 | DISS/1298 | DISS/1298 | DISS/1298 | UMID/1231 |
| Storage | Time | (% claim) | (% claim) | (% claim) | (% claim) | (% claim) | (% w/w) |
| Condition | (Months) | (Range) | (Mean) | (Range) | (Mean) | (Range) | (Mean) |
| Specification | | Report | Report | Report | Report | Report | Report |
| | | Results | Results | Results | Results | Results | Results |
| Initial | 0 | 95-97 | 98 | 97-99 | 102 | 101-104 | 3.7 |
| 25° C./60% RH | 3 | 89-101 | 99 | 92-103 | 103 | 100-104 | 2.9 |
| Packaged | | | | | | | |
| 40° C./75% RH | 3 | 93-98 | 98 | 95-100 | 103 | 102-104 | 3.3 |
| Packaged | | | | | | | |

Table 55 (contd.)

| | | | | | |
|---|---|---|---|---|---|
| | | Strength | Strength | Strength | Strength |
| | | RESR/1001 (N) | RESR/1001 (N) | RESR/1001 (Kp) | RESR/1001 (Kp) |
| Storage Condition | Time | (Mean) | (Range) | (Mean) | (Range) |
| Specification | (Months) | Report Results | Report Results | Report Results | Report Results |
| Initial | 0 | 92 | 81-99 | 9.36 | 8.26-10.10 |
| 25° C./60% RH Packaged | 3 | 115 | 110-120 | 1.70 | 11.22-12.24 |
| 40° C./75% RH Packaged | 3 | 114 | 102-127 | 11.59 | 10.40-12.95 |

TABLE 56
Long-term and accelerated storage condition results for FDC1 technical batch
no. A4459/31/02 (with desiccant)

| | | | | | | | MONO- |
|---|---|---|---|---|---|---|---|
| | | | OBICETRAPIB | | ETHYL | | BN FREE |
| | | | (TA-8995) | DIASTEREOISOMER | ESTER | FROL | Amount |
| | | Appearan./ | Amount | Amount | Amount | Amount | HPLC/ |
| | | Colour | HPLC/2730 | HPLC/2730 | HPLC/2730 | HPLC/2730 | 2730 |
| Storage | Time | APP/1001 | (% claim) | (% claim) | (% claim) | (% claim) | (% claim) |
| Condition | (Months | (Individual) | (Mean) | (Mean) | (Mean) | (Mean) | (Mean) |
| Specification | NA | White to off- | 90.0-110.0% | NGT 0.3% | NGT 0.3% | NGT 0.3% | NGT 0.3% |
| | | white round | claim | claim | claim | claim | claim |
| | | coated tablet | | | | | |
| Initial | 0 | White round | 96.7 | <0.05 | <0.05 | 0.11 | 0.15 |
| | | coated tablet | | | | | |
| 25° C./60% | 3 | White round | 98.0 | <0.05 | <0.05 | 0.11 | 0.16 |
| RH | | coated tablet | | | | | |
| Packaged | | | | | | | |
| 40° C./75% | 3 | White round | 98.1 | <0.05 | <0.05 | 0.11 | 0.16 |
| RH | | coated tablet | | | | | |
| Packaged | | | | | | | |

Table 56(contd.)

| | | | | Total | | | |
|---|---|---|---|---|---|---|---|
| | | | | degradation | | | |
| | | | | products | | EZETIMIBE | EZETIMIBE |
| | | IMP_RRT0.93 | IMP_RRT0.94 | equal/greater | EZETIMIBE | CYCLIC | KETONE |
| | | Amount | Amount | than 0.05% | Amount | Amount | Amount |
| | | HPLC/2730 | HPLC/2730 | claim | HPLC/2731 | HPLC/2731 | HPLC/2731 |
| Storage | Time | (% claim) | (% claim) | (% claim) | (% claim) | (% claim) | (% claim) |
| Condition | (Months) | (Mean) | (Mean) | (Mean) | (Mean) | (Mean) | (Mean) |
| Specification | | NGT 0.3% | NGT 0.3% | NGT 2.0% | 90.0-110.0% | NGT 0.5% | NGT 0.5% |
| | | claim | claim | claim | claim | claim | claim |
| Initial | 0 | 0.07 | <0.05 | 0.33 | 100.8 | <0.05 | 0.05 |
| 25° C./60% RH | 3 | 0.06 | <0.05 | 0.34 | 104.7 | <0.05 | <0.05 |
| Packaged | | | | | | | |
| 40° C./75% RH | 3 | 0.07 | <0.05 | 0.34 | 105.3 | <0.05 | <0.05 |
| Packaged | | | | | | | |
| | | | | Total | Time | Time | Time |
| | | | | degradation | point(min) 15 | point(min) 15 | point(min) 30 |
| | | IMP_RRT0.73 | IMP_RRT0.79 | products | OBICETRAPIB | OBICETRAPIB | OBICETRAPIB |
| | | Amount | Amount | greater/equal | (TA-8995) | (TA-8995) | (TA-8995) |
| | | HPLC/2731 | HPLC/2731 | than | DISS/1295 | DISS/1295 | DISS/1295 |
| Storage | Time | (% claim) | (% claim) | 0.05% claim | (% claim) | (% claim) | (% claim) |
| Condition | (Months) | (Mean) | (Mean) | (Mean) | (Mean) | (Range) | (Mean) |
| Specification | | NGT 0.5% | NGT 0.5% | NGT 2.0% | Report Results | Report Results | Report Results |
| | | claim | claim | claim | | | |
| Initial | 0 | <0.05 | 0.05 | 0.10 | 90 | 89-92 | 96 |
| 25° C./60% | 3 | <0.05 | 0.05 | 0.05 | 89 | 88-90 | 97 |
| RH | | | | | | | |
| Packaged | | | | | | | |
| 40° C./75% | 3 | <0.05 | 0.05 | 0.05 | 88 | 87-89 | 95 |
| RH | | | | | | | |
| Packaged | | | | | | | |
| | | Time | Time | Time | Time | Time | Time |
| | | point(min) 30 | point(min) 45 | point(min) 45 | point(min) 60 | point(min) 60 | point(min) 70 |
| | | OBICETRAPIB | OBICETRAPIB | OBICETRAPIB | OBICETRAPIB | OBICETRAPIB | OBICETRAPIB |
| | | (TA-8995) | (TA-8995) | (TA-8995) | (TA-8995) | (TA-8995) | (TA-8995) |
| | | DISS/1295 | DISS/1295 | DISS/1295 | DISS/1295 | DISS/1295 | DISS/1295 |
| Storage | Time | (% claim) | (% claim) | (% claim) | (% claim) | (% claim) | (% claim) |
| Condition | (Months) | (Range) | (Mean) | (Range) | (Mean) | (Range) | (Mean) |
| Specificati | | Report Results | Complies with | Report Results | Report Results | Report Results | Report Results |
| on | | | EP, USP, JP | | | | |
| | | | where | | | | |
| | | | Q = 75% at 45 | | | | |
| | | | minutes | | | | |
| Initial | 0 | 94-97 | 97 | 95-98 | 97 | 96-98 | 97 |
| 25° C./60% | 3 | 96-98 | 98 | 97-98 | 98 | 97-98 | 98 |
| RH | | | | | | | |
| Packaged | | | | | | | |
| 40° C./75% | 3 | 94-97 | 96 | 95-97 | 96 | 95-98 | 96 |
| RH | | | | | | | |
| Packaged | | | | | | | |

Table 56(contd.)

| | | Time | Time | Time | Time | Time | Time |
|---|---|---|---|---|---|---|---|
| | | point(min) 70 | point(min) | point(min) | point(min) | point(min) | point(min) |
| | | OBICETRAPIB | 15 | 15 | 30 | 30 | 45 |
| | | (TA-8995) | EZETIMIBE | EZETIMIBE | EZETIMIBE | EZETIMIBE | EZETIMIBE |
| | | DISS/1295 (% | DISS/1298 | DISS/1298 | DISS/1298 | DISS/1298 | DISS/1298 |
| Storage | Time | claim) | (% claim) | (% claim) | (% claim) | (% claim) | (% claim) |
| Condition | (Months) | (Range) | (Mean) | (Range) | (Mean) | (Range) | (Mean) |
| Specification | | Report Results | Report | Report | Report | Report | Complies |
| | | | Results | Results | Results | Results | with EP, USP, |
| | | | | | | | JP where |
| | | | | | | | Q = 75% at 45 |
| | | | | | | | minutes |
| Initial | 0 | 96-98 | 84 | 82-87 | 96 | 93-99 | 99 |
| 25° C./60% RH | 3 | 97-99 | 92 | 90-94 | 100 | 98-102 | 104 |
| Packaged | | | | | | | |
| 40° C./75% RH | 3 | 95-97 | 90 | 87-92 | 102 | 98-104 | 105 |
| Packaged | | | | | | | |
| | | Time | Time | Time | Time | Time | |
| | | point(min) | point(min) | point(min) | point(min) | point(min) | |
| | | 45 | 60 | 60 | 70 | 70 | Water |
| | | EZETIMIBE | EZETIMIBE | EZETIMIBE | EZETIMIBE | EZETIMIBE | content |
| | | DISS/1298 | DISS/1298 | DISS/1298 | DISS/1298 | DISS/1298 | UMID/1231 |
| Storage | Time | (% claim) | (% claim) | (% claim) | (% claim) | (% claim) | (% w/w) |
| Condition | (Months) | (Range) | (Mean) | (Range) | (Mean) | (Range) | (Mean) |
| Specification | | Report | Report | Report | Report | Report | Report |
| | | Results | Results | Results | Results | Results | Results |
| Initial | 0 | 97-103 | 102 | 99-105 | 104 | 101-107 | 4.0 |
| 25° C./60% RH | 3 | 102-105 | 106 | 104-107 | 108 | 106-109 | 3.5 |
| Packaged | | | | | | | |
| 40° C./75% RH | 3 | 102-109 | 107 | 103-109 | 107 | 105-110 | 3.7 |
| Packaged | | | | | | | |

| | | Strength | Strength | Strength | Strength |
|---|---|---|---|---|---|
| | Time | RESR/1001 (N) | RESR/1001 (N) | RESR/1001 (Kp) | RESR/1001 (Kp) |
| Storage Condition | (Months) | (Mean) | (Range) | (Mean) | (Range) |
| Specification | NA | Report Results | Report Results | Report Results | Report Results |
| Initial | 0 | — | — | — | — |
| 25° C./60% RH Packaged | 3 | 77 | 69-84 | 7.89 | 7.04-8.57 |
| 40° C./75% RH Packaged | 3 | 77 | 69-82 | 7.86 | 7.04-8.36 |

N/A: Not Applicable

### Example 9: Study to Evaluate the Comparative Bioavailability of Two Fixed-Dose Combination Formulations of Obicetrapib/Ezetimibe 10 mg/10 mg (FDC1 and FDC2) with Obicetrapib, 10 mg Co-Administered with Ezetimibe, 10 mg in Healthy Adult Subjects Under Fasted Conditions

### Study Design

This was an open-label, single-dose, randomized, three-treatment, three-period, six-sequence crossover study comparing the two test products and coadministration of the reference products under fasted conditions. In each of the study periods, the subjects received either Treatment T1 (1×obicetrapib, 10 mg and ezetimibe, 10 mg FDC1 tablet [Formulation #1]), Treatment T2 (1×obicetrapib, 10 mg and ezetimibe, 10 mg FDC2 tablet [Formulation #2]), or Treatment R (1×obicetrapib tablet, 10 mg co-administered with 1×ZETIA® (ezetimibe) tablet, 10 mg) following an overnight fast of at least 10 hours. The order of administration followed a six sequence randomization schedule. Blood samples were collected at pre-dose and at intervals over 336 hours after dosing in each study period. Subjects were confined at the clinical facility from at least 10 horns before dosing until 24 hours after dosing in each study period and returned to the clinical facility for the 48-, 72-, 96-, 144-, 192-, 240-, and 336-hour post-dose blood sample collections. The interval between doses were at least 49 days.

The plasma concentrations of obicetrapib, ezetimibe and its metabolite, ezetimibe glucuronide were measured by fully validated analytical methods. Statistical analysis using average bioequivalence methodology were performed to evaluate the bioavailability of each of the test formulations relative to that of the coadministration of the reference products.

### Selection of Study Population

The subject population included 36 healthy, non-tobacco-, non-nicotine-using, adult male and female subjects.

### Treatment Administration

The subjects received Treatment T1, Treatment T2, or Treatment R according to a three treatment, three-period, six-sequence randomization schedule (Table 57) under direct observation following an overnight fast of at least 10 hours.
Treatment T1: 1×obicetrapib, 10 mg and ezetimibe, 10 mg FDC Tablet (Formulation #1)
Treatment T2: 1×obicetrapib, 10 mg and ezetimibe, 10 mg FDC Tablet (Formulation #2)
Treatment R: 1×obicetrapib Tablet, 10 mg co-administered with 1×ZETIA® (ezetimibe) Tablet, 10 mg

TABLE 57

| Sequence | Period 1 | Period 2 | Period 3 |
|---|---|---|---|
| 1 | T1 | T2 | R |
| 2 | T2 | TI | R |
| 3 | R | T1 | T2 |
| 4 | T1 | T2 | R |
| 5 | T2 | T1 | R |
| 6 | R | T1 | T2 |

Each dose was administered with 240 mL of room temperature water. Subjects were instructed to swallow the tablet(s) whole without chewing or biting.

### Sample Collection, Handling and Bioanalytical Plans

### Sample Size

4 mL collections (K2EDTA vacutainers) for analysis of obicetrapib
4 mL collections (K2EDTA vacutainers) for analysis of ezetimibe and ezetimibe glucuronide

### Collection Times

Pre-dose samples were collected within 60 minutes before dosing. All times are relative to the dosing minute.

For analysis of obicetrapib: Pre-dose (0-hour) and at 0.50, 1.0, 1.5, 2.0, 2.5, 3.0, 3.5, 4.0, 4.5, 5.0, 6.0, 7.0, 9.0, 12.0, 16.0, 20.0, 24.0, 48.0*, 72.0*, 96.0*, 144.0*, 192.0*, 240.0* and 336.0* hours post-dose (*return sample)

For analysis of ezetimibe and ezetimibe glucuronide: Pre-dose (0-hour) and at 0.25, 0.50, 0.75, 1.0, 1.333, 1.667, 2.0, 2.5, 3.0, 3.5, 4.0, 4.5, 5.0, 6.0, 7.0, 9.0, 12.0, 16.0, 20.0, 24.0, 48.0*, 72.0* and 96.0* hours post-dose (*return sample)

Total number of collections per period/per subject: 49

Total blood volume per subject: The total volume of blood collected for pharmacokinetic sampling was approximately 588 mL.

Sample Processing for analysis of obicetrapib: Blood samples were collected in room temperature 4 mL K₂EDTA vacutainers. After collection, the samples were mixed by gently inverting the tube several (i.e., 8-10) times and placed in an ice/water bath. The samples were then placed in the centrifuge and spin at 3000 rpm for 10 minutes at 4° C. The resulting plasma was separated into two aliquots (at least 1.0 mL in Aliquot 1 and remainder in aliquot 2) and transferred into polypropylene sample storage tubes and stored at −70° C. (±10° C.) until ready for shipment to the bioanalytical laboratory. The plasma aliquots were placed in the freezer within 30 minutes after sample collection. After collection until placement in the freezer, blood/plasma samples were kept cooled in an ice/water bath.

Sample Processing for analysis of ezetimibe and ezetimibe glucuronide: Blood samples were collected in room temperature 4 mL K2EDT A vacutainers. After collection, the samples were mixed gently by inverting the tube several (at least 8) times and placed in an ice/water bath. The samples were then placed in the centrifuge and spin at 3000 rpm for 10 minutes at 4° C. The resulting plasma was separated into two aliquots (at least 1.0 mL in Aliquot 1 and remainder in Aliquot 2) and transferred into polypropylene sample storage tubes (e.g., Sarstedt #60.546) and stored at −70° C. (or colder) until ready for shipment to the bioanalytical laboratory. After collection until placement in the freezer, blood/plasma samples were kept cooled in an ice/water bath.

### Pharmacokinetic Analysis

For all treatments, the following pharmacokinetic parameters were calculated for obicetrapib, ezetimibe and its metabolite, ezetimibe glucuronide.

### Primary PK Parameters

Cmax: Maximum measured plasma concentration.

AUC₀₋ₜ: Area under the plasma concentration versus time curve from time zero to the last measurable plasma concentration, as calculated by linear trapezoidal method.

AUC_{0-∞}: Area under the plasma concentration versus time curve from time zero to infinity where AUCo-∞=AUCo-t+Ct/λz. Ct is the last measurable concentration and λz is the terminal disposition rate constant.

### Secondary PK Parameters

Tmax: Time of the maximum measured plasma concentration. If the maximum value occurred at more than one time point, Tmax is defined as the first time point with this value.

λz: Apparent first-order terminal disposition rate constant. This parameter was calculated from the negative of the slope of the dataset with the best-fit least-squares linear regression analysis of the terminal in-linear concentration-time data. The number of data points (3 or more) in the terminal phase (not including Cmax) was included in the final regression analysis for an evaluable λz was determined from the dataset that has the highest adjusted Rsquared

(R²) value of 0.7 or more. λz was considered non-evaluable if (1) the last three terminal points were used to determine λz and either the middle or the last point was higher than the preceding point or (2) the resulting adjusted R² value was less than 0.7.

An evaluable λz was considered not reliable and not reportable if the resulting apparent first-order terminal half-life (t½) value was longer than the time interval over which λz was estimated. If the resulting t½, value was longer than the time interval over which λz was determined, an interval that was longer than the estimated t½, was explored. The interval with the next highest adjusted R² value was chosen and the decrease in the adjusted R² value was assessed to determine if a reliable estimation of the λz was possible. If λz was deemed not reliable then no t½, and AUCo-∞ values were reported for that dataset.

t½: The first-order terminal disposition half-life was calculated as ln(2)/λz

### Data Set for Analysis and Statistical Methods

Linear and semi-logarithmic graphs of the concentration-time profiles for each subject were provided, using the actual times of sample collections. Actual sample collection time were used for calculating the pharmacokinetic parameters. Plasma concentration data from all evaluable subjects with no significant protocol deviation(s) were used for estimation of Cmax and/or AUCs from at least two periods of the study, one of which includes Treatment R.

PK parameters from any subject who experienced emesis within two times the median Tmax of obicetrapib or ezetimibe, respectively, calculated from the observed data of the specific treatment arm were excluded from the statistical analysis for the respective analyte.

Analyses of Variance was performed on In-transformed AUCo-t, AUCo-∞, and Cmax using an analysis of variance model (ANOVA). The ANOVA was conducted separately for Treatment T1 versus Treatment R analysis and for Treatment T2 versus Treatment R analysis, using an incomplete block design. Treatment T2 was excluded from ANOVA for comparison of Treatments T1 versus R and Treatment T1 was excluded from ANOVA for comparison of Treatments T2 versus R.

Confidence intervals (90%) on the geometric mean ratios (obtained from logarithmic transformed data) for AUCo-t, AUCo-∞ and Cmax for the comparison of each of the FDC formulations (T1 and T2) to Treatment R was constructed to test two one-sided hypotheses at the a=0.05 level of significance.

### Results

Confidence intervals (90%) on the geometric mean ratios for AUCo-t, AUCo-∞ and Cmax for obicetrapib, ezetimibe and ezetimibe glucoronide (from T1 and T2 for formulation #1 as well as formulation #2) were found to be within a range of 75%-125%, preferably 80%-125%, and more preferably 90%-110% of AUCo-t, AUCo-∞ and Cmax of obicetrapib, ezetimibe and ezetimibe glucoronide, respectively. The test formulations #1 and #2 were found to be bioequivalent with reference treatment arm (R). The adverse events observed with T1 or T2 arm of the treatment were statistically not significantly different from the R arm.

### Example 10: Phase 2B Clinical Trial (ROSE2; NCT05266586)

### 1 Introduction and Background Information

Dyslipidemias are disorders of lipoprotein metabolism, including lipoprotein overproduction or deficiency, which may be manifested by elevation of the levels of the serum total cholesterol, low-density lipoprotein (LDL) cholesterol (LDL-C) and triglyceride (TG) concentrations, and a decrease in the levels of high-density lipoprotein (HDL) cholesterol (HDL-C) concentration. These disorders are generally diagnosed by measuring the serum lipids and classified by the pattern of elevation/reduction in the lipid/lipoprotein fractions. Dyslipidemia itself does not generally cause any symptoms, but it can lead to symptomatic vascular disease including coronary artery disease and peripheral arterial disease. It is acknowledged that while there are a number of genetic and lifestyle factors which contribute to the development of vascular disease, dyslipidemia is 1 of the most prominent risk factors and normalization of the lipid profile has been a major target in cardiovascular (CV) protection strategies.

Statins are generally the drug of first choice in treating dyslipidemia. Statins are considered as the most potent, most effective, and best tolerated drugs for reducing LDL-C levels. Many patients, despite treatment with high-intensity statin therapy, do not achieve acceptable levels of LDL-C with statins alone.

There is a need for chronic therapies which will robustly reduce elevated LDL-C levels when used adjunctive to high-intensity statin therapy.

### 1.1 Cholesteryl Ester Transfer Protein Inhibitors

Cholesteryl ester transfer protein (CETP) is a plasma glycoprotein produced in the liver and adipose tissue. It circulates in the blood, bound primarily to HDL-C, and is involved in the transfer of cholesteryl esters and TG between lipoproteins. In particular, it mediates the transfer of cholesteryl esters from HDL to apolipoprotein B (ApoB)-containing particles, eg, very low-density lipoprotein and LDL-C, in exchange for TG. As a result, cholesteryl ester from HDL can be taken up by the liver through scavenger receptor class B type 1; this action also leads to decreased HDL-C and ultimately to increased LDL-C.

Inhibition of CETP activity reduces ApoB and LDL-C and increases HDL-C. CETP-inhibiting therapies were originally developed based on the premise that increasing HDL-C levels would prevent CV events. However, clinical study results and Mendelian randomization data have revealed that these effects are caused by changes in the concentration of ApoB-containing particles (including LDL particles) rather than changes in the HDL-C levels. Therefore, the LDL-C and ApoB-lowering effects, which arise from CETP inhibition and occur through upregulation of the LDL receptor, will benefit patients with elevated LDL-C and increased CV risk.

Ference and colleagues have recently investigated the association between changes in LDL-C levels (and other lipoproteins) and the risk of CV events related to variants in the CETP gene alone and in combination with variants in the 3-hydroxy-3-methylglutaryl-coenzyme A reductase (HMGCR) gene.

The results of these Mendelian randomization analyses demonstrate that treatment with a CETP inhibitor has the potential to effectively reduce the risk of CV events. Both genetic and therapeutic inhibition of CETP leads to quantitatively concordant changes in LDL-C and ApoB levels. A further Mendelian randomization analysis concluded that the clinical benefit of lower LDL-C levels per unit difference may specifically be related to the absolute reduction in ApoB-containing lipoprotein particles.

### 1.2 Obicetrapib (TA-8995)

Obicetrapib (TA-8995) is a selective CETP inhibitor. Inhibition of CETP by obicetrapib blocks the transfer of cholesteryl ester from non-atherogenic HDL particles to particles in lipoprotein fractions (including LDL) that cause atherosclerosis and reduces the concentration of cholesterol in LDL, as well as other atherogenic lipoproteins. Obicetrapib also has several additional compound-specific activities that are hypothesized to be beneficial in patients. In a recent study, obicetrapib treatment not only reduced the number of ApoB-containing particles that constitute LDL-C, it also increased apolipoprotein E (ApoE), which led to the removal of cholesterol via the liver and also reduced lipoprotein (a) (Lp[a]).5 Finally, obicetrapib not only potently increases HDL-C and the concentration of apolipoprotein A1 (ApoA1)-containing lipoproteins but has been demonstrated to be a potent inducer of cholesterol efflux, which is the main driver of reverse cholesterol transport. This effect is considered important because it is expected to reduce established atheroma burden.

### 1.3 Clinical Development of Obicetrapib

Both single ascending dose (TA-8995-01) and multiple ascending dose (TA-8995-02) studies of obicetrapib have been conducted in healthy volunteers. A formal, thorough QT/QTc study (TA-8995-04) demonstrated that obicetrapib has no effect on the QTcF. A drug-drug interaction study (TA-8995-05) showed no significant effect of obicetrapib on P-glycoprotein activity, but showed that obicetrapib is a mild inducer of cytochrome P450 3A4. A mass balance study in healthy males concluded that obicetrapib is steadily absorbed, and the principal route of excretion was in the feces (TA-8995-07). Finally, bioequivalence between obicetrapib capsule and tablet formulations was investigated (TA-8995-08).

The first patient study conducted was a Phase 2 clinical study (TA-8995-03) in Denmark and The Netherlands where the aim was to evaluate the optimal dose of obicetrapib alone and in combination with statins in patients with mild dyslipidemia. This study concluded that a 10 mg daily dose of obicetrapib therapy resulted in an LDL-C reduction of 45.4%, an HDL-C increase of 179.0%, an ApoA1 increase of 63.4%, and a significant increase of HDL-C efflux capacity.

Furthermore, given on top of atorvastatin 20 mg, obicetrapib 10 mg resulted in an additional 50.3% reduction in LDL-C. A second patient study (TA-8995-06) showed a statistically significant reduction in Lp(a) levels following 12 weeks of obicetrapib treatment.

Two Phase 2 studies of obicetrapib (TA-8995-303 and TA-8995-201) are currently nearing completion. The first study, TA-8995-303, is evaluating the LDL-lowering effects of obicetrapib 5 mg in combination with ezetimibe 10 mg in participants with mild dyslipidemia. The second study, TA-8995-201, is evaluating the LDL-lowering effects of obicetrapib (both 5 mg and 10 mg) as an adjunct to high intensity statin therapy in participants with dyslipidemia who are on high-intensity statins. Two Phase 3 studies of obicetrapib 10 mg investigating the treatment of elevated LDL-C levels in participants with established atherosclerotic CV disease (ASCVD), as well as heterozygous familial hypercholesterolemia, are currently in development. One study (TA-8995-301) will include participants on maximally tolerated lipid-lowering therapy, including maximally tolerated doses of statins with an LDL-C inclusion criterion of ≥100 mg/dL. The other study (TA-8995-302) will also include participants who are on maximally tolerated lipid-lowering therapy, including maximally tolerated doses of statins with an LDL-C inclusion criterion of ≥70 mg/dL and <100 mg/dL. Together, these 2 pivotal studies will evaluate the effects of obicetrapib 10 mg in populations requiring further LDL-C reduction across a range of baseline LDL-C values relevant to contemporary clinical practice.

A third Phase 3 study (TA-8995-304) will investigate the effect of obicetrapib 10 mg on clinical outcomes (ie, major adverse CV events, including CV death, non-fatal myocardial infarction, non-fatal stroke, or non-elective coronary revascularization).

### 1.4 Rationale

Chronic LDL-C elevation leads to progressive accumulation of arterial atherosclerotic lesions that require long-term management. While lifestyle changes are the primary intervention, these measures seldom reduce plasma LDL-C by more than 15%, and pharmacologic treatments are required to adequately treat hyperlipidemia.

Statins are considered as first-line therapy for reducing LDL-C levels. However, despite lipid lowering therapy with statins, many patients are unable to achieve acceptable levels of LDL-C.

Proprotein convertase subtilisin kexin type 9 (PCSK9) inhibitors are one alternative to statins. However, there are several limitations with these therapies, including high costs, limited long-term outcome data relative to statins, and muscle-related events. In addition, because PCSK9 inhibitors are injectable, this poses a less attractive option for patients who prefer oral medications.

Accordingly, there remains an unmet need for therapies to effectively reduce elevated LDL-C levels and CV risk at an acceptable cost, a convenient dosage form, and a more favorable safety profile to encourage long-term use and patient compliance. Obicetrapib, an oral CETP inhibitor, has demonstrated safety and efficacy in the reduction of LDL-C, in addition to other beneficial effects. The combination of obicetrapib and ezetimibe, an oral cholesterol absorption inhibitor, could be a valuable alternative to a PCSK9 inhibitor in patients who require additional LDL-C lowering despite high-intensity statin therapy.

### 1.4.1 Rationale for Obicetrapib and Ezetimibe Combination Therapy

Ezetimibe selectively inhibits intestinal cholesterol absorption. Ezetimibe used as monotherapy for patients with hypercholesterolemia significantly reduces serum LDL-C levels, as evidenced by a meta-analysis of 8 randomized, double-blind, placebo-controlled studies, with a statistically significant mean reduction in LDL-C of 18.58% compared with placebo. Ezetimibe in combination with statin therapy further reduces LDL-C levels. A meta-analysis of 27 studies, including more than 21,000 patients, demonstrated a 15.1% greater reduction in LDL-C in patients treated with statin and ezetimibe in combination compared with statin alone. The IMPROVE-IT study, in which simvastatin 40 mg daily was compared with a combination of simvastatin 40 mg plus ezetimibe 10 mg in 18,144 patients with acute coronary syndrome, demonstrated a modest but statistically significant further reduction in future CV events (a 2% absolute risk reduction over 7 years) in the combination group compared with statins alone. These large long-term studies have also demonstrated an excellent safety profile for ezetimibe. Importantly, additional Mendelian randomization studies have revealed that the CETP inhibitor HMGCR inhibitor interaction does not occur when a CETP inhibitor is combined with ezetimibe.

A Phase 2 study is currently underway evaluating the LDL-lowering effects of obicetrapib 5 mg in combination with ezetimibe 10 mg in participants with mild dyslipidemia and no prior CV events (TA-8995-303).

To evaluate the potential of a higher dose of obicetrapib in combination with ezetimibe to augment reduction in LDL-C and ApoB, this study will evaluate the effect of obicetrapib 10 mg in combination with ezetimibe 10 mg in participants currently receiving high-intensity statin therapy (atorvastatin 40 or 80 mg or rosuvastatin 20 or 40 mg) as part of their guideline-directed medical therapy.

### 1.4.2 Dose Selection Rationale for Obicetrapib

In clinical studies in healthy volunteers, obicetrapib was generally well tolerated in single doses up to 150 mg and multiple doses up to 25 mg/day for 21 days. In clinical studies in patients, obicetrapib was also well tolerated after daily dosing of 10 mg for 12 weeks, both alone and in combination with 2 different statins. Near maximal effects were observed with the 10 mg obicetrapib dose. At this dose level, CETP activity and concentrations were reduced, HDL-C levels were increased, and LDL-C levels were decreased. There were no dose-related adverse events (AEs) identified and no clinically significant changes in vital signs, electrocardiograms (ECGs), or hematology or biochemistry parameters in any clinical studies. A statistically significant reduction in Lp(a) levels from baseline was also observed at the 10 mg obicetrapib dose level. Therefore, the present study will utilize a dose of 10 mg obicetrapib in participants with ASCVD who are not adequately controlled despite maximally tolerated lipid-modifying therapies.

### 1.5 Risk/Benefit

The primary pharmacology in in vitro, ex vivo, and in vivo studies have demonstrated that obicetrapib has the ability to inhibit CETP, decrease LDL-C levels, increase HDL-C levels, and importantly, reduce the number of atherogenic ApoB-containing particles in a way that is useful in the treatment of dyslipidemia.

The safety pharmacology studies have demonstrated that obicetrapib has no adverse effect on critical physiological systems (eg, central nervous system, respiratory system, gastric emptying, urinary tract, and steroidal hormonal production [including aldosterone levels]) at doses up to 300 mg/kg in rats.

In clinical studies in patients, obicetrapib was also well tolerated after daily dosing of 10 mg for 12 weeks, both alone and in combination with 2 different statins. There were no dose-related AEs identified and no clinically significant changes in vital signs, ECGs, or hematology or biochemistry parameters in any clinical studies.

### 2 Study Objectives

### 2.1 Primary Objective

The primary objective of this study is to evaluate the effect of obicetrapib 10 mg+ezetimibe 10 mg combination therapy compared with placebo, when used as an adjunct to high-intensity statin therapy, on LDL-C at Day 84.

### 2.2 Secondary Objectives

The secondary objectives of this study include the following, in hierarchical order:
To evaluate the effect of obicetrapib 10 mg monotherapy compared with placebo, when used as an adjunct to high-intensity statin therapy, on LDL-C at Day 84;
To evaluate the effect of obicetrapib 10 mg+ezetimibe 10 mg combination therapy compared with placebo, when used as an adjunct to high-intensity statin therapy, on ApoB at Day 84; and
To evaluate the effect of obicetrapib 10 mg monotherapy compared with placebo, when used as an adjunct to high-intensity statin therapy, on ApoB at Day 84.

2.3 Exploratory Objectives

The exploratory objectives of this study include the following:
To evaluate the effect of obicetrapib 10 mg+ezetimibe 10 mg combination therapy and obicetrapib 10 mg monotherapy, when used as an adjunct to high-intensity statin therapy, on non-high-density lipoprotein cholesterol (non-HDL-C), very low-density lipoprotein cholesterol (VLDL-C), HDL-C, TG, ApoE, Lp(a), and HDL-ApoE at Day 84;
To evaluate the effect of obicetrapib 10 mg+ezetimibe 10 mg combination therapy and obicetrapib 10 mg monotherapy, when used as an adjunct to high-intensity statin therapy, on the proportion of participants achieving predefined LDL-C targets at Day 84;
To assess the mean trough plasma levels of obicetrapib 10 mg+ezetimibe 10 mg combination therapy and obicetrapib 10 mg monotherapy at steady state on Days 28, 84, and 112; and
To evaluate the safety and tolerability profiles of obicetrapib 10 mg+ezetimibe 10 mg combination therapy and obicetrapib 10 mg monotherapy, when used as an adjunct to high-intensity statin therapy, assessed by clinical laboratory values and incidence of AEs.

### 3 Study Description

### 3.1 Summary of Study Design

This study will be a placebo-controlled, double-blind, randomized, Phase 2 study to evaluate the efficacy, safety, and tolerability of obicetrapib 10 mg, both in combination with ezetimibe 10 mg and as monotherapy, as an adjunct to high-intensity statin therapy. This study will take place at approximately 20 sites in the United States.

### 3.1.1 Screening Period

At the Screening Visit, participants will be required to sign an informed consent form (ICF) before any study-related procedures are performed. After signing the ICF, participants will be assessed for study eligibility.

### 3.1.2 Treatment Period

Up to 2 weeks after the Screening Visit, participants will return to the site on Day 1 (Visit 2) and confirm study eligibility before being randomized and beginning treatment. Approximately 114 eligible participants (38 participants per treatment group) will be randomized in a 1:1:1 ratio to 1 of the following treatment groups:
Combination therapy: Obicetrapib 10 mg+ezetimibe 10 mg (administered as one 10 mg obicetrapib tablet and one 10 mg ezetimibe capsule);
Obicetrapib monotherapy: Obicetrapib 10 mg (administered as one 10 mg obicetrapib tablet and 1 placebo capsule); or.
Placebo (administered as 1 placebo tablet and 1 placebo capsule).

During the 12-week Treatment Period, the assigned study drugs will be administered by the participants orally with water, once daily on Day 1 to Day 84 at approximately the same time each morning. Participants will return to the site on Day 28 (±2 days), Day 84 (±2 days), and Day 112 (±2 days) for efficacy, safety, and pharmacokinetic (PK) assessments. Participants, Investigators, the Clinical Research Organization (CRO), and the Sponsor will be blinded to all lipid results from Day 1 (Visit 2) for the first participant until all enrolled participants complete the Day 84 (End of Treatment) visit or are withdrawn from the study, in order to protect blinding to treatment assignment.

### 3.1.3 Safety Follow-Up Period

Participants will return to the site for a Safety Follow-up Visit (Visit 5) approximately 4 weeks after the end of the Treatment Period for safety and PK assessments.

### 3.1.4 Coronavirus Disease 2019 Contingency Measures

In cases of COVID-19 limitations, it is the Investigator's responsibility to assure the safety of participants. If necessary, the Sponsor will implement and document mitigation strategies. At the Investigator's discretion, the study visit(s) can be conducted in-clinic or virtually. If conducted virtually, the visit will include alternative methods for safety, efficacy, and distribution/collection of study drug, including but not limited to phone/video contact, alternative location for biologic sample collection, alternative secure delivery of study drug, home health care (if available), and a secured way of transferring participant data from and to home health services and the site.

If these contingency measures occur, the Sponsor will document the changes made, communicate recommendations about such changes in a timely fashion to minimize or prevent disruptions to the study, and support sites in implementing these changes. Documentation of these cases and the site's management of participants should be recorded in the Investigator study files. In the absence of a COVID-19 impact, it is expected that Investigators and participants follow the protocol requirements as set forth.

### 3.2 Study Indication

The indication for this study is dyslipidemia.

### 4 Selection and Withdrawal of Participants

### 4.1 Inclusion Criteria

Participants who meet all of the following criteria will be eligible to participate in the study:
1. Understanding of the study procedures, willingness to adhere to the study schedules, and agreement to participate in the study by giving written informed consent prior to screening procedures;
2. Men or women 18 to 75 years of age, inclusive, at the Screening Visit; Women may be enrolled if all 3 of the following criteria are met:
   They are not pregnant;
   They are not breastfeeding; and
   They do not plan on becoming pregnant during the study.
Women of childbearing potential must have a negative urine pregnancy test at the
Screening Visit. Note: Women are not considered to be of childbearing potential if they.
meet 1 of the following criteria as documented by the Investigator:
   They have had a hysterectomy or tubal ligation at a minimum of 1 cycle prior to signing the ICF; or
   They are postmenopausal, defined as ≥1 year since their last menstrual period for women ≥55 years of age or ≥1 year since their last menstrual period and have a follicle-stimulating hormone (FSH) level in the postmenopausal range for women <55 years of age.
Women of childbearing potential must agree to use an effective method of avoiding pregnancy from screening to 90 days after the last visit. Men whose partners are of childbearing potential must agree to use an effective method of avoiding pregnancy from screening to 90 days after the last visit. Effective methods of avoiding pregnancy are contraceptive methods with a Pearl index of <1 used consistently and correctly (including implantable contraceptives, injectable contraceptives, oral contraceptives, transdermal contraceptives, intrauterine devices, diaphragm with spermicide, male or female condoms with spermicide, or cervical cap) or a sterile sexual partner.
3. Fasting LDL-C levels >70 mg/dL and TG levels <400 mg/dL at the Screening Visit; and
4. Currently receiving high-intensity statin therapy (atorvastatin 40 or 80 mg or rosuvastatin 20 or 40 mg) at a stable dose for 8 weeks prior to screening and intending to remain at the same stable dose throughout the study duration.

### 4.2 Exclusion Criteria

Participants who meet any of the following criteria will be excluded from participation in the study:
1. Body mass index □40 kg/m² at the Screening Visit;
2. Current clinically significant CV disease, including but not limited to:
   Major adverse CV event within 3 months prior to randomization; or Note: A major adverse CV event is defined as CV death, non-fatal myocardial infarction, non-fatal stroke, or non-elective coronary revascularization.
   New York Heart Association Functional Classification Class III or IV heart failure.
3. Glycosylated hemoglobin (HbA1c)□010% at the Screening Visit;
4. Uncontrolled hypertension, ie, sitting systolic blood pressure >160 mmHg and/or sitting diastolic blood pressure >90 mmHg. One retest will be allowed, at which point if the retest result is no longer exclusionary, the participant may be randomized;
5. Active muscle disease or persistent creatine kinase concentration >3×the upper limit of normal (ULN). One retest will be allowed after 1 week to verify the result, at which point if the retest result is no longer exclusionary, the participant may be randomized;
6. Estimated glomerular filtration rate <60 mL/min, calculated using the Chronic Kidney Disease Epidemiology Collaboration equation;
7. Hepatic dysfunction as evidenced by any laboratory abnormality as follows: gamma-glutamyl transferase, alanine aminotransferase, or aspartate aminotransferase >2×ULN, or total bilirubin >1.5×ULN;
8. Anemia, defined as hemoglobin concentration <11 g/dL for men and hemoglobin concentration <9 g/dL for women;
9. History of malignancy within 5 years prior to screening, with the exception of non-melanoma skin cancers;
10. History of alcohol and/or drug abuse within 5 years prior to screening;
11. Treatment with other investigational products or devices within 30 days or 5 half-lives, whichever is longer, prior to screening;
12. History of participation in any clinical trial evaluating obicetrapib;
13. Treatment with any PCSK9 inhibitor within 10 weeks prior to randomization or bempedoic acid within 2 weeks prior to randomization;
14. Evidence of any other clinically significant, non-cardiac disease or condition that, in the opinion of the Investigator, would preclude participant in the study; or
15. Known CETP inhibitor allergy or intolerance.

### 4.3 Retesting

If laboratory abnormalities during screening are considered by the Investigator to be transient, then the laboratory tests may be repeated once during screening. The Investigator's rationale for be randomized.

### 4.4 Rescreening

A participant who is screened and does not meet the study eligibility criteria may be considered for rescreening upon Sponsor and/or Medical Monitor consultation and approval. Rescreened participants will be assigned a new participant number. Rescreening should occur no less than 5 days after the last Screening Visit.

### 4.5 Withdrawal Criteria

Participation in this study may be discontinued for any of the following reasons:
The participant withdraws consent or requests discontinuation from the study for any reason;
Occurrence of any medical condition or circumstance that exposes the participant to substantial risk and/or does not allow the participant to adhere to the requirements of the protocol;
Any serious AE (SAE), clinically significant AE, severe laboratory abnormality, intercurrent illness, or other medical condition, which indicates to the Investigator that continued participation is not in the best interest of the participant;
Pregnancy;
Requirement of prohibited concomitant medication;
Participant failure to comply with protocol requirements or study-related procedures; or Termination of the study by the Sponsor or the regulatory authority.

Unless the participant withdraws consent, participants who discontinue study drug early should be encouraged to complete the full panel of assessments scheduled for the Early Termination Visit promptly and complete a Safety Follow-up Visit (Visit 5) 4 weeks after the last dose of study drug. PK samples will not be collected during the Safety Follow-up Visit for participants who discontinue study drug early without withdrawing consent or for participants who withdraw prematurely from the study. The reason for participant withdrawal must be documented in the electronic case report form (eCRF).

In the case of a participant lost to follow-up, attempts to contact the participant must be made and documented in the participant's medical records.

Withdrawn participants will not be replaced.

### 5 Study Treatments

### 5.1 Treatment Groups

Participants will be randomized in a 1:1:1 ratio to 1 of the following treatment groups:
Combination therapy: Obicetrapib 10 mg+ezetimibe 10 mg (administered as one 10 mg obicetrapib tablet and one 10 mg ezetimibe capsule);
Obicetrapib monotherapy: Obicetrapib 10 mg (administered as one 10 mg obicetrapib tablet and 1 placebo capsule); or.
Placebo (administered as 1 placebo tablet and 1 placebo capsule).

### 5.2 Rationale for Dosing

In clinical studies in healthy volunteers, obicetrapib was generally well tolerated in single doses up to 150 mg and multiple doses up to 25 mg/day for 21 days. In clinical studies in patients, obicetrapib was also well tolerated after daily dosing of 10 mg for 12 weeks, both alone and in combination with 2 different statins. Near maximal effects were observed with the 10 mg obicetrapib dose. At this dose level, CETP activity and concentrations were reduced, HDL-C levels were increased, and LDL-C levels were decreased. There were no dose-related AEs identified and no clinically significant changes in vital signs, ECGs, or hematology or biochemistry parameters in any clinical studies. A statistically significant reduction in Lp(a) levels from baseline was also observed at the 10 mg obicetrapib dose level. Therefore, the present study will utilize a dose of 10 mg obicetrapib in participants with ASCVD who are not adequately controlled despite maximally tolerated lipid-modifying therapies. The ezetimibe dose of 10 mg is the current FDA-approved dose.

### 5.3 Randomization and Blinding

Participants who meet all eligibility criteria will be randomized into the study. Participants will be randomized in a 1:1:1 ratio to receive combination therapy (obicetrapib 10 mg+ezetimibe 10 mg [administered as one 10 mg obicetrapib tablet and one 10 mg ezetimibe capsule]), obicetrapib monotherapy (obicetrapib 10 mg [administered as one 10 mg obicetrapib tablet and 1 placebo capsule]); or placebo (administered as 1 placebo tablet and 1 placebo capsule).

At randomization, participants will be stratified according to their Screening Visit LDL-C level (≥100 or <100 mg/dL). An automated interactive response technology (IRT) system will be used to assign the participant to 1 of the 3 treatment groups.

Participants, Investigators, the CRO, and the Sponsor will be blinded to all lipid results from Day 1 (Visit 2) for the first participant until all enrolled participants complete the Day 84 (End of Treatment) visit or are withdrawn from the study, in order to protect blinding to treatment assignment.

### 5.5 Drug Supplies

### 5.5.1 Formulation and Packaging

The study drugs consist of 10 mg obicetrapib tablets or matching placebo tablets and over-encapsulated 10 mg ezetimibe tablets or matching placebo capsules. All study drugs are manufactured in accordance with International Council for Harmonisation (ICH) current Good Manufacturing Practice.

Obicetrapib tablets are round, white film-coated tablets, with no identifying markings, containing 10 mg of obicetrapib calcium drug substance. The excipients present in the tablet cores are microcrystalline cellulose, mannitol, sodium starch glycollate, colloidal silicon dioxide, and magnesium stearate. A commercially available film-coating formula (Opadry II white, ex Colorcon) is applied to the cores.

Placebo tablets for obicetrapib are matching round, white film-coated tablets, with no identifying markings. The excipients present in the tablet cores are microcrystalline cellulose, mannitol, sodium starch glycollate, colloidal silicon dioxide, and magnesium stearate. A commercially available film-coating formula (Opadry II white, ex Colorcon) is applied to the cores.

Ezetimibe capsules are 10 mg ezetimibe tablets filled into capsule shells, 1 tablet per capsule. Each capsule also contains an excipient material, common to the tablets, as a filler to prevent the tablet from rattling in the capsule shell.

Placebo capsules to match the ezetimibe capsules are the identical capsule shells filled with the excipient filler material only (no tablets).

Obicetrapib and placebo tablets and ezetimibe and placebo capsules will be packaged into kits providing the 2 study drugs for each treatment group. The kits will be clearly labelled to indicate which tablets/capsules to use on each day. Each kit will provide a sufficient supply for 28 days of dosing, with enough for an extra 4 days of dosing in case the participant needs to postpone the next visit. The shelf-life will be assigned based on the stability of the individual study drugs and will not be greater than the expiry date of the input ezetimibe tablets. The kits will be stored below 25° C.

The physical, chemical, and pharmaceutical formulation properties and characteristics of the obicetrapib tablets are described in the Investigator's Brochure. All study drugs will be labelled in accordance with all applicable local regulatory requirements.

### 5.5.3 Study Drug Administration

Study drugs (1 tablet and 1 capsule) will be administered by the participant orally with water, once daily on Day 1 to Day 84 at approximately the same time each morning. On days with visits scheduled, study drugs should be administered with water following all fasted blood samples. At Visits 3 and 4, participants will dose from the kit received at the previous visit (Visits 2 and 3, respectively).

If a participant forgets to take study drug on a given day, they should take the next dose as normal and should not take a double dose to make up for the forgotten dose.

### 5.5.4 Treatment Compliance

Compliance to the study drug regimen will be evaluated by counting unused tablets and capsules. Participants will be instructed to bring all unused study drugs to the site at Visits 3 and 4. During the Treatment Period, if compliance is not between 80% and 120%, inclusive, the participant will be counselled about the importance of compliance to the regimen. If the limits are exceeded at 2 consecutive visits, a decision will be made by the Investigator and Sponsor as to whether the participant should be withdrawn from the study.

### 5.5.5 Storage and Accountability

All study drugs must be stored below 25° C. (77° F.) in a secure area with access limited to the Investigator and authorized site personnel. In accordance with regulatory requirements, the Investigator or designated site personnel must document the amount of study drug dispensed and/or administered to participants, the amount returned by participants, and the amount received from and returned to the Sponsor (or representative) when applicable. Study drug accountability records must be maintained throughout the course of the study. The accountability unit for this study is a tablet or capsule. Discrepancies are to be reconciled or resolved. Procedures for final disposition of unused study drug will be provided in the appropriate study manual.

5.6 Prior and Concomitant Medications and/or Procedures
5.6.1 Excluded Medications and/or Procedures

Participants must not receive treatment with other investigational products or devices within 30 days or 5 half-lives, whichever is longer, prior to screening. Participants must abstain from taking any PCSK9 inhibitor within 10 weeks prior to randomization or bempedoic acid within 2 weeks prior to randomization.

5.6.2 Allowed Medications and/or Procedures

Participants must be currently receiving high-intensity statin therapy (atorvastatin 40 or 80 mg or rosuvastatin 20 or 40 mg) at a stable dose for 8 weeks prior to screening and intending to remain at the same stable dose throughout the study duration.

5.6.3 Documentation of Prior and Concomitant Medication Use Medications used within 28 days prior to the Screening Visit will be recorded. Any medications administered in addition to the study drugs, whether allowed per the protocol or not, must be documented on the concomitant medication eCRF.

### 7 Efficacy and Pharmacokinetic Assessments

### 7.1 Efficacy Assessments

The primary efficacy endpoint is the percent change from Day 1 to Day 84 in LDL-C for the obicetrapib 10 mg+ezetimibe 10 mg combination treatment group compared with the placebo group.

The secondary efficacy endpoints include the following, in hierarchical order:
Percent change from Day 1 to Day 84 in LDL-C for the obicetrapib 10 mg monotherapy.
treatment group compared with the placebo group;
Percent change from Day 1 to Day 84 in ApoB for the obicetrapib 10 mg+ezetimibe 10 mg
combination treatment group compared with the placebo group; and
Percent change from Day 1 to Day 84 in ApoB for the obicetrapib 10 mg monotherapy treatment group compared with the placebo group.

The exploratory efficacy endpoints include the following:
Percent change from Day 1 to Day 84 in non-HDL-C, VLDL-C, HDL-C, TG, ApoE, Lp(a), and HDL-ApoE for the obicetrapib 10 mg+ezetimibe 10 mg combination treatment group compared with the placebo group and for the obicetrapib 10 mg monotherapy treatment group compared with the placebo group; and
Proportion of participants at Day 84 that achieve LDL-C <2.6 mmol/L (<100 mg/dL), LDL-C <1.8 mmol/L (<70 mg/dL), and LDL-C <1.3 mmol/L (<50 mg/dL) for the obicetrapib 10 mg+ezetimibe 10 mg combination treatment group compared with the placebo group and for the obicetrapib 10 mg monotherapy treatment group compared with the placebo group.

Blood samples for the lipid profile must be obtained under fasting conditions (ie, after the participant has fasted for approximately 10 hours). For the purposes of this study, fasting will be defined as nothing by mouth except water and any essential medications. If a participant is not fasting, the Investigator should reschedule the visit as soon as possible. LDL-C level will be calculated using the Friedewald equation unless TG≥400 mg/dL or LDL-C ≤50 mg/dL; in both cases, LDL-C level will be measured directly by preparative ultracentrifugation, also referred to as beta quantification. Additionally, LDL-C will be measured by preparative ultracentrifugation, also referred to as beta quantification, at baseline (Visit 2) and at the end of the 12-week Treatment Period (Visit 4) for all participants.

### 7.2 Pharmacokinetic Assessments

Plasma obicetrapib concentrations, both in combination with ezetimibe and as monotherapy, will be assessed at the scheduled PK collection times as indicated in Appendix A. At visits with dosing scheduled (ie, Visits 2, 3, and 4), PK samples will be collected with fasting laboratory samples (pre-dose). At Visits 3 and 4, the time between the last dose of study drug and PK sampling will be >24 hours. The subsequent post-dose PK sample at Visit 5 should be collected at approximately the same time as the previous visits.

### 8 Safety Assessments

The safety and tolerability profile of obicetrapib 10 mg, both in combination with ezetimibe 10 mg and as monotherapy, will be assessed by clinical laboratory assessments (chemistry and hematology), vital signs, physical examinations, and the incidence of AEs.

### 8.1 Adverse Events

An AE is defined as any untoward medical occurrence in a clinical investigation participant administered a pharmaceutical product, which does not necessarily have a causal relationship with this treatment. An AE can therefore be any unfavorable and/or unintended sign (including an abnormal laboratory finding), symptom, or disease temporally associated with the use of an investigational medicinal product, whether or not related to the investigational medicinal product. All AEs, including observed or volunteered problems, complaints, or symptoms, are to be recorded on the appropriate eCRF.

AEs, which include clinical laboratory assessment variables, will be monitored and documented from the time of first dose of study treatment until completion of Visit 5. Participants should be instructed to report any AE that they experience to the Investigator, whether or not they think the event is due to study drug. Beginning at the date of the first dose of study treatment, Investigators should make an assessment for AEs at each visit and record the event on the appropriate AE eCRF.

Wherever possible, a specific disease or syndrome rather than individual associated signs and symptoms should be identified by the Investigator and recorded on the eCRF. However, if an observed or reported sign or symptom is not considered a component of a specific disease or syndrome by the Investigator, it should be recorded as a separate AE on the eCRF. Additionally, the condition that led to a medical or surgical procedure (eg, surgery, endoscopy, tooth extraction, or transfusion) should be recorded as an AE, not the procedure itself.

Any medical condition already present at the date of the first dose of study treatment should be recorded as medical history and not be reported as an AE unless the medical condition or signs or symptoms present at baseline changes in severity, frequency, or seriousness at any time during the study. In this case, it should be reported as an AE.

Clinically significant abnormal laboratory or other examination findings (eg, ECG) that are detected during the study or are present at the date of the first dose of study treatment and significantly worsen during the study should be reported as AEs, as described below. The Investigator will exercise his or her medical and scientific judgment in deciding whether an abnormal laboratory finding or other abnormal assessment is clinically significant. Clinically significant abnormal laboratory values occurring during the study will be followed until repeat tests return to normal, stabilize, or are no longer clinically significant. Abnormal test results that are determined to be an error should not be reported as an AE. Laboratory abnormalities or other abnormal clinical findings (eg, ECG abnormalities) should be reported as an AE if any of the following are applicable:
If an intervention is required as a result of the abnormality;
If action taken with the study drug is required as a result of the abnormality; or.
Based on the clinical judgment of the Investigator.

8.1.1 Adverse (Drug) Reaction

All noxious and unintended responses to a medicinal product related to any dose should be considered an adverse drug reaction. “Responses” to a medicinal product means that a causal relationship between a medicinal product and an AE is at least a reasonable possibility, ie, the relationship cannot be ruled out.

### 8.1.2 Unexpected Adverse Drug Reaction

An Unexpected Adverse Drug Reaction is defined as an adverse reaction, the nature or severity of which is not consistent with the applicable product information.

### 8.1.3 Assessment of Adverse Events by the Investigator

The Investigator will assess the severity (intensity) of each AE as mild, moderate, or severe, and will also categorize each AE as to its potential relationship to study drug using the categories of yes or no.

### Assessment of Severity

Mild—An event that is easily tolerated and generally not interfering with normal daily activities.

Moderate—An event that is sufficiently discomforting to interfere with normal daily activities.

Severe—An event that is incapacitating with inability to work or perform normal daily activities.

### Causality Assessment

The relationship of an AE to the administration of the study drug is to be assessed according to the following definitions:

No (unrelated, not related, unlikely to be related)—The time course between the administration of study drug and the occurrence or worsening of the AE rules out a causal relationship and another cause (concomitant drugs, therapies, complications, etc) is suspected.

Yes (possibly, probably, or definitely related)—The time course between the administration of study drug and the occurrence or worsening of the AE is consistent with a causal relationship and no other cause (concomitant drugs, therapies, complications, etc) can be identified.

The definition implies a reasonable possibility of a causal relationship between the event and the study drug. This means that there are facts (evidence) or arguments to suggest a causal relationship.

The following factors should also be considered:
The temporal sequence from study drug administration—
   The event should occur after the study drug is given. The length of time from study drug exposure to event should be evaluated in the clinical context of the event.
Underlying, concomitant, intercurrent diseases—
   Each report should be evaluated in the context of the natural history and course of the disease being treated and any other disease the participant may have.
Concomitant drug—
   The other drugs the participant is taking or the treatment the participant receives should be examined to determine whether any of them might be recognized to cause the event in question.
Known response pattern for this class of study drug—
   Clinical and/or preclinical data may indicate whether a particular response is likely to be a class effect.
Exposure to physical and/or mental stresses—
   The exposure to stress might induce adverse changes in the recipient and provide a logical and better explanation for the event.
The pharmacology and PK of the study drug—
   The known pharmacologic properties (absorption, distribution, metabolism, and excretion) of the study drug should be considered.

### 8.2 Serious Adverse Events

An AE or adverse reaction is considered serious if, in the view of either the Investigator or Sponsor, it results in any of the following outcomes:
Death;
A life-threatening AE;
   Note: An AE or adverse reaction is considered “life-threatening” if, in view of either the Investigator or Sponsor, its occurrence places the participant at immediate risk of death. It does not include an event that, had it occurred in a more severe form, might have caused death.
Requires hospitalization or prolongation of existing hospitalizations;
   Note: Any hospital admission with at least 1 overnight stay will be considered an inpatient hospitalization. An emergency room or urgent care visit without hospital admission will not be recorded as a SAE under this criterion, nor will hospitalization for a procedure scheduled or planned before signing of informed consent, or elective treatment of a pre-existing condition that did not worsen from baseline. However, unexpected complications and/or prolongation of hospitalization that occur during elective surgery should be recorded as AEs and assessed for seriousness. Admission to the hospital for social or situational reasons (ie, no place to stay, live too far away to come for hospital visits, respite care) will not be considered inpatient hospitalizations.
A persistent or significant disability/incapacity or substantial disruption of the ability to conduct normal life functions;
A congenital anomaly/birth defect; or
An important medical event.
   Note: Important medical events that do not meet any of the above criteria may be considered an SAE when, based upon appropriate medical judgment, they may jeopardize the participant and may require medical or surgical intervention to prevent 1 of the outcomes listed above. Examples of such medical events include allergic bronchospasm requiring intensive treatment in an emergency room or at home, blood dyscrasias or convulsions that do not result in inpatient hospitalizations, or the development of drug dependency.

### 9 Statistics

### 9.1 Analysis Populations

The Intent-to-Treat (ITT) Population will include all participants randomized into the study. Treatment classification will be based on the randomized treatment.

The Modified ITT (mITT) Population will include all participants in the ITT Population who receive at least 1 dose of any study drug and have a baseline value for the LDL-C assessment. Any efficacy measurement obtained during the Safety Follow-up Visit (Visit 5) after a participant permanently discontinues the study drug or after a participant receives an excluded medication and/or procedure will be removed from the mITT analysis. Treatment classification will be based on the randomized treatment. The mITT Population will be used for the primary analysis of all efficacy endpoints.

The Per-Protocol (PP) Population will include all participants in the mITT Population who have a baseline value for the LDL-C assessment, have a Day 84 value for the LDL-C assessment, and who do not experience a major protocol deviation that could potentially impact the primary efficacy endpoint. The PP Population, along with the reason for exclusion, will be finalized prior to study unblinding.

The PK Population will include all participants in the mITT Population who have sufficient blood samples collected for valid estimation of PK parameters.

The Safety Population will include all participants who receive at least 1 dose of any study drug. Treatment classification will be based on the actual treatment received. The Safety Population will be the primary population used for the safety analyses.

### 10 Results

The topline results of the clinical study are summarized in the following tables 14.1.1.1-14.3.1.1.

TABLE 14.1.1.1
Disposition All Randomized Participants

| | | | Obicetrapib | | Obicetrapib | | | |
|---|---|---|---|---|---|---|---|---|
| | Placebo | | 10 mg | | 10 mg + Ezetimibe | | Total | |
| | (N = 40) | | (N = 39) | | 10 mg (N = 40) | | (N = 119) | |
| | n | (%) | n | (%) | n | (%) | n | (%) |
| Randomized | 40 | (100.0) | 39 | (100.0) | 40 | (100.0) | 119 | (100.0) |
| Completed treatment | 36 | (90.0) | 36 | (92.3) | 38 | (95.0) | 110 | (92.4) |
| Discontinued treatment | 4 | (10.0) | 3 | (7.7) | 2 | (5.0) | 9 | (7.6) |
| Primary reason for treatment | | | | | | | | |
| discontinuation: | | | | | | | | |
| ADVERSE EVENT | 2 | (5.0) | 2 | (5.1) | 1 | (2.5) | 5 | (4.2) |
| WITHDRAWAL BY SUBJECT | 1 | (2.5) | 0 | (0.0) | 1 | (2.5) | 2 | (1.7) |
| NON-COMPLIANCE WITH | 0 | (0.0) | 1 | (2.6) | 0 | (0.0) | 1 | (0.8) |
| STUDY DRUG | | | | | | | | |
| OTHER | 1 | (2.5) | 0 | (0.0) | 0 | (0.0) | 1 | (0.8) |
| Completed the study (with PK) | 35 | (87.5) | 37 | (94.9) | 39 | (97.5) | 111 | (93.3) |
| Discontinued study early | 5 | (12.5) | 2 | (5.1) | 1 | (2.5) | 8 | (6.7) |
| Primary reason for early study | | | | | | | | |
| discontinuation: | | | | | | | | |
| WITHDRAWAL BY SUBJECT | 2 | (5.0) | 0 | (0.0) | 1 | (2.5) | 3 | (2.5) |
| ADVERSE EVENT | 1 | (2.5) | 1 | (2.6) | 0 | (0.0) | 2 | (1.7) |
| LOST TO FOLLOW-UP | 1 | (2.5) | 1 | (2.6) | 0 | (0.0) | 2 | (1.7) |
| OTHER | 1 | (2.5) | 0 | (0.0) | 0 | (0.0) | 1 | (0.8) |

Table 14.1.4.1 Demographic and Baseline Characteristics mITT Population

| | | | Obicetrapib | |
|---|---|---|---|---|
| | | Obicetrapib | 10 mg + | |
| | Placebo | 10 mg | Ezetimibe | Total |
| | (N = 40) | (N = 39) | 10 mg (N = 40) | (N = 119) |
| Age (years) | 40 | 39 | 40 | 119 |
| n | 60.6 | 63.5 | 63.8 | 62.6 |
| Mean | 8.46 | 7.19 | 8.56 | 8.17 |
| Standard deviation | 62.5 | 63.0 | 66.0 | 64.0 |
| Median | 45 | 47 | 40 | 40 |
| Minimum | 75 | 75 | 74 | 75 |
| Maximum | | | | |
| Sex, n (%) | | | | |
| Male | 26 (65.0) | 25 (64.1) | 24 (60.0) | 75 (63.0) |
| Female | 14 (35.0) | 14 (35.9) | 16 (40.0) | 44 (37.0) |
| Undifferentiated | 0 (0.0) | 0 (0.0) | 0 (0.0) | 0 (0.0) |
| Unknown | 0 (0.0) | 0 (0.0) | 0 (0.0) | 0 (0.0) |
| Childbearing Potential, | | | | |
| n (%) [1] | | | | |
| Yes | 2 (14.3) | 1 (7.1) | 0 (0.0) | 3 (6.8) |
| No | 12 (85.7) | 13 (92.9) | 16 (100.0) | 41 (93.2) |
| LDL-C Category, n (%) [2] | | | | |
| <100 mg/dL | 20 (50.0) | 19 (48.7) | 20 (50.0) | 59 (49.6.0) |
| >=100 mg/dL | 20 (50.0) | 20 (51.3) | 20 (50.0) | 60 (50.4) |
| Ethnicity, n (%) Hispanic or | | | | |
| Latino | | | | |
| Hispanic or Latino | 4 (10.0) | 7 (17.9) | 7 (17.5) | 18 (15.1) |
| Not Hispanic or Latino | 35 (87.5) | 32 (82.1) | 33 (82.5) | 100 (84.0) |
| Not Reported | 1 (2.5) | 0 (0.0) | 0 (0.0) | 1 (0.8) |
| Unknown | 0 (0.0) | 0 (0.0) | 0 (0.0) | 0 (0.0) |
| Race, n (%) | | | | |
| White | 30 (75.0) | 34 (87.2) | 36 (90.0) | 100 (84.0) |
| Black or African American | 9 (22.5) | 5 (12.8) | 3 (7.5) | 17 (14.3) |
| Asian | 1 (2.5) | 0 (0.0) | 1 (2.5) | 2 (1.7) |
| American Indian or Alaska | 0 (0.0) | 0 (0.0) | 0 (0.0) | 0 (0.0) |
| Native | | | | |
| Native Hawaiian or Other | 0 (0.0) | 0 (0.0) | 0 (0.0) | 0 (0.0) |
| Pacific Islander | | | | |
| Other | 0 (0.0) | 0 (0.0) | 0 (0.0) | 0 (0.0) |
| Height (cm) | | | | |
| n | 40 | 39 | 40 | 119 |
| Mean | 172.4 | 169.1 | 169.0 | 170.2 |
| Standard deviation | 10.51 | 9.91 | 10.07 | 10.21 |
| Median | 174.0 | 170.0 | 169.0 | 171.0 |
| Minimum | 142 | 150 | 147 | 142 |
| Maximum | 196 | 185 | 188 | 196 |
| Weight (kg) | | | | |
| n | 40 | 39 | 40 | 119 |
| Mean | 92.13 | 88.26 | 90.66 | 90.37 |
| Standard deviation | 19.141 | 16.924 | 19.265 | 18.400 |
| Median | 88.90 | 92.00 | 91.60 | 91.40 |
| Minimum | 48.6 | 50.9 | 56.8 | 48.6 |
| Maximum | 150.7 | 116.1 | 129.9 | 150.7 |
| Body Mass Index (kg/m²) | | | | |
| n | 40 | 39 | 40 | 119 |
| Mean | 30.75 | 30.72 | 31.57 | 31.02 |
| Standard deviation | 4.286 | 4.611 | 5.230 | 4.703 |
| Median | 30.35 | 31.20 | 31.75 | 31.20 |
| Minimum | 23.3 | 19.6 | 20.8 | 19.6 |
| Maximum | 39.9 | 39.6 | 39.2 | 39.9 |

Table 14.2.1.1.1 Summary of LDL-C
(mg/dL) mITT Population

| | | | Obicetrapib |
|---|---|---|---|
| | | | 10 mg + |
| | | Obicetrapib | Ezetimibe |
| Visit | Placebo | 10 mg | 10 mg |
| Statistic | (N = 40) | (N = 39) | (N = 40) |
| Baseline | | | |
| n | 40 | 39 | 40 |
| Mean | 99.1 | 103.5 | 102.5 |
| Standard deviation | 27.06 | 35.34 | 27.64 |
| Median | 95.5 | 97.0 | 100.0 |
| Minimum | 60 | 35 | 62 |
| Maximum | 211 | 189 | 156 |
| Day 28 | | | |
| n | 38 | 39 | 39 |
| Mean | 93.3 | 70.3 | 47.4 |
| Standard deviation | 22.03 | 41.15 | 33.86 |
| Median | 91.0 | 56.0 | 41.0 |
| Minimum | 52 | 16 | 9 |
| Maximum | 170 | 169 | 197 |
| Percent Change from | | | |
| Baseline to Day 28 (%) | | | |
| n′ | 38 | 39 | 39 |
| Mean | −3.58 | −32.21 | −54.87 |
| Standard deviation | 19.221 | 32.446 | 23.202 |
| Median | −5.80 | −41.70 | −56.90 |
| Minimum | −44.5 | −82.0 | −89.0 |
| Maximum | 72.1 | 70.3 | 26.3 |
| Change from Baseline | | | |
| to Day 28 | | | |
| n′ | 38 | 39 | 39 |
| Mean | −5.8 | −33.2 | −54.1 |
| Standard deviation | 20.92 | 34.69 | 28.60 |
| Median | −5.0 | −35.0 | −54.0 |
| Minimum | −94 | −121 | −117 |
| Maximum | 44 | 64 | 41 |
| Day 84 | | | |
| n | 36 | 38 | 38 |
| Mean | 95.9 | 82.7 | 53.0 |
| Standard deviation | 29.93 | 49.21 | 37.08 |
| Median | 88.0 | 65.0 | 45.0 |
| Minimum | 55 | 21 | 15 |
| Maximum | 188 | 196 | 162 |
| Percent Change from | | | |
| Baseline to Day 84 (%) | | | |
| n′ | 36 | 38 | 38 |
| Mean | −0.83 | −18.92 | −49.24 |
| Standard deviation | 24.514 | 47.339 | 27.343 |
| Median | −6.35 | −32.35 | −58.85 |
| Minimum | −36.4 | −78.4 | −83.7 |
| Maximum | 96.7 | 161.5 | 36.1 |
| Change from Baseline | | | |
| to Day 84 | | | |
| n′ | 36 | 38 | 38 |
| Mean | −2.5 | −21.3 | −47.1 |
| Standard deviation | 19.54 | 42.51 | 30.07 |
| Median | −5.5 | −29.5 | −50.5 |
| Minimum | −36 | −112 | −113 |
| Maximum | 59 | 98 | 43 |
| | | | Obicetrapib |
| | | | 10 mg + |
| | | Obicetrapib | Ezetimibe |
| | Placebo | 10 mg | 10 mg |
| Visit Statistic | (N = 40) | (N = 26) | (N = 31) |

Table 14.2.1.1.1a Summary of LDL-C
(mg/dL) Exploratory Population

| | | | |
|---|---|---|---|
| Baseline | | | |
| n | 40 | 26 | 31 |
| Mean | 99.1 | 100.6 | 96.0 |
| Standard deviation | 27.06 | 33.65 | 25.62 |
| Median | 95.5 | 100.0 | 87.0 |
| Minimum | 60 | 35 | 62 |
| Maximum | 211 | 189 | 152 |
| Day 28 | | | |
| n | 38 | 26 | 31 |
| Mean | 93.3 | 57.6 | 39.0 |
| Standard deviation | 22.03 | 28.77 | 22.72 |
| Median | 91.0 | 51.5 | 39.0 |
| Minimum | 52 | 16 | 9 |
| Maximum | 170 | 136 | 105 |
| Percent Change from | | | |
| Baseline to Day 28 (%) | | | |
| n′ | 38 | 26 | 31 |
| Mean | −3.58 | −43.59 | −59.79 |
| Standard deviation | 19.221 | 16.890 | 18.675 |
| Median | −5.80 | −43.90 | −61.40 |
| Minimum | −44.5 | −82.0 | −89.0 |
| Maximum | 72.1 | −10.4 | −21.7 |
| Change from Baseline | | | |
| to Day 28 | | | |
| n′ | 38 | 26 | 31 |
| Mean | −5.8 | −43.0 | −57.0 |
| Standard deviation | 20.92 | 20.45 | 23.19 |
| Median | −5.0 | −45.0 | −55.0 |
| Minimum | −94 | −87 | −109 |
| Maximum | 44 | −11 | −21 |
| Day 84 | | | |
| n | 36 | 26 | 31 |
| Mean | 95.9 | 60.7 | 39.7 |
| Standard deviation | 29.93 | 31.99 | 19.29 |
| Median | 88.0 | 55.5 | 39.0 |
| Minimum | 55 | 21 | 15 |
| Maximum | 188 | 148 | 96 |
| Percent Change from | | | |
| Baseline to Day 84 (%) | | | |
| n′ | 36 | 26 | 31 |
| Mean | −0.83 | −39.35 | −59.07 |
| Standard deviation | 24.514 | 22.602 | 14.814 |
| Median | −6.35 | −43.50 | −63.40 |
| Minimum | −36.4 | −78.4 | −83.7 |
| Maximum | 96.7 | 22.6 | −29.7 |
| Change from Baseline | | | |
| to Day 84 | | | |
| n′ | 36 | 26 | 31 |
| Mean | −2.5 | −39.9 | −56.4 |
| Standard deviation | 19.54 | 28.48 | 19.84 |
| Median | −5.5 | −38.5 | −54.0 |
| Minimum | −36 | −112 | −113 |
| Maximum | 59 | 24 | −22 |

Table 14.2.2.1 Summary of ApoB
(mg/dL)mITT Population

| | | | |
|---|---|---|---|
| Baseline | | | |
| n | 40 | 39 | 40 |
| Mean | 89.8 | 92.3 | 91.0 |
| Standard deviation | 18.74 | 22.95 | 19.05 |
| Median | 89.0 | 86.0 | 88.0 |
| Minimum | 52 | 33 | 56 |
| Maximum | 146 | 154 | 138 |
| Day 28 | | | |
| n | 38 | 39 | 39 |
| Mean | 88.6 | 73.4 | 61.0 |
| Standard deviation | 19.62 | 25.49 | 22.15 |
| Median | 90.5 | 66.0 | 57.0 |
| Minimum | 59 | 32 | 35 |
| Maximum | 142 | 140 | 152 |
| Percent Change from | | | |
| Baseline to Day 28 (%) | | | |
| n′ | 38 | 39 | 39 |
| Mean | −0.14 | −19.16 | −32.32 |
| Standard deviation | 16.915 | 23.542 | 18.124 |
| Median | −1.95 | −23.10 | −31.20 |
| Minimum | −37.0 | −62.0 | −63.6 |
| Maximum | 48.1 | 64.9 | 24.6 |
| Change from Baseline | | | |
| to Day 28 | | | |
| n′ | 38 | 39 | 39 |
| Mean | −1.1 | −18.9 | −29.3 |
| Standard deviation | 15.29 | 23.51 | 19.05 |
| Median | −1.5 | −20.0 | −27.0 |
| Minimum | −54 | −85 | −65 |
| Maximum | 39 | 50 | 30 |
| Day 84 | | | |
| n | 36 | 38 | 38 |
| Mean | 88.4 | 82.4 | 63.7 |
| Standard deviation | 21.40 | 29.19 | 22.95 |
| Median | 83.5 | 72.0 | 58.5 |
| Minimum | 62 | 30 | 36 |
| Maximum | 159 | 147 | 147 |
| Percent Change from | | | |
| Baseline to Day 84 (%) | | | |
| n′ | 36 | 38 | 38 |
| Mean | 0.51 | −9.50 | −28.76 |
| Standard deviation | 19.784 | 31.352 | 18.804 |
| Median | −2.05 | −17.35 | −29.15 |
| Minimum | −30.9 | −44.8 | −54.3 |
| Maximum | 76.9 | 100.0 | 34.8 |
| Change from Baseline | | | |
| to Day 84 | | | |
| n′ | 36 | 38 | 38 |
| Mean | −0.7 | −10.4 | −25.7 |
| Standard deviation | 14.84 | 25.64 | 18.90 |
| Median | −1.5 | −14.5 | −27.0 |
| Minimum | −29 | −52 | −62 |
| Maximum | 40 | 67 | 31 |

Table 14.2.2.1 Summary of ApoB
(mg/dL)Exploratory Population

| | | | |
|---|---|---|---|
| Baseline | | | |
| n | 40 | 26 | 31 |
| Mean | 89.8 | 89.6 | 87.7 |
| Standard deviation | 18.74 | 20.90 | 17.50 |
| Median | 89.0 | 85.0 | 85.0 |
| Minimum | 52 | 33 | 56 |
| Maximum | 146 | 130 | 130 |
| Day 28 | | | |
| n | 38 | 26 | 31 |
| Mean | 88.6 | 65.4 | 56.5 |
| Standard deviation | 19.62 | 16.41 | 15.29 |
| Median | 90.5 | 64.5 | 54.0 |
| Minimum | 59 | 32 | 35 |
| Maximum | 142 | 110 | 93 |
| Percent Change from | | | |
| Baseline to Day 28 (%) | | | |
| n′ | 38 | 26 | 31 |
| Mean | −0.14 | −26.00 | −34.90 |
| Standard deviation | 16.915 | 11.866 | 15.344 |
| Median | −1.95 | −26.00 | −33.00 |
| Minimum | −37.0 | −47.4 | −58.7 |
| Maximum | 48.1 | −3.0 | −6.7 |
| Change from Baseline | | | |
| to Day 28 | | | |
| n′ | 38 | 26 | 31 |
| Mean | −1.1 | −24.2 | −31.3 |
| Standard deviation | 15.29 | 13.14 | 16.35 |
| Median | −1.5 | −23.0 | −29.0 |
| Minimum | −54 | −55 | −65 |
| Maximum | 39 | −1 | −5 |
| Day 84 | | | |
| n | 36 | 26 | 31 |
| Mean | 88.4 | 69.1 | 56.5 |
| Standard deviation | 21.40 | 18.81 | 11.84 |
| Median | 83.5 | 65.0 | 56.0 |
| Minimum | 62 | 30 | 36 |
| Maximum | 159 | 123 | 83 |
| Percent Change from | | | |
| Baseline to Day 84 (%) | | | |
| n′ | 36 | 26 | 31 |
| Mean | 0.51 | −21.73 | −34.66 |
| Standard deviation | 19.784 | 16.057 | 11.796 |
| Median | −2.05 | −24.20 | −34.40 |
| Minimum | −30.9 | −44.8 | −54.3 |
| Maximum | 76.9 | 27.1 | −14.7 |
| Change from Baseline | | | |
| to Day 84 | | | |
| n′ | 36 | 26 | 31 |
| Mean | −0.7 | −20.5 | −31.2 |
| Standard deviation | 14.84 | 16.17 | 13.78 |
| Median | −1.5 | −21.5 | −29.0 |
| Minimum | −29 | −52 | −62 |
| Maximum | 40 | 19 | −10 |

Table 14.2.8.1 Summary of Lp(a)
(nmol/L)mITT Population

| | | | |
|---|---|---|---|
| Baseline | | | |
| n | 40 | 39 | 40 |
| Mean | 122.72 | 99.39 | 81.50 |
| Standard deviation | 148.345 | 112.884 | 121.345 |
| Median | 47.05 | 42.30 | 22.05 |
| Minimum | 2.8 | 2.8 | 2.8 |
| Maximum | 551.6 | 387.4 | 488.7 |
| Day 84 | | | |
| n | 36 | 38 | 38 |
| Mean | 128.67 | 77.96 | 81.09 |
| Standard deviation | 148.907 | 97.118 | 134.908 |
| Median | 52.55 | 26.25 | 12.75 |
| Minimum | 3.7 | 2.8 | 2.8 |
| Maximum | 533.6 | 327.7 | 455.6 |
| Percent Change from | | | |
| Baseline to Day 84 (%) | | | |
| n′ | 36 | 38 | 38 |
| Mean | 7.51 | −15.16 | −19.26 |
| Standard deviation | 49.648 | 34.757 | 40.054 |
| Median | 2.20 | −11.95 | −13.30 |
| Minimum | −51.6 | −84.6 | −88.2 |
| Maximum | 281.1 | 103.4 | 39.3 |
| Change from Baseline | | | |
| to Day 84 | | | |
| n′ | 36 | 38 | 38 |
| Mean | −1.02 | −19.71 | −4.47 |
| Standard deviation | 23.724 | 34.519 | 46.368 |
| Median | 0.70 | −4.50 | −2.25 |
| Minimum | −109.4 | −148.8 | −154.8 |
| Maximum | 42.4 | 22.0 | 124.1 |

Table 14.2.8.1 Summary of Lp(a)
(nmol/L)Exploratory Population

| | | | |
|---|---|---|---|
| Baseline | | | |
| n | 40 | 26 | 31 |
| Mean | 122.72 | 102.40 | 93.78 |
| Standard deviation | 148.345 | 117.825 | 133.102 |
| Median | 47.05 | 36.05 | 22.80 |
| Minimum | 2.8 | 2.8 | 2.8 |
| Maximum | 551.6 | 387.4 | 488.7 |
| Day 84 | | | |
| n | 36 | 26 | 31 |
| Mean | 128.67 | 74.75 | 89.38 |
| Standard deviation | 148.907 | 97.756 | 146.282 |
| Median | 52.55 | 20.85 | 9.20 |
| Minimum | 3.7 | 2.8 | 2.8 |
| Maximum | 533.6 | 327.7 | 455.6 |
| Percent Change from | | | |
| Baseline to Day 84 (%) | | | |
| n′ | 36 | 26 | 31 |
| Mean | 7.51 | −24.44 | −19.09 |
| Standard deviation | 49.648 | 36.246 | 43.743 |
| Median | 2.20 | −22.75 | −8.00 |
| Minimum | −51.6 | −84.6 | −88.2 |
| Maximum | 281.1 | 103.4 | 39.3 |
| Change from Baseline | | | |
| to Day 84 | | | |
| n′ | 36 | 26 | 31 |
| Mean | −1.02 | −27.66 | −4.40 |
| Standard deviation | 23.724 | 37.784 | 51.453 |
| Median | 0.70 | −10.95 | −2.10 |
| Minimum | −109.4 | −148.8 | −154.8 |
| Maximum | 42.4 | 3.0 | 124.1 |

TABLE 14.3.1.1
Overview of Adverse Events
Safety Population

| | | | | Obicetrapib | | | Obicetrapib | | | | | |
|---|---|---|---|---|---|---|---|---|---|---|---|---|
| | Placebo | | | 10 mg | | | 10 mg + Ezetimibe | | | | | |
| | (N = 40) | | | (N = 39) | | | 10 mg (N = 40) | | | Total (N = 119) | | |
| Any adverse events (AEs) | n | (%) | e | n | (%) | e | n | (%) | e | n | (%) | e |
| Any treatment- | 16 | (40.0) | 21 | 8 | (20.5) | 20 | 11 | (27.5) | 17 | 35 | (29.5) | 58 |
| emergent AEs | | | | | | | | | | | | |
| (TEAEs) | | | | | | | | | | | | |
| Any TEAEs by | | | | | | | | | | | | |
| maximum severity | | | | | | | | | | | | |
| Mild | 16 | (40.0) | 21 | 8 | (20.5) | 20 | 11 | (27.5) | 17 | 35 | (29.5) | 58 |
| Moderate | 12 | (30.0) | | 3 | (7.7) | | 7 | (17.5) | | 22 | (18.5) | |
| Severe | 2 | (5.0) | | 4 | (10.3) | | 4 | (10.0) | | 10 | (8.4) | |
| Any study drug- | 2 | (5.0) | | 1 | (2.6) | | 0 | (0.0) | | 3 | (2.5) | |
| related TEAEs | | | | | | | | | | | | |
| Any study drug- | | | | | | | | | | | | |
| related TEAEs by | | | | | | | | | | | | |
| maximum severity | | | | | | | | | | | | |
| Mild | 2 | (5.0) | 6 | 4 | (10.3) | 4 | 5 | (12.5) | 6 | 11 | (9.2) | 16 |
| Moderate | 0 | (0.0) | | 2 | (5.1) | | 3 | (7.5) | | 5 | (4.2) | |
| Severe | 1 | (2.5) | | 2 | (5.1) | | 2 | (5.0) | | 5 | (4.2) | |
| Any TEAEs | 1 | (2.5) | | 0 | (0.0) | | 0 | (0.0) | | 1 | (0.8) | |
| leading to | | | | | | | | | | | | |
| discontinuation | | | | | | | | | | | | |
| of study drug | | | | | | | | | | | | |
| Any study | 2 | (5.0) | 6 | 2 | (5.1) | 5 | 1 | (2.5) | 1 | 5 | (4.2) | 12 |
| drug-related | | | | | | | | | | | | |
| TEAEs leading | | | | | | | | | | | | |
| to | | | | | | | | | | | | |
| discontinuation | | | | | | | | | | | | |
| of study drug | | | | | | | | | | | | |
| Any serious | 2 | (5.0) | 6 | 2 | (5.1) | 2 | 1 | (2.5) | 1 | 5 | (4.2) | 9 |
| TEAEs (TESAEs) | | | | | | | | | | | | |
| Any study drug- | 1 | (2.5) | 1 | 1 | (2.6) | 1 | 0 | (0.0) | 0 | 2 | (1.7) | 2 |
| related TESAEs | | | | | | | | | | | | |
| Any TEAEs | 0 | (0.0) | 0 | 0 | (0.0) | 0 | 0 | (0.0) | 0 | 0 | (0.0) | 0 |
| leading to death | | | | | | | | | | | | |
| Any TEAEs | 0 | (0.0) | 0 | 0 | (0.0) | 0 | 0 | (0.0) | 0 | 0 | (0.0) | 0 |
| leading to | | | | | | | | | | | | |
| discontinuation of | | | | | | | | | | | | |
| study drug | | | | | | | | | | | | |

### Example 11: Synthesis of Amorphous Obicetrapib Hemicalcium

The Examples in this section are offered by way of illustration, and not by way of limitation. The examples can represent only some embodiments, and it should be understood that the following examples are illustrative and not limiting. All substituents, unless otherwise specified, are as previously defined. The reagents and starting materials are readily available to one of ordinary skill in the art. The specific synthetic steps for each of the routes described may be combined in different ways, or in conjunction with steps from different schemes, to prepare the compounds described herein.

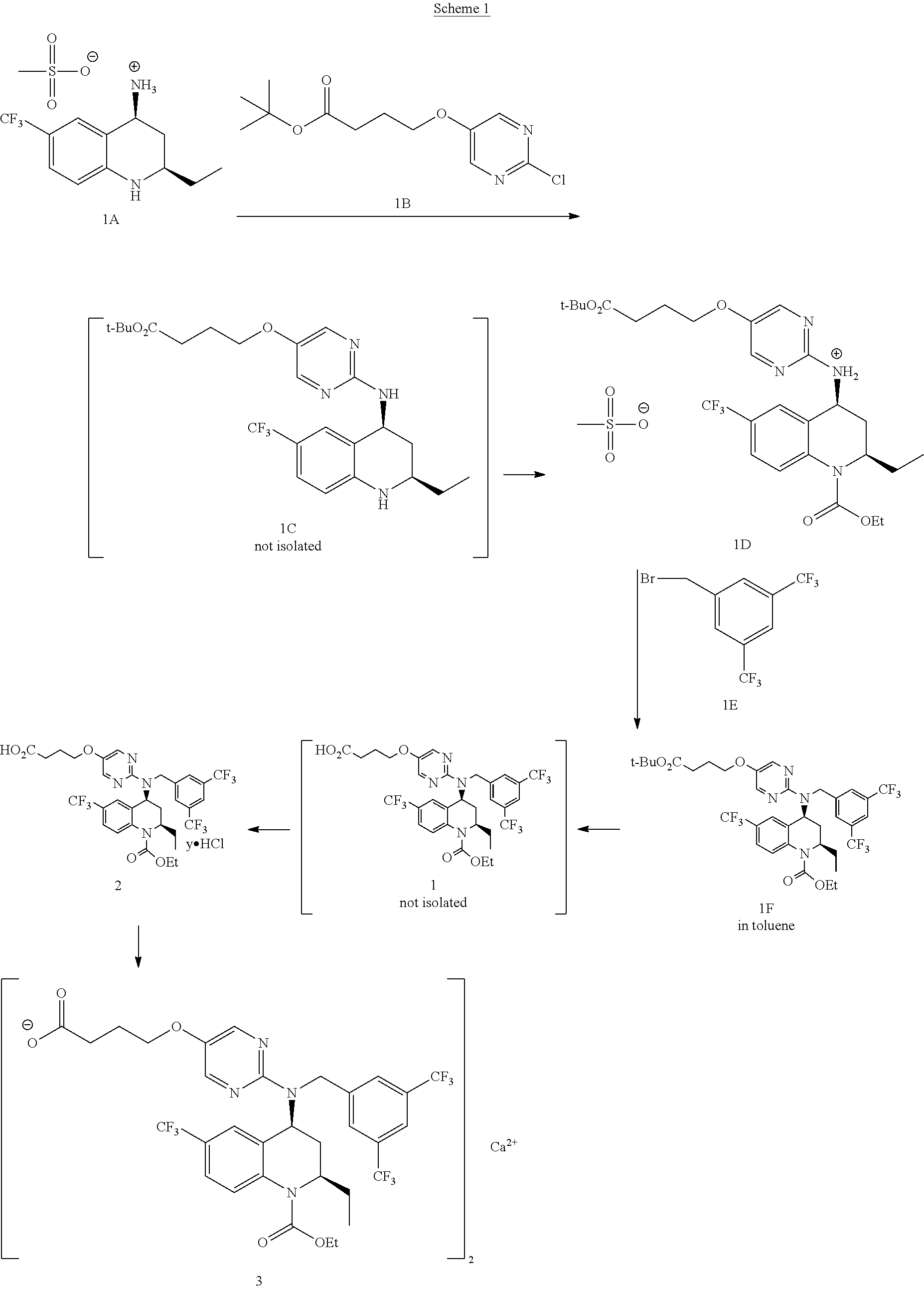

With reference to Scheme 1, amorphous obicetrapib hemicalcium (compound 3) was prepared in six chemical steps and three isolations from the mesylate salt of (2R,4S)-4-amino-2-ethyl-6-trifluoromethyl-3,4-dihydro-2H-quinoline (compound 1A), t-butyl-4-(2-chloropyrimidin-5-yloxy)-butyrate (compound 1B), and 3,5-bis(trifluoromethyl)benzyl bromide (compound 1E). Compound 1A was coupled with compound 1B through a palladium-catalyzed reaction to produce a solution of (2R,4S)-4-[5-(3-t-butoxycarbonylpropoxy)pyrimidin-2-yl)]amino-2-ethyl-6-trifluoromethyl-3,4-dihydro-2H-quinoline (compound 1C), which was not isolated but directly reacted with excess ethyl chloroformate in the presence of pyridine to produce (2R,4S)-4-[5-(3-t-butoxycarbonylpropoxy)pyrimidin-2-yl)]amino-2-ethyl-6-trifluoromethyl-3,4-dihydro-2H-quinoline-1-carboxylic acid ethyl ester, which was isolated as a crystalline mesylate salt (Compound 1D). The crystalline mesylate salt, Compound 1D was alkylated with 3,5 bis(trifluoromethyl)benzyl bromide (compound 1E) under strongly basic conditions to produce a solution of (2R,4S)-4-{[3,5-bis(trifluoromethyl)benzyl]-[5-(3-t-butoxycarbonylpropoxy)pyrimidin-2-yl]amino}-2-ethyl-6-trifluoromethyl-3,4-dihydro-2H-quinoline-1-carboxylic acid ethyl ester (compound 1F) in toluene. Compound IF was then subjected to an acidic cleavage of the tert-butyl ester to produce a solution of (2R,4S)-4-{[3,5-bis(trifluoromethyl)benzyl]-[5-(3-carboxypropoxy)pyrimidin-2-yl]amino}-2-ethyl-6-trifluoromethyl-3,4-dihydro-2H-quinoline-1-carboxylic acid ethyl ester (compound 1). Compound 1 was then converted to compound 2, which is a solvate of (2R,4S)-4-{[3,5-bis(trifluoromethyl)benzyl]-[5-(3-carboxypropoxy)pyrimidin-2-yl]amino}-2-ethyl-6-trifluoromethyl-3,4-dihydro-2H-quinoline-1-carboxylic acid ethyl ester (compound 2). Finally, compound 2 was converted to the amorphous hemicalcium salt (compound 3) and milled to the target particle size. Compound 2 is crystalline obicetrapib HCl and compound 3 is amorphous obicetrapib hemicalcium. An FT-IR spectrum of milled amorphous obicetrapib hemicalcium can be found in FIG. 52. A solution-state ¹H-NMR spectrum consistent with chemical structure of obicetrapib hemicalcium can be found at FIG. 53. Each of the steps in the manufacturing process for (2R,4S)-4-{[3,5-bis(trifluoromethyl)benzyl]-[5-(3-carboxypropoxy)pyrimidin-2-yl]amino}-2-ethyl-6-trifluoromethyl-3,4-dihydro-2H-quinoline-1-carboxylic acid ethyl ester (compound 1), the intermediate HCl intermediate (compounds 2), and the corresponding amorphous calcium salt (compound 3) will be described in more detail in Examples 11.1-11.16 below.

Examples 11.1-11.3, 11.5, 11.7, 11.9, 11.11-11.12 describe a method for manufacturing steps in the process for preparing amorphous obicetrapib hemicalcium (compound 3); and Examples 11.4, 11.6, 11.8, 11.10 and 11.13 provide additional methods of preparing the compounds indicated. The methods in these examples sometimes represent more than one batch prepared of the indicated compounds made.

Examples 11.14-11.15 describe methods for milling amorphous obicetrapib hemicalcium (compound 3); and Example 11.16 describes a method for preparing crystalline obicetrapib hemicalcium.

### Example 11.1—Preparation of (2R,4S)-4-amino-2-ethyl-6-trifluoromethyl-3,4-dihydro-2H-quinoline (Compound 1A Free Base)

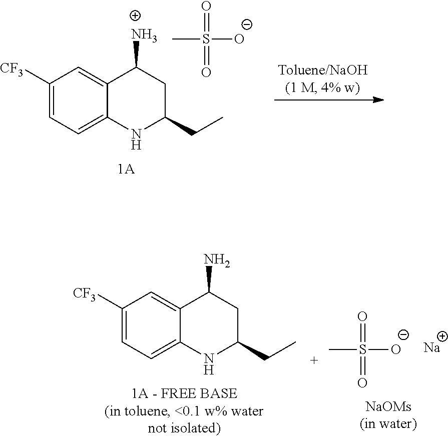

(2R,4S)-4-amino-2-ethyl-6-trifluoromethyl-3,4-dihydro-2H-quinoline (compound 1A) (62 kg, 182 mol, 1.00 equiv.) was added to a reaction vessel fitted with a reflux condenser along with toluene (375 L). The resulting slurry was stirred at 52° C. and 1 M aqueous sodium hydroxide solution (322 L, 5.2 vol.) was added. The reaction mixture was stirred until all solid was dissolved and then cooled to 20° C. The stirring was halted and the reaction mixture was allowed to split into two phases. The bottom aqueous phase was drained, and an aqueous solution of sodium chloride (310 L, 5.0 vol.) was added. The reaction mixture was then stirred at 20° C. for 30 minutes. The stirring was once again halted and the reaction mixture was allowed to split into two phases. The bottom aqueous phase was drained, and deionized water (310 L, 5.0 vol.) was added. The reaction mixture was then stirred at 20° C. for 30 minutes. The stirring was once again halted and the rection mixture was allowed to split into two phases. The bottom aqueous phase was separated. The resulting organic solution was then distilled under vacuum at an internal temperature of 65° C. or less. Distillation was continued until a final visual volume of 4.0 volumes (250 L) was reached. The reaction vessel was then cooled to 20° C. to provide a solution of (2R,4S)-4-amino-2-ethyl-6-trifluoromethyl-3,4-dihydro-2H-quinoline (Compound 1A-FREE BASE) in toluene with a small amount of water present. Compound 1A-FREE BASE was not isolated but used directly in Example 11.2.

### Example 11.2—Preparation of (2R,4S)-4-[5-(3-t-butoxycarbonylpropoxy)pyrimidin-2-yl)]amino-2-ethyl-6-trifluoromethyl-3,4-dihydro-2H-quinoline (Compound 1C)

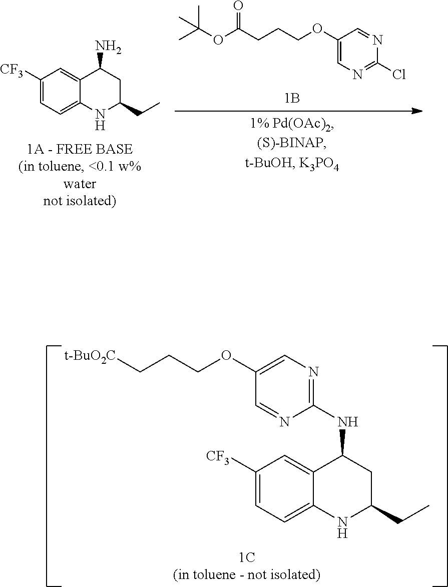

Additional toluene (107 L, 1.5 vol.) was added to the reaction vessel (“vessel A”) containing the (2R,4S)-4-amino-2-ethyl-6-trifluoromethyl-3,4-dihydro-2H-quinoline (Compound 1A-FREE BASE) in toluene with <1000 ppm water from the previous step. T-Butyl-4-(2-chloropyrimidin-5-yloxy)-butyrate (compound 1B) (54.6 kg, 200 mol, 1.10 equiv.) was then added to vessel A along with t-BuOH (122 L, 1.55 vol.). The reaction mixture was stirred and sparged with nitrogen. Meanwhile, palladium acetate (410 g, 1.8 mol, 1 mol %) was added under nitrogen to a second reaction vessel (“vessel B”). (S)-BINAP (2.48 kg, 4.0 mol, 2.2 mol %) and toluene (107 L, 1.5 vol.) were further added to vessel B and the resulting mixture was stirred to form a red/orange Pd-BINAP solution. The orange/red Pd-BINAP solution of reaction vessel B was transferred to vessel A. K₃PO₄ (85 kg, 400 mol, 2.20 equiv.) was further added to vessel A and the resulting reaction mixture was heated to an Internal temperature of 72° C. and stirred for at least 2 hours. The mixture was then cooled to 20° C., deionized water was carefully added (124 L) and the mixture was stirred for 30 minutes. Stirring was then halted and layers were allowed to split into two phases. The bottom aqueous phase was separated, and an aqueous solution of 1M HCl was added (123 L) with stirring. After 30 minutes, the stirring was once again stopped and the layers were allowed to split into two phases. The bottom aqueous phase was separated, and an aqueous solution of sodium chloride (326 kg, 5.26 vol.) was added with stirring. After 30 minutes, the stirring was once again stopped and the layers were allowed to split into two phases. The bottom aqueous phase was separated, and deionized water (248 L, 4.0 vol.) was added with stirring. After 30 minutes, the stirring was once again stopped and the layers were allowed to split into two phases. The bottom aqueous phase was separated. The resulting reaction mixture was then treated with ethylenediamine (1.60 kg, 0.15 equiv.) and stirred at 20° C. for 80 minutes. The reaction mixture was then filtered over a charcoal cartridge and the filtrate returned to a clean vessel. Mixture was then distilled under a partial vacuum at an internal temperature of 60° C. or less. Distillation was continued until approximately 2.50 volumes by visual observation in reactor (155 L) remained, then acetonitrile (394 L, 5.0 vol.) was added. The mixture was then distilled under vacuum at an internal temperature of 60° C. or less. Distillation was continued until approximately 2.50 volumes by visual observation in reactor (155 L), then the contents were cooled to 20° C. The reaction vessel was then charged with acetonitrile (394 L, 5.0 vol. vol., to reach 11 volumes by visual observation (approximately 620 L)) to obtain (2R,4S)-4-[5-(3-t-butoxycarbonylpropoxy)pyrimidin-2-yl)]amino-2-ethyl-6-trifluoromethyl-3,4-dihydro-2H-quinoline (compound 1C) dissolved in acetonitrile. Compound 1C was not isolated but used directly in Example 11.3.

### Example 11.3—Preparation of (2R,4S)-4-[5-(3-t-butoxycarbonylpropoxy)pyrimidin-2-yl)]amino-2-ethyl-6-trifluoromethyl-3,4-dihydro-2H-quinoline-1-carboxylic acid ethyl ester, as a Crystalline Mesylate Salt (Compound 1D)

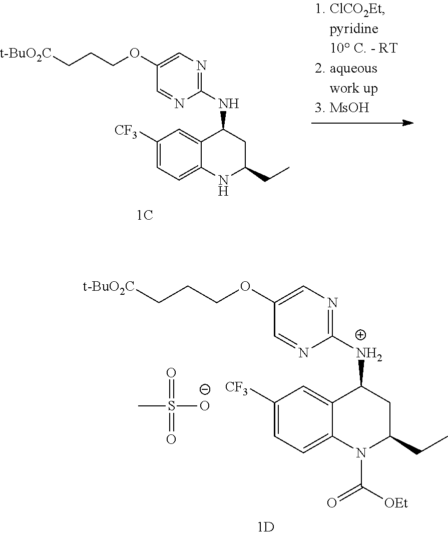

(2R,4S)-4-[5-(3-t-butoxycarbonylpropoxy)pyrimidin-2-yl)]amino-2-ethyl-6-trifluoromethyl-3,4-dihydro-2H-quinoline (compound 1C) in acetonitrile (approximately 620 L) was cooled to an internal temperature of <10° C. and pyridine (72 L, 900 mol, 4.9 equiv.) was added. Ethyl chloroformate (136 L, 1428 mol, 7.84 equiv.) was then added through an addition funnel while keeping the internal temperature of the reactor contents <10° C. The internal temperature of the reaction mixture was then increased linearly to 20° C. over the course of 3.5 hours. The mixture was then distilled under vacuum at an internal temperature of 60° C. or less. Distillation was continued until approximately 2.50 volumes by visual observation (155 L). Isopropyl acetate (471 L, 6.6 vol.) was then added to the reaction vessel and distillation was continued under vacuum at an internal temperature of 60° C. or less until roughly 2.50 volumes remained by visual observation (155 L). Then isopropyl acetate (471 L, 6.6 vol.), 1M hydrochloric acid (307 L, 5.0 vol.), and 26% aqueous sodium chloride (63 L, 1.2 vol.) were added to the reaction vessel. The resulting mixture was stirred for 30 minutes, then separated into two phases. The bottom aqueous phase was separated, and saturated aqueous sodium bicarbonate solution (132 L, 2.3 vol.) was added. The resulting mixture was stirred for 30 minutes, then separated into two phases. The bottom aqueous phase was separated and the remaining mixture was distilled under vacuum and at 60° C. or less to reach a total volume of roughly 4.0 volumes by visual observation (250 L) to obtain (2R,4S)-4-[5-(3-t-butoxycarbonylpropoxy)pyrimidin-2-yl)]amino-2-ethyl-6-trifluoromethyl-3,4-dihydro-2H-quinoline-1-carboxylic acid ethyl ester (corresponding free base of Compound 1D) in isopropyl acetate based on the weight of the solution.

Additional isopropyl acetate (86 L, 1.4 vol.) and methyl t-butylether (MTBE, 593 L, 9.6 vol) were added to (corresponding free base of Compound 1D) in isopropyl acetate and the jacket temperature was set to 20° C. Methanesulfonic acid (MsOH, 17.6 kg, 1.0 equiv. based on mmol of compound (corresponding free base of Compound 1D) was then added to the reaction mixture over 60 minutes. The resulting slurry was then agitated for 8 hours. The slurry was then filtered under vacuum at 20° C. The solid cake was then washed with 75/25 v/v isopropyl acetate (78 L, 1.1 vol.) and methyl t-butyl ether solution (236 L, 2.8 vol.) then dried under vacuum and at 20° C. to obtain isolated (2R,4S)-4-[5-(3-t-butoxycarbonylpropoxy)pyrimidin-2-yl)]amino-2-ethyl-6-trifluoromethyl-3,4-dihydro-2H-quinoline-1-carboxylic acid ethyl ester, as a crystalline mesylate salt (Compound 1D) with a yield of 74%, based on the number of moles of compound 1A. The purity of the crystalline Compound 1D obtained was >99%.

### Example 11.4—Additional Preparation of Compound 1C and Compound 1D

The preparation described below was generally used to prepare multiple batches of Compound 1C and Compound 1D. In some preparations, for example, seeding with Compound 1D was done and others not as further discussed below.

Pd(OAc)₂ and(S)-BINAP were dissolved in toluene and stirred to form the corresponding Pd-BINAP-complex (color change to red) (the “catalyst solution”). Toluene, Compound 1B, Compound 1A and K₃PO₄ were added to the reactor and stirred. A target content for water is on the order of 6%. Catalyst solution was added to the reactor mixture, and the reaction mixture was heated up to 70-75° C. and stirred.

After washing with HCl, brine and water, subsequent phase separation, EDA and toluene were charged and the solution was stirred for approximately 90 min. The solution was passed via a cartridge loaded with activated carbon (Begerow, F-9120) for removing palladium. Afterwards, the solvent was switched from toluene to acetonitrile (MeCN) by distillation to obtain Compound 1C.

To the Compound 1C solution, pyridine was added and cooled to lower than 10° C. prior to ethyl chloroformate addition. Ethyl chloroformate was dosed in one or two portions to the Compound 1C solution, while the temperature was controlled to NMT 10° C. The reaction mixture was then stirred for approximately 1 hour at 17-27° C., converting Compound 1C to the free base of Compound 1D (Compound 1D-FB). A solvent switch from acetonitrile to isopropyl acetate (iPrOAc) was done by distillation, and the organic phase was washed with HCl (1 M), brine and aqueous NaHCO₃(NaOH may also be used) followed by volume reduction by distillation.

To the Compound 1D-FB solution in iPrOAc, methanesulfonic acid (MsOH) and MTBE was added and stirred. In some cases, previously made seed crystals of Compound 1D were added, but that is not required. Whether seeds are added or not, crystallization of Compound 1D followed. The solid product was filtered, washed with MTBE/iPrOAc (75/25) and dried on a filter.

### Example 11.5—Preparation of (2R,4S)-4-{[3,5-bis(trifluoromethyl)benzyl]-[5-(3-tbutoxycarbonylpropoxy)pyrimidin-2-yl]amino}-2-ethyl-6-trifluoromethyl-3,4-dihydro-2Hquinoline-1-carboxylic acid ethyl ester (Compound 1F)

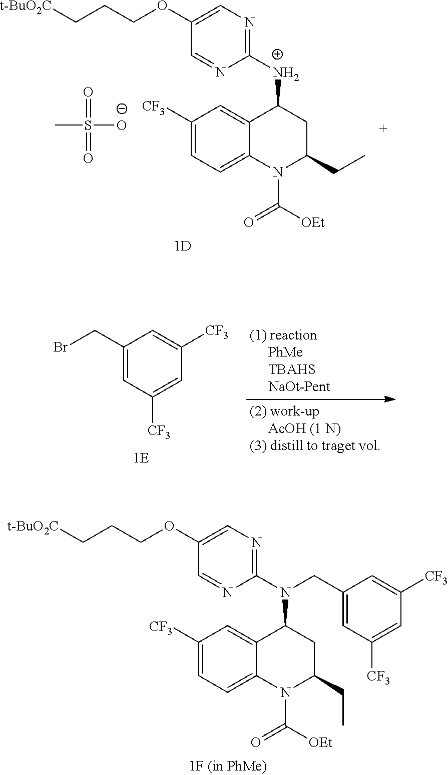

(2R,4S)-4-[5-(3-t-butoxycarbonylpropoxy)pyrimidin-2-yl)]amino-2-ethyl-6-trifluoromethyl-3,4-dihydro-2H-quinoline-1-carboxylic acid ethyl ester, as a crystalline mesylate salt (Compound 1D) (42 kg) and toluene (465 kg, 12.7 vol.) was added to a reaction vessel at a temperature of 5° C. Tetrabutylammonium hydrogensulfate (3.5 kg, 0.16 equiv.) and sodium tert-pentoxide (34.5 kg, 4.8 equiv.) were then added and the resulting reaction mixture was stirred for 10 minutes and degassed with nitrogen. 3,5-bis(trifluoromethyl)benzyl bromide (Compound 1E) (28 kg, 1.41 equiv.) was then added to the reaction mixture and stirring was continued for 6.5 hours at 5° C. The reaction mixture was then treated with IN acetic acid solution (320 kg) and allowed to stir for approximately 30 minutes at 20° C. After which time, the stirring was stopped and the mixture was allowed to separate into two phases. The lower aqueous phase was discarded and the reaction mixture was concentrated under vacuum at an internal temperature 60° C. or less until approximately 3.3 volumes (137 L) remained, to obtain a solution of 36.8 weight percent (2R,4S)-4-{[3,5-bis(trifluoromethyl)benzyl]-[5-(3-tbutoxycarbonylpropoxy)pyrimidin-2-yl]amino}-2-ethyl-6-trifluoromethyl-3,4-dihydro-2Hquinoline-1-carboxylic acid ethyl ester (Compound 1F) in toluene, based on the weight of the solution, 97% yield based on the number of moles of Compound 1D).

### Example 11.6—Additional Preparation of Compound 1F

Compound 1E was charged to a toluene solution containing Compound 1D and tetrabutylammonium hydrogensulfate. Under cooling, sodium tert-pentoxide in toluene was added. The resulting reaction mixture was quenched with dilute acetic acid. The aqueous layer was separated and the product in the toluene layer was treated with charcoal and concentrated in vacuum (Compound 1F.)

### Example 11.7—(2R,4S)-4-{[3,5-bis(trifluoromethyl)benzyl]-[5-(3-carboxypropoxy)pyrimidin-2-yl]amino}-2-ethyl-6-trifluoromethyl-3,4-dihydro-2H-quinoline-1-carboxylic acid ethyl ester (Compound 1)

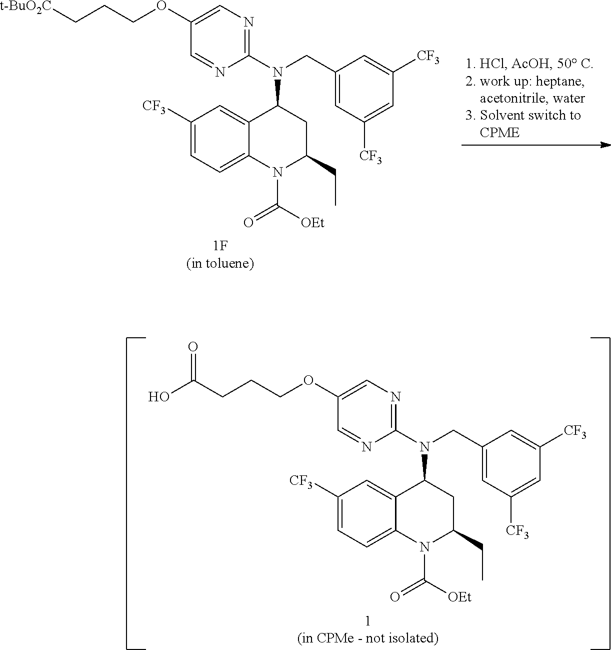

A solution of 37 wt. % (2R,4S)-4-{[3,5-bis(trifluoromethyl)benzyl]-[5-(3-tbutoxycarbonylpropoxy)pyrimidin-2-yl]amino}-2-ethyl-6-trifluoromethyl-3,4-dihydro-2Hquinoline-1-carboxylic acid ethyl ester (compound 1F) in toluene (128.4 kg of the 37 wt. % solution, equivalent to 47.5 kg compound 1F) was diluted to 32 wt % with additional toluene and then mixed with acetic acid (253 kg, 5.33 wt.), and 6 M HCl (109.9 kg, 2.32 wt, prepared in situ with 66.1 kg of conc. HCl and 43.8 kg of water). The resulting reaction mixture was vigorously agitated and warmed to 48° C. for 3 hours. The reaction mixture was then cooled to 21° C., then n-heptane (159.8 kg, 3.36 wt.), acetonitrile (73.8 kg, 1.55 wt.) and water (170 kg, 3.58 wt.) were added. The resulting mixture was agitated for 34 minutes and then allowed to separate into two phases. The lower aqueous phase was then further treated with water (90 kg, 1.89 wt.), n-heptane (95 kg, 2.00 wt.), acetonitrile (38 kg, 0.80 wt.) and toluene (42 kg, 0.88 wt.) and once again agitated for 20 minutes before separating the organic phase and discharging the lower aqueous phase. The combined organic phases were then treated with water (240 kg, 5.05 wt.) and agitated for an additional 30 minutes before separating into two phases. The lower aqueous phase was discarded and the upper organic phase was treated with 5% w/w sodium citrate tribasic dihydrate (34 kg, 0.72 wt.) and water (205 kg, 4.32 wt.). The resulting mixture was vigorously agitated for 30 minutes and then allowed to separate into two phases before discarding the lower aqueous phase. The remaining organic phase was treated once again with water (240 kg, 5.05 wt.) and agitated for 30 minutes before allowing to separate into two phases and discharging the lower aqueous phase. The organic phase was then concentrated to approximately 3 volumes (approximately 149 L) in-vacuo maintaining an internal temperature of 50° C. or less. The reaction mixture was diluted with cyclopentyl methyl ether (CPME, 250 kg, 5.26 wt.) and agitated. The solution was then concentrated to approximately 3 volumes (approximately 165 L) in-vacuo maintaining an internal temperature of 50° C. or less. CPME (250 kg, 5.26 wt.) was then added and the mixture concentrated to approximately 2.5 volumes (approximately 124 L) in-vacuo, maintaining an internal temperature of 50° C. or less to obtain a solution of 33.7 weight percent of (2R,4S)-4-{[3,5-bis(trifluoromethyl)benzyl]-[5-(3-carboxypropoxy)pyrimidin-2-yl]amino}-2-ethyl-6-trifluoromethyl-3,4-dihydro-2H-quinoline-1-carboxylic acid ethyl ester (compound 1, free base form) in cyclopentyl methyl ether (CMPE) having 1 weight percent toluene, less than 1 weight percent n-heptane, based on the weight of the solution.

### Example 11.8—Additional Preparation of Compound 1

Compound 1F (in solution in toluene) was mixed with acetic acid and 6 M aq. HCl. The biphasic mixture was intensively stirred at 45-50° C. and subsequently cooled to 20° C. After addition of water, acetonitrile and n-Heptane, the mixture was extracted, and the layers were separated.

The aqueous layer of the first extraction was diluted with water and extracted with Acetonitrile, n-heptane, and toluene a second time. The two obtained organic extracts were combined. The organic phase was washed with water and 5% sodium citrate solution added so that the pH was ≥3.5. A water wash was performed, and the organic layer was treated with activated charcoal. A solvent switch from toluene and n-Heptane to CPME was performed by repeated vacuum distillation and charging CPME to obtain Compound 1.

### Example 11.9—(2R,4S)-4-{[3,5-bis(trifluoromethyl)benzyl]-[5-(3-carboxypropoxy)pyrimidin-2-yl]amino}-2-ethyl-6-trifluoromethyl-3,4-dihydro-2H-quinoline-1-carboxylic acid ethyl ester hydrochloride (Compound 2)

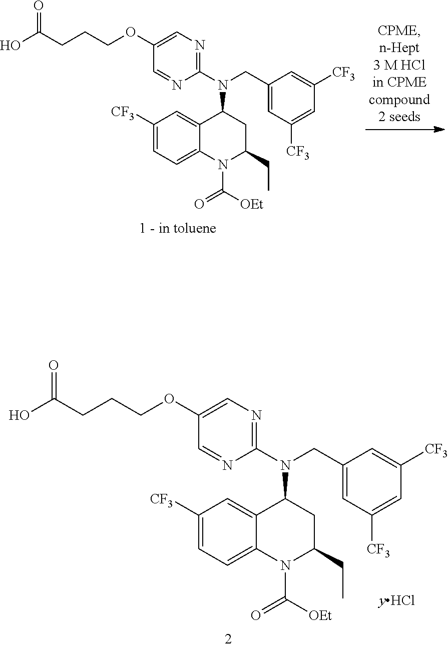 [SMILES: CCOC(=O)N1c2ccc(C(F)(F)F)cc2[C@@H](N(Cc2cc(C(F)(F)F)cc(C(F)(F)F)c2)c2ncc(OCCCC(=O)O)cn2)C[C@H]1CC ; CCOC(=O)N1c2ccc(C(F)(F)F)cc2[C@@H](N(Cc2cc(C(F)(F)F)cc(C(F)(F)F)c2)c2ncc(OCCCC(=O)O)cn2)C[C@H]1CC]

The 33.7 weight percent solution of (2R,4S)-4-{[3,5-bis(trifluoromethyl)benzyl]-[5-(3-carboxypropoxy)pyrimidin-2-yl]amino}-2-ethyl-6-trifluoromethyl-3,4-dihydro-2H-quinoline-1-carboxylic acid ethyl ester (compound 1, free base form, 115.6 kg, 59.2 mol) in cyclopentyl methyl ether (CPME) from the previous step was added to a clean reaction vessel under nitrogen with a jacket temperature of 22° C. After dilution with CPME (27.8 kg/0.58 wt.), n-heptane was then added (54.8 kg, 1.15 wt.) and the internal reaction temperature was increased to 39° C. 3.0 M HCl in CPME (17.6 kg, 0.37 wt.) was then added at a constant rate while maintaining an internal reaction temperature of 39° C. After the addition of HCl was complete, the internal temperature was increased to 52° C. Additional n-heptane was then added (133.2 kg, 2.80 wt.) at a constant rate while maintaining an internal reaction temperature of 51° C. The reaction mixture was heated to 55° C. and then it was cooled to 49° C. An aliquot of the reaction mixture was removed, cooled to 11° C. at a linear cooling rate until a slurry formed containing crystals of compound 2 in CPME/n-heptane (referred to herein as “seed crystal slurry”). A seed crystal slurry of compound 2 (169 g, 0.43 weight percent) in CPME/n-heptane was then added at 49° C. and this temperature was held for 105 minutes. The opaque reaction mixture was then cooled to 11° C. over the course of 12 hours at a linear cooling rate. The reaction mixture was then filtered under vacuum at 11° C. to collect the solid wet HCl intermediate (compound 2). A mixture of CPME and n-heptane (56.6 kg CPME, 179 kg n-heptane) was then added to the reaction vessel and cooled to 11° C. Half the mixture was then poured through the filter dryer as a chromatography wash. The second half was passed through the filter as a slurry wash. Compound 2 was not unloaded from the filter dryer but was further purified by recrystallization according to the following procedure. Compound 2 in cyclopentyl methyl ether (CPME) (77.6 kg) was added into a filter dryer containing compound 2 and heated to 25° C. The dissolved compound 2 was then transferred to a reaction vessel with a reactor jacket temperature set at 25° C. under nitrogen, and the internal temperature was increased to 38° C. 3.1 M HCl in CPME (6.4 kg) was added so that a total of 1.07 equiv. HCl was achieved based on assay of compound 1 in compound 2 crude and assay of HCl in compound 2 crude. N-Heptane was then added (139.4 kg and the internal reaction temperature was increased to 51° C. A seed crystal slurry of compound 2 (291 g, 0.87 weight percent) in CPME/n-heptane was then added at 50° C. and this temperature was held for 105 minutes. The opaque reaction slurry was then cooled to 11° C. over 12 hours at a linear cooling rate. The slurry was then filtered under vacuum at 9° C. using a filter dryer. 20 vol. % of CPME in n-heptane (57.4 kg CPME, 180 kg n-heptane) was then added to the reaction vessel and cooled to 11° C. Half the mixture was then poured through the filter dryer as a chromatography wash. The second half was passed through the filter dryer as a slurry wash. The wet filter cake was then dried in vacuo in steps of jacket temperature 25, 35, 46, 54° C. to provide compound 2 in 64% yield (from compound 1F) with 99.6 area % purity and residual solvents 0.3% w CPME and <0.1% w n-heptane.

### Example 11.10—Additional Preparation of Compound 2

Compound 1 in solution was further diluted with n-Heptane and heated to 40° C. At this temperature approximately 3 M HCl in CPME (1.1 meq regarding Compound 1F) were added. The solution was further heated to 48-53° C. and a second portion of n-Heptane was charged. The clear solution was cooled to 53° C. for optional seeding with Compound 2 seeding crystals (seeding is optional but preferred in a manufacturing context). If seeded, the solution is desaturated at 53° C. and then cooled to 10° C. over 12 hours.

A crystal curing procedure (a repeated heating and cooling cycle procedure) can be performed to improve the color of the resulting filtered Compound 2. The product suspension was filtered, washed once with cooled CPME/n-Heptane (20:80 vol) and once with cooled n-Heptane and then dried in vacuum.

### Example 11.11—(2R,4S)-4-{[3,5-bis(trifluoromethyl)benzyl]-[5-(3-carboxypropoxy)pyrimidin-2-yl]amino}-2-ethyl-6-trifluoromethyl-3,4-dihydro-2H-quinoline-1-carboxylic acid ethyl ester (Compound 3)

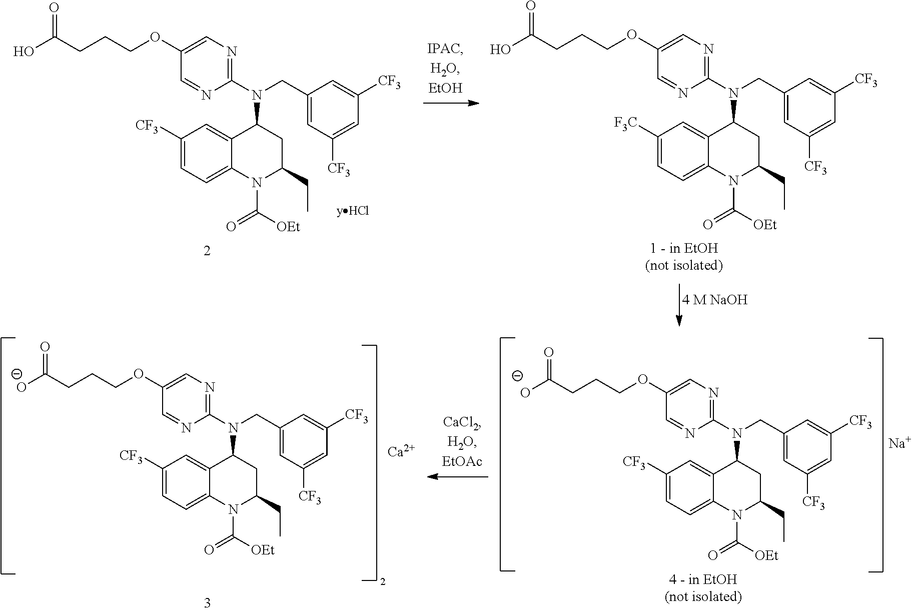

(2R,4S)-4-{[3,5-bis(trifluoromethyl)benzyl]-[5-(3-carboxypropoxy)pyrimidin-2-yl]amino}-2-ethyl-6-trifluoromethyl-3,4-dihydro-2H-quinoline-1-carboxylic acid ethyl ester hydrochloride (compound 2, 35.0 kg, 48.4 mol) was added to isopropyl acetate (IPAC, 214 kg, 6.11 wt.) to an inert reactor and stirred at 22° C. to achieve dissolution. Deionized water (245 kg, 7.00 wt.) was added, the reaction mixture was stirred at 23° C. for 35 minutes, then the stirring was stopped, the phases were separated, and the lower aqueous phase was removed. The process of adding deionized water (245 kg, 7 wt.), stirring, and removing the lower aqueous phase was repeated further 3 times. The organic phase was then concentrated under reduced pressure to approximately 71 L (approximately 2 vol.) maintaining an internal temperature of 55° C. or less. Ethanol (115 kg, 3.29 wt.) was then added, and the reaction mixture was concentrated under reduced pressure to approximately 78 L (approximately 2 vol.) maintaining an internal temperature of 55° C. or less. The process of adding ethanol (115 kg, 3.29 wt.) and concentrating was repeated twice more. The reaction mixture was then cooled to 25° C. and subjected to a charcoal treatment via a cartridge. The cartridge was then rinsed with ethanol (100 kg, 2.86 wt.) and concentrated to 147 L (approximately 3.8 vol.) at 55° C. or less in vacuo followed by addition of 35 L of EtOH (1.0 vol.) to provide the free base form of (2R,4S)-4-{[3,5-bis(trifluoromethyl)benzyl]-[5-(3-carboxypropoxy)pyrimidin-2-yl]amino}-2-ethyl-6-trifluoromethyl-3,4-dihydro-2H-quinoline-1-carboxylic acid ethyl ester (compound 1) in ethanol. The 14% wt. NaOH solution (15.8 kg, 1.13 eq.) was then added to the reaction vessel containing compound 1 in ethanol maintaining a reaction temperature of 20° C. The reaction mixture was stirred at 20° C. for 5 hours to achieve full conversion.

34% wt. Calcium chloride (aq.) (10.8 kg) was added to an inert reactor. Deionized water (336 L, 9.61 wt. relative to compound 1) and ethyl acetate (15 kg, 0.43 wt. relative to compound 1) was then added and the mixture was stirred for 30 minutes to provide “Solution B.”

Solution B was then cooled to 9° C. with agitation. Solution A (see above) was then added via a filter to Solution B over 90 minutes, maintaining a temperature of 10° C. The Solution A vessel was then rinsed forward to solution B with additional ethanol (50 kg, 1.43 wt. relative to compound 1). The resulting slurry was stirred for 1 hour at 9° C. The solids were then collected by filtration and rinsed with deionized water (2×175 kg, 5 wt. relative to compound 1). The solids were then dried in vacuo at 50° C. for 21 hours to obtain 27.6 kg of amorphous obicetrapib hemicalcium (compound 3) with <1 weight percent water (77% yield, based the number of moles of compound 2). The compound 3 was reworked as described below in Example 11.12.

### Example 11.12—Rework of Compound 3

Compound 3 (27.6 kg) was dissolved in ethanol (55.2 kg 2 wt. relative to compound 3) at 45-48° C. and subsequently cooled to 11° C. The solution was filtered into a pre-cooled (approximately 10° C.) mixture of an aqueous CaCl₂) solution (8.2 kg of 33-35 weight percent, 0.3 wt.), water (262 kg, 9.5 wt.) and ethyl acetate (12.6 kg, 0.46 wt.). The resulting suspension was filtered off and washed with water (2×5 wt., 138 kg per washing step) and the solid was dried in vacuo maintaining an internal temperature of 45° C. or less for 23 hours to obtain 24.8 kg (91% yield) of the amorphous hemicalcium salt of (2R,4S)-4-{[3,5-bis(trifluoromethyl)benzyl]-[5-(3-carboxypropoxy)pyrimidin-2-yl]amino}-2-ethyl-6-trifluoromethyl-3,4-dihydro-2H-quinoline-1-carboxylic acid ethyl ester (compound 3) with <1 weight percent water and a purity of 97.5% wt. and >99.9 area %.

### Example 11.13—Additional Preparation of Compound 3

Compound 2 was neutralized and dissolved with aqueous NaOH in EtOH. The solution was filtered through activated carbon. Vacuum distillation was performed to concentrate the solution. Aqueous NaOH solution was dosed to obtain the sodium salt of Compound 1 in solution and for saponification of esters, which were formed in this and previous steps. Subsequently, a mixture of aqueous CaCl₂) solution and EtOAc was prepared in a second vessel. The Na-salt of Compound 1 from the first vessel was then dosed into this mixture, whereby Compound 3 precipitated. Optionally, the suspension may be heated to NMT 25° C. and subsequent cooled to 8° C. The solid Compound 3 was filtered off at 8° C., washed with water and dried in vacuo.

### Example 11.14—Milling of Reworked Compound 3

Compound 3 was jet-milled using an 8-inch spiral mill. Feed rate, venturi pressure, and mill pressure were adjusted within the ranges listed below to allow the production of micronized compound 3 in compliance with particle size acceptance criteria (D90=6-15 μm).
Feed rate: 17-20 kg/h
Mill pressure: 20 PSI/1.4 bar
Venturi pressure: 100 PSI/6.9 bar
Process gas: Nitrogen
Analytics: Mastersizer 3000.

### Example 11.15—Milling of Another Compound 3 Preparation

Particle size distribution was adjusted via micronization on a Spiral Jetmill, 8 inch Jet Mill, 8005 and KT4 LIW feeder to target parameters d90: 6-15 microns. Three samples were jet milled with the following results:
d90: 8 microns, 8 microns, and 9 microns
d50: 4 microns, 3 microns, and 4 microns
d10: 2 microns, 1 micron, 1 micron

### Example 11.16—Crystalline Obicetrapib Hemicalcium

2 g of amorphous obicetrapib hemicalcium was added to Acetonitrile (ACN)/Methyl tert butyl ketone (MIBK), 6:1 ratio at 200 mg/ml and the sample was heated to 50° C. for 5 minutes, until all the solids dissolved. The sample was then placed in a water bath and cooled from 50° C. to 5° C. over 48 hours at 0.9° C./min. The samples were kept at 5° C. for 3 days and then transferred to −20° C. for 30 mins prior to isolation of solids. The solids were air dried for 2 hours prior to further characterization. The process resulted in the formation of crystalline obicetrapib hemicalcium.

### Example 11.17—Polarized Light Microscopy (PLM)

Polarized light microscopic pictures were captured using a Nikon DS-Fi2 upright microscope at room temperature. Samples (2 mg) were mounted on a glass slide and covered with a drop of silicone oil with a cover slip on top of the sample for analysis. Samples were not protected from light.

### Example 11.18—X-ray Powder Diffraction (XRPD)

XRPD was performed with Panalytical X'Pert3 Powder diffractometer using an incident beam of Cu radiation produced using an Empyran tube, fine focused source, on a silicon zero-background holder. Prior to the analysis, a silicon standard (NIST SRM 640d) was analyzed to verify that the Si 111 peak position is consistent with the NIST-certified position. Approximately 5 to 10 mg of sample was placed on a silicon zero-background holder and flattened manually using an aluminum spatula to minimize difference in the overall sample height. The holder was then loaded on the instrument for analysis. The XRPD parameters used are listed in the following Table.

Parameters for XRPD test

| Parameters | Reflection Mode |
|---|---|
| X-Ray wavelength | Cu, kα |
| | Kα1 (Å): 1.540598, |
| | Kα2 (Å): 1.544426, |
| X-Ray tube setting | Kα2/Ka1 intensity ratio: 0.50 |
| | 45 kV, 40 mA |
| Divergence slit | Fixed ⅛° |
| Scan mode | Continuous |
| Scan range (° 2TH) | 3-40 |
| Scan step time [s] | 18.87 |
| Step size (° 2TH) | 0.0131 |
| Test Time | 4 min 15 s |

### Example 11.19—X-ray Powder Diffraction Pattern

A PANalytical x-ray powder diffractometer was used with the following measurement conditions, with data acquisition by DataViewer and data evaluation by X′Pert High Score Plus:

| | |
|---|---|
| X-ray tube | Cu LFF HR |
| Geometry | Transmission |
| X-ray mirror | Focusing X-ray mirror W/Si |
| Soller slit | 0.02 rad |
| Detector | Pixel 1D |
| Detector active length | 1.69° |
| Divergence slit | Fixed |
| Divergence slit size | ½° |
| X-ray tube excitation | 40 mA, 40 kV |
| 2Theta range | 2° to 40° |
| Measurement mode | Continuous |
| Time per Step | 300 s |
| Step size | 0.013° (2Theta) |
| Rotation | 1 Rev/s |

### Example 11.20—X-ray Powder Diffraction Pattern

The diffraction pattern for FIG. 51 was measured using an Empyrean powder diffractometer in transmission mode from Malvern PANalytical. The sample was prepared as a thin layer between two Kapton foils and measured in continuous mode. The detector measures from approx. 2°2θ to 40°2θ. Peaks can be seen at signals at about 3.4°2θ, about 7.0°2θ and about 9.2°2θ. The peak at about 5.6°2θ is assigned to Kapton foil.

### Example 11.21—X-Ray Powder Diffraction Methodology for Crystalline Obicetrapib HCl/Compound 1D

Diffraction patterns were measured using a Thermo Fisher Scientific ARL Equinox 1000 powder diffractometer. The diffractometer is equipped with a copper source and a germanium (111) monochromator providing monochromatic Cu Kal radiation, and a position sensitive gas-ionization detector.

Samples were measured in reflection mode using an A1 sample holder without any further preparation (i.e., grinding). The detector measures over the entire angle range from approx. 2°2θ to 120°2θ simultaneously; in the case of HCl obicetrapib, discernible signals useful for phase identification are seen up to approx. 45°2θ. The temperature in the diffractometer is typically around 30° C. during measurements.

### Example 11.22—X-Ray Powder Diffraction Methodology for Crystalline Obicetrapib HCl

A Rigaku SmartLab X-Ray Diffractometer was configured in Bragg-Brentano reflection geometry equipped with a beam stop and knife edge to reduce incident beam and air scatter. Data collection parameters are shown in the following.

X-Ray Powder Diffraction Parameters

| Parameter | Value | Parameter | Value |
|---|---|---|---|
| Geometry | Bragg-Brentano | Receiving Slit 1 (mm) | 18 |
| Tube Anode | Cu | Receiving Slit 2 (mm) | open |
| Tube Type | Long Fine Focus | Start Angle 2θ (°) | 2 |
| Tube Voltage (kV) | 40 | End Angle 2θ (°) | 40 |
| Tube Current (mA) | 44 | Step Size (°) | 0.02 |
| Detector | D/teX Ultra 250 (XR1 or | Scan Speed (°/min) | 6 |
| | XR5) HyPix-3000 (XR4) | | |
| Monochromator | Ni foil Cu Kβ Filter | Spinning (rpm) | 11 |
| Incident Slit (°) | ⅓ | Sample Holder | Low-background Si |

### Example 11.23—FT-IR Spectroscopy

The FTIR spectrum a sample of amorphous obicetrapib hemicalcium is set forth in FIG. 52. The FTIR spectrum was acquired using a Bruker Tensor 27 spectrometer with a Platinum ATR-QL-Diamond unit. The milled sample was placed onto the ATR unit without any pretreatment.

### Example 11.24—¹H-NMR Spectroscopy

The NMR spectra of a solution made from a sample of amorphous obicetrapib hemicalcium is set forth in FIG. 53. The NMR spectrum was obtained using a 600 MHz AVANCE NEO Bruker and in deuterated MeOH as solvent using tetramethylsilane (TMS) as the internal reference for chemical shift at 0.0 ppm. The spectral shifts are consistent with the chemical structure.

### Example 11.25—Modulated Differential Scanning Calorimetry (mDSC)

Samples with mDSC thermograms are set forth at FIG. 60 and FIG. 62 were prepared with Tzero aluminum pan with a pinhole. The ramp rate was from 25° C. to 225° C. at 2° C./min with modulation of ±0.5° C. every 60 seconds. The Instrument used was a TA Q2500 DSC from TA Instruments.

### Example 11.26—Modulated Differential Scanning Calorimetry (mDSC)

Using a TA Instruments DSC2500, the starting temperature was 25° C. and the sample was heated with 2° C. per minute, modulating by +0.5° C. every 60 seconds up to 225° C. Tzero aluminium pans and Tzero hermetic lids with factory pinholes, which were additionally pierced through and enlarged, were employed for this testing. An integrated thermogram (displaying reversing heat flow) from the sample is included in FIG. 61. The sample exhibited a glass transition with a Tg of approx. 111° C.

### Example 11.27—Methods to Assess Stability

Stability study was conducted on crystalline and amorphous forms of obicetrapib at 70° C./75% relative humidity (RH). The solids were placed into a 4.0 ml glass vial without caps (open condition) and stored at 70° C./75% RH. Samples were pulled out of the stability chamber at day 1 (24 hours) and at 7-day time point. The solids were analyzed by XRPD for physical stability and by HPLC for chemical purity. The sample collected at each time point was dissolved in methanol before HPLC analysis. In order to minimize the effect of potential analyte adsorption on the filter, the initial 0.5 mL of supernatant passing through the filter was discarded prior to collecting sample for HPLC analysis. Purity for each sample was determined based on peak area percent and compared to the sample at T=0.

### Example 11.28—Method Used to Assess Kinetic Solubility in Biorelevant Media

Kinetic solubility study was conducted on crystalline and amorphous forms of obicetrapib in biorelevant media including Fed state simulated intestinal fluid (FeSSIF) at pH 5.0 and Fasted state simulated intestinal fluid (FaSSIF) at pH 6.5 at 37° C. The solids were magnetically stirred at 600 RPM in a shaker bath and samples were drawn with a 1.0 ml syringe at T=15 mins, 30 mins, 60 mins, 90 mins and 120 mins. The solubility was measured using HPLC method provided by the client. The compound was added to 4.0 ml glass vials at approximately 20.0 mg/ml. Samples were agitated with a vortex mixer for about 5 minutes to ensure the presence of undissolved excess powder. The sample collected at each time point was centrifuged at 1200 RPM, filtered with 0.45 μm Polytetrafluoroethylene (PTFE) filters and diluted with methanol before HPLC analysis. In order to minimize the effect of potential analyte adsorption on the filter, the initial 0.5 mL of supernatant passing through the filter was discarded prior to collecting sample for HPLC analysis.

While the invention has been particularly shown and described with reference to a preferred embodiment and various alternate embodiments, it will be understood by persons skilled in the relevant art that various changes in form and details can be made therein without departing from the spirit and scope of the invention.

### Example 12—Obicetrapib and Ezetimibe Synergistically Reduce Non-HDL-Cholesterol by Enhanced LDL-Receptor-Mediated VLDL Clearance and Increased Net Fecal Sterol Excretion in ApoE*3-Leiden.CETP Mice

Obicetrapib is a selective cholesteryl ester transfer protein (CETP) inhibitor currently in clinical development for the treatment of hypercholesterolemia and reduction of cardiovascular risk that strongly reduces Apolipoprotein B (ApoB) and low-density lipoprotein cholesterol (LDL-C) and concomitantly increases plasma high-density lipoprotein cholesterol (HDL-C). Ezetimibe reduces absorption of biliary and dietary cholesterol from the small intestine, thereby reducing LDL-C levels. The current study was carried out to elucidate the mechanism of action responsible for the observed decrease in non-HDL-C by obicetrapib monotherapy and in combination with ezetimibe in a translational mouse model for hyperlipidemia and atherosclerosis.

### Methods

The experimental protocol is illustrated in FIG. 71, with time (t) measured in weeks. Female ApoE*3-Leiden.CETP transgenic mice were fed a Western diet with 0.05% w/w cholesterol (equivalent to daily human intake) with (i) no drug (control), or the same diet containing (ii) obicetrapib alone (2 mg/kg/day), (iii) ezetimibe alone (1 mg/kg/day) or (iv) the combination of obicetrapib and ezetimibe. In the experiment, the effects of the treatments on, amongst other things, plasma blood lipids and fecal bile acids and sterols were determined.

### Results

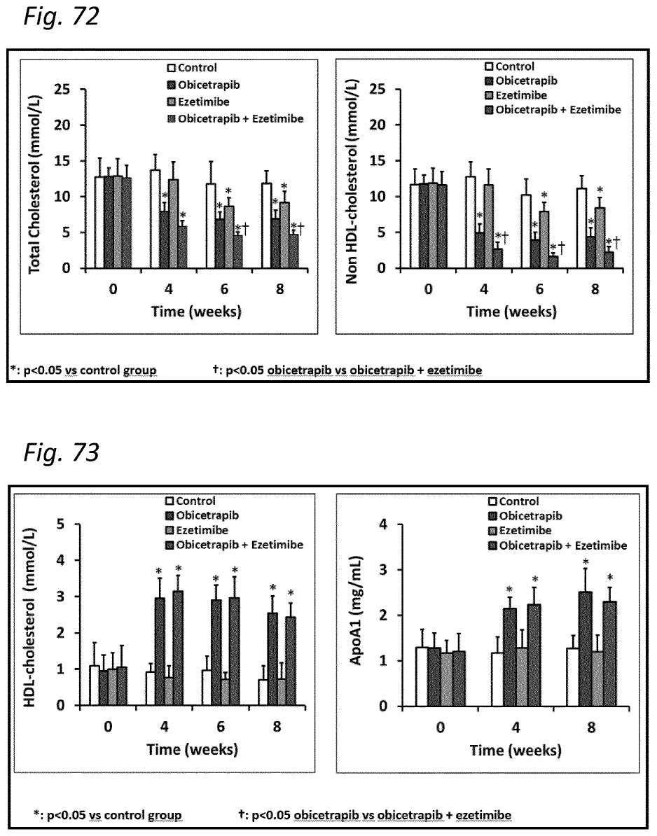

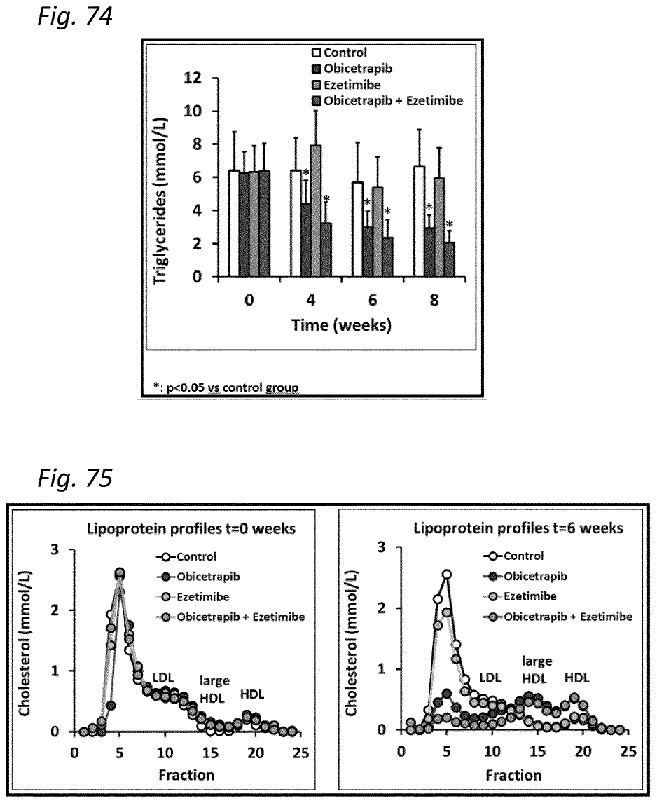

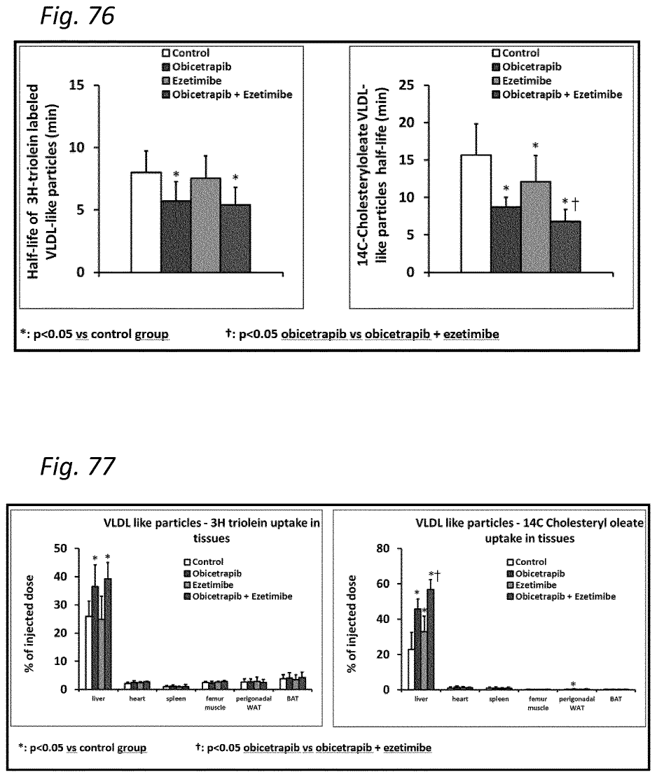

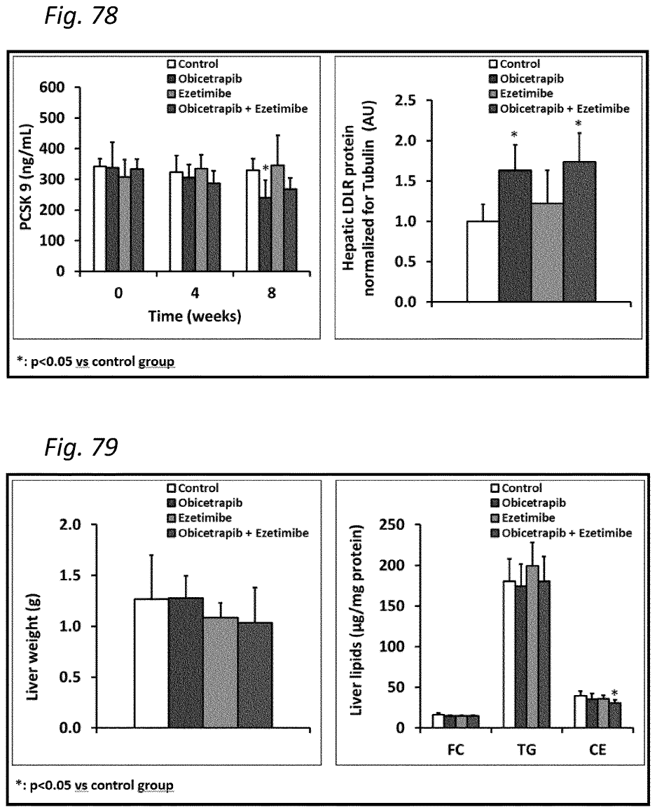

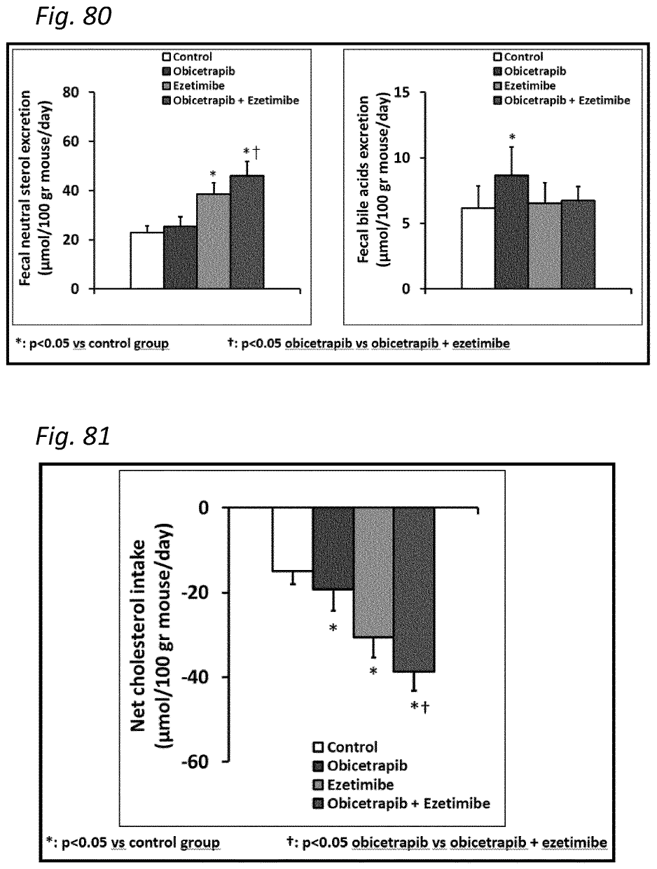

In the study, it was shown that obicetrapib completely blocks cetp activity and does not affect cetp mass (data not shown). FIG. 72 shows that obicetrapib alone as well as the combination treatment decreased total cholesterol levels (left panel) and non HDL-cholesterol levels (right panel), with the strongest effects for the combination treatment. As shown in FIG. 73, obicetrapib alone as well as the combination treatment increased HDL-cholesterol (left panel) and ApoA1 levels (right panel). As shown in FIG. 74, obicetrapib alone as well as the combination treatment decreased triglycerides levels, with the strongest effect for the combination treatment. FIG. 75 shows the effect of the treatments of lipoprotein profiles at baseline and after 6 weeks of treatment. Obicetrapib as well as the combination treatment increased clearance of VLDL like particles (data not shown) and decreased half-life of VLDL like particles (FIG. 76), with the strongest effect for the combination treatment. As shown in FIG. 77, obicetrapib as well as the combination treatment increased liver uptake of VLDL like particles. As shown in FIG. 78, obicetrapib (as well as the combination treatment) resulted in decreased plasma pcsk-9 levels and increased hepatic LDL receptor levels. FIG. 79 shows the effects of obicetrapib and the combination treatment on liver weight and liver lipids. No significant effect on hepatic pcsk9 and lrp-1 protein levels were observed (data not shown). As shown in FIG. 80, obicetrapib alone increased fecal bile acids, but on top of ezetimibe it increased fecal neutral sterols. As shown in FIG. 81, obicetrapib decreased cholesterol balance. Ezetimibe decreased biliary cholesterol levels but showed no significant effects on biliary cholesterol flow (not shown). Obicetrapib and ezetimibe did not affect biliary bile acids and biliary bile acid flow (not shown). Obicetrapib and ezetimibe did not affect biliary bile acids and biliary bile acid flow (not shown). Obicetrapib and ezetimibe did not affect biliary phospholipids and biliary phospholipid flow (not shown).

### Conclusions

In conclusion, the study showed that Obicetrapib, ezetimibe and the combination thereof reduced total plasma cholesterol levels (−42%, −23% and −62%), mainly attributed to a decrease in non-HDL-C levels (−61%, −24% and −80%). Obicetrapib alone and in combination with ezetimibe nearly completely blocked CETP activity (−99% and −100%) resulting in increased HDL-C (+260% and +245%) and ApoA1 levels (98% and 81%). Obicetrapib, ezetimibe and to a larger extent the combination thereof enhanced clearance of VLDL-like particles (half-life: −44%, −23% and −57%) and enhanced hepatic LDL receptor expression (+63% and +74%). Fecal analysis demonstrated increased bile acid excretion in obicetrapib-treated mice (+41%) and increased neutral sterol excretion in ezetimibe-treated mice, which was even more pronounced in combination with obicetrapib (+68% and +100%), resulting in a net fecal sterol loss. Obicetrapib alone and the combination with ezetimibe reduce non-HDL-C levels by increased VLDL lipolysis, increased VLDL clearance and elevated LDL receptor levels accompanied by an enhanced fecal bile acid and neutral sterol excretion.

### Example 13—Obicetrapib Alone and in Combination with Ezetimibe Reduces Atherosclerotic Lesion Prevalence, Size, and Severity in ApoE*3-Leiden.CETP Mice

Obicetrapib is a potent and selective cholesteryl ester transfer protein (CETP) inhibitor currently in clinical development for the treatment of hypercholesterolemia and reduction of cardiovascular risk. It strongly reduces plasma levels of apolipoprotein B (ApoB) and low-density lipoprotein cholesterol (LDL-C) and concomitantly increases plasma high-density lipoprotein cholesterol (HDL-C) levels. Ezetimibe is a potent and selective inhibitor of biliary and dietary cholesterol absorption from the small intestine, thereby also reducing LDL-C levels. The current study was carried out to evaluate the effect of obicetrapib monotherapy and a combination of obicetrapib+ezetimibe on atherosclerosis development in a translational mouse model for hyperlipidemia and atherosclerosis.

### Methods

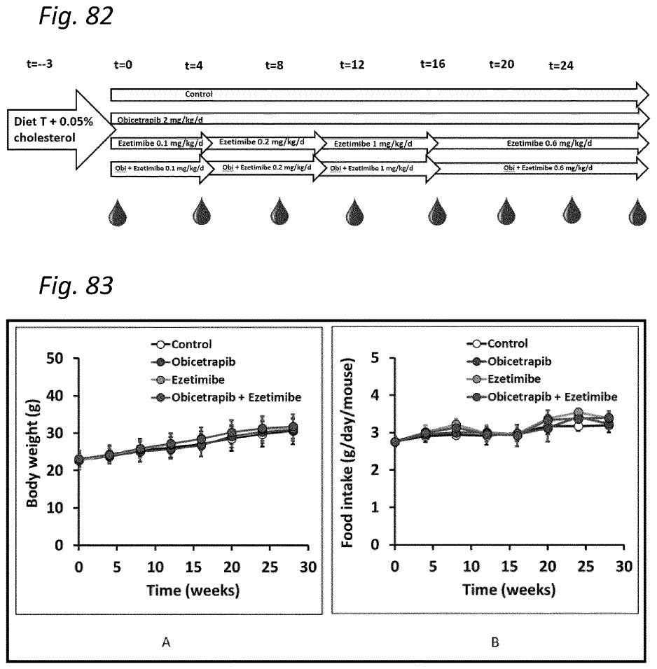

The experimental protocol is illustrated in FIG. 82, with time (t) measured in weeks. Female ApoE*3-Leiden.CETP transgenic mice were fed a Western diet with 0.05% w/w cholesterol (equivalent to daily human intake) with (i) no drug (control), or the same diet containing (ii) obicetrapib alone (2 mg/kg/day), (iii) ezetimibe alone (on average 0.6 mg/kg/day) or (iv) the combination of obicetrapib and ezetimibe. After 28 weeks of treatment, atherosclerosis development was measured in the aortic roots of the mice by anatomic sectioning, as illustrated in FIG. 88, and histologic examination and assessment according AHA classification (Stary et al., Circulation 92:1355-1374, 1995).

### Results

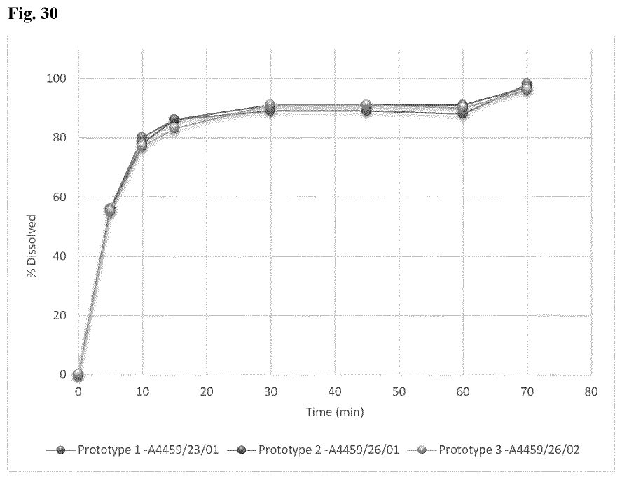

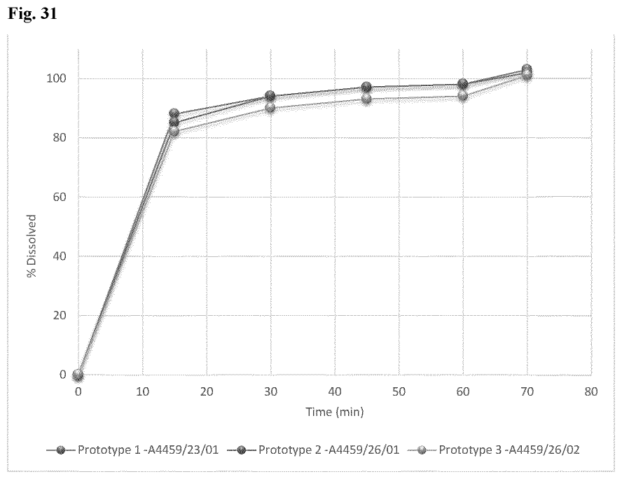

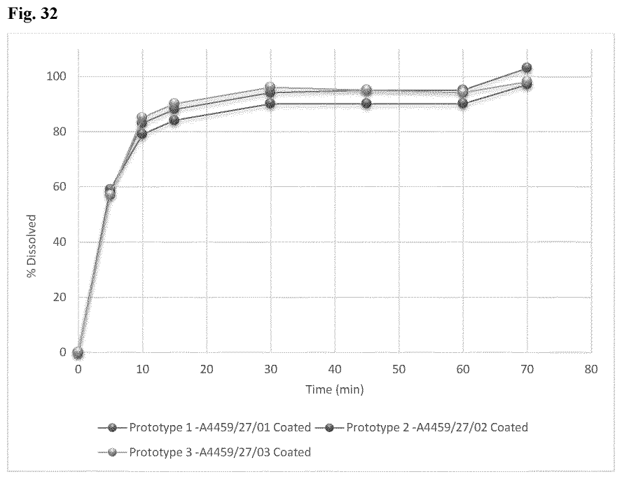

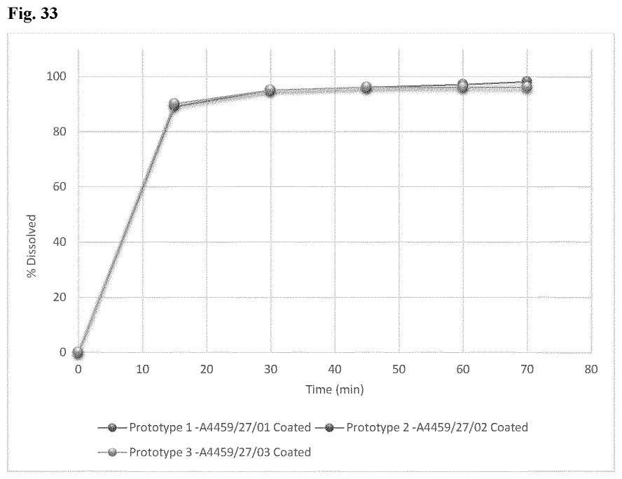

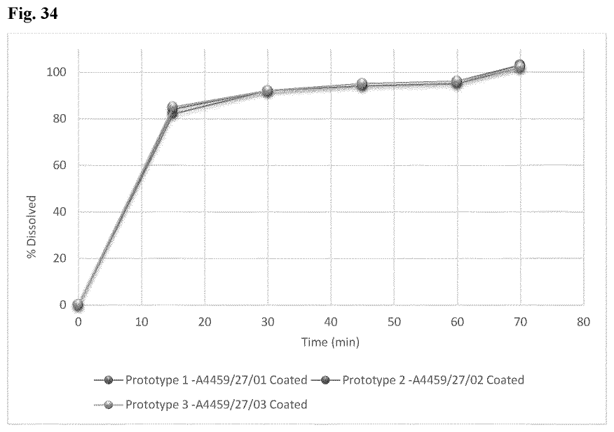

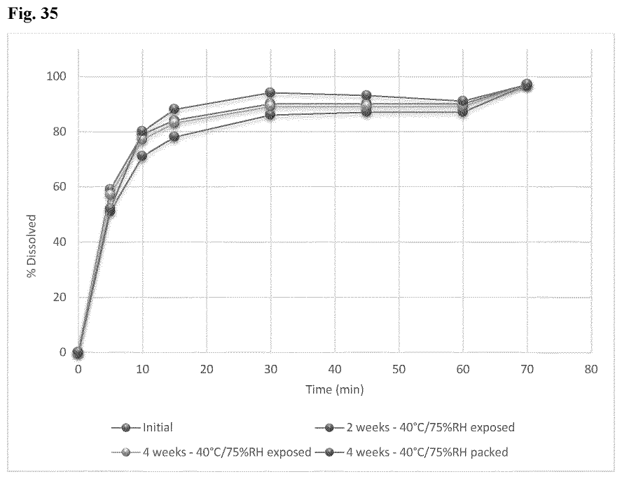

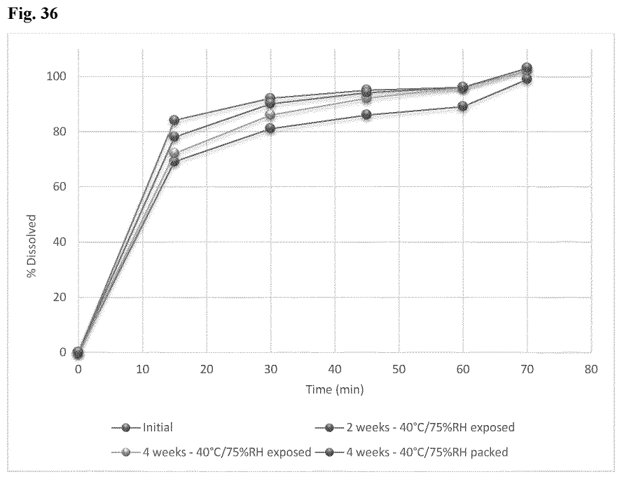

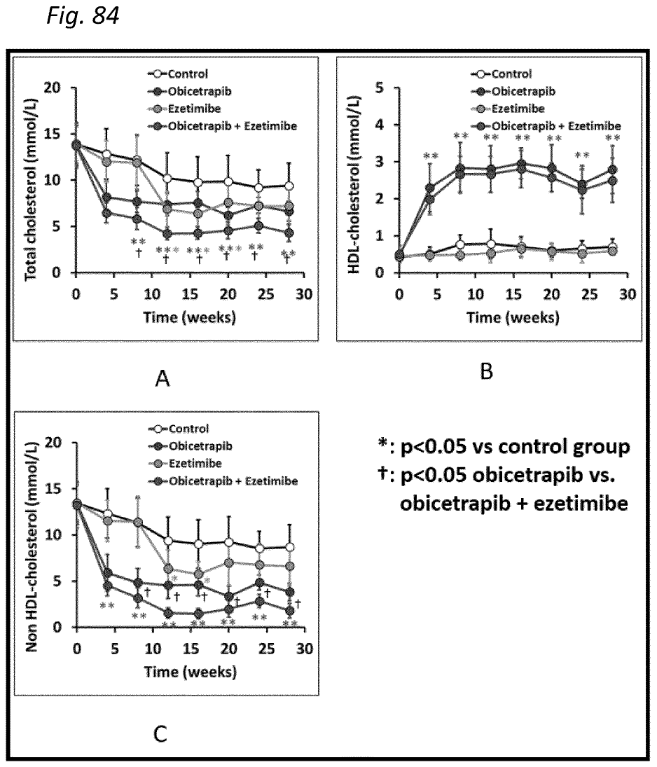

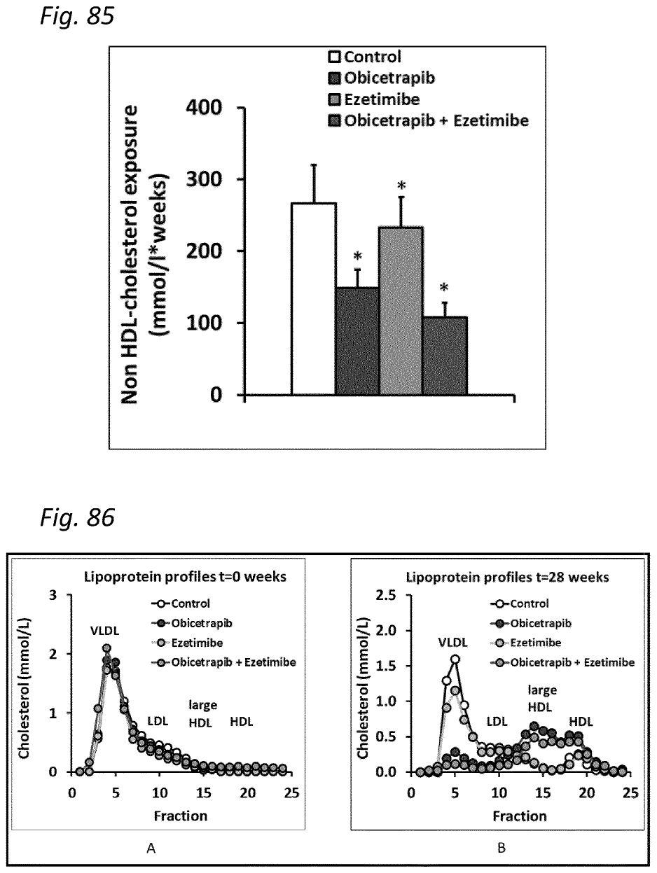

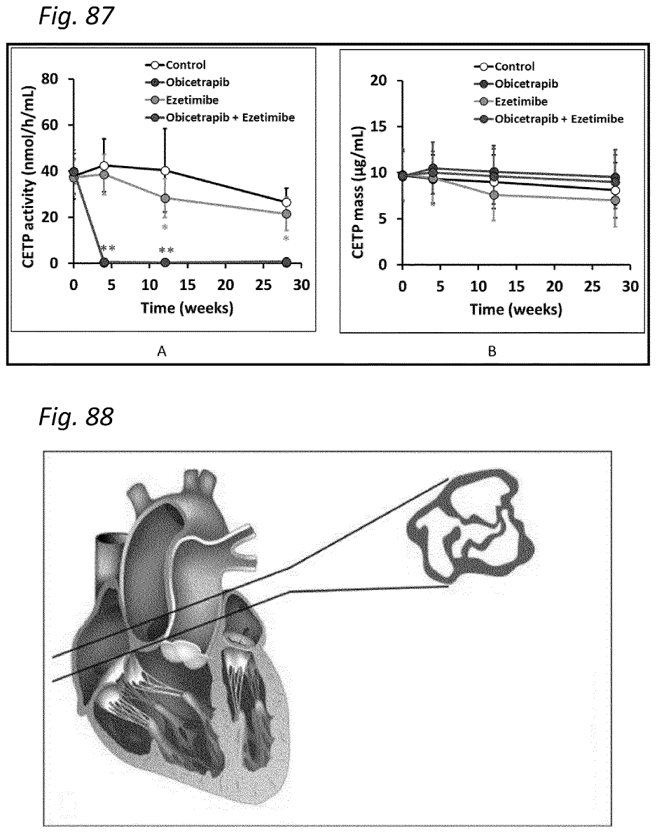

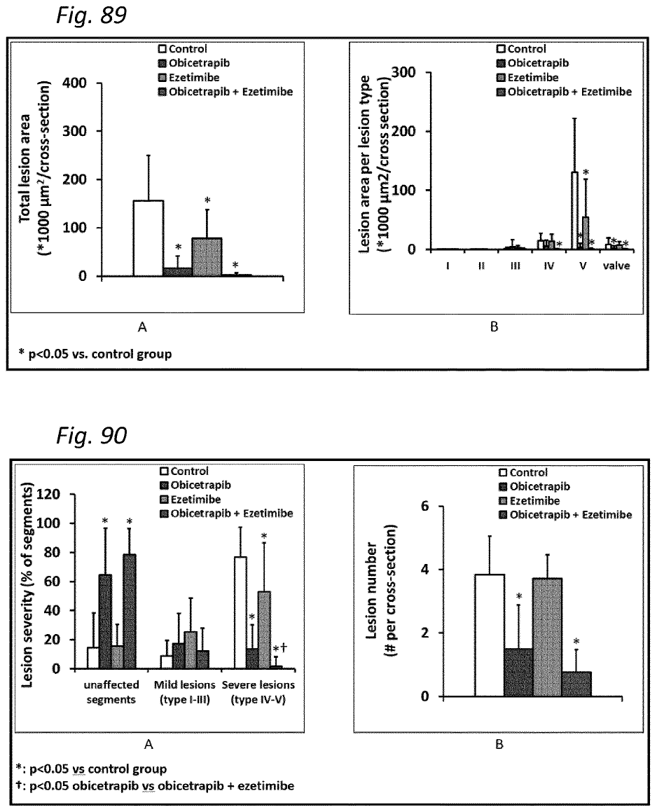

FIG. 30 confirms that there was no significant difference in total body weight (FIG. 83A) or food intake (FIG. 83B) among the 4 groups of mice during the course of the experiment. As shown in FIG. 87, obicetrapib monotherapy and combination treatment with ezetimibe nearly completely blocked CETP activity (−98% and −98%) (FIG. 87A), without statistically significant changes in CETP mass. As shown in FIG. 84A, obicetrapib, ezetimibe and the combination of obi+eze reduced total plasma cholesterol levels (−31%, −19% and −53%), mainly attributed to a decrease in non-HDL-C levels (−53%, −19% and −75%) (FIG. 84C). In the ezetimibe-only group, there was no increase in HDL-cholesterol (FIG. 84B). In contrast, both the obicetrapib-only and obicetrapib+ezetimibe groups showed increases in HDL-cholesterol (FIG. 84B), with the decrease in non-HDL cholesterol (FIG. 84C) outweighing the increase in HDL-c to lead to a significant decrease in total cholesterol (FIG. 84A). FIGS. 86A and 86B show lipoprotein profiles of the 4 groups of mice at baseline (t=0) (FIG. 86A) and at 28 weeks (FIG. 86B). FIG. 85 shows the total non HDL-cholesterol exposure across the entire experiment, by experimental group; all three experimental groups showed statistically significant reductions in non HDL-cholesterol exposure as compared to the control group. As shown in FIG. 89A, obicetrapib alone, ezetimibe alone, and the combination of obicetrapib+ezetimibe reduced total atherosclerotic lesion size by −90%, −50% and −98%, respectively. All reductions were statistically significant as compared to controls (p<0.05 versus control). As shown in FIG. 89B, which subsets lesion area by lesion severity type, severity was decreased by obicetrapib alone, ezetimibe alone, and to a larger extent by the combination of obicetrapib+ezetimibe, with a reduction of severe lesions (types IV-V) of −82%, −31% and −98%, respectively. FIG. 90A shows an increase in the percentage of unaffected segments by obicetrapib and the combination treatment with obicetrapib+ezetimibe of +347% and +442%, respectively. FIG. 90B shows no reduction in lesion number in the ezetimibe-only group, whereas both the obicetrapib-only and obicetrapib+ezetimibe groups show statistically significant reductions in lesion number as compared to the control animals.

### Conclusions

Obicetrapib alone and the combination of obicetrapib+ezetimibe robustly lowers non-HDL-C levels and impedes atherosclerosis development through a large decrease in atherosclerotic lesion size and severity.

### List of Abbreviations

API Active Pharmaceutical Ingredient
° C. degree Celsius
CC Compaction compressibility (=100×(TBD−IBD)/TBD)
CFM cubic feet per minute
CoA Certificate of analysis
DBD Dynamic bulk density (=(TBD−IBD)²/TBD]+IBD)
e.g for example
eze/EZE Ezetimibe
FDC1 Formulation development composition 1
FDC2 Formulation development composition 2
g gram
H Hausner ratio (=TBD/IBD)
HPLC High Performance Liquid Chromatography
IBD Initial bulk density (=mass/initial volume)
KF Karl Fisher
kp Kilopound
KN Kilonewton
LC Label claim
LLS Laser Light Scattering
Lt Liter
mg milligram
min minute
mL millilitre
mm millimeter
N.R Not recorded
N.D Not detected
Obi/OBI Obicetrapib (Known also as TA-8995)
pH −log [H⁺] or pH=−log a_{H}⁺
PSD Particle Size Distribution
RH Relative Humidity (a_{w}*100)
rpm rotation per minute
RRT Relative retention time
RSD Relative standard deviation
sec second
SD Standard deviation
SLS Kolliphor SLS Fine, EP & USP/NF & JP
T Temperature (° C.)
TBD Tapped bulk density (=mass/tapped volume)
VS versus
w/w weight/weight
XRPD X-Ray Powder Diffraction
μm microns
Ø diameter
% a/a % area of impurity to the total area of peaks not being present in the blank

1. A fixed dose pharmaceutical composition comprising obicetrapib or a pharmaceutically acceptable salt, solvate or co-crystal thereof; ezetimibe or a pharmaceutically acceptable salt, solvate or co-crystal thereof, and, pharmaceutically acceptable excipients, for use in a method of synergistically lowering LDL-C plasma levels in a subject in need thereof, said method comprising the administration of the fixed dose pharmaceutical composition.

2. A fixed dose pharmaceutical composition for use according to claim 1, wherein the subject is a subject suffering from heterozygous familial hypercholesterolaemia (HeFH), mixed dyslipidemia, or established atherosclerotic cardiovascular disease (ASCVD).

3. A fixed dose pharmaceutical composition for use according to claim 1, wherein the subject is a subject suffering from non-familial hypercholesterolaemia.

4. A fixed dose pharmaceutical composition for use according to any one of claims 1-3, wherein the subject is unable to reach an LDL-C plasma level of <70 mg/dL with the maximum tolerated dose of a statin in addition to ezetimibe.

5. A fixed dose pharmaceutical composition for use according to any one of claims 1-3, wherein the subject is a subject that is intolerant to statins or for whom statins are contraindicated, and is unable to reach an LDL-C plasma level of <70 mg/dL with ezetimibe alone.

6. A fixed dose pharmaceutical composition for use according to any one of claims 1-5, wherein the subject is a subject requiring additional lowering of LDL-C as an adjunct to diet and/or to maximally tolerated lipid-lowering therapy other than obicetrapib/ezetimibe combination therapy.

7. A fixed dose pharmaceutical composition for use according to any one of the preceding claims, wherein the method comprises the oral administration of obicetrapib at a daily dose of about 10 mg and the oral administration of ezetimibe at a daily dose of about 10 mg, during a period of at least three months.

8. A fixed dose pharmaceutical composition for use according to 7, wherein said method results in a reduction of LDL-C plasma levels of at least 40%, from baseline, wherein baseline is defined as start of the treatment with obicetrapib and ezetimibe, more preferably at least 45%, most preferably at least 50%.

9. A fixed dose pharmaceutical composition for use according to claim 7, wherein said method results in a reduction of LDL-C plasma levels, with at least 40 mg/dL, from baseline, wherein baseline is defined as start of the treatment with obicetrapib and ezetimibe, more preferably at least 45 mg/dL, more preferably at least 50 mg/dL.

10. A fixed dose pharmaceutical composition for use according to claim 7, wherein said method results in a reduction of LDL-C plasma levels to a level below 60 mg/dL, preferably below 55 mg/dL, more preferably below 50 mg/dL.

11. A fixed dose pharmaceutical composition for use according to any one of claims 1-4, wherein the method further comprises the concurrent treatment with a HMG CoA reductase inhibitor.

12. A fixed dose pharmaceutical composition for use according to claim 11, wherein the method comprises the administration of rosuvastatin at a daily dosage of 20 mg or 40 mg or the administration of atorvastatin at a daily dosage of 40 or 80 mg.

13. A fixed dose pharmaceutical composition for use according to any one of the preceding claims 1-12, wherein obicetrapib is present in the form of an amorphous calcium salt of obicetrapib.

14. A fixed dose pharmaceutical composition for use according to claim 13, wherein the amorphous calcium salt of obicetrapib is amorphous obicetrapib hemicalcium.

15. A fixed dose pharmaceutical composition comprising obicetrapib or a pharmaceutically acceptable salt, solvate or co-crystal thereof; ezetimibe or a pharmaceutically acceptable salt, solvate or co-crystal thereof, and, pharmaceutically acceptable excipients, for use in a method of synergistically slowing the development and/or progression of CVD, more in particular ASCVD, and/or synergistically reducing the risk and/or occurrence of CVD related events, in particular ASCVD related events, in a subject in need thereof, said method comprising the administration of the fixed dose pharmaceutical composition in a subject in need thereof, said method comprising the administration of the fixed dose pharmaceutical composition.

16. A fixed dose pharmaceutical composition for use according to claim 15, wherein the subject is a subject suffering from heterozygous familial hypercholesterolaemia (HeFH), mixed dyslipidemia, or established atherosclerotic cardiovascular disease (ASCVD).

17. A fixed dose pharmaceutical composition for use according to claim 15, wherein the subject is a subject suffering from non-familial hypercholesterolaemia.

18. A fixed dose pharmaceutical composition for use according to any one of claims 15-17, wherein the subject is unable to reach an LDL-C plasma level of <70 mg/dL with the maximum tolerated dose of a statin in addition to ezetimibe.

19. A fixed dose pharmaceutical composition for use according to any one of claims 15-17, wherein the subject is a subject that is intolerant to statins or for whom statins are contraindicated, and is unable to reach an LDL-C plasma level of <70 mg/dL with ezetimibe alone.

20. A fixed dose pharmaceutical composition for use according to any one of claims 15-17, wherein the subject is a subject requiring additional lowering of LDL-C as an adjunct to diet and/or to maximally tolerated lipid-lowering therapy other than obicetrapib/ezetimibe combination therapy.

21. A fixed dose pharmaceutical composition for use according to any one of claims 15-20, wherein the method comprises the oral administration of obicetrapib at a daily dose of about 10 mg and the oral administration of ezetimibe at a daily dose of about 10 mg, during a period of at least three months.

22. A fixed dose pharmaceutical composition for use according to 21, wherein said method results in a reduction of LDL-C plasma levels of at least 40%, from baseline, wherein baseline is defined as start of the treatment with obicetrapib and ezetimibe, more preferably at least 45%, most preferably at least 50%.

23. A fixed dose pharmaceutical composition for use according to claim 21, wherein said method results in a reduction of LDL-C plasma levels, with at least 40 mg/dL, from baseline, wherein baseline is defined as start of the treatment with obicetrapib and ezetimibe, more preferably at least 45 mg/dL, more preferably at least 50 mg/dL.

24. A fixed dose pharmaceutical composition for use according to claim 21, wherein said method results in a reduction of LDL-C plasma levels to a level below 60 mg/dL, preferably below 55 mg/dL, more preferably below 50 mg/dL.

25. A fixed dose pharmaceutical composition for use according to any one of claims 15-19, wherein the method further comprises the concurrent treatment with a HMG CoA reductase inhibitor.

26. A fixed dose pharmaceutical composition for use according to claim 25, wherein the method comprises the administration of rosuvastatin at a daily dosage of 20 mg or 40 mg or the administration of atorvastatin at a daily dosage of 40 or 80 mg.

27. A fixed dose pharmaceutical composition for use according to any one of the preceding claims 15-26, wherein obicetrapib is present in the form of an amorphous calcium salt of obicetrapib.

28. A fixed dose pharmaceutical composition for use according to claim 27, wherein the amorphous calcium salt of obicetrapib is amorphous obicetrapib hemicalcium.
